(19) United States
(12) Patent Application Publication
(10) Pub. No.: US 2026/0240937 A1
(43) Pub. Date: Aug. 20, 2026

(54) METHODS, COMPOUNDS, COMPOSITIONS, AND RELATED USES FOR MEDICAL MANAGEMENT OF SUBJECTS WITH MUSCULAR DYSTROPHY

(71) Applicant: Stealth BioTherapeutics Inc., Needham, MA (US)

(72) Inventor: David BROWN, Needham, MA (US)

(73) Assignee: Stealth BioTherapeutics Inc., Needham, MA (US)

(21) Appl. No.: 19/443,236

(22) Filed: Jan. 8, 2026

Related U.S. Application Data
(60) Provisional application No. 63/743,609, filed on Jan. 9, 2025.
(60) Provisional application No. 63/925,031, filed on Nov. 25, 2025.

(51) Int. Cl. A61K 38/07; A61K 31/138; A61K 31/165; A61K 31/18; A61K 31/196; A61K 31/216; A61K 31/353; A61K 31/401; A61K 31/403; A61K 31/404; A61K 31/4178; A61K 31/4184; A61K 31/4245; A61K 31/44; A61K 31/513; A61K 31/573; A61K 31/585; A61K 31/635; A61K 31/7088; A61K 38/17; A61K 48/00; A61P 21/00

(52) U.S. Cl. CPC A61K 38/07; A61K 31/138; A61K 31/165; A61K 31/18; A61K 31/196; A61K 31/216; A61K 31/353; A61K 31/401; A61K 31/403; A61K 31/404; A61K 31/4178; A61K 31/4184; A61K 31/4245; A61K 31/44; A61K 31/513; A61K 31/573; A61K 31/585; A61K 31/635; A61K 31/7088; A61K 38/1709; A61K 48/0033; A61K 48/005; A61P 21/00

(57) ABSTRACT

The disclosure provides peptides, mixtures of peptides, compositions (e.g., formulations or medicaments), methods and related uses for the medical management of mammalian subjects with muscular dystrophy (MD), such as Duchenne muscular dystrophy (DMD) or Becker muscular dystrophy (BMD), and including loss of ambulation, respiratory distress and the various types of cardiomyopathies associated therewith. The methods and uses comprise administering to a subject an effective amount of a peptide, or a mixture of peptides, of Formula A, or a pharmaceutically acceptable salt, hydrate, solvate, and/or tautomer thereof, including compositions or medicaments comprising said peptide or mixture of peptides of Formula A, in any of its/their various forms, optionally in combination with at least one additional therapeutic agent such as, for example, a drug administered to augment dystrophin production in the subject to which it is administered.

### CROSS-REFERENCE TO RELATED APPLICATIONS

This application claims the benefit of and priority to U.S. Provisional Patent Application No. 63/743,609, filed Jan. 9, 2025, and U.S. Provisional Patent Application No. 63/925,031, filed Nov. 25, 2025, the contents of each of which are incorporated herein by reference in their entirety for any and all purposes.

### TECHNICAL FIELD

The present disclosure relates generally to compounds (e.g., peptides), compositions (e.g., medicaments or formulations), methods, and related uses for treating, preventing, inhibiting, ameliorating, or delaying the onset of muscular dystrophy such as, for example, Duchenne muscular dystrophy (DMD) or Becker's muscular dystrophy (BMD) in a subject in need thereof. The present technology relates to administering an effective amount of a peptide, mixture of peptides, or a composition, formulation, or medicament comprising the peptide or mixture of peptides as described herein; or a pharmaceutically acceptable salt, hydrate, solvate, stereoisomer and/or tautomer of said peptide or peptides (including peptides in their C-terminal carboxylate form), optionally in combination with an effective amount of an additional therapeutic agent such as a phosphorodiamidate morpholino oligomer (PMO), a peptide-conjugated PMO (PPMO), an adeno-associated virus (AAV) vector-based gene therapy, a corticosteroid, an angiotensin-converting enzyme (ACE) inhibitor, an angiotensin receptor blocker (ARB), a beta blocker, a diuretic, a mineralocorticoid receptor antagonist, or any two or more of the foregoing additional therapeutic agents to a subject suffering from muscular dystrophy.

### INTRODUCTION

The following description is provided to assist the understanding of the reader. None of the information provided or references cited is admitted as prior art to the compositions and methods disclosed herein.

Muscular dystrophy (MD) is a group of inherited non-inflammatory but progressive muscle disorders. Duchenne muscular dystrophy (DMD) is the most common muscular dystrophy affecting 1 in about 3500 males born worldwide. Becker's muscular dystrophy (BMD) is milder than DMD and primarily causes heart disease. BMD affects only males (1 in about 30,000), and usually first appears between the ages of 2 and 16 years but can appear as late as age 25. Both DMD and BMD result from abnormal or deficient production of the protein, dystrophin.

DMD begins with progressive muscle weakness that evolves to loss of ambulation and further progresses to early morbidity and mortality. DMD is caused by mutations in the dystrophin gene at locus Xp21, located on the short arm of the X chromosome. Dystrophin encodes a 427-kD protein that plays an integral role in the structural stability of the myofiber. The loss of dystrophin disrupts the muscle membrane and fibers. Without dystrophin, muscle fibers are susceptible to mechanical injury and necrotic/apoptotic cell death.

DMD is a progressive disease which eventually affects all voluntary muscles as well as cardiac and breathing muscles (e.g., the diaphragm) in later stages. The disease is most prevalent in males. While female carriers of the DMD mutation are largely asymptomatic, some (20-30%) present with mild to moderate muscle weakness and are at increased risk for developing dilated cardiomyopathy (DCM). Boys generally present with symptoms between the ages of three to five years. These symptoms generally worsen over time leading to loss of ambulation and the need for a wheelchair by early adolescence. Further progression of DMD leads to respiratory distress and cardiomyopathies, which are present in almost all males by the age of 18. The average life expectancy for individuals afflicted with DMD is around age 25.

Signs and symptoms of DMD include progressive proximal weakness with onset in the legs and pelvis, hyperlordosis with wide-based gait, hypertrophy of weak muscles, pseudohypertrophy (enlargement of calf and deltoid muscles with fat and fibrotic tissue), reduced muscle contractility on electrical stimulation in advanced stages of the disease, delayed motor milestones, progressive inability to ambulate, heel cord contractures, paralysis, fatigue, skeletal deformities including scoliosis, muscle fiber deformities, cardiomyopathy, congestive heart failure or arrhythmia, muscular atrophy, respiratory disorders, bladder or bowel dysfunction, sensory disturbance, febrile illness, or any two or more of the foregoing. Weakness of skeletal muscle can contribute to cardiopulmonary complications. Scoliotic deformity from paraspinal muscle asymmetric atrophy can impair pulmonary and gastrointestinal function, predisposing individuals to pneumonia, respiratory failure, and poor nutrition. Smooth muscle dysfunction as a result of abnormal or absent dystrophin, along with inactivity, leads to gastrointestinal dysmotility, causing constipation and diarrhea.

DMD can be diagnosed in several ways. A clinical diagnosis may be made when a male child has progressive symmetrical muscle weakness. Muscle biopsy is an important tool for quantifying the amount of muscle dystrophin as well as for detecting asymptomatic female carriers of DMD. Immunostaining of the muscle using antibodies directed against the rod domain, carboxy-terminals, and amino-terminals of dystrophin protein shows absence of the usual sarcolemma staining in boys with DMD. A combination of clinical findings, family history, blood concentration of creatine phosphokinase and muscle biopsy with dystrophin studies confirms the diagnosis (Creatine phosphokinase is normally present in high concentrations in the muscle cells of human subjects with DMD). For example, DMD patients exhibit creatine phosphokinase levels that are 50-100 times the reference range (as high as 20,000 mU/mL) during the early stages of the disease. Electromyography, electrocardiogram and echocardiogram, and lung monitoring tests may be used for confirmatory diagnosis of DMD. The progression of DMD occurs in 5 stages: presymptomatic, early ambulatory, late ambulatory, early nonambulatory, and late nonambulatory.

As with other aspects of DMD, the cardiomyopathies are progressive but generally end with heart (cardiac) failure. Ultrasonography can detect structural changes in the myocardium well before the onset of systolic dysfunction and overt cardiomyopathy. Despite the high incidence of heart failure, the majority of children with DMD are relatively asymptomatic until late in the disease course, probably because of their inability to exercise. Heart failure and arrhythmias may develop in the late stages of the disease, especially during intercurrent infections or surgery. The late-stage cardiomyopathy is characterized by extensive fibrosis of the posterobasal left ventricular wall followed by spread of the fibrosis to the lateral free wall of the left ventricle. The continued progression of the cardiomyopathy often leads to output failure and pulmonary congestion. Alternatively, cardiac fibrosis can include cardiomyopathy and conduction abnormalities, which can induce fatal arrhythmias. Heart (cardiac) failure is the most common cause of death of persons afflicted with DMD.

Myocardial energy homeostasis is disrupted in DMD, with dysfunctional mitochondria being a central factor. Impaired mitochondrial function in the dystrophic heart is observed early in both animal DMD models and human studies, often before observable declines in cardiac function. The lack of dystrophin leads to cellular membrane fragility and heightened susceptibility to membrane rupture.

The loss of cell membrane integrity induces “leaky” fluxes of ions, enzymes, and metabolites. The influx of calcium is particularly problematic in the heart, which requires tightly regulated calcium cycling for pump function. Calcium content is normally three to four orders of magnitude lower in the cytosol compared to the outside of the cell. Heightened calcium within DMD myocytes causes sarcomeric disruption and calcium overload in mitochondria.

Mitochondrial calcium overload leads to several inter-related problems in the DMD heart. Calcium overload opens the mitochondrial permeability transition pore, a non-specific mitochondrial channel that can initiate apoptotic cell death. Opening of this pore can be catastrophic for mitochondria, as it collapses electrochemical and metabolite gradients that are crucial for ATP generation. DMD mitochondria have heightened production of reactive oxygen species (ROS), which can exacerbate cellular damage. Ruptured mitochondrial fragments can leak out of cells and contribute to inflammatory signaling cascades. Finally, mitochondrial structure, which is directly related to bioenergetic function, is compromised in DMD.

Mitochondrial dysfunction in DMD is a key contributor to cellular death. As the regenerative capacity of the heart is very low, the loss of myocytes places an increased burden on the surviving cells. Mitochondria within viable cells are under heightened pressure to meet the constant ATP demands of the heart. Futile pathological cycles continue to overwhelm cellular defense mechanisms as the disease progresses. Ensuing cardiac remodeling leads to higher propensity for electromechanical dysfunction and ultimately compromised cardiac function.

Historically, DMD and BMD patients have been treated for their symptoms. The standard of care has been treatment with corticosteroids to increase muscle function, angiotensin-converting enzyme (ACE) inhibitors, angiotensin receptor blockers (ARBs), and beta blockers to address the progressive cardiomyopathy, and assistive devices to address ambulatory needs. Diuretics and mineralocorticoid receptor antagonists are also often used in the medical management of subjects with DMD.

Phosphorodiamidate morpholino oligomers (PMOs), which are drugs based on an exon skipping mechanism of action that are directed to upregulation in the expression of the protein, dystrophin, in DMD patients (i.e., Exondys 51® (eteplirsen), Vyondys 53™ (golodirsen), Viltepso® (viltolarsen), or Amondys 45™ (casimersen)), have been approved by the United States Food and Drug Administration (FDA)). While these drugs appear to increase the expression of dystrophin in skeletal muscle, a key limitation to the efficacy of PMOs is variability in their potency, tissue uptake and retention, and tissue specificity (notably low in cardiac tissue).

In order to improve cellular uptake, classic PMOs have been modified with cell penetrating peptides to thereby create conjugates referred to as peptide phosphorodiamidate morpholino oligomers or PPMOs. It is believed that these PPMOs will gain easier access to the cells/tissues of a subject and thereby be more effective in their treatment effects.

Another strategy for better delivery of PMOs and related gene therapy modalities involves conjugating the active agent to an adeno-associated virus (AAV) vector. Said AAV vector in theory will permit the active PMO to better penetrate cells/tissues of the subject and thereby be more effective in their treatment effects.

Collectively, PPMOs and AAV vector modified PMOs represent a new generation of PMO technology. Regardless, there is yet to be evidence to suggest that these new technologies address some fundamental limitations of the PMO technology, including providing improvements in treating cardiomyopathies associated with muscular dystrophy.

In summary, although there are current treatments for symptoms and recent advances in medicine have begun to address the molecular underpinnings of the disease, MD (including, without limitation, DMD and BMD) remains an incurable illness for which additional treatments and therapies are desperately needed. The following discussion addresses some of those needs.

### SUMMARY

The present disclosure relates to methods for treating muscular dystrophy (e.g., DMD and BMD) by administration of peptides, mixtures of peptides and compositions, formulations or medicaments comprising said peptides or mixtures of peptides. The peptides disclosed herein target, inter alia, mitochondrial dysfunction and are particularly useful in treating the cardiomyopathies associated with muscular dystrophy. Notwithstanding the foregoing, because muscular dystrophy manifests itself in all muscle types, because mitochondria are present in all muscle types, and because the peptides disclosed herein can penetrate muscle to provide efficacious dosing, these peptides may be suitable for addressing various other signs, symptoms and conditions associated with muscular dystrophy, including cardiomyopathies, loss of ambulation, and loss of respiratory function (e.g., respiratory distress).

Therefore, in some embodiments, this disclosure pertains to a method for treating, preventing, inhibiting, ameliorating, or delaying the onset of muscular dystrophy in a mammalian subject in need thereof, comprising administering to the subject a therapeutically effective amount of a peptide of Formula A, or a mixture of peptides of Formula A:

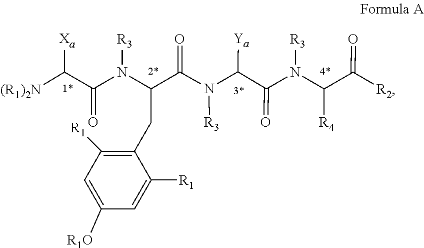

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

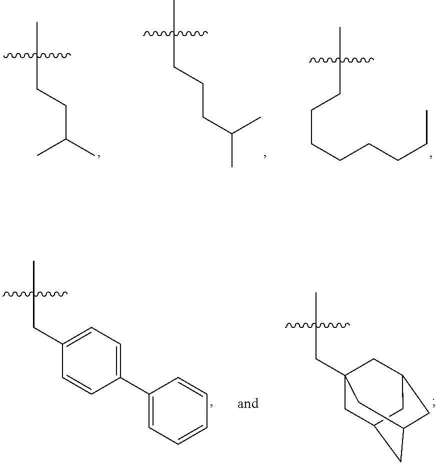

Xₐ is selected from

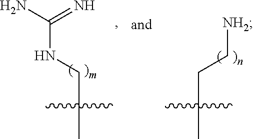

Yₐ is selected from

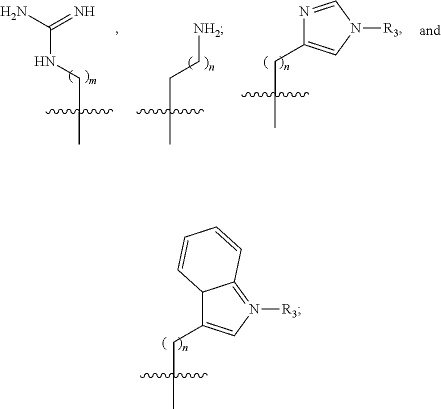

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is substituted with a deuterium or fluorine atom. In some embodiments, the peptide or peptides of Formula A is/are a peptide/peptides of Formula A-1 or Formula A-2:

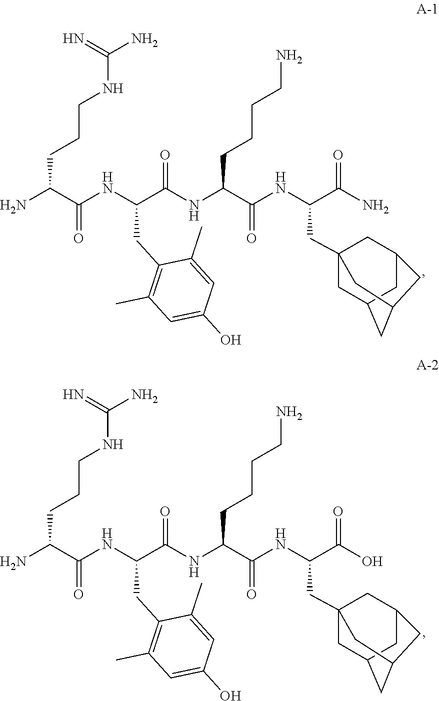 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

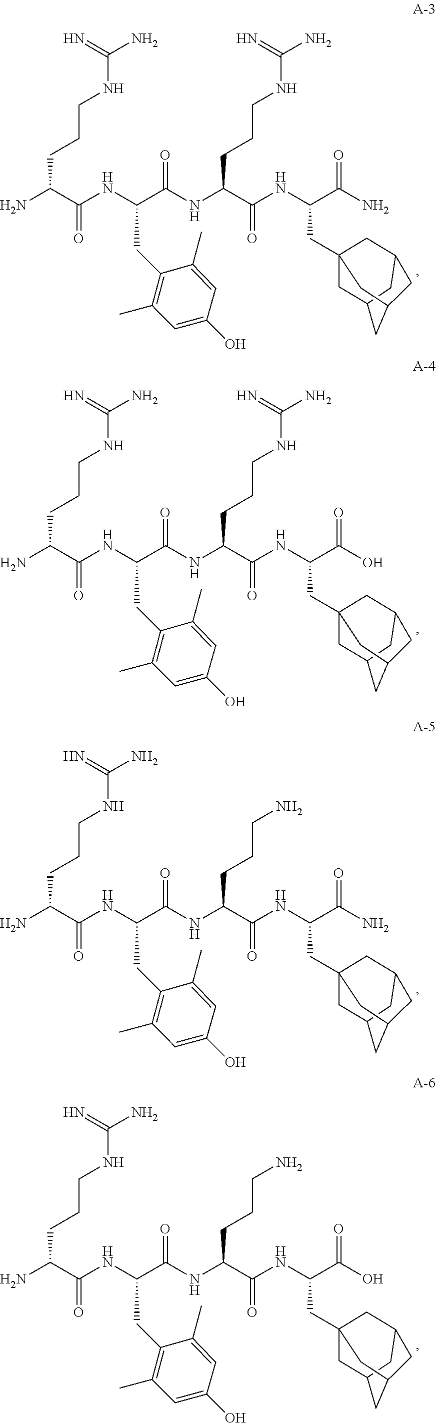 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

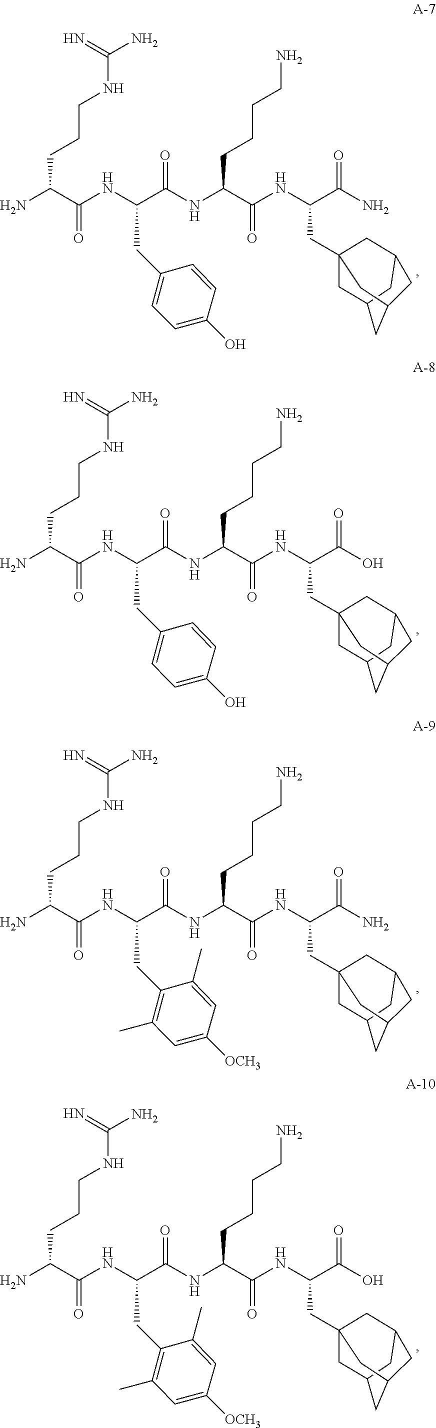 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

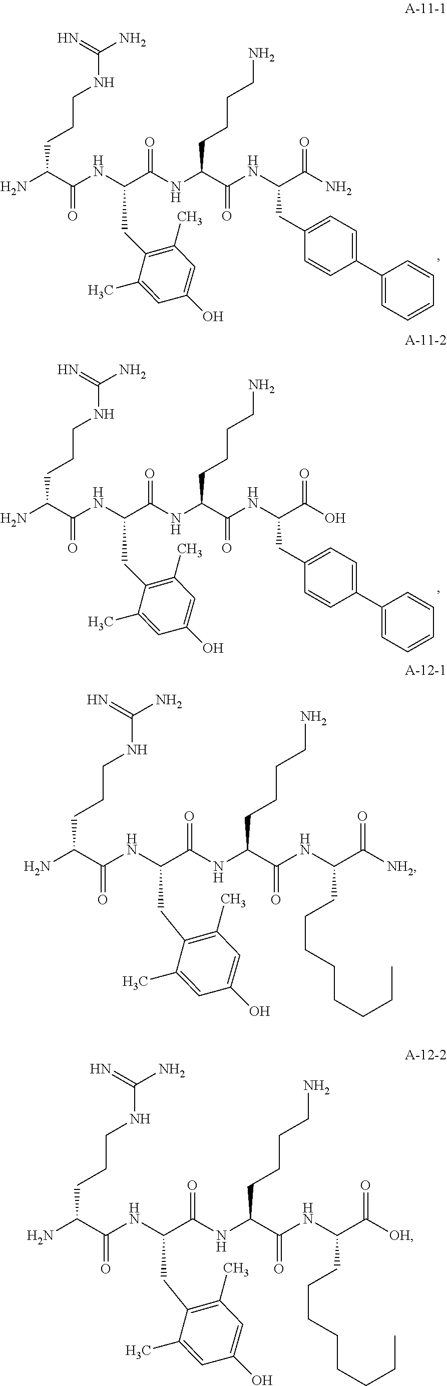 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

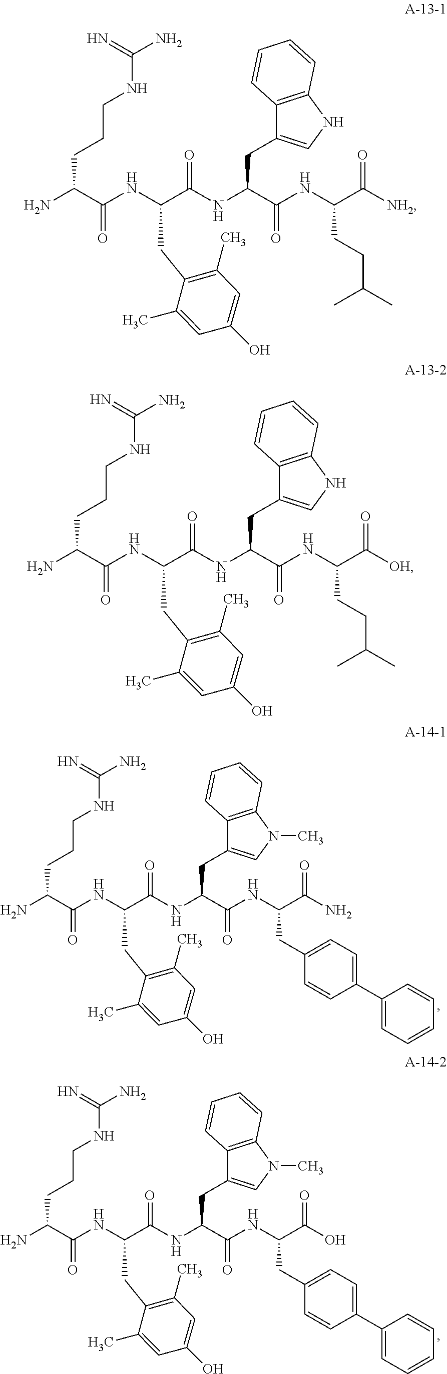 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

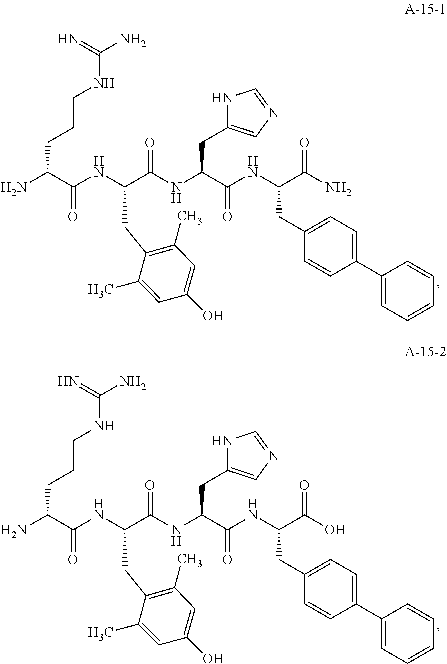 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the method, administration of the peptide or peptides ameliorates, inhibits, delays the onset of, and/or delays the progression of one or more signs or symptoms of muscular dystrophy in the subject as compared with an untreated control subject or untreated control group that is not administered the peptide or peptides.

In some embodiments of the method, administration of the peptide or peptides ameliorates, inhibits, delays the onset of, and/or delays the progression of a cardiomyopathy associated with muscular dystrophy in the subject as compared with an untreated control subject or untreated control group that is not administered the peptide or peptides. In some embodiments of the method, the cardiomyopathy is systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, heart (cardiac) failure, or a combination of any two or more of the foregoing in the subject.

In some embodiments of the method, administration of the peptide or peptides ameliorates, inhibits, delays the onset of, and/or delays the progression of muscular fibrosis in the subject as compared with an untreated control subject or untreated control group that is not administered the peptide or peptides. In some embodiments of the method, administration of the peptide or peptides ameliorates, delays the onset of, and/or delays the progression of muscular fibrosis of the diaphragm of the subject as compared with an untreated control subject or untreated control group that is not administered the peptide or peptides. In some embodiments of the method, administration of the peptide or peptides ameliorates, delays the onset of, and/or delays the progression of muscular fibrosis of skeletal muscle of the subject as compared with an untreated control subject or untreated control group that is not administered the peptide or peptides.

In some embodiments of the method, administration of the peptide or peptides increases muscle force of a muscle or muscle group the subject can exert as compared with an untreated control subject or untreated control group that is not administered the peptide or peptides. For example, the muscle/muscle group can be the extensor digitorum longus (EDL).

In some embodiments of the method, administration of the peptide or peptides ameliorates, inhibits, delays the onset of, and/or delays progression of contraction-induced damage in muscle tissue, locomotor-skeletal muscle weakness, respiratory muscle weakness, cardiac muscle weakness, sarcolemmal weakening, or sarcolemmal tearing in the subject as compared with an untreated control subject or untreated control group that is not administered the peptide or peptides.

In some embodiments of the method, the peptide or peptides is/are administered daily for: (i) 12 weeks or more; (ii) 24 weeks or more; (iii) 48 weeks or more; (iv) 72 weeks or more; or (v) 96 weeks or more. In some embodiments of the method, the peptide or peptides is/are administered subcutaneously or intravenously. In some embodiments of the method, the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the method, the subject is human.

In some embodiments of the method, the muscular dystrophy is Duchenne muscular dystrophy. In some embodiments of the method, the muscular dystrophy is Becker's muscular dystrophy.

In some embodiments of the method, practice of the method further comprises separately, sequentially, or simultaneously administering an additional therapeutic agent to the subject.

In some embodiments of the method, the additional therapeutic agent is a phosphorodiamidate morpholino oligomer (PMO) or a peptide-conjugated phosphorodiamidate morpholino oligomer (PPMO). In some embodiments of the method, the PMO is selected from the group consisting of eteplirsen (Exondys 51®), golodirsen (Vyondys 53®), viltolarsen (Viltepso®), and casimersen (Amondys 45™) or the PPMO is selected from vesleteplirsen (a.k.a., SRP-5051) and PGN-EDO51 (a.k.a., CONNECT1-ED051).

In some embodiments of the method, the additional therapeutic agent is an adeno-associated virus (AAV) vector-based gene therapy. In some embodiments of the method, the adeno-associated virus (AAV) vector-based gene therapy is delandistrogene moxeparvovec-rokl (Elevidys®).

In some embodiments of the method, the additional therapeutic agent is a corticosteroid, an angiotensin-converting enzyme (ACE) inhibitor, an angiotensin receptor blocker (ARB) a beta blocker, a diuretic, or a mineralocorticoid receptor antagonist. In some embodiments of the method, the corticosteroid is vamorolone (aGamree®), prednisone, or deflazacort. In some embodiments of the method, the angiotensin-converting enzyme (ACE) inhibitor is captopril, enalapril, lisinopril, benazepril, or ramipril. In some embodiments of the method, the angiotensin receptor blocker (ARB) is azilsartan, candesartan, eprosartan, irbesartan, telmisartan, valsartan, losartan, olmesartan, Entresto® (sacubitril/valsartan) or Byvalson™ (nebivolol/valsartan). In some embodiments of the method, the beta blocker is carvedilol, bisoprolol, metoprolol succinate, atenolol, esmolol, nebivolol, or propranolol. In some embodiments of the method, the diuretic is bumetanide, furosemide, or torsemide. In some embodiments of the method, the mineralocorticoid receptor antagonist is spironolactone or eplerenone.

In some embodiments of the method, the additional therapeutic agent is mavacamten (Camzyos®).

In some embodiments of the method, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the method, the peptide or peptides is/are formulated for administration from its/their tris —HCl salt.

In another aspect, the present disclosure pertains to a method for treating, preventing, inhibiting, ameliorating or delaying the onset of a cardiomyopathy in a mammalian subject in need thereof, wherein the subject is suffering from muscular dystrophy, comprising administering to the subject a therapeutically effective amount of a peptide of Formula A, or a mixture of peptides of Formula A:

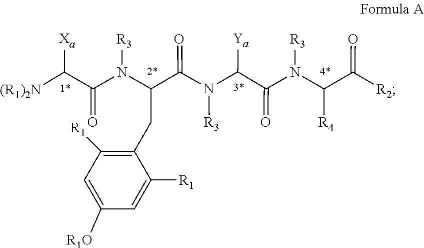

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

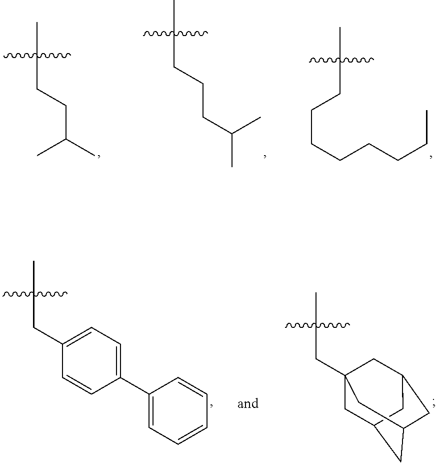

Xₐ is selected from

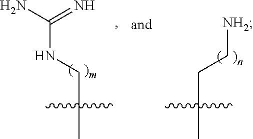

Yₐ is selected from

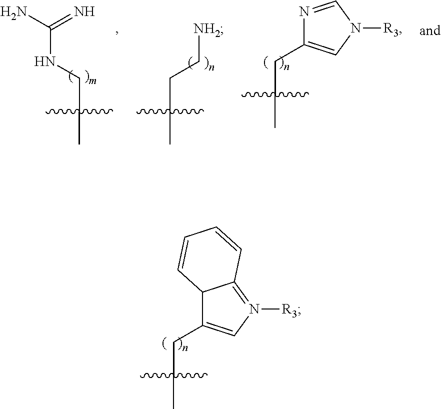

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is substituted with a deuterium or fluorine atom. In some embodiments of the method, the peptide or peptides of Formula A is/are a peptide/peptides of Formula A-1 or Formula A-2:

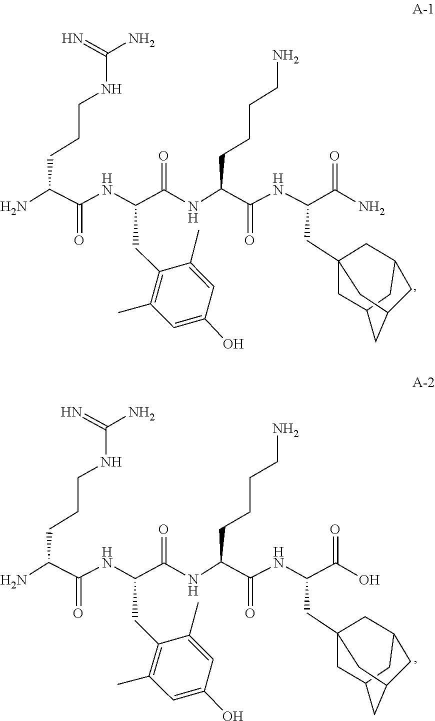 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the method, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

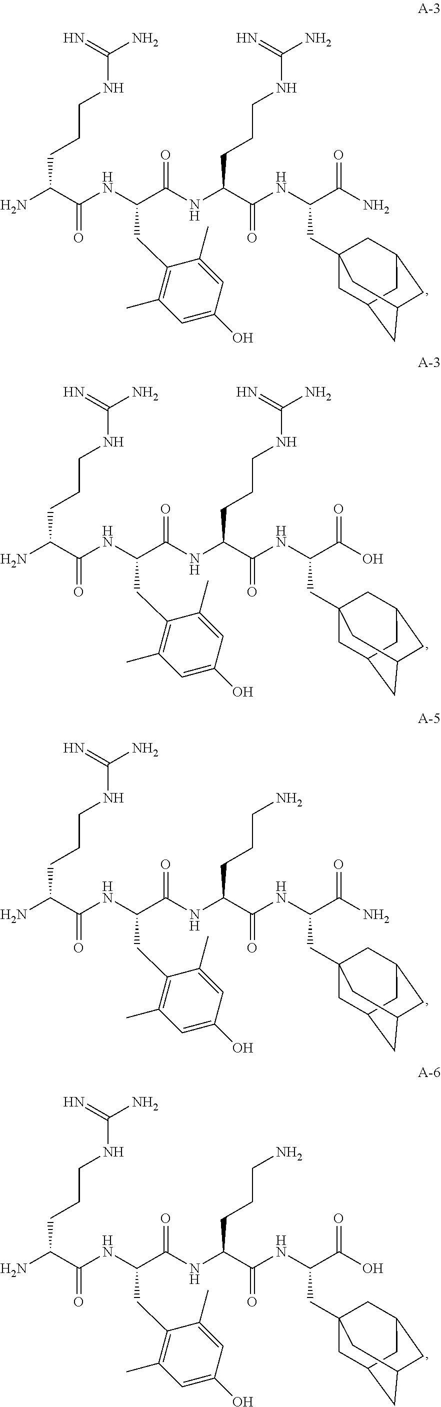 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

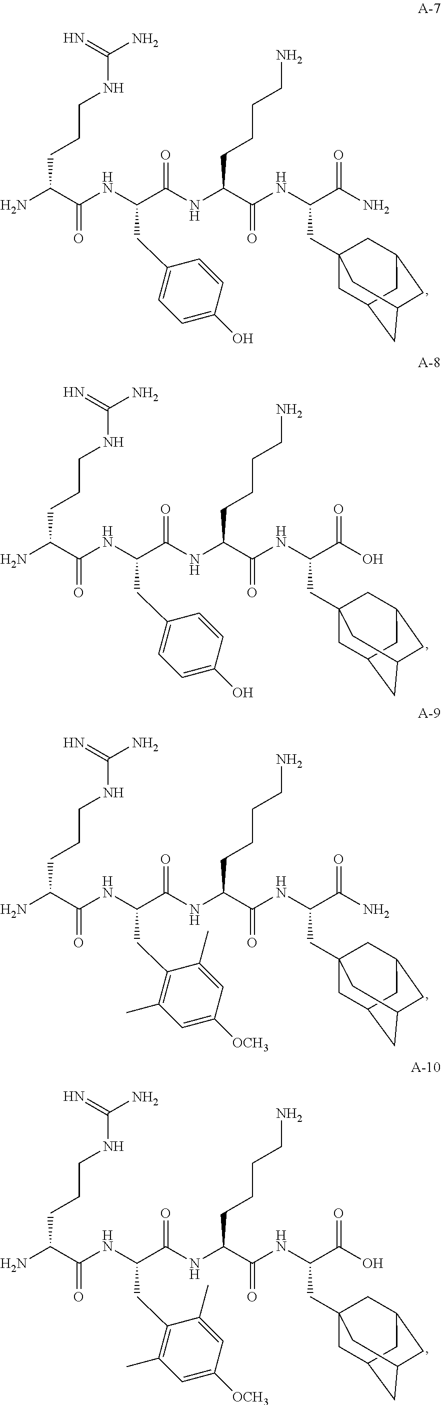 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

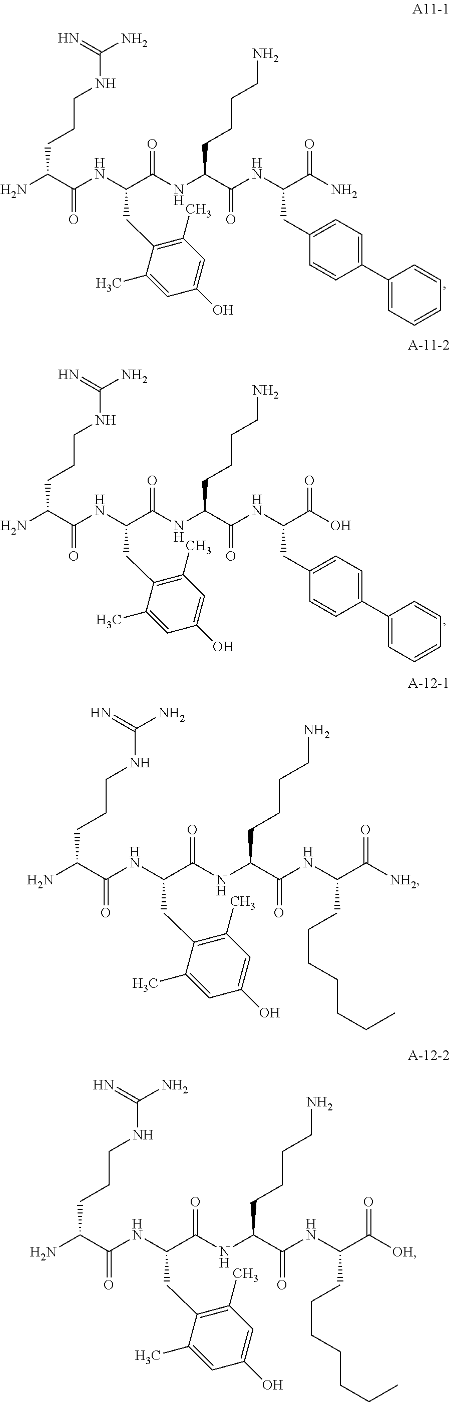 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCC[C@H](NC(=O)[C@H](CCCCN)NC(=O)[C@H](Cc1c(C)cc(O)cc1C)NC(=O)[C@H](N)CCCNC(=N)N)C(=O)O]

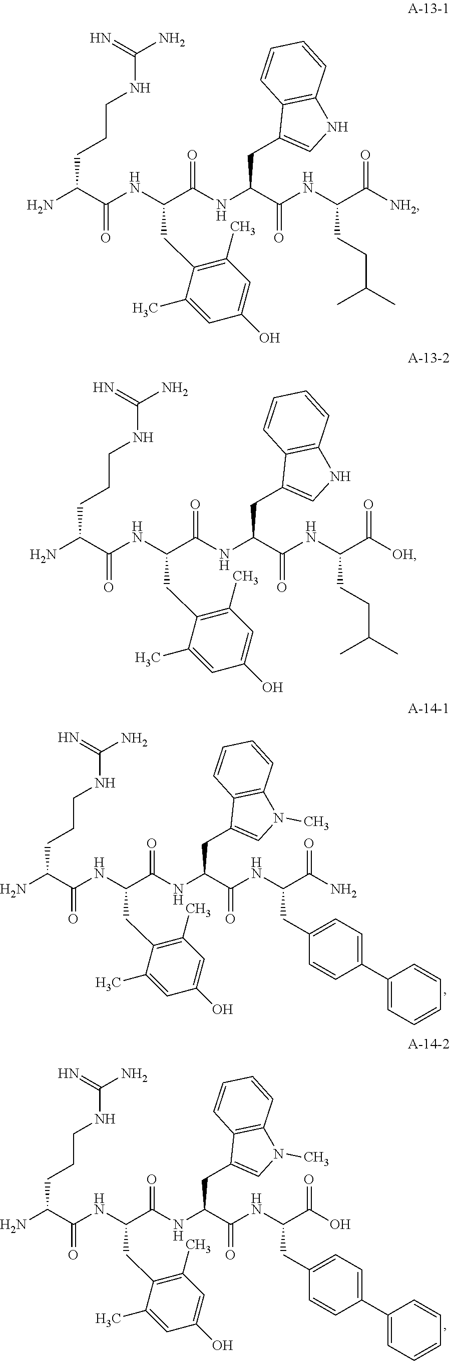 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

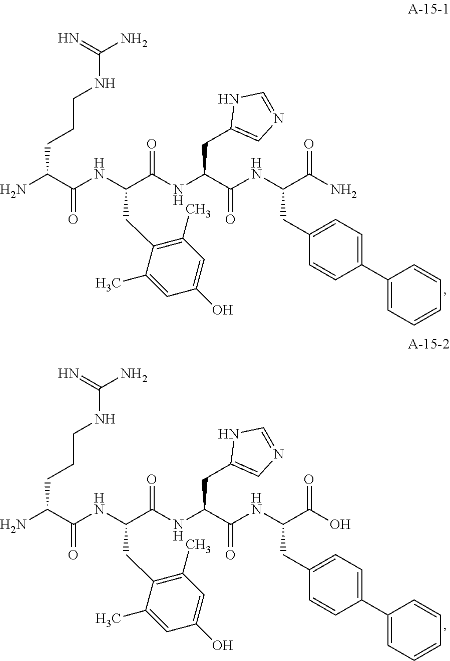 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the method, administration of the peptide or peptides ameliorates, inhibits, delays the onset of, and/or delays the progression of calcium overload, systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, or heart (cardiac) failure in the subject as compared with an untreated control subject or untreated control group that is not administered the peptide or peptides.

In some embodiments of the method, administration of the peptide or peptides ameliorates, inhibits, delays the onset of, and/or delays the progression of calcium overload, systolic disfunction, or diastolic dysfunction in the subject as compared with an untreated control subject or untreated control group that is not administered the peptide or peptides.

In some embodiments of the method, administration of the peptide or peptides ameliorates, inhibits, delays the onset of, and/or delays the progression of ventricular remodeling, including left ventricular remodeling, in the subject as compared with an untreated control subject or untreated control group that is not administered the peptide or peptides.

In some embodiments of the method, administration of the peptide or peptides ameliorates, inhibits, delays the onset of and/or delays the progression of cardiac (myocardial) fibrosis in the subject as compared with an untreated control subject or untreated control group that is not administered the peptide or peptides.

In some embodiments of the method, administration of the peptide or peptides ameliorates, inhibits, delays the onset of, and/or delays the progression of fatty acid infiltration in the subject as compared with an untreated control subject or untreated control group that is not administered the peptide or peptides.

In some embodiments of the method, administration of the peptide or peptides ameliorates, inhibits, delays the onset of, and/or delays the progression of hypertrophic cardiomyopathy in the subject as compared with an untreated control subject or untreated control group that is not administered the peptide or peptides.

In some embodiments of the method, wherein administration of the peptide or peptides ameliorates, inhibits, delays the onset of, and/or delays the progression of dilated cardiomyopathy in the subject as compared with an untreated control subject or untreated control group that is not administered the peptide or peptides.

In some embodiments of the method, administration of the peptide or peptides ameliorates, inhibits, delays the onset of, and/or delays the progression of an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia) in the subject. In some embodiments of the method, administration of the peptide or peptides ameliorates, inhibits, delays the onset of, and/or delays the progression of reduced left ventricular ejection fraction in the subject as compared with an untreated control subject or untreated control group that is not administered the peptide or peptides.

In some embodiments of the method, administration of the peptide or peptides, ameliorates, inhibits, delays the onset of, and/or delays the progression of fractional shortening in the subject as compared with an untreated control subject or untreated control group that is not administered the peptide or peptides.

In some embodiments of the method, administration of the peptide or peptides ameliorates, inhibits, delays the onset of, and/or delays the progression of heart (cardiac) failure in the subject as compared with an untreated control subject or untreated control group that is not administered the peptide or peptides.

In some embodiments of the method, administration of the peptide or peptides results in normalization of the ejection fraction, fractional shortening, stroke volume, and/or cardiac output of the subject as compared with an untreated control subject or control group that is not administered the peptide or peptides.

In some embodiments of the method, the peptide or peptides is/are administered daily for: (i) 12 weeks or more; (ii) 24 weeks or more; (iii) 48 weeks or more; (iv) 72 weeks or more; or (v) 96 weeks or more. In some embodiments of the method, the peptide or peptides is/are administered subcutaneously or intravenously. In some embodiments of the method, the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the method, the subject is human.

In some embodiments of the method, the muscular dystrophy is Duchenne muscular dystrophy.

In some embodiments of the method, the muscular dystrophy is Becker's muscular dystrophy.

In some embodiments of the method, practice of the method further comprises separately, sequentially, or simultaneously administering an additional therapeutic agent to the subject.

In some embodiments of the method, the additional therapeutic agent is a phosphorodiamidate morpholino oligomer (PMO) or a peptide-conjugated phosphorodiamidate morpholino oligomer (PPMO). In some embodiments of the method, the PMO is selected from the group consisting of eteplirsen (Exondys 51®), golodirsen (Vyondys 53®), viltolarsen (Viltepso®), and casimersen (Amondys 45®) or the PPMO is selected from vesleteplirsen (SRP-5051) and PGN-EDO51 (a.k.a., CONNECT1-ED051).

In some embodiments of the method, the additional therapeutic agent is an adeno-associated virus (AAV) vector-based gene therapy. In some embodiments of the method, the adeno-associated virus (AAV) vector-based gene therapy is delandistrogene moxeparvovec-rokl (Elevidys®).

In some embodiments of the method, the additional therapeutic agent is a corticosteroid, an angiotensin-converting enzyme (ACE) inhibitor, an angiotensin receptor blocker (ARB), a beta blocker, a diuretic, or a mineralocorticoid receptor antagonist. In some embodiments of the method, the corticosteroid is vamorolone (aGamree®), prednisone, or deflazacort. In some embodiments of the method, the angiotensin-converting enzyme (ACE) inhibitor is captopril, enalapril, lisinopril, benazepril, or ramipril. In some embodiments of the method, the angiotensin receptor blocker (ARB) is azilsartan, candesartan, eprosartan, irbesartan, telmisartan, valsartan, losartan, olmesartan, Entresto® (sacubitril/valsartan) or Byvalson™ (nebivolol/valsartan). In some embodiments of the method, the beta blocker is carvedilol, bisoprolol, metoprolol succinate, atenolol, esmolol, nebivolol, or propranolol. In some embodiments of the method, the diuretic is bumetanide, furosemide, or torsemide. In some embodiments of the method, the mineralocorticoid receptor antagonist is spironolactone or eplerenone.

In some embodiments of the method, the additional therapeutic agent is mavacamten (Camzyos®).

In some embodiments of the method, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the method, the peptide or peptides is/are formulated for administration from its/their tris —HCl salt.

In still another aspect, the present disclosure pertains to a composition or medicament for use in treating, preventing, inhibiting, ameliorating or delaying the onset of muscular dystrophy or its signs or symptoms in a mammalian subject in need thereof, wherein the composition or medicament comprises a peptide of Formula A, or a mixture of peptides of Formula A:

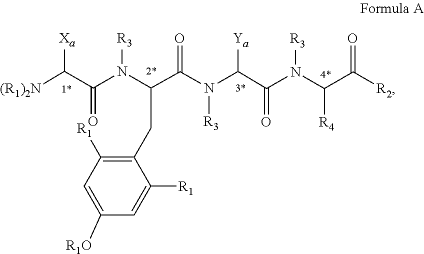

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

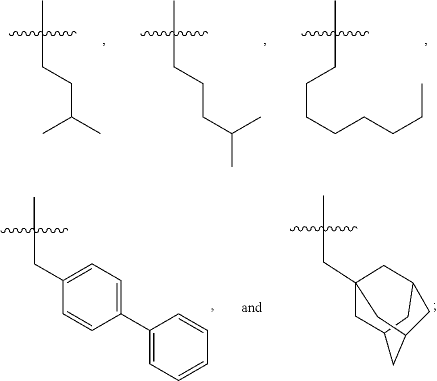

Xₐ is selected from

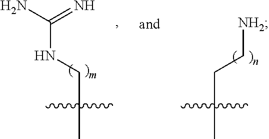

### Yₐ

is selected from

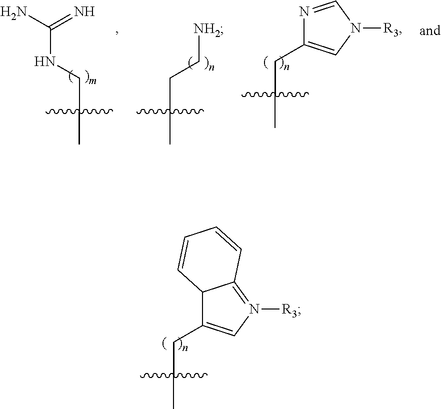

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is substituted with a deuterium or fluorine atom. In some embodiments of the composition or medicament, the peptide or peptides of Formula A is/are a peptide/peptides of Formula A-1 or Formula A-2:

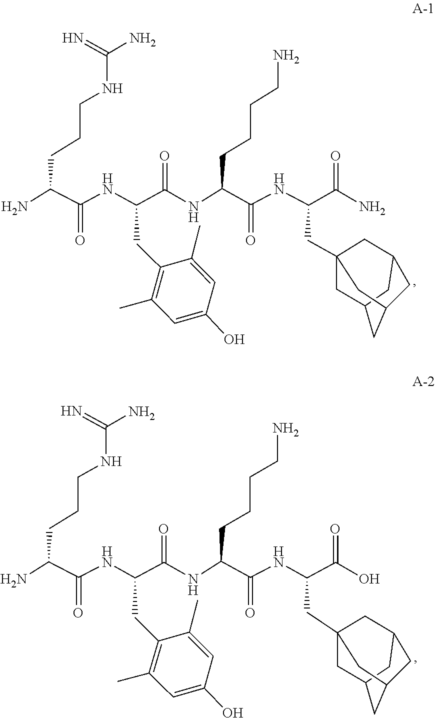 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the composition or medicament, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

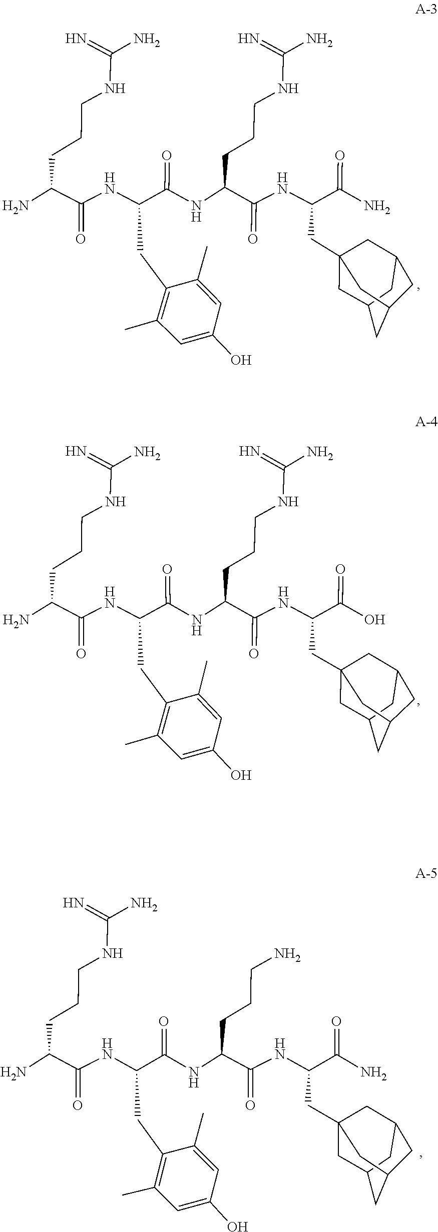 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

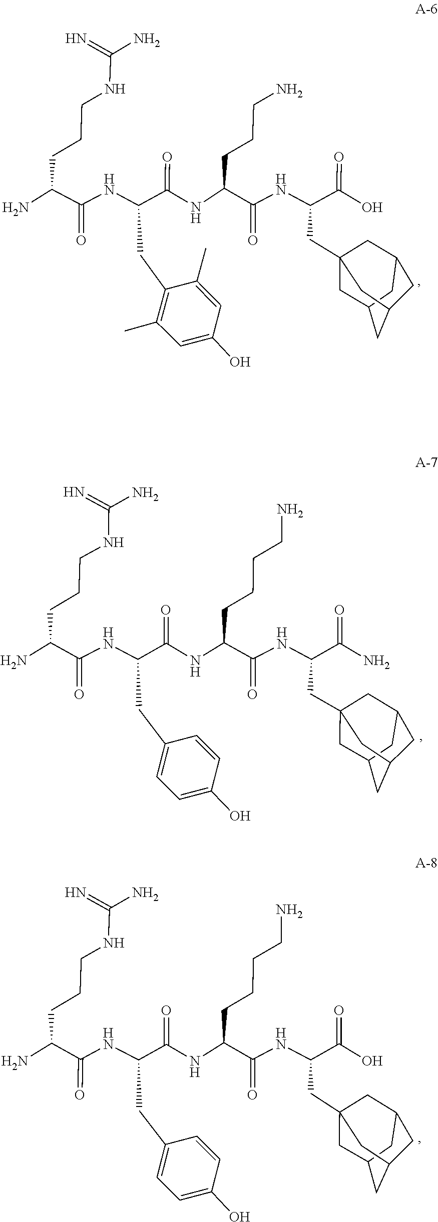 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

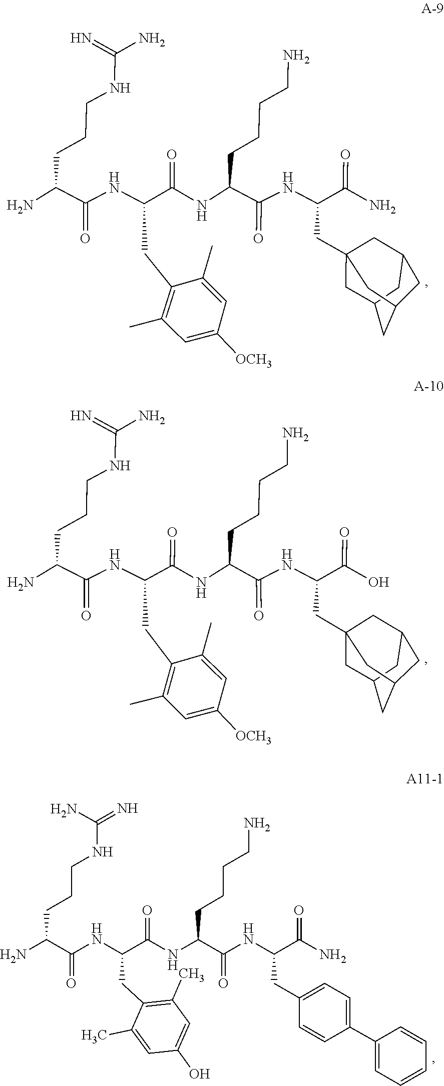 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O]

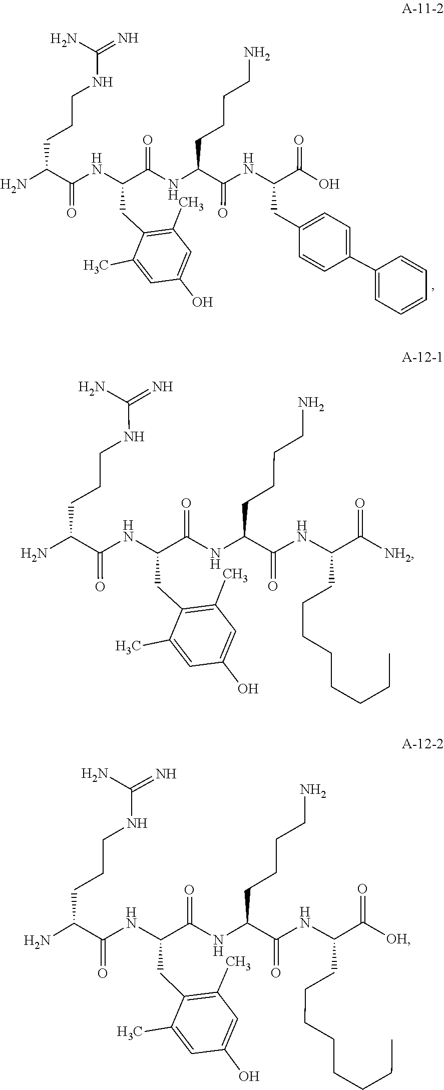 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

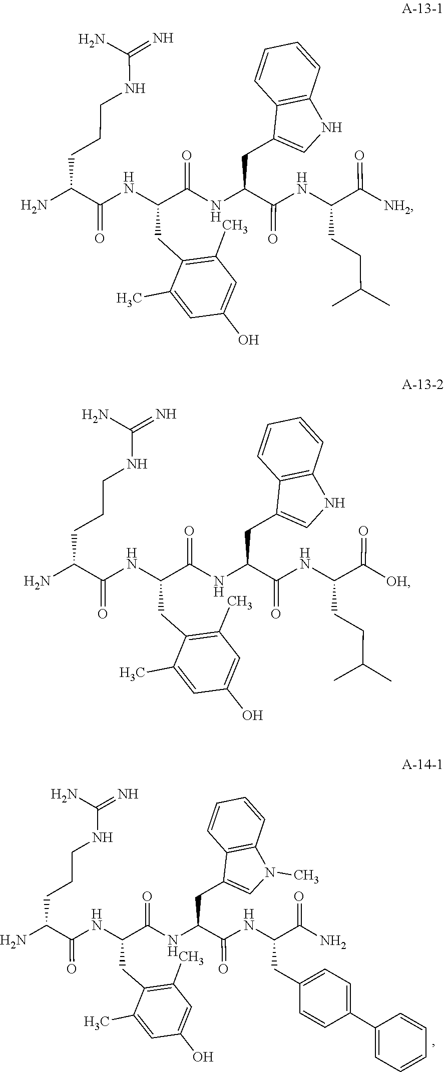 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O]

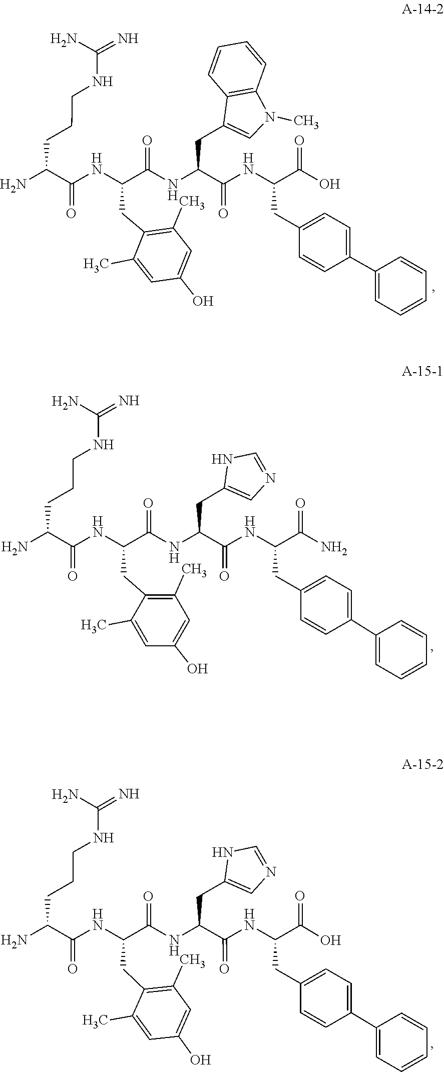 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the composition or medicament, administration of the composition or medicament to the subject ameliorates, inhibits, delays the onset of, and/or delays the progression of one or more signs or symptoms of muscular dystrophy in the subject as compared with an untreated control subject or untreated control group that is not administered the composition or medicament.

In some embodiments of the composition or medicament, administration of the composition or medicament to the subject ameliorates, inhibits, delays the onset of, and/or delays the progression of the progression of a cardiomyopathy associated with muscular dystrophy in the subject as compared with an untreated control subject or untreated control group that is not administered the composition or medicament. In some embodiments of the composition or medicament, administration of the composition or medicament to the subject ameliorates, inhibits, delays the onset of, and/or delays the progression of calcium overload, systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, or heart (cardiac) failure in the subject as compared with an untreated control subject or untreated control group that is not administered the composition or medicament.

In some embodiments of the composition or medicament, administration of the composition or medicament ameliorates, inhibits, delays the onset of, and/or delays the progression of contraction-induced damage in muscle tissue, locomotor-skeletal muscle weakness, respiratory muscle weakness, cardiac muscle weakness, sarcolemmal weakening, or sarcolemmal tearing in the subject as compared with an untreated control subject or untreated control group that is not administered the composition or medicament.

In some embodiments of the composition or medicament, the composition or medicament is administered daily for: (i) 12 weeks or more; (ii) 24 weeks or more; (iii) 48 weeks or more; (iv) 72 weeks or more; or (v) 96 weeks or more. In some embodiments of the composition or medicament, the composition or medicament is administered subcutaneously or intravenously. In some embodiments of the composition or medicament, the composition or medicament is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the composition or medicament is administered to a human subject.

In some embodiments of the composition or medicament, the muscular dystrophy is Duchenne muscular dystrophy. In some embodiments of the composition or medicament, muscular dystrophy is Becker's muscular dystrophy.

In some embodiments of the composition or medicament, the pharmaceutically acceptable salt of the peptide or peptides of the composition or medicament comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, wherein the peptide or peptides of the composition or medicament is/are formulated for administration from its/their tris —HCl salt.

In still another aspect, the present disclosure pertains to composition or medicament for use in treating, preventing, inhibiting, ameliorating or delaying the onset of a cardiomyopathy in a mammalian subject in need thereof, wherein the subject is suffering from muscular dystrophy, wherein the composition or medicament comprises a peptide of Formula A, or a mixture of peptides of Formula A:

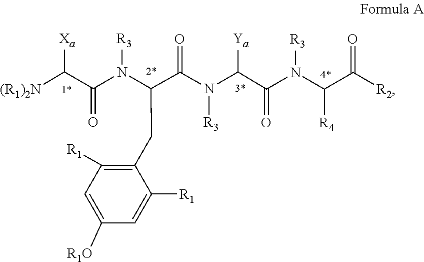

or a pharmaceutically acceptable salt, hydrate, solvate, and/or tautomer thereof, wherein, each R₁ is independently H, —CH₃, or —CH₂CH₃; R₂ is —OH, —NH₂, —NHR₁ or —N(R₁)₂; each R₃ is independently H, or —CH₃; R₄ is selected from:

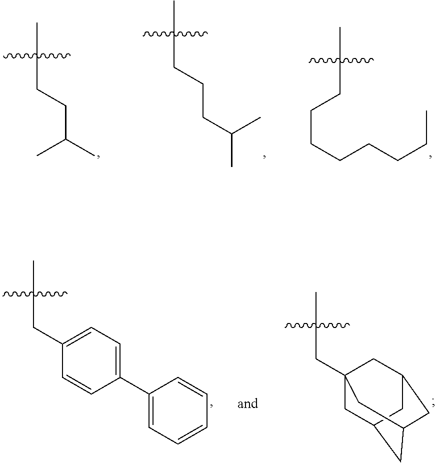

Xₐ is selected from

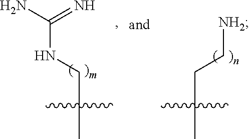

Yₐ is selected from

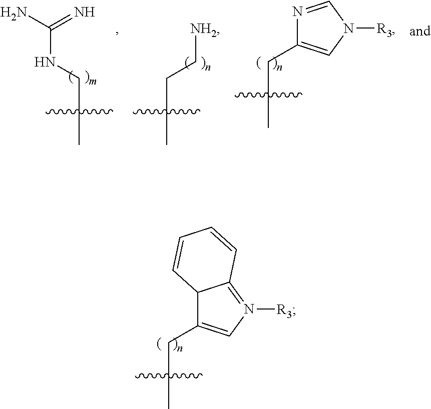

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom. In some embodiments of the composition or medicament, the peptide or peptides of Formula A is/are a peptide/peptides of Formula A-1 or Formula A-2:

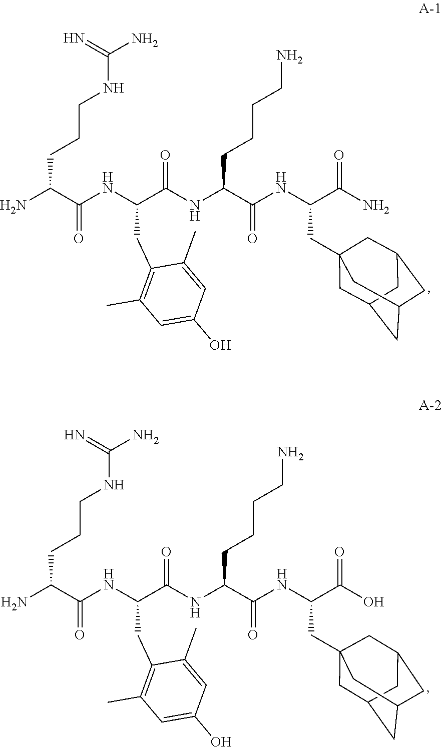 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the composition or medicament, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

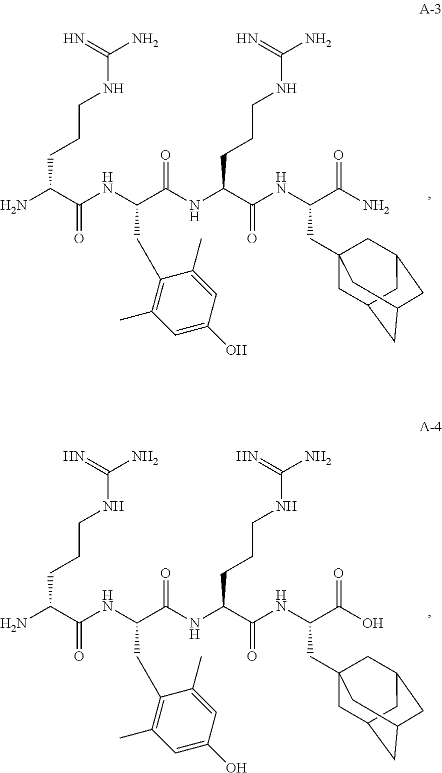 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O]

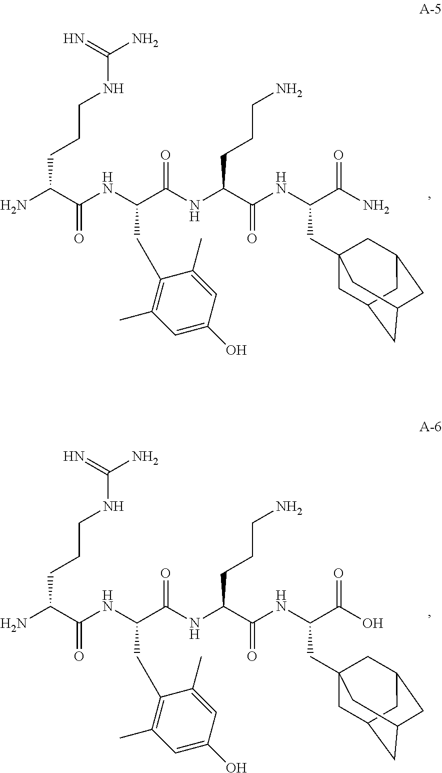 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

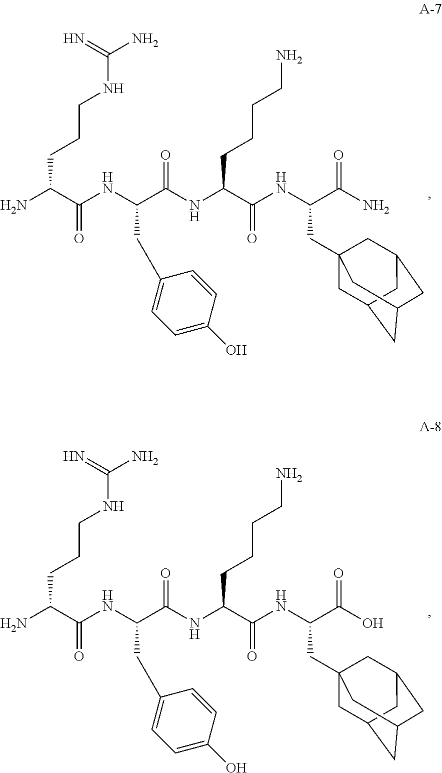 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

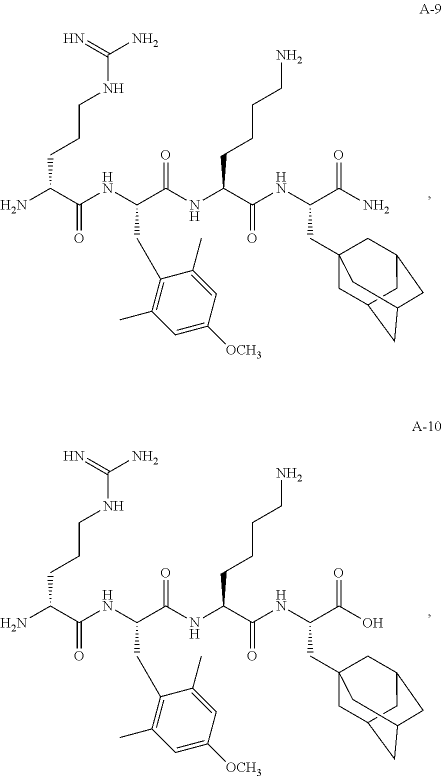 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

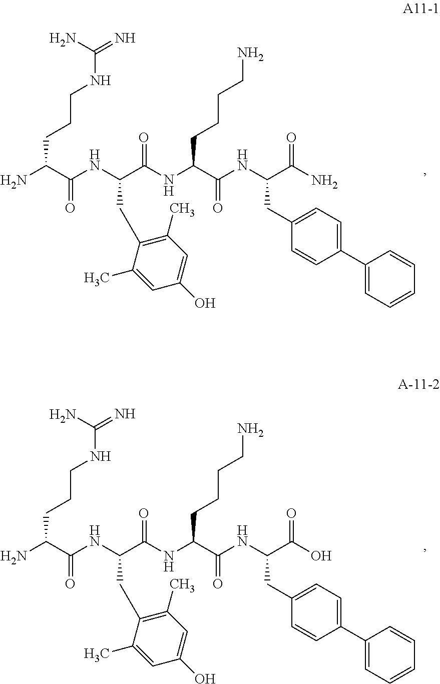 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

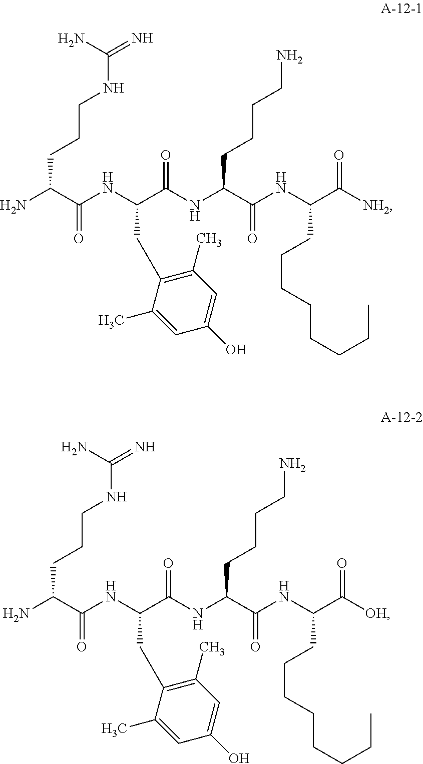 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

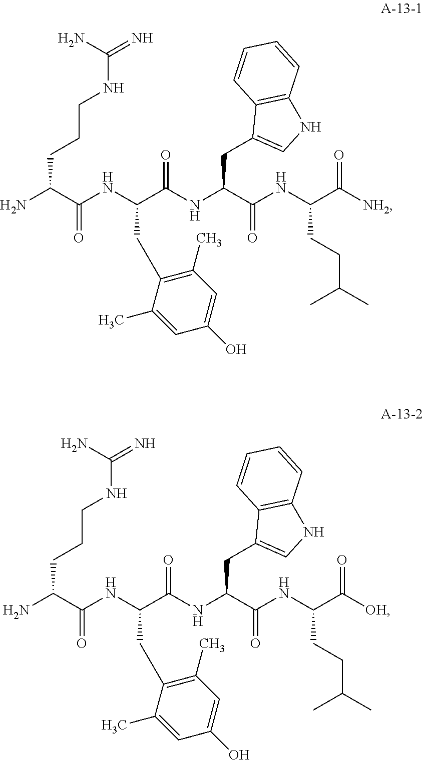 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

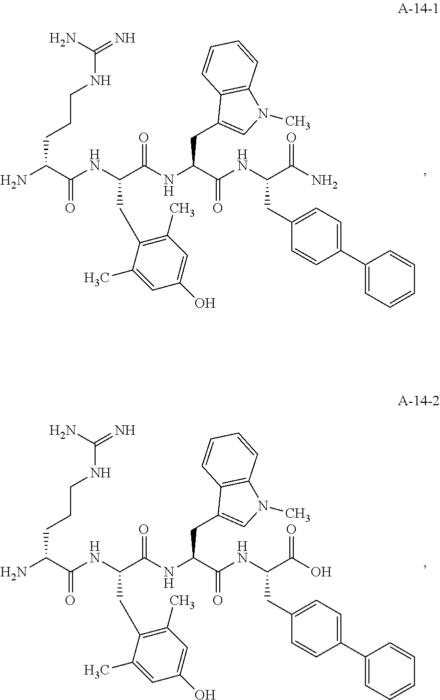 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

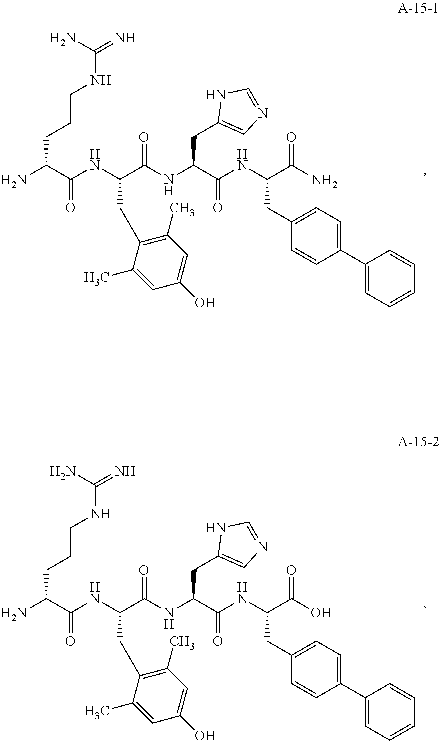 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the composition or medicament, administration of the composition or medicament to the subject ameliorates, inhibits, delays the onset of, and/or delays the progression of calcium overload, systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, heart (cardiac) failure, or any two or more of the foregoing, in the subject as compared with an untreated control subject or control group that is not administered the composition or medicament.

In some embodiments of the composition or medicament, administration of the composition or medicament to the subject ameliorates, inhibits, delays the onset of, and/or delays the progression of calcium overload, systolic disfunction, or diastolic dysfunction of the subject as compared with an untreated control subject or control group that is not administered the composition or medicament.

In some embodiments of the composition or medicament, administration of the composition or medicament to the subject ameliorates, inhibits, delays the onset of, and/or delays the progression of ventricular remodeling, including left ventricular remodeling, of the subject as compared with an untreated control subject or control group that is not administered the composition or medicament.

In some embodiments of the composition or medicament, administration of the composition or medicament to the subject ameliorates, inhibits, delays the onset of and/or delays the progression of cardiac (myocardial) fibrosis of the subject as compared with an untreated control subject or control group that is not administered the composition or medicament.

In some embodiments of the composition or medicament, administration of the composition or medicament to the subject ameliorates, inhibits, delays the onset of, and/or delays the progression of fatty acid infiltration of the subject as compared with an untreated control subject or control group that is not administered the composition or medicament.

In some embodiments of the composition or medicament, administration of the composition or medicament to the subject ameliorates, inhibits, delays the onset of, and/or delays the progression of hypertrophic cardiomyopathy of the subject as compared with an untreated control subject or control group that is not administered the composition or medicament.

In some embodiments of the composition or medicament, administration of the composition or medicament to the subject ameliorates, inhibits, delays the onset of, and/or delays the progression of dilated cardiomyopathy of the subject as compared with an untreated control subject or control group that is not administered the composition or medicament.

In some embodiments of the composition or medicament, administration of the composition or medicament to the subject ameliorates, inhibits, delays the onset of, and/or delays the progression of an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia) of the subject as compared with an untreated control subject or control group that is not administered the composition or medicament.

In some embodiments of the composition or medicament, administration of the composition or medicament to the subject ameliorates, inhibits, delays the onset of, and/or delays the progression of reduced left ventricular ejection fraction of the subject as compared with an untreated control subject or control group that is not administered the composition or medicament.

In some embodiments of the composition or medicament, administration of the composition or medicament to the subject ameliorates, inhibits, delays the onset of, and/or delays the progression of fractional shortening of the subject as compared with an untreated control subject or control group that is not administered the composition or medicament.

In some embodiments of the composition or medicament, administration of the composition or medicament to the subject ameliorates, inhibits, delays the onset of, and/or delays the progression of heart (cardiac) failure of the subject as compared with an untreated control subject or control group that is not administered the composition or medicament.

In some embodiments of the composition or medicament, administration of the composition or medicament to the subject results in normalization of the ejection fraction, fractional shortening, stroke volume, and/or cardiac output of the subject as compared with an untreated control subject or control group that is not administered the composition or medicament.

In some embodiments of the composition or medicament, the composition or medicament is administered daily for: (i) 12 weeks or more; (ii) 24 weeks or more; (iii) 48 weeks or more; (iv) 72 weeks or more; or (v) 96 weeks or more. In some embodiments of the composition or medicament, the composition or medicament is administered subcutaneously or intravenously. In some embodiments of the composition or medicament, the composition or medicament is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the composition or medicament is administered to a human subject.

In some embodiments of the composition or medicament, the muscular dystrophy is Duchenne muscular dystrophy. In some embodiments of the composition or medicament, the muscular dystrophy is Becker's muscular dystrophy.

In some embodiments of the composition or medicament, administration of the composition or medicament further comprises separately, sequentially, or simultaneously administering an additional therapeutic agent to the subject.

In some embodiments of the composition or medicament, the additional therapeutic agent is a phosphorodiamidate morpholino oligomer (PMO) or a peptide-conjugated phosphorodiamidate morpholino oligomer (PPMO). In some embodiments of the composition or medicament, the PMO is selected from the group consisting of eteplirsen (Exondys 51®), golodirsen (Vyondys 53®), viltolarsen (Viltepso®), and Casimersen (Amondys 45®) or the PPMO is selected from vesleteplirsen (a.k.a., SRP-5051) and PGN-EDO51 (a.k.a., CONNECT1-ED051).

In some embodiments of the composition or medicament, the additional therapeutic agent is an adeno-associated virus (AAV) vector-based gene therapy. In some embodiments of the composition or medicament, the adeno-associated virus vector-based gene therapy is delandistrogene moxeparvovec-rokl (Elevidys®).

In some embodiments of the composition or medicament, the additional therapeutic agent is a corticosteroid, an angiotensin-converting enzyme (ACE) inhibitor, an angiotensin receptor blocker (ARB), a beta blocker, a diuretic, or a mineralocorticoid receptor antagonist. In some embodiments of the composition or medicament, the corticosteroid is vamorolone (aGamree®), prednisone, or deflazacort. In some embodiments of the composition or medicament, the angiotensin-converting enzyme (ACE) inhibitor is captopril, enalapril, lisinopril, benazepril, or ramipril. In some embodiments of the composition or medicament, the angiotensin receptor blocker (ARB) is azilsartan, candesartan, eprosartan, irbesartan, telmisartan, valsartan, losartan, olmesartan, Entresto® (sacubitril/valsartan) or Byvalson™ (nebivolol/valsartan). In some embodiments of the composition or medicament, the beta blocker is carvedilol, bisoprolol, metoprolol succinate, atenolol, esmolol, nebivolol, or propranolol. In some embodiments of the composition or medicament, the diuretic is bumetanide, furosemide, or torsemide. In some embodiments of the composition or medicament, the mineralocorticoid receptor antagonist is spironolactone or eplerenone.

In some embodiments of the composition or medicament, the additional therapeutic agent is mavacamten (Camzyos®).

In some embodiments of the composition or medicament, the pharmaceutically acceptable salt of the peptide or peptides of the composition or medicament comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, the peptide or peptides of the composition or medicament is/are formulated for administration from its/their tris —HCl salt.

In still another aspect, the present disclosure pertains to the use of a peptide (or mixture of peptides) or a composition or medicament comprising the peptide (or mixture of peptides) for treating, preventing, inhibiting, ameliorating or delaying the onset of muscular dystrophy in a mammalian subject in need thereof, wherein the peptide, peptides of the mixture, the composition or the medicament is/are a peptide of Formula A:

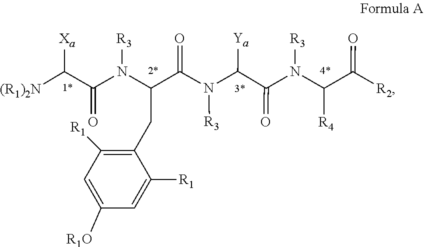

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

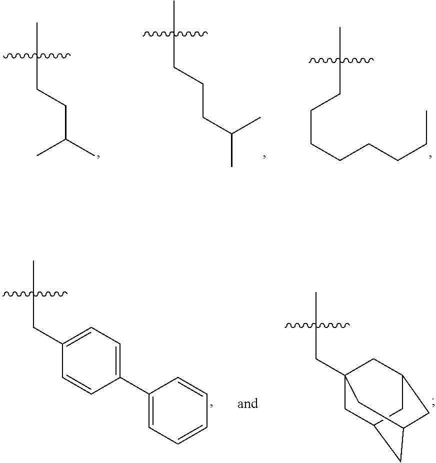

Xₐ is selected from

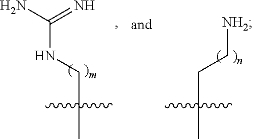

### Yₐ

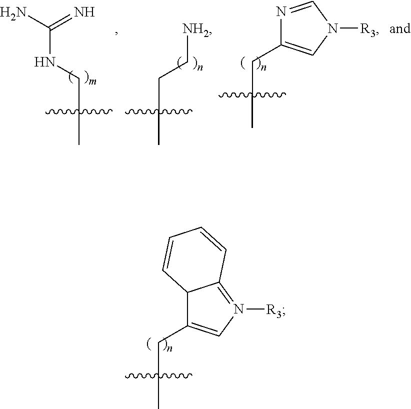

is selected from each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide or peptides of Formula A is/are substituted with a deuterium or fluorine atom.

In still another aspect, the present disclosure pertains to the use of a peptide, mixture of peptides or a composition or medicament comprising the peptide (or mixture of peptides) for treating, preventing, inhibiting, ameliorating or delaying the onset of a cardiomyopathy in a mammalian subject in need thereof, wherein the subject is suffering from muscular dystrophy, wherein the peptide or peptides of the mixture is/are a peptide of Formula A:

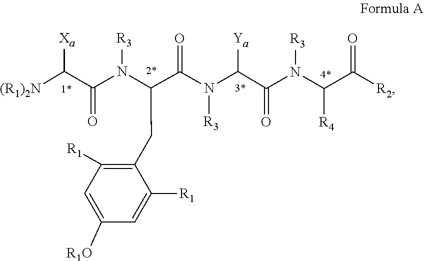

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

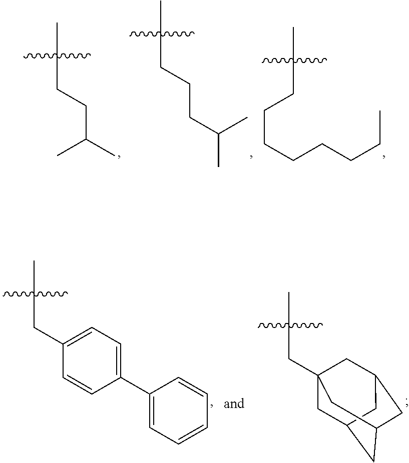

Xₐ is selected from

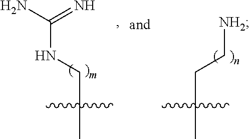

Yₐ is selected from

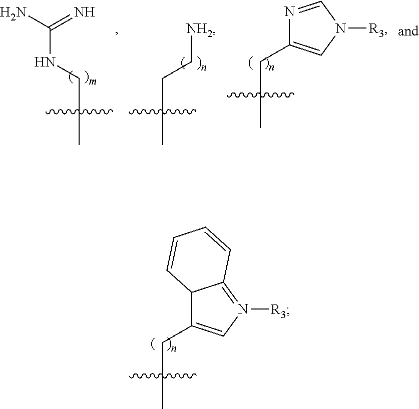

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide or peptides of Formula A is/are substituted with a deuterium or fluorine atom.

In still another aspect, the present disclosure pertains to a method for treating, preventing, inhibiting, ameliorating or delaying the onset of loss of ambulation in a mammalian subject in need thereof, wherein the subject is having, or suspected of having, muscular dystrophy, comprising administering to the subject a therapeutically effective amount of a peptide of Formula A:

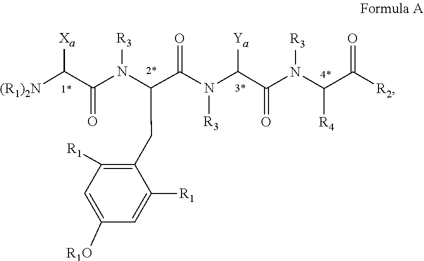

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

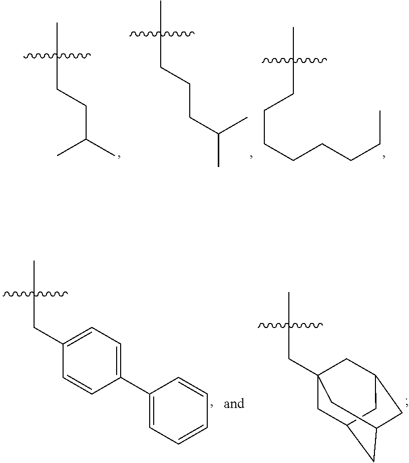

Xₐ is selected from

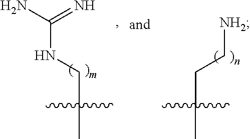

Yₐ is selected from

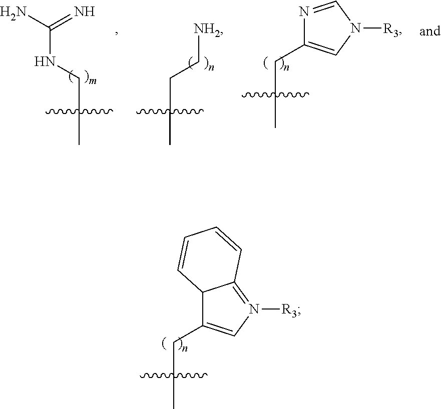

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom. In some embodiments of the method, the peptide or peptides of Formula A is/are a peptide/peptides of Formula A-1 or Formula A-2:

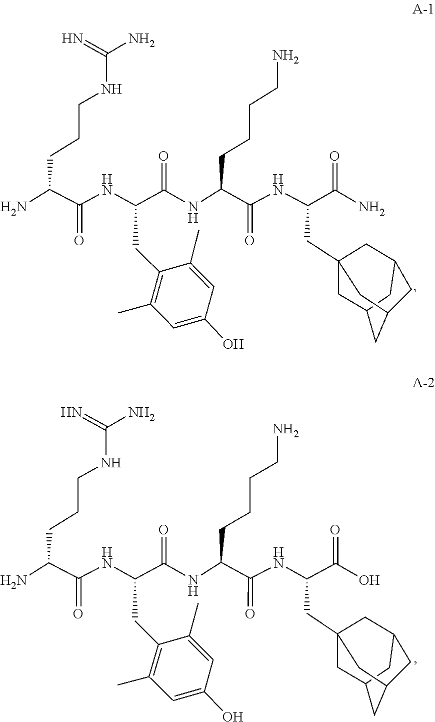 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the method, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

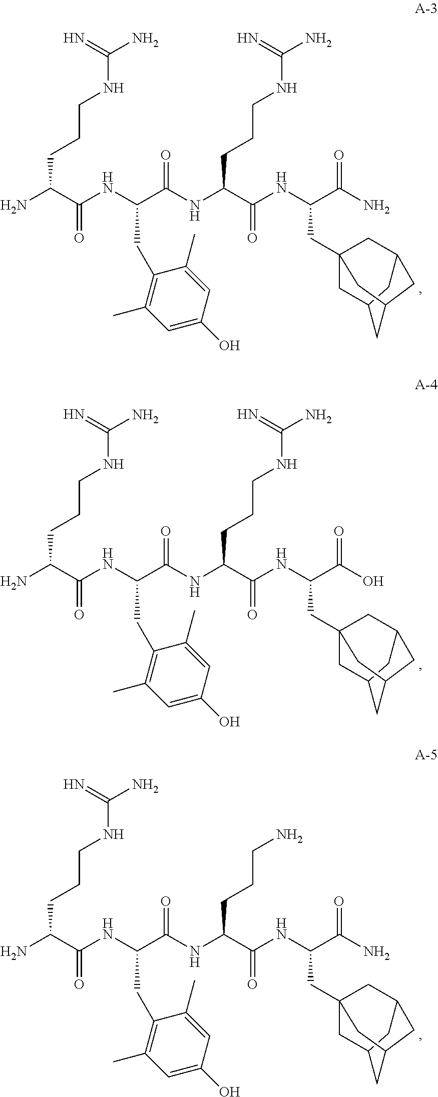 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

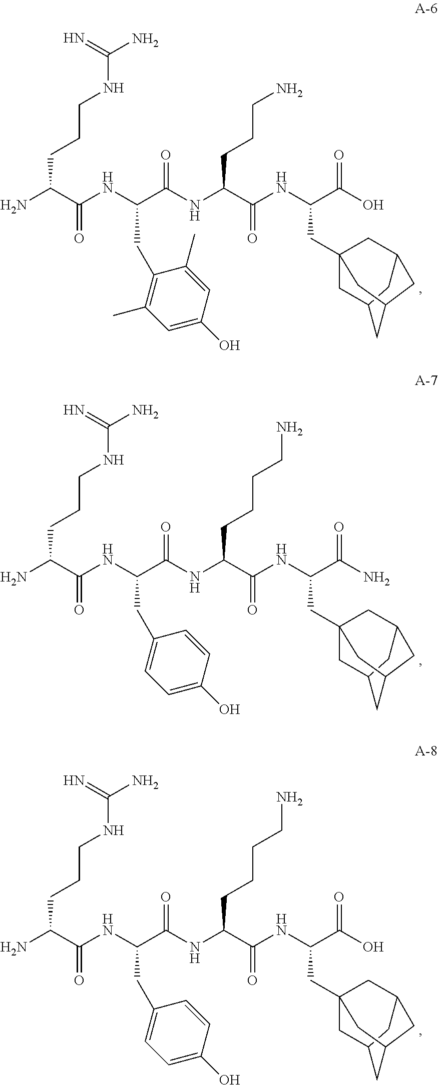 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

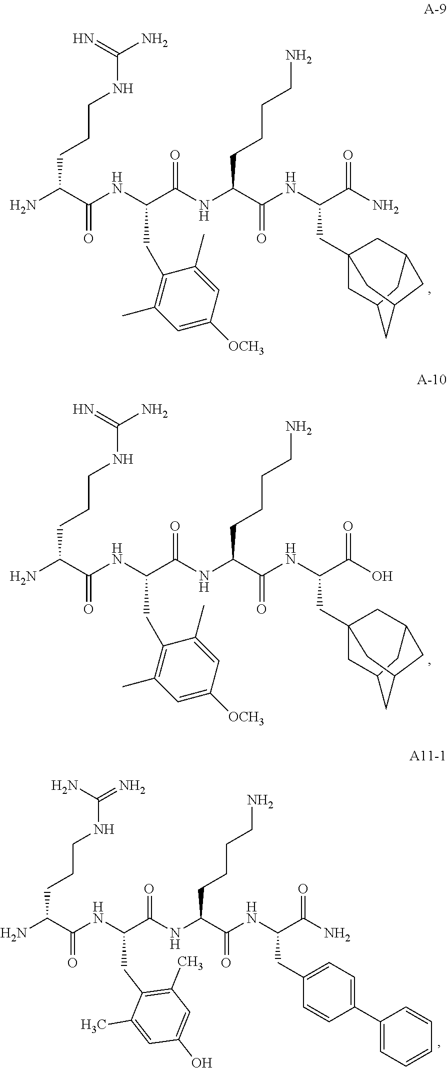 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O]

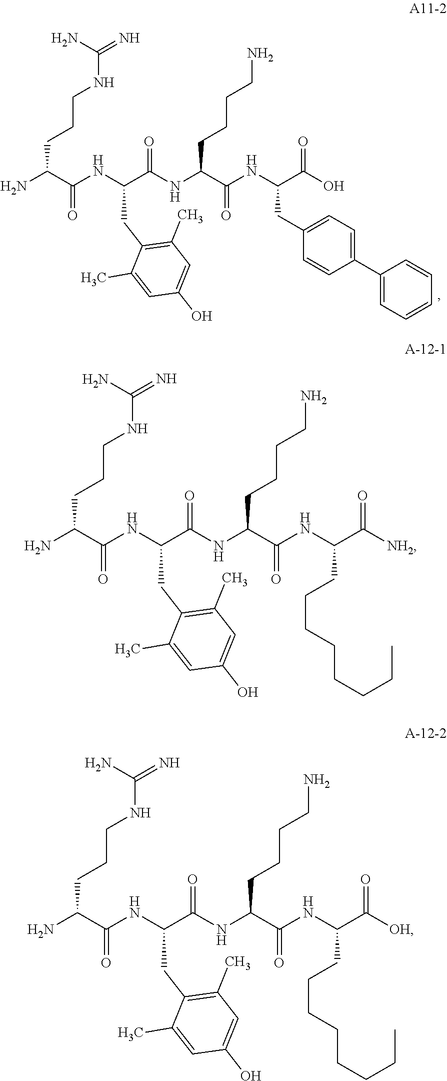 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

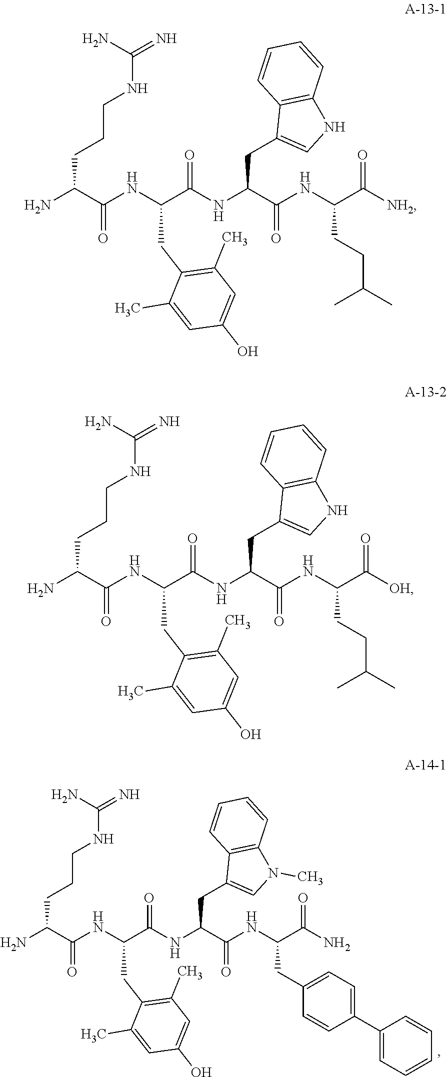 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O]

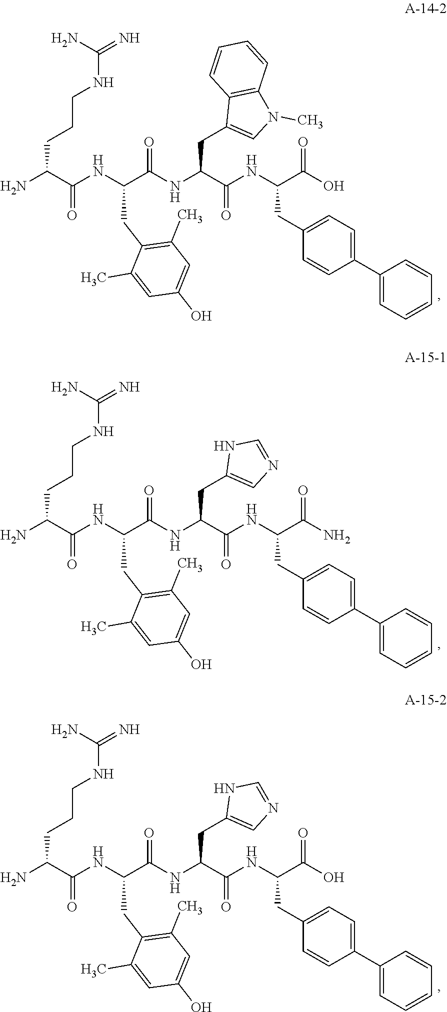 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In still another aspect, the present disclosure pertains to a method for treating, preventing, inhibiting, ameliorating or delaying the onset of respiratory distress in a mammalian subject in need thereof, wherein the subject is having, or suspected of having, muscular dystrophy, comprising administering to the subject a therapeutically effective amount of a peptide of Formula A, or mixture of peptides of Formula A:

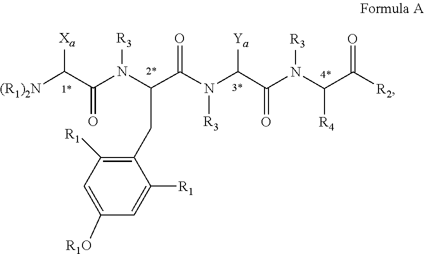

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

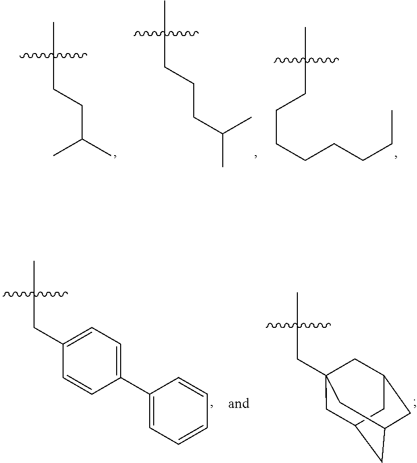

Xₐ is selected from

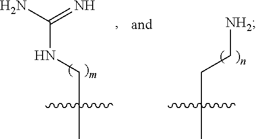

Yₐ is selected from

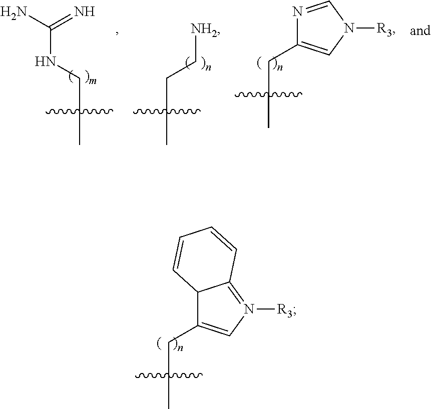

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom. In some embodiments of the method, the peptide or peptides of Formula A is/are a peptide/peptides of Formula A-1 or A-2:

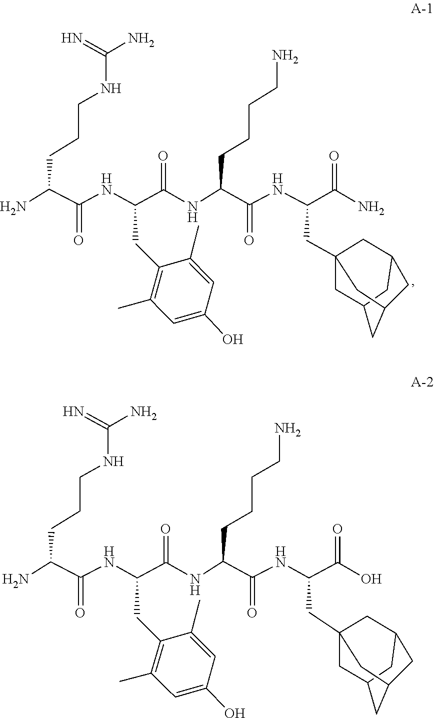 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the method, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

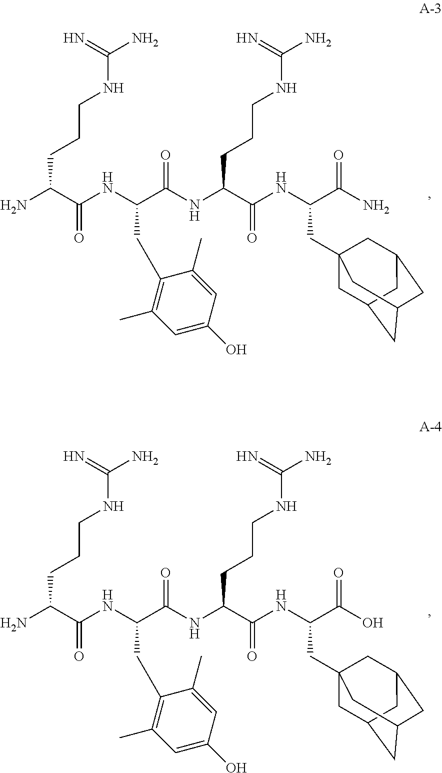 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O]

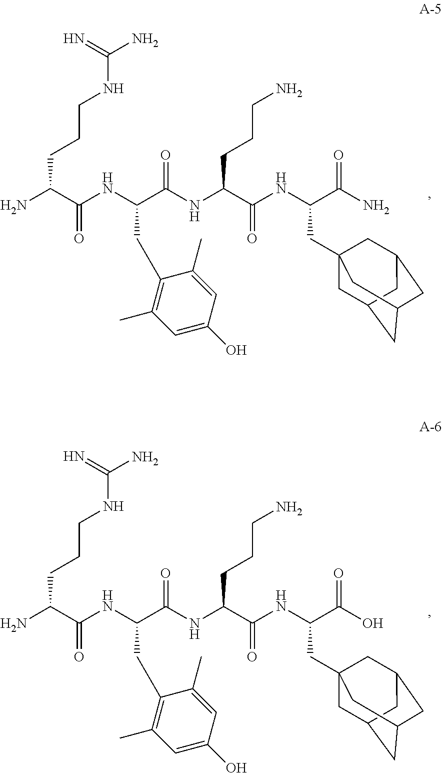 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

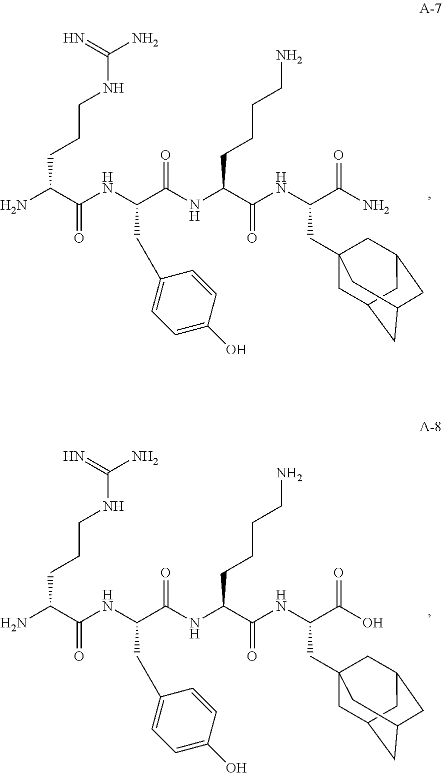 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

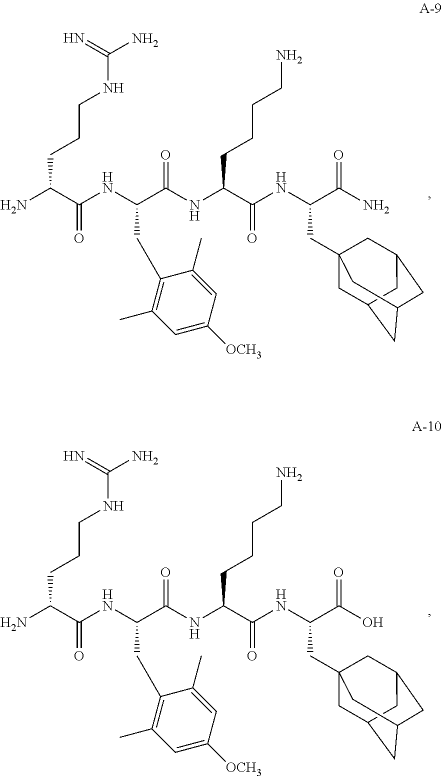 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

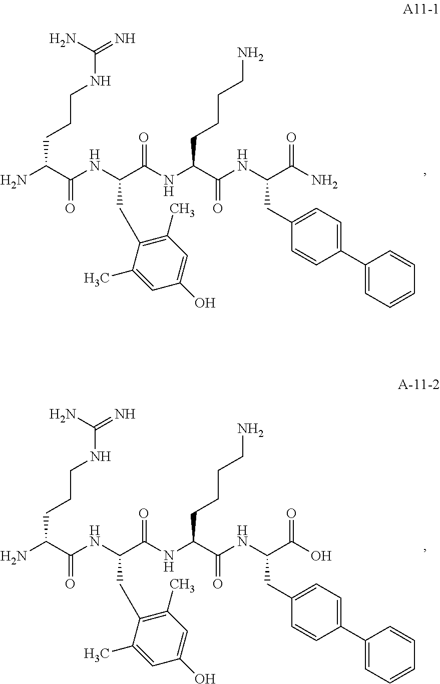 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

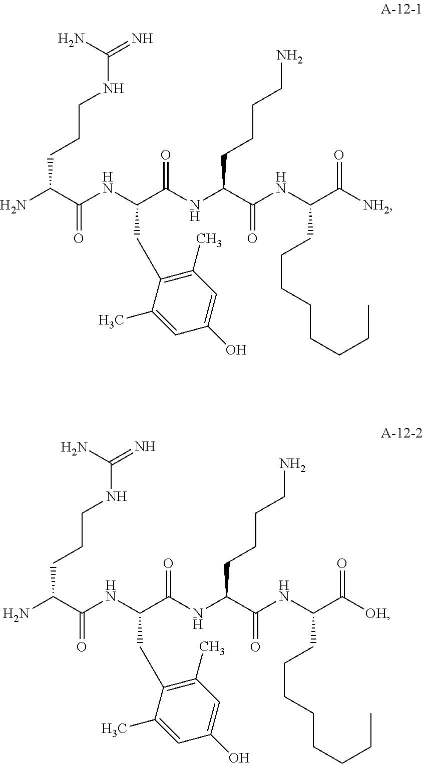 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

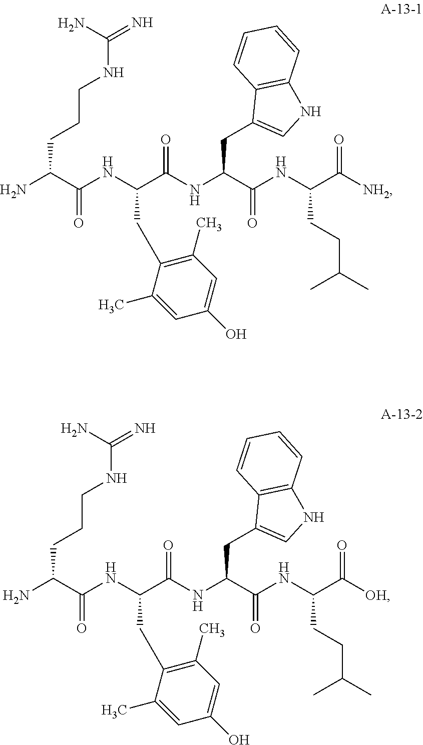 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

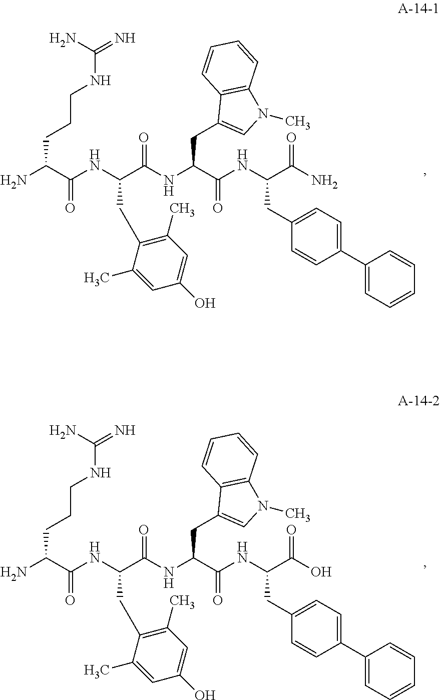 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

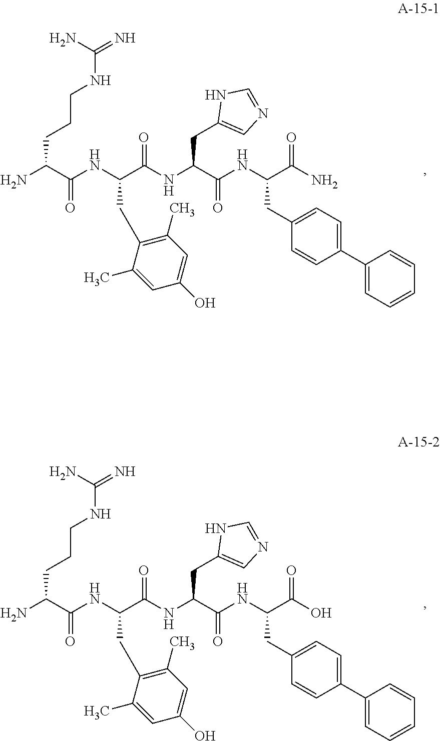 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

### BRIEF DESCRIPTION OF THE DRAWINGS

FIG. 1 is an illustration of a generic structure for peptides of Formula A and the structure for two specific examples of specific peptides (peptides of Formula A-1 and Formula A-2) within the scope of Formula A as defined herein.

FIG. 2 is an illustration of various salt forms of the peptide of Formula A-2.

FIG. 3 is an illustration of various salt forms of the peptide of Formula A-1.

FIG. 4 is a scheme for the synthesis of the peptide of Formula A-1.

FIG. 5A is a graphic presentation of the data for a time course examination of blood plasma concentrations of elamipretide (a.k.a., SS-31) and the peptide of Formula A-1 in C57BL/6 mice treated interperitoneally with a single 5 mg/kg dose of each compound as described in Example 1.

FIG. 5B is a graphic presentation of the data for a time course examination of the concentration of the peptide of Formula A-1 in: (i) blood plasma (same data as presented in FIG. 5A), (ii) heart tissue, and (iii) muscle tissue of C57BL/6 mice treated interperitoneally with a single 5 mg/kg dose as described in Example 1.

FIG. 6 is a bar graph presentation of data for the geometric mean tissue concentrations (ng/g) of elamipretide and the peptide of Formula A-1 in muscle tissue, heart tissue, and liver tissue as described in Example 2.

FIG. 7A is a graphic presentation of cardiac data of the right ventricle end-systolic pressure (ESP) for: (i) an untreated wild-type mouse line (Wildtype), (ii) a Duchenne Muscular Dystrophy (DMD) mouse model treated with vehicle (Vehicle), (iii) a Duchenne Muscular Dystrophy (DMD) mouse model treated with a low dose (1 mg/kg) of the peptide of Formula A-1 (Low Dose); and (iv) a Duchenne Muscular Dystrophy (DMD) mouse model treated with a high dose (5 mg/kg) of the peptide of Formula A-1 as described in Example 3 (High Dose).

FIG. 7B is a graphic presentation of cardiac data of the right ventricle end-systolic pressure (EDP) for: (i) an untreated wild-type mouse line (Wildtype), (ii) a Duchenne Muscular Dystrophy (DMD) mouse model treated with vehicle (Vehicle), (iii) a Duchenne Muscular Dystrophy (DMD) mouse model treated with a low dose (1 mg/kg) of the peptide of Formula A-1 (Low Dose); and (iv) a Duchenne Muscular Dystrophy (DMD) mouse model treated with a high dose (5 mg/kg) of the peptide of Formula A-1 as described in Example 3 (High Dose).

FIG. 8 is a graphic presentation of diaphragm fibrosis data for: (i) an untreated wild-type mouse line (WT), (ii) a Duchenne Muscular Dystrophy (DMD) mouse model treated with vehicle (V), (iii) a Duchenne Muscular Dystrophy (DMD) mouse model treated with a low dose (1 mg/kg) of the peptide of Formula A-1 (LD); and (iv) a Duchenne Muscular Dystrophy (DMD) mouse model treated with a high dose (5 mg/kg) of the peptide of Formula A-1 as described in Example 3 (HD).

FIG. 9 is a graphic presentation of cardiac data (specifically the left ventricle ejection fraction or LVEF) of: (i) an untreated wild-type rabbit (WT), (ii) a Duchenne Muscular Dystrophy (DMD) rabbit model treated with vehicle (KO-Vehicle), (iii) a Duchenne Muscular Dystrophy (DMD) rabbit model treated with the peptide of Formula A-1 treated for 4 weeks (KO-Formula A-1 (4 weeks)), and (iv) a Duchenne Muscular Dystrophy (DMD) rabbit model treated with the peptide of Formula A-1 treated for 8 weeks as described in Example 4 (KO-Formula A-1 (8 weeks)).

FIG. 10A presents the structure of a peptide of Formula A-11-1 and accompanying graphic data presented for: (i) the white necrotic tissue/risk, and (ii) total risk assessed for mouse hearts treated with: (a) vehicle, (b) elamipretide, and (c) the peptide of Formula A-11-1; all subjected to the Langendorff procedure as described in Example 5.

FIG. 10B presents the structure of a peptide of Formula A-1 and accompanying graphic data presented for: (i) the white necrotic tissue/risk, and (ii) total risk assessed for mouse hearts treated with: (a) vehicle, (b) elamipretide, and (c) the peptide of Formula A-1; all subjected to the Langendorff procedure as described in Example 5.

FIG. 10C presents the structure of a peptide of Formula A-12-1 and accompanying graphic data presented for: (i) the white necrotic tissue/risk, and (ii) total risk assessed for mouse hearts treated with: (a) vehicle, (b) elamipretide, and (c) the peptide of Formula A-12-1; all subjected to the Langendorff procedure as described in Example 5.

FIG. 10D presents the structure of a peptide of Formula A-13-1 and accompanying graphic data presented for: (i) the white necrotic tissue/risk, and (ii) total risk assessed for mouse hearts treated with: (a) vehicle, (b) elamipretide, and (c) the peptide of Formula A-13-1; all subjected to the Langendorff procedure as described in Example 5.

FIG. 10E presents the structure of a peptide of Formula A-14-1 and accompanying graphic data presented for: (i) the white necrotic tissue/risk, and (ii) total risk assessed for mouse hearts treated with: (a) vehicle, (b) elamipretide, and (c) the peptide of Formula A-14-1; all subjected to the Langendorff procedure as described in Example 5.

FIG. 10F presents the structure of a peptide of Formula A-15-1 and accompanying graphic data presented for: (i) the white necrotic tissue/risk, and (ii) total risk assessed for mouse hearts treated with: (a) vehicle, (b) elamipretide, and (c) the peptide of Formula A-15-1; all subjected to the Langendorff procedure as described in Example 5.

FIG. 10G presents the structure of a peptide of Formula A-16 and accompanying graphic data presented for: (i) the white necrotic tissue/risk, and (ii) total risk assessed for mouse hearts treated with: (a) vehicle, (b) elamipretide, and (c) the peptide of Formula A-16; all subjected to the Langendorff procedure as described in Example 5.

FIG. 11 is an illustration of study protocol used to treat D2.WT mice and D2.mdx mice with μ-dystrophin gene therapy (μDys) and optionally with the additional administration of a peptide of Formula A-1 as described in Example 6.

FIG. 12A is a graphic illustration of data obtained for left ventricular (LV) end-systole diameter (mm) for D2.WT mice and D2.mdx mice treated as described in Example 6.

FIG. 12B is a graphic illustration of data obtained for left ventricular (LV) end-diastole diameter (mm) for D2.WT mice and D2.mdx mice treated as described in Example 6.

FIG. 13A is a graphic illustration of data obtained for left ventricular (LV) end-systole volume (μL) for D2.WT mice and D2.mdx mice treated as described in Example 6.

FIG. 13B is a graphic illustration of data obtained for left ventricular (LV) end-diastole volume (μL) for D2.WT mice and D2.mdx mice treated as described in Example 6.

FIG. 14A is a graphic illustration of data obtained for left ventricular (LV) anterior wall thickness systole (mm) for D2.WT mice and D2.mdx mice treated as described in Example 6.

FIG. 14B is a graphic illustration of data obtained for left ventricular (LV) posterior wall thickness systole (mm) for D2.WT mice and D2.mdx mice treated as described in Example 6.

FIG. 15A is a graphic illustration of data obtained for stroke volume (μL) for D2.WT mice and D2.mdx mice treated as described in Example 6.

FIG. 15B is a graphic illustration of data obtained for ejection fraction (%) for D2.WT mice and D2.mdx mice treated as described in Example 6.

FIG. 15C is a graphic illustration of data obtained for fractional shortening (%) for D2.WT mice and D2.mdx mice treated as described in Example 6.

FIG. 15D is a graphic illustration of data obtained for cardiac output (mL/min.) for D2.WT mice and D2.mdx mice treated as described in Example 6.

FIG. 16A is a graphic illustration of data obtained for the maximum force (mN) generated by the extensor digitorum longus (EDL) muscle for D2.WT mice and D2.mdx mice treated as described in Example 6.

FIG. 16B is a graphic illustration of data obtained for the specific force (N/cm²) generated by the extensor digitorum longus (EDL) muscle for D2.WT mice and D2.mdx mice treated as described in Example 6.

FIG. 16C is a graphic illustration of data obtained for the eccentric (ECC) contractions of the extensor digitorum longus (EDL) muscle for D2.WT mice and D2.mdx mice treated as described in Example 6.

### DEFINITIONS

Definitions of specific functional groups and chemical terms are described in more detail below. The chemical elements are identified in accordance with the Periodic Table of the Elements, GAS version, Handbook of Chemistry and Physics, 7Sh Ed., inside cover. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

It is to be appreciated that certain aspects, modes, embodiments, variations and features of the technology are described below in various levels of detail in order to provide a substantial understanding of the present disclosure. The definitions of certain terms as used in this specification are provided below. Unless defined otherwise, all technical and scientific terms used herein generally have the same meaning as commonly understood by one of ordinary skill in the art to which this technology belongs.

As used in this specification and the appended claims, the singular forms “a,” “an,”and “the” include plural references unless the content clearly dictates otherwise. For example, reference to “a cell” includes a combination of two or more cells, and the like.

As used herein, the “administering” or the “administration” of a therapeutic agent (e.g., a peptide or mixture of peptides, individually or as formulated) or drug (e.g., PMO, PPMO or AAV vector conjugated PMO) to a subject refers to any route of introducing or delivering to a subject a compound (e.g., peptide, mixture of peptides, or combination of peptide(s), PMO, PPMO or AAV conjugated PMO and/or other therapeutic agent(s)) to perform its/their intended function. Administration can be carried out by any suitable route, such as oral administration. Administration can be carried out subcutaneously. Administration can be carried out intravenously. Administration can be carried out intraocularly. Administration can be carried out retro-orbitally. Administration can be carried out systemically. Administration may be carried out topically. Administration may be carried out intranasally. Administration may be carried out intraperitoneally. Administration may be carried out intradermally. Administration may be carried out ophthalmically. Administration may be carried out intrathecally. Administration may be carried out intracerebroventricularly. Administration may be carried out iontophoretically. Administration may be carried out transmucosally. Administration may be carried out intravitreally. Administration may be carried out intramuscularly. Administration includes self-administration, the administration by another or the administration by a device (e.g., a pump).

As used herein, to “ameliorate” or “ameliorating” a disease, disorder or condition refers to results that, in a statistical sample or specific subject, make the occurrence of the disease, disorder or condition (or a sign or symptom thereof) better or more tolerable in a subject administered a therapeutic agent(s) relative to a control sample, control subject or control group of subjects not administered the therapeutic agent(s).

As used herein, the term “amino acid” refers to naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein the term or phrase “carrier” and “pharmaceutically acceptable carrier” refer to a diluent, adjuvant, excipient, or vehicle with which a therapeutic agent (e.g., a peptide/mixture of peptides/compound/composition) is administered or formulated for administration. Non-limiting examples of such pharmaceutically acceptable carriers include liquids, such as water, saline, and oils; and solids, such as gum acacia, gelatin, starch paste, talc, keratin, colloidal silica, silica particles (nanoparticles or microparticles), urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the phrase “delaying the onset of” refers to, in a statistical sample, postponing, hindering, or causing a disease, disorder or condition (or a sign, symptom or condition thereof) to occur more slowly than normal in a sample or subject administered a therapeutic agent(s) relative to a control sample, control subject or control group of subjects not administered the therapeutic agent(s).

As used herein, the term “effective amount” refers to a quantity of the peptide(s) of Formula A (including all specific embodiments thereof disclosed herein) and/or any other therapeutic agent(s) sufficient to achieve a desired therapeutic and/or prophylactic effect, e.g., an amount that treats, inhibits, reduces, ameliorates, or delays the onset of muscular dystrophy, or a cardiomyopathy, loss of ambulation, or respiratory distress associated with the muscular dystrophy in a subject so diagnosed, or the signs symptoms or conditions associated therewith when “administered” or “co-administered,” where, for example, the peptide(s) and PMO(s) and/or PPMO(s) may be administered simultaneously, sequentially, or by separate administration. In the context of therapeutic or prophylactic applications, in some embodiments, the amount of a composition administered to the subject will depend on the type and severity of the disease and on the characteristics of the individual, such as general health, age, sex, body weight, and tolerance to drugs. The skilled artisan will be able to determine appropriate dosages depending on these and other factors. The peptide(s) and other therapeutic agent(s) disclosed herein can be administered in an effective amount prior to the onset of one or more symptoms associated with muscular dystrophy (e.g., DMD or BMD), or in response to a symptom that occurs in a subject suffering from muscular dystrophy (e.g., DMD or BMD). The combination of peptide(s) and other therapeutic agent(s) disclosed herein can be administered simultaneously, sequentially, or by separate administration as would be appropriate, for example, based on accepted practice and their label as approved by a regulatory authority (e.g., the FDA).

In the methods described herein, therapeutic compounds, or pharmaceutically acceptable salts, stereoisomers, mixtures of stereoisomers, tautomers, hydrates, and/or solvates thereof, may be administered to a subject diagnosed as having muscular dystrophy (e.g., DMD, or BMD.) In some embodiments, the methods described herein, therapeutic compounds, or pharmaceutically acceptable salts, stereoisomers, mixtures of stereoisomers, tautomers, hydrates, and/or solvates thereof, may be administered to a subject to address one or more signs, symptoms, or risk factors associated with muscular dystrophy in a patient diagnosed with or suspected of having muscular dystrophy. For example, a “therapeutically effective amount” of therapeutic compounds (e.g., a peptide, mixture of peptides, PMO(s) and/or PPMO(s)) and/or other therapeutic agent(s) includes levels at which the presence, frequency, or severity of one or more signs, symptoms, or risk factors of muscular dystrophy are inhibited, reduced or eliminated. In some embodiments, a therapeutically effective amount of a/the therapeutic compound(s) augments the production of dystrophin in the subject when compared to a control subject not administered a/the therapeutic compound(s).

As used herein, the term “hydrate” refers to a compound (e.g., a peptide or mixture of peptides) which is associated with water. The number of the water molecules contained in a hydrate of a compound may be (or may not be) in a definite ratio to the number of the compound molecules in the hydrate.

As used herein, the term “inhibit,” “inhibits,” or “inhibiting” refers to reduction in an objectively measurable amount or degree of a disease, disorder or condition (or a sign, or symptom thereof) to occur more slowly than normal in a sample or subject administered a therapeutic agent(s) relative to a control sample, control subject or control group of subjects not administered the therapeutic agent(s). In some embodiments, to “inhibit” or “inhibiting” means reducing by at least 1-5 percent compared to a sample or subject administered a therapeutic agent(s) relative to a control sample, control subject or control group of subjects not administered the therapeutic agent(s). In various individual embodiments, inhibit or inhibiting means reducing by at least 2, 3, 4, 5, 6, 7, 8, 9, 10, 15, 20, 25, 30, 33, 40, 50, 60, 67, 70, 75, 80, 90, 95, or 99 percent compared to a sample or subject administered a therapeutic agent(s) relative to a control sample, control subject or control group of subjects not administered the therapeutic agent(s).

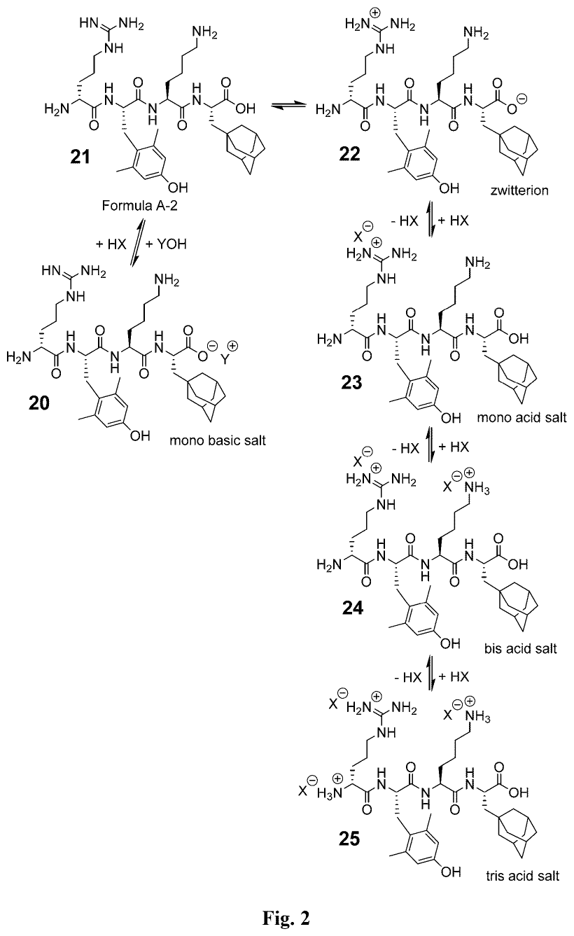

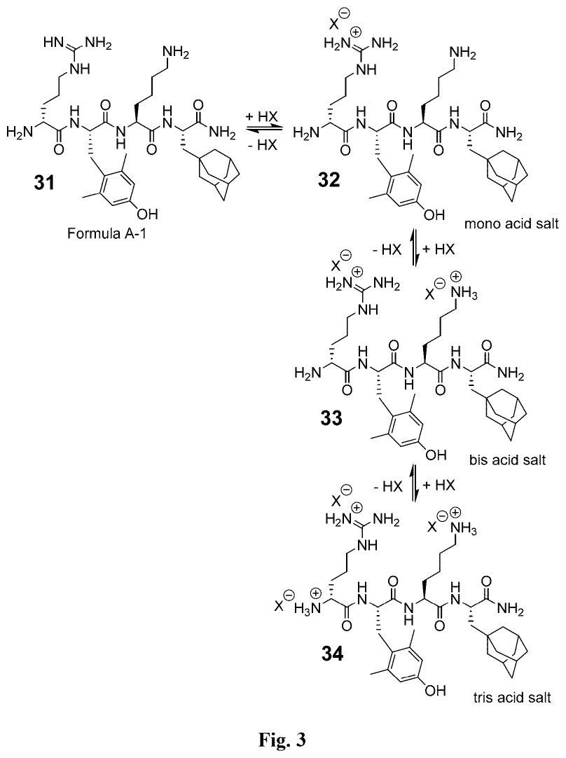

As used herein, the term “pharmaceutically acceptable salt” refers to a salt of a therapeutically active compound (e.g., a peptide, mixture of peptides, a PMO and/or PPMO) that can be prepared with relatively nontoxic acids or bases, depending on the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic and p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66:1-19 (1977)). Certain specific compounds of the present application may contain both basic and acidic functionalities that allow the compounds to be converted into either base or acid addition salts. These salts may be prepared by methods known to those skilled in the art. Other pharmaceutically acceptable carriers known to those of skill in the art are suitable for the present technology. In some embodiments, the compound is a zwitterion (an intramolecular salt). Exemplary salt forms of the peptide H-D-Arg-2′6′-Dmt-Lys-Phe-OH (A-2) are illustrated in FIG. 2. Exemplary salt forms of the peptide H-D-Arg-2′6′-Dmt-Lys-Phe-NH₂ (A-1) are illustrated in FIG. 3.

As used herein, “peptide-conjugated PMOs (PPMOs)” refers to PMOs to which a cell penetrating peptide is linked in order to improve cellular uptake of the PMO. See: Tsoumpra et al. (2019) “Peptide-conjugated antisense based splice-correction for Duchenne muscular dystrophy and other neuromuscular diseases” EBioMedicine, 45, 630-645; doi.org/10.1016/j.ebiom.2019.06.036.

As used herein, “phosphorodiamidate morpholino oligomers (PMOs)” refer to synthetic oligomers comprising a natural nucleobase linked to methylenemorpholine rings linked through phosphorodiamidate groups instead of a phosphate backbone. See: Summerton JE (2017). “Invention and Early History of Morpholinos: From Pipe Dream to Practical Products”. Morpholino Oligomers. Methods in Molecular Biology. 1565. Humana Press (Springer). pp. 1-15. In some embodiments, the PMO comprises an appropriately designed exon-skipping oligomer that is relevant to the dystrophin lesion in a subject in need thereof, wherein the subject has been diagnosed with or is suspected of having a muscular dystrophy, such as DMD or BMD. By way of example, but not by limitation, the PMO comprises an antisense oligomer of about 20-50 nucleotides in length, or a pharmaceutically acceptable salt thereof, capable of binding a selected target in human dystrophin pre-mRNA to induce exon skipping in the human dystrophin gene, wherein the antisense oligomer comprises a sequence of bases that specifically hybridizes to a dystrophin exon target region. By way of example, but not by limitation, the PMO may be chemically linked to a cell penetrating peptide that improves cellular uptake of the PMO (e.g., a PPMO). Illustrative, non-limiting examples of PMOs include any one or more the PMOs selected from Eteplirsen (Exondys 51®), Golodirsen (Vyondys 53™), Viltolarsen (Viltepso®), and Casimersen (Amondys 45™). Two or more antisense oligomers may be used together to induce exon skipping of single or multiple exons.

As used herein the term “prevent”, “prevents”, “preventing” or “prevention” refers to, in a statistical sample, reducing the occurrence of a disease state, disorder, symptom, or condition in a sample or subject administered a therapeutic agent(s) relative to a control sample, control subject or control group of subjects not administered the therapeutic agent(s).

As used herein the term “progression” refers to the process of moving gradually towards a more advanced state of a disease, disorder, or condition (or a sign or symptom thereof) in a subject.

As used herein the term “prophylactic” refers to an action intended to prevent a disease state, disorder, symptom, or condition from occurring.

As used herein, the term “separate” with respect to a therapeutic use refers to an administration of at least two active ingredients (i.e., at least two difference therapeutic agents) at the same time or at substantially the same time by different routes. The “active ingredients” can, for example, be a peptide or mixture of peptides. The “active ingredients” can, for example, be at least one PMO (such as Eteplirsen (Exondys 51®), Golodirsen (Vyondys 53™), Viltolarsen (Viltepso®), or Casimersen (Amondys 45™)) and/or PPMO as disclosed herein. In some embodiments, the active ingredients can, for example, be an angiotensin-converting enzyme (ACE) inhibitor, an angiotensin receptor blocker (ARB), a beta blocker, a diuretic, or a mineralocorticoid receptor antagonist.

As used herein, the term “sequential” with respect to a therapeutic use refers to administration of at least two active ingredients (i.e., at least two difference therapeutic agents) at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this definition.

As used herein, the term “simultaneous” with respect to a therapeutic use refers to the administration of at least two active ingredients (i.e., at least two difference therapeutic agents) by the same or different route but at the same time or at substantially the same time.

As used herein, the term “solvate” refers to forms of a compound (e.g., a peptide or mixture of peptides) that are associated with a solvent, usually by a solvolysis reaction. This physical association may include hydrogen bonding. Conventional solvents include water, methanol, ethanol, isopropanol, acetic acid, ethyl acetate, acetone, hexane(s), dimethyl sulfoxide (DMSO), tetrahydrofuran (THF), diethyl ether, and the like.

As used herein, the term “subject” and “patient” refers to a living animal. In various embodiments, a subject is a mammal. In some embodiments, a subject is a non-human mammal, including, without limitation, a mouse, rat, hamster, guinea pig, rabbit, sheep, goat, cat, dog, pig, minipig, horse, cow, or non-human primate. In some embodiments, the subject is a human. As used herein, the terms “subject” and “patient” are or can be used interchangeably.

It is also to be appreciated that the various modes of treatment or prevention of medical conditions as described herein, in some embodiments, are intended to mean “substantial,” which includes total but also less than total treatment or prevention, and wherein some biologically or medically relevant result is achieved.

As used herein the term “synergistic therapeutic effect” refers to a greater-than-additive therapeutic effect that is produced by a combination of at least two therapeutic agents, and which exceeds that which would otherwise result from the individual administration of the agents. For example, lower doses of two or more therapeutic agents may be used in treating muscular dystrophy, resulting in increased therapeutic efficacy and decreased side-effects of one or both of the therapeutic agents. For example, the co-administration of a peptide or mixture of peptides as disclosed herein (e.g., a peptide of Formula A, A-1, A-2, A-3, A-4, A-5, A-6, A-7, A-8, A-9, A-10, A-11-1, A-11-2, A-12-1, A-12-2, A-13-1, A-13-2, A-14-1, A-14-2, A-15-1 or A-15-2) may enhance the effectiveness of the administration of an additional therapeutic agent such as a PMO, or PPMO, particularly with respect to treating the subject's cardiomyopathy, loss of ambulation and/or respiratory distress.

As used herein, the term “tautomer” refers to compounds (e.g., a peptide or mixture of peptides) that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

As used herein, the term “treat”, “treats”, “treating” or “treatment” refers to therapeutic treatment, wherein the object is to reduce, alleviate or delay onset of the progression or advancement of, and/or reverse the progression of the targeted pathological disease, disorder or condition or its associated signs and/or symptoms in a sample or subject administered a therapeutic agent(s) relative to a control sample, control subject or control group of subjects not administered the therapeutic agent(s).

### DETAILED DESCRIPTION

The present disclosure relates generally to compounds (e.g., peptides), compositions (e.g., medicaments or formulations comprising the compounds and/or peptides), methods, and uses for treating, preventing, inhibiting, ameliorating, or delaying the onset of muscular dystrophy (e.g., DMD or BMD), or the signs, symptoms or conditions associated therewith, in a mammalian subject in need thereof. In some embodiments, the subject suffering from muscular dystrophy (e.g., DMD or BMD), is administered a therapeutically effective amount of a peptide or mixture of peptides, optionally in combination with one or more PMOs and/or PPMOs as described in more detail below, or a pharmaceutically acceptable salt, hydrate, solvate and/or tautomer thereof. Alternatively or additionally, the mammalian subject is optionally administered an additional therapeutic agent or agents, such as, a corticosteroid, an angiotensin-converting enzyme (ACE) inhibitor, an angiotensin receptor blocker (ARB), a beta blocker, a diuretic, a mineralocorticoid receptor antagonist or any two or more of the foregoing.

Generally, the mammalian subject to be treated according to the methods disclosed herein will harbor a genetic permutation that affects the production and/or function of dystrophin protein. In some embodiments of the method, the genetic permutation is an insert, deletion, duplication, frameshift, or nonsense mutation related to the production of dystrophin protein but regardless, the genetic mutation causes a progressive debilitating condition that eventually will severely impact the subject's life as discussed above in the Introduction and ultimately lead to death of the subject. In some embodiments of the method, administering the peptide or mixture of peptides, is effective in treating, preventing, ameliorating, inhibiting or delaying the onset of muscular dystrophy, the cardiomyopathies associated with muscular dystrophy, the difficulties associated with ambulation associated with muscular dystrophy, or the respiratory distress associated with muscular dystrophy, or its/their associated conditions, signs or symptoms. In some embodiments of the method, administering the peptide or mixture of peptides, optionally in combination with one or more PMOs and/or PPMOs, to the subject results in a measurable benefit to the subject as compared to a control subject not administered the peptide or mixture of peptides, optionally in combination with the PMO(s) and/or PPMO(s). Said peptide or mixture of peptides, alone or in combination with one or more PMOs and/or PPMOs can be administered alone, in a composition or formulation (e.g., medicament), and/or in combination with one or more additional therapeutic agents such as a corticosteroid, an angiotensin-converting enzyme (ACE) inhibitor, an angiotensin receptor blocker (ARB), a beta blocker, a diuretic, or a mineralocorticoid receptor antagonist. In some embodiments, the subject is human.

In some embodiments, the peptide, or peptides of a mixture, is/are a peptide/peptides of Formula A:

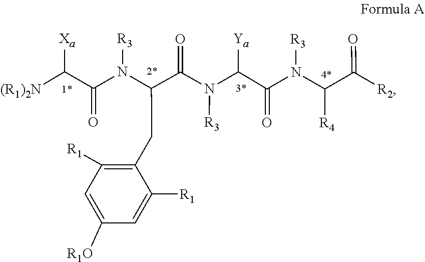

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

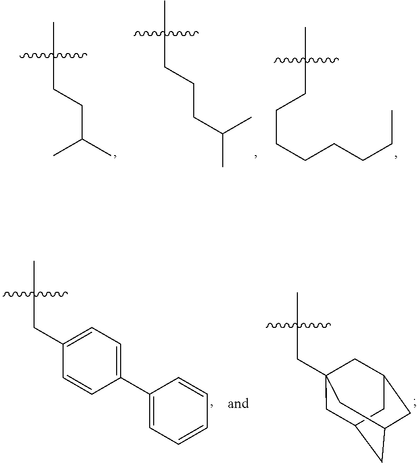

Xₐ is selected from

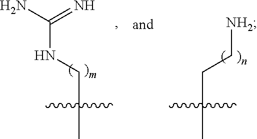

Yₐ is selected from

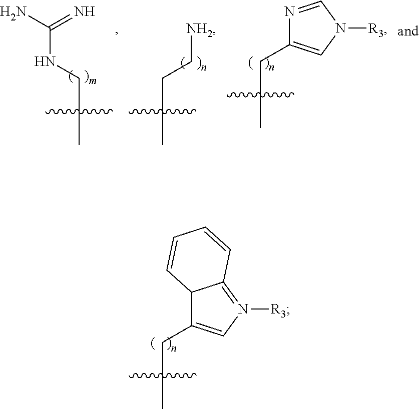

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom.

The peptide or a peptide within a mixture of peptides of Formula A can be of Formula A-1 (H-D-Arg-2,6-Dmt-Lys-ADM-NH₂; wherein Dmt is 2,6-dimethyltyrosine and ADM is adamantylalanine):

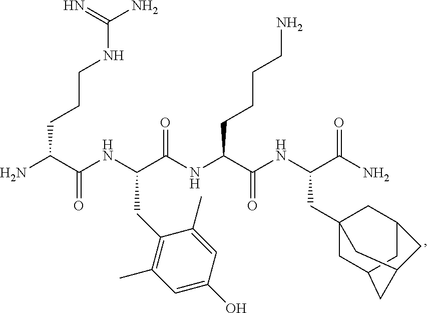 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O]

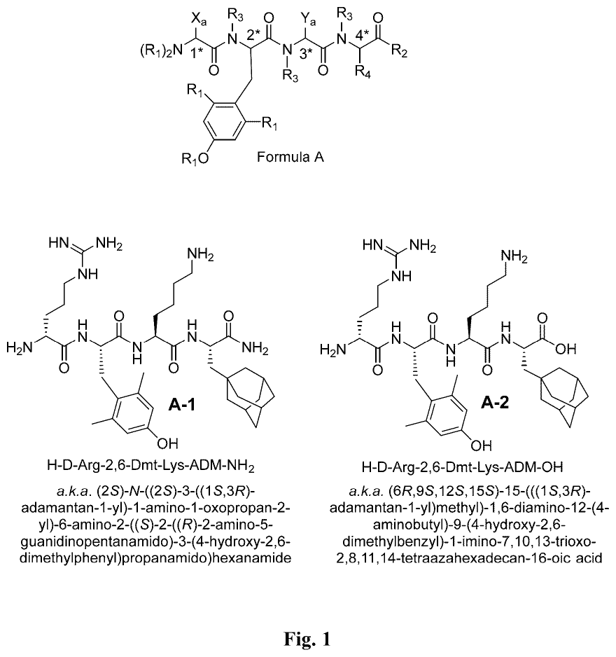

or a pharmaceutically acceptable salt, hydrate, solvate and/or tautomer thereof (See also FIG. 1 for names of this peptide). The peptide or a peptide within a mixture of peptides of Formula A can be of Formula A-2 (H-D-Arg-2,6-Dmt-Lys-ADM-OH):

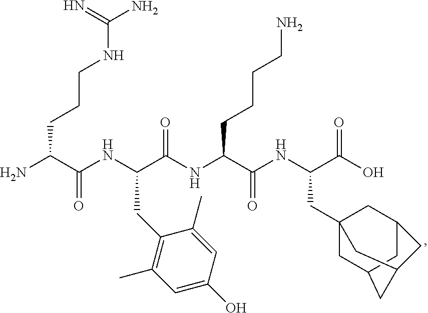 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate and/or tautomer thereof (See also FIG. 1 for names of this peptide). The peptide or a peptide within a mixture of peptides of Formula A can be a peptide of Formula A-3 (D-Arg-Dmt-Arg-AMD-NH₂):

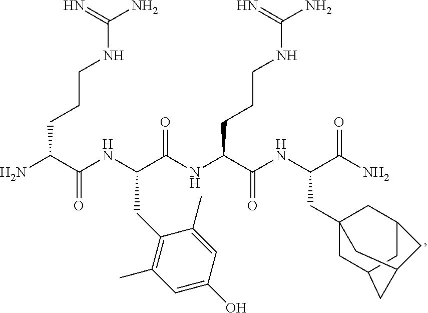 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O]

or a pharmaceutically acceptable salt, hydrate, solvate and/or tautomer thereof. The peptide or a peptide within a mixture of peptides of Formula A can be a peptide of Formula A-4 (D-Arg-Dmt-Arg-AMD-OH):

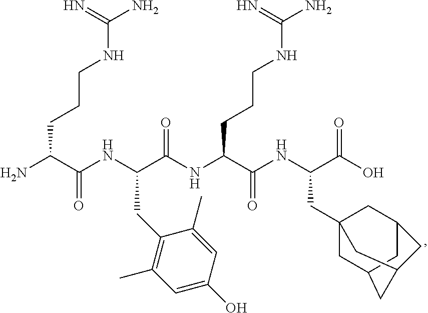 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O]

or a pharmaceutically acceptable salt, hydrate, solvate and/or tautomer thereof. The peptide or a peptide within a mixture of peptides of Formula A can be a peptide of Formula A-5 (D-Arg-Dmt-Orn-AMD-NH₂, wherein Orn is the amino acid ornithine):

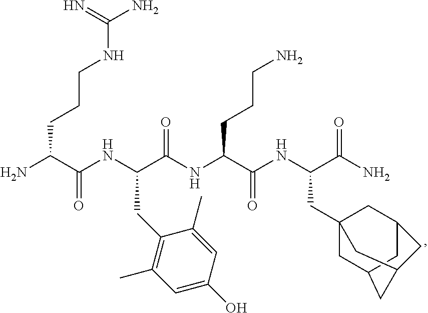 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

or a pharmaceutically acceptable salt, hydrate, solvate and/or tautomer thereof. The peptide or a peptide within a mixture of peptides of Formula A can be a peptide of Formula A-6 (D-Arg-Dmt-Orn-AMD-OH):

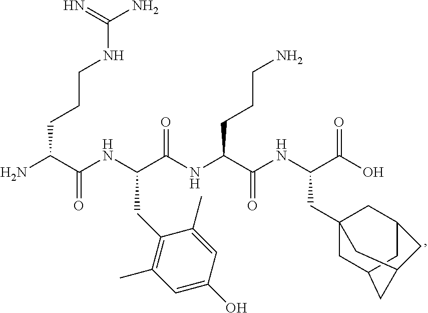 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

or a pharmaceutically acceptable salt, hydrate, solvate and/or tautomer thereof. The peptide or a peptide within a mixture of peptides of Formula A can be a peptide of Formula A-7 (D-Arg-Tyr-Lys-AMD-NH₂):

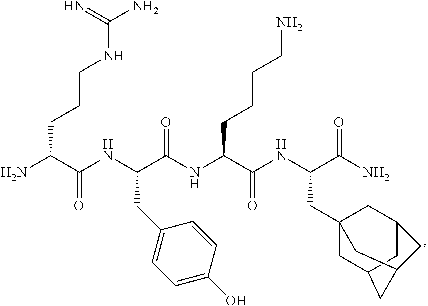 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O]

or a pharmaceutically acceptable salt, hydrate, solvate and/or tautomer thereof. The peptide or a peptide within a mixture of peptides of Formula A can be a peptide of Formula A-8 (D-Arg-Tyr-Lys-AMD-OH):

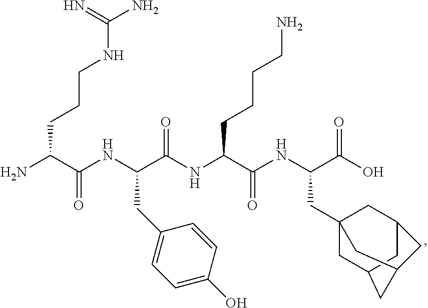 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

or a pharmaceutically acceptable salt, hydrate, solvate and/or tautomer thereof. The peptide or a peptide within a mixture of peptides of Formula A can be a peptide of Formula A-9 (D-Arg-4-Me-Dmt-Lys-AMD-NH₂, wherein 4-Me-Dmt is 4-methyl-2,6-dimethyltyrosine):

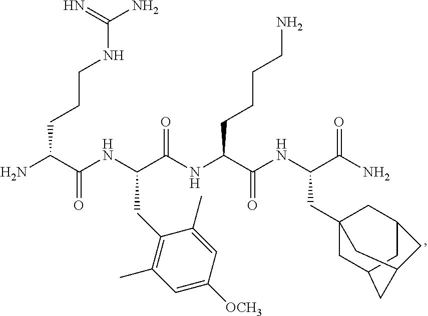 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1]

or a pharmaceutically acceptable salt, hydrate, solvate and/or tautomer thereof. The peptide or a peptide within a mixture of peptides of Formula A can be a peptide of Formula A-10 (D-Arg-4-Me-Dmt-Lys-AMD-OH):

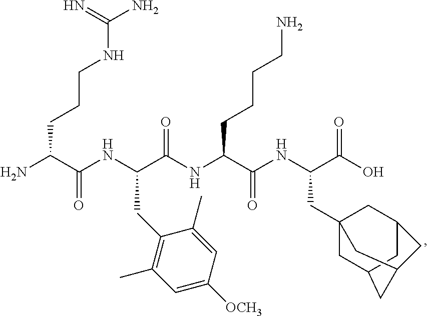 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

or a pharmaceutically acceptable salt, hydrate, solvate and/or tautomer thereof. The peptide or a peptide within a mixture of peptides of Formula A can be a peptide of Formula A-11-1 (D-Arg-Dmt-Lys-BPA-NH ₂, wherein BPA is biphenylalanine):

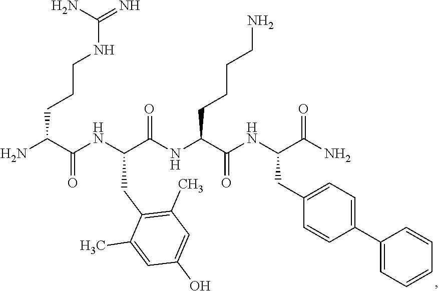 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O]

or a pharmaceutically acceptable salt, hydrate, solvate and/or tautomer thereof. The peptide or a peptide within a mixture of peptides of Formula A can be a peptide of Formula A-11-2 (D-Arg-Dmt-Lys-BPA-OH):

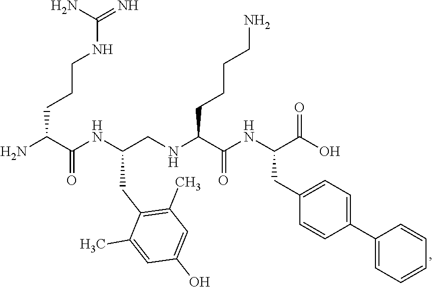 [SMILES: Cc1cc(O)cc(C)c1C[C@@H](CN[C@@H](CCCCN)C(=O)N[C@@H](Cc1ccc(-c2ccccc2)cc1)C(=O)O)NC(=O)[C@H](N)CCCNC(=N)N]

or a pharmaceutically acceptable salt, hydrate, solvate and/or tautomer thereof. The peptide or a peptide within a mixture of peptides of Formula A can be a peptide of Formula A-12-1 (D-Arg-Dmt-Lys-n-HA-NH₂, wherein n-HA is n-heptylalanine):

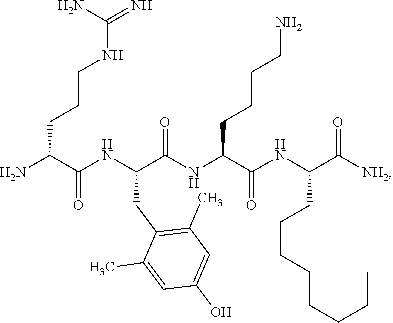 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O]

or a pharmaceutically acceptable salt, hydrate, solvate and/or tautomer thereof. The peptide or a peptide within a mixture of peptides of Formula A can be a peptide of Formula A-12-2 (D-Arg-Dmt-Lys-n-HA-OH):

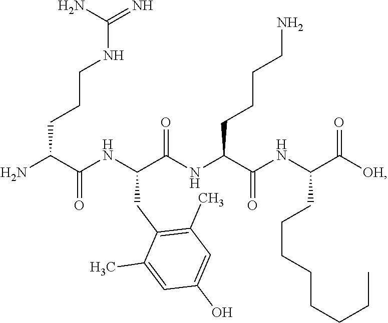 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

or a pharmaceutically acceptable salt, hydrate, solvate and/or tautomer thereof. The peptide or a peptide within a mixture of peptides of Formula A can be a peptide of Formula A-13-1 (D-Arg-Dmt-Trp-IBA-NH₂, wherein IBA is isobutylalanine):

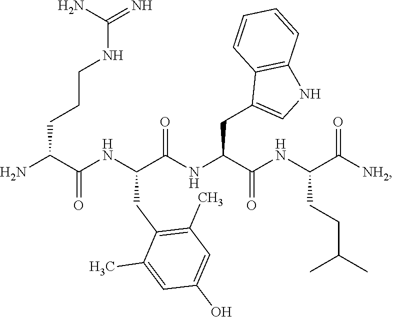 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O]

or a pharmaceutically acceptable salt, hydrate, solvate and/or tautomer thereof. The peptide or a peptide within a mixture of peptides of Formula A can be a peptide of Formula A-13-2 (D-Arg-Dmt-Trp-IBA-OH):

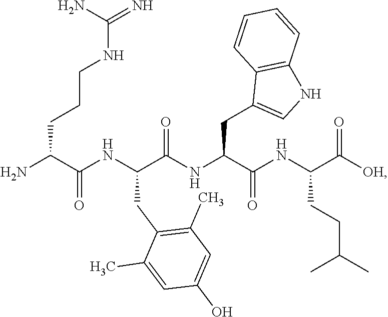 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

or a pharmaceutically acceptable salt, hydrate, solvate and/or tautomer thereof. The peptide or a peptide within a mixture of peptides of Formula A can be a peptide of Formula A-14-1 (D-Arg-Dmt-n-Me-Trp-BPA-NH₂, wherein n-Me-Trp is N-methyltryptophan):

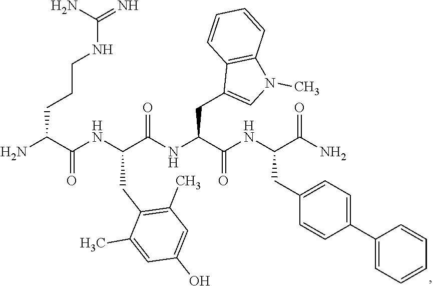 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O]

or a pharmaceutically acceptable salt, hydrate, solvate and/or tautomer thereof. The peptide or a peptide within a mixture of peptides of Formula A can be a peptide of Formula A-14-2 (D-Arg-Dmt-n-Me-Trp-BPA-OH):

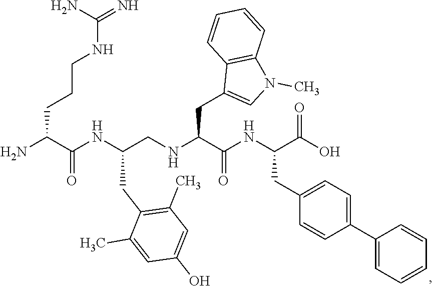 [SMILES: Cc1cc(O)cc(C)c1C[C@@H](CN[C@@H](Cc1cn(C)c2ccccc12)C(=O)N[C@@H](Cc1ccc(-c2ccccc2)cc1)C(=O)O)NC(=O)[C@H](N)CCCNC(=N)N]

or a pharmaceutically acceptable salt, hydrate, solvate and/or tautomer thereof. The peptide or a peptide within a mixture of peptides of Formula A can be a peptide of Formula A-15-1 (D-Arg-Dmt-His —BPA-NH₂,):

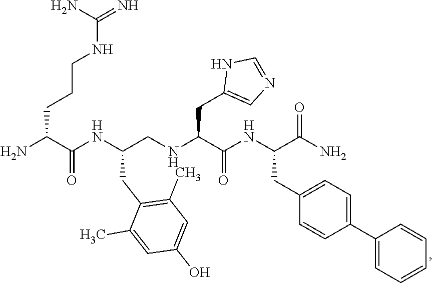 [SMILES: Cc1cc(O)cc(C)c1C[C@@H](CN[C@@H](Cc1cnc[nH]1)C(=O)N[C@@H](Cc1ccc(-c2ccccc2)cc1)C(N)=O)NC(=O)[C@H](N)CCCNC(=N)N]

or a pharmaceutically acceptable salt, hydrate, solvate and/or tautomer thereof. The peptide or a peptide within a mixture of peptides of Formula A can be a peptide of Formula A-15-2 (D-Arg-Dmt-His —BPA-OH):

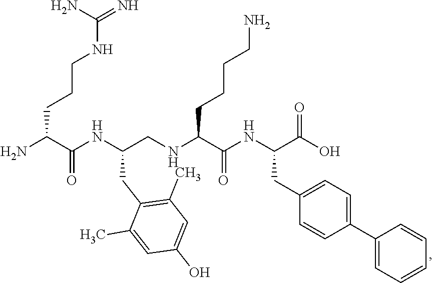 [SMILES: Cc1cc(O)cc(C)c1C[C@@H](CN[C@@H](CCCCN)C(=O)N[C@@H](Cc1ccc(-c2ccccc2)cc1)C(=O)O)NC(=O)[C@H](N)CCCNC(=N)N]

or a pharmaceutically acceptable salt, hydrate, solvate and/or tautomer thereof. In some embodiments, one or more hydrogen atoms of the peptide or peptides of Formulas A (e.g., peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2) are optionally substituted with a deuterium or fluorine atom.

The peptide(s) can be administered individually or as a mixture comprising two or more of the peptides as defined herein. The peptide or mixture of peptides can be administered alone, in a formulation (e.g. medicament) or in combination with one or more other therapeutic agents. In some embodiments, the pharmaceutically acceptable salt of the peptide or peptides can be selected from a hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate or trifluoroacetate salt. In some embodiments, the composition, formulation or medicament can be prepared by formulating the tris —HCl salt of the peptide or peptides.

In some embodiments, mixtures of two or more of the above described peptides are used as a/the therapeutic agent. Such mixtures may be present intentionally (e.g., by mixing the peptides post synthesis) or fortuitously (e.g., by the hydrolysis of a C-terminal amide to a C-terminal carboxylic acid). Whenever reference is made herein to a peptide or mixture of peptides, it is implied and intended that each individual peptide of said peptide or mixture of peptides can exist as a free acid/base, in zwitterionic form or in any salt form, including in a pharmaceutically acceptable salt form (e.g., See: FIG. 2 and FIG. 3 for various salt forms of the peptides of Formula A-1 & A-2).

For example, FIG. 2 illustrates various forms that the peptide of Formula A-2 can take and FIG. 3 illustrates various forms that the peptide of Formula A-1 can take. With reference to FIG. 2, (20) illustrates a mono-basic salt form of the peptide of Formula A-2, wherein the C-terminal carboxylate has been ionized as its base-salt. As illustrated, the basic generic salt represented by YOH can ionize to produce Y+ and OH− and thereby ionize the peptide of Formula A-2 (21) to form (20). The generic basic salt represented by YOH could be, for example, sodium hydroxide (NaOH), potassium hydroxide (KOH) or lithium hydroxide (LiOH). The mono-basic salt form (20) can be protonated with acid (e.g., represented by HX wherein H+ is the proton and X⁻ represents the counterion and, for example, is embodied by acids such as HCl, HBr or HI) to form the peptide of Formula A-2 (21). However, the peptide of Formula A-2 (21) can also be represented in zwitterionic form (22) resulting from the internal distribution of a proton between the carboxylate and one of the basic groups. The peptide of Formula A-2 ((21) or (22)) can be further protonated with a single equivalent of acid (i.e., HX) to thereby produce a mono-acid salt (23). The mono-acid salt (23) can be further acidified with another equivalent of acid (i.e., HX) to thereby produce a bis —acid salt (24). The bis —acid salt (24) can be further acidified with another equivalent of acid (i.e., HX) to thereby produce a (tri- or) tris —acid salt (25). A similar analysis of FIG. 3 can be done to determine the various salt forms of the peptide of Formula A-3. In some embodiments, the peptide or peptides of the mixture of peptides is/are formulated from a mono-acid salt form. In some embodiments, the peptide or peptides of the mixture of peptides is/are formulated from a bis —acid salt form. In some embodiments, the peptide or peptides of the mixture of peptides is/are formulated from a tri-acid (a.k.a., a tris —acid) salt form.

One of skill in the art will appreciate that these transitions between the free-base and various salt forms are easily accomplished by using an appropriate amount of acid or base. One of skill in the art will further appreciate that such transitions between salt forms are also applicable to any peptides represented by Formula A, including without limitation the peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2. The peptide may be formulated as a pharmaceutically acceptable salt as defined herein.

Certain compound(s)/peptide(s) disclosed in the present disclosure can exist in unsolvated forms as well as solvated forms, including hydrated forms. Solvated forms can exist, for example, because it is difficult or impossible to remove all the solvent from the peptide post synthesis. In general, the solvated forms are equivalent to unsolvated forms and are encompassed within the scope of the present disclosure.

Certain compound(s)/peptide(s) of the present disclosure may exist in crystalline form, multiple crystalline forms, amorphous forms, polymorphous forms or any combination of the foregoing. Certain compound(s)/peptide(s) of the present disclosure may exist in various tautomeric forms. Certain compound(s)/peptide(s) of the present disclosure may exist in various salt forms or mixtures of salt forms. In general, all physical forms of the compound(s)/peptide(s) disclosed herein are deemed equivalent for the uses contemplated by the present disclosure and are intended to be within the scope of the present disclosure.

Certain compound(s)/peptide(s) disclosed in the present disclosure can exist in various tautomeric forms. In general, all tautomeric forms of the peptides disclosed herein are equivalent with respect to their application to the embodiments of the present disclosure and deemed to be encompassed within the scope of the present disclosure.

### Chiral/Stereochemistry Considerations

Peptides/compounds described herein can comprise one or more asymmetric centers, and thus can exist in various isomeric forms, e.g., as enantiomers and/or diastereomers in relation to others of the peptides disclosed herein (i.e., stereoisomers). Chiral centers in illustrated structures (including the claims) may be identified herein by use of an asterisk (*). For example, the compounds described herein can be in the form of an individual enantiomer, diastereomer or geometric isomer, or can be in the form of a mixture of stereoisomers, including racemic mixtures and mixtures enriched in one or more stereoisomer, unless otherwise specifically illustrated as a particular stereoisomer. Stereoisomers can be isolated from mixtures by methods known to those skilled in the art, including chiral high-pressure liquid chromatography (HPLC) and the formation and crystallization of chiral salts. Alternatively, preferred stereoisomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure of the present application additionally encompasses compounds described herein as individual isomers (or stereoisomers) substantially free of other isomers (or stereoisomers), and alternatively in some embodiments, as mixtures of various isomers (or stereoisomers). Any of the compounds disclosed herein can be prepared as substantially pure stereoisomers (e.g., substantially pure stereoisomer) if desired.

As used herein, a pure enantiomeric compound is substantially free from other enantiomers or stereoisomers of the compound (i.e., in enantiomeric excess (ee) of the other stereoisomer); as purity is a relative term in the sense that it is exceedingly difficult to achieve 100% purity. In other words, an “S” form of the compound is substantially free from the “R” form of the compound and is, thus, in enantiomeric excess of the “R” form. With respect to common amino acids found in nature (which are more commonly described in terms of “D” and “L” enantiomer), it is to be understood that for a “D”-amino acid the configuration is “R” and for an “L”-amino acid, the configuration is “S”, with the known exception of L-cysteine which is R-cysteine and vice-versa. The measure of stereoisomeric purity is typically referred to using the designation “ee”, which refers to enantiomeric excess. The higher the “ee” value, the greater the stereochemical purity of the substance/compound. In some embodiments, ‘substantially free’ or ‘substantially pure,’ refers to: (i) an aliquot of an “R” form of a peptide containing a specified chiral center contains less than 2% “S” form at that specified chiral center; or (ii) an aliquot of an “S” form of a peptide containing a specified chiral center contains less than 2% “R” form at that specified chiral center. In some embodiments, the term “enantiomerically pure” or “pure enantiomer” denotes that the compound or peptide comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the particularly identified enantiomer (e.g., as compared with the other enantiomer). Typically, when used as a therapeutic agent, the peptides disclosed herein will be substantially free of other stereoisomers (enantiomers or diastereoisomers) of the designated peptide. It is to be understood that when administered to a human subject, the peptide or peptides selected for use in methods, formulation of compositions or medicaments, or presented for uses will often be substantially free of enantiomeric or distereomeric impurity peptides.

In certain embodiments, the weights are based upon total weight of all enantiomers or stereoisomers of the compound. When illustrated herein, a composition drawn in a particular stereoisomeric form would be considered to be in substantially pure form (i.e., substantially free of undesired enantiomeric or diastereomeric impurities) for administration to a subject when such level of stereoisomeric purity is approved for administration by a medicines regulating authority of a country for administration of such a therapeutic agent/composition for treating a particular indication or condition.

In the compositions provided herein, an enantiomerically pure compound (e.g., a peptide) can be present with other active or inactive ingredients. For example, a pharmaceutical composition comprising enantiomerically pure “R” form compound (i.e., the is substantially free of other stereoisomers) can comprise, for example, about 90% excipient and about 10% enantiomerically pure “R” form of the compound/peptide. In certain embodiments, the therapeutic agent (i.e., compound or peptide) can be formulated with little or no excipient or carrier.

### Peptide Synthesis

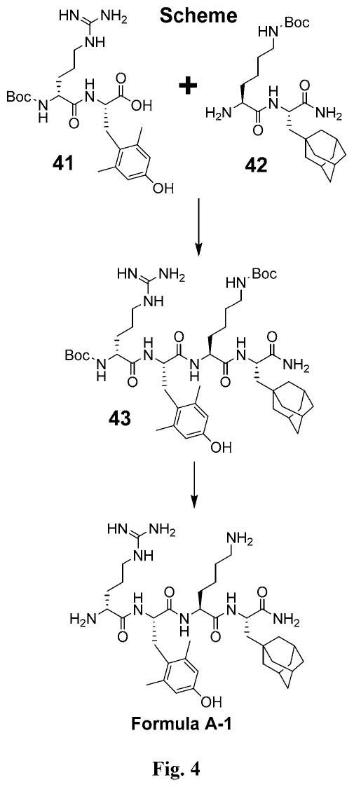

The peptides may be synthesized by any of the methods well known in the art. The peptides can be prepared using solid-phase synthesis methodology. The peptides can be synthesized by using solution-phase methodology. Suitable methods for chemically synthesizing the peptides include, for example, those described in any of WO 2004/070054, and/or WO 2020/131283. For example, the tetrapeptide of Formula A-1 can be prepared by at 2+2 synthesis by preparing the partially protected dipeptides 41 and 42 (FIG. 4) followed by coupling the dipeptides to produce the protected tetrapeptide, followed by deprotection; essentially as described in WO2016/144905, Examples 5, wherein the phenylalanine amide is replaced with the appropriate adamantyl containing amino acid amide. Also see: WO2020/131283. In some embodiments, the peptides are C-terminal amides and in some embodiments the peptides are C-terminal carboxylic acids. The methodology used to prepare the peptide may be influenced by the desired C-terminal modification.

For example, the peptides disclosed herein can be prepared using any peptide synthesis method, such as conventional liquid-phase peptide synthesis or solid-phase peptide synthesis, or by peptide synthesis by means of an automated peptide synthesizer (Kelley et al., Genetics Engineering Principles and Methods, Setlow, J. K. eds., Plenum Press NY. (1990) Vol. 12, pp. 1 to 19; Stewart et al., Solid-Phase Peptide Synthesis (1989) W. H.; Houghten, Proc. Natl. Acad. Sci. USA (1985) 82: p.5132; Stuart and Young in Solid Phase Peptide Synthesis, Second Edition, Pierce Chemical Company (1984), and in Methods Enzymol., 289, Academic Press, Inc., New York (1997)). The peptide thus produced can be collected or purified by a routine method, for example, chromatography, such as gel filtration chromatography, ion exchange column chromatography, affinity chromatography, reverse phase column chromatography, and HPLC, ammonium sulfate fractionation, ultrafiltration, and immunoadsorption. The peptides disclosed herein can be prepared as described in published WIPO/PCT application WO2022/131283 (Published on Jun. 25, 2020). The tetrapeptide of Formula A-1 was prepared using a solution phase methodology whereby two suitably protected dimers were coupled and then deprotected according to Scheme set forth in FIG. 4.

In a solid-phase peptide synthesis, peptides are typically synthesized from the carbonyl group side (C-terminus) to amino group side (N-terminus) of the amino acid chain. In certain embodiments, an amino-protected amino acid is covalently bound to a solid support material through the carboxyl group of the amino acid, typically via an ester or amido bond and optionally via a linking group. The amino group may be deprotected and reacted with (i.e., “coupled” with) the carbonyl group of a second amino-protected amino acid using a coupling reagent, yielding a dipeptide bound to a solid support. Typically in solid phase synthesis, after coupling, a capping step is performed to cap (render unreactive) any unreacted amine groups. These steps (i.e., deprotection, coupling, and optionally capping) may be repeated to form the desired peptide chain. Once the desired peptide chain is complete, the peptide may be cleaved from the solid support and purified to the desired degree of purity.

In certain embodiments, the protecting groups used on the amino groups of the amino acid residues include 9-fluorenylmethyloxycarbonyl group (Fmoc) and t-butyloxycarbonyl (Boc). The Fmoc group is removed from the amino terminus with base while the Boc group is removed with acid. In alternative embodiments, the amino protecting group may be formyl, acrylyl (Acr), benzoyl (Bz), acetyl (Ac), trifluoroacetyl, substituted or unsubstituted groups of aralkyloxycarbonyl type, such as the benzyloxycarbonyl (Z, cbz or Cbz), p-chlorobenzyloxycarbonyl, p-bromobenzyloxycarbonyl, p-nitrobenzyloxycarbonyl, p-methoxybenzyloxycarbonyl, benzhydryloxycarbonyl, 2 (p-biphenylyl) isopropyloxycarbonyl, 2-(3,5-dimethoxyphenyl) isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methylsulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group.

Many amino acids bear reactive functional groups in the side chain. In certain embodiments, such functional groups are protected in order to prevent the functional groups from reacting with the incoming amino acid. The protecting groups used with these functional groups must be stable to the conditions of peptide synthesis, but may be removed before, after, or concomitantly with cleavage of the peptide from the solid support. Further reference is also made to: Isidro-Llobet, A., Alvarez, M., Albericio, F., “Amino Acid-Protecting Groups”; Chem. Rev., 109:2455-2504 (2009) as a comprehensive review of protecting groups commonly used in peptide synthesis.

In certain embodiments, the solid support material used in the solid-phase peptide synthesis method is a gel-type support such as polystyrene, polyacrylamide, or polyethylene glycol. Alternatively, materials such as pore glass, cellulose fibers, or polystyrene may be functionalized at their surface to provide a solid support for peptide synthesis.

Coupling reagents that may be used in the solid-phase or solution-phase peptide synthesis discussed herein are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC), and its HCl salt (EDC.HCl), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis [4-(2,2 -dimethyl-1,3-dioxolyl)]-methyl]carbodiimide (BDDC), and N,N-dicyclopentylcarbodiimide. DCC is a preferred coupling reagent. Other coupling agents include (1-[Bis (dimethylamino)methylene]-1H-1,2,3-triazolo[4,5-b]pyridinium 3-oxide hexafluorophosphate (HATU) and (2-(1H-benzotriazol-1-yl)-1,1,3,3-tetramethyluronium hexafluorophosphate (HBTU), generally used in combination with an organic base such as N,N-diisopropylethylamine (DIEA) and a hindered pyridine-type base such as lutidine or collidine.

In some embodiments, the amino acids can be activated toward coupling by forming N-carboxyanhydrides as described in Fuller et al., Urethane-Protected α-Amino Acid N-Carboxyanhydrides and Peptide Synthesis, Biopolymers (Peptide Science), Vol. 40, 183-205 (1996); and WO 2018/034901. Such methods of peptide synthesis may be used to produce the peptides disclosed herein either by solution-phase or solid-phase methodology.

### Determination of the Biological Effect of the Peptides or Mixtures of Peptides

In various embodiments, suitable in vitro or in vivo assays are performed to determine the effect of a peptide or mixture of peptides and therefore whether their administration is indicated for treatment. In various embodiments, in vitro assays can be performed with representative animal models, to determine if a given peptide or mixture of peptides exerts the desired effect on the disease. Compounds (e.g., a peptide or mixture of peptides) for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

Animal models of Duchenne muscular dystrophy (DMD) are known in the art, including, for example Golden retriever muscular dystrophy (GRMD) dogs, CXMDJ beagle dogs, hypertrophic feline muscular dystrophy (hfmd) cats, and mdx mice. See: Spurney C., Muscle Nerve 44 (1): 8-19 (2011); Willmann R. et al., Neuromuscular Disorders 19:241-249 (2009); Partridge TA, FEBS J. 280 (17): 4177-86 (2013) and Coley et al., “Effect of genetic background on the dystrophic phenotype in mdx mice”, Human Molecular Genetics, 2016, Vol. 25, No. 1, 130-145. More recently a rabbit model has been created that exhibits a very similar cardiac pathology to that observed in humans. See: Sui, T, et al., “A novel rabbit model of Duchenne muscular dystrophy generated by CRISPR/Cas9”, Disease Models & Mechanisms (2018) 11, dmm032201. Such models may be used to demonstrate the biological effect of the peptides and mixtures of peptides disclosed herein on the onset, incidence, severity and progression of heart disease and cardiomyopathies associated with muscular dystrophy (including DMD and Becker muscular dystrophy (BMD)) in subjects, including humans.

### Discussion of Examples Supporting the Present Disclosure and Claims

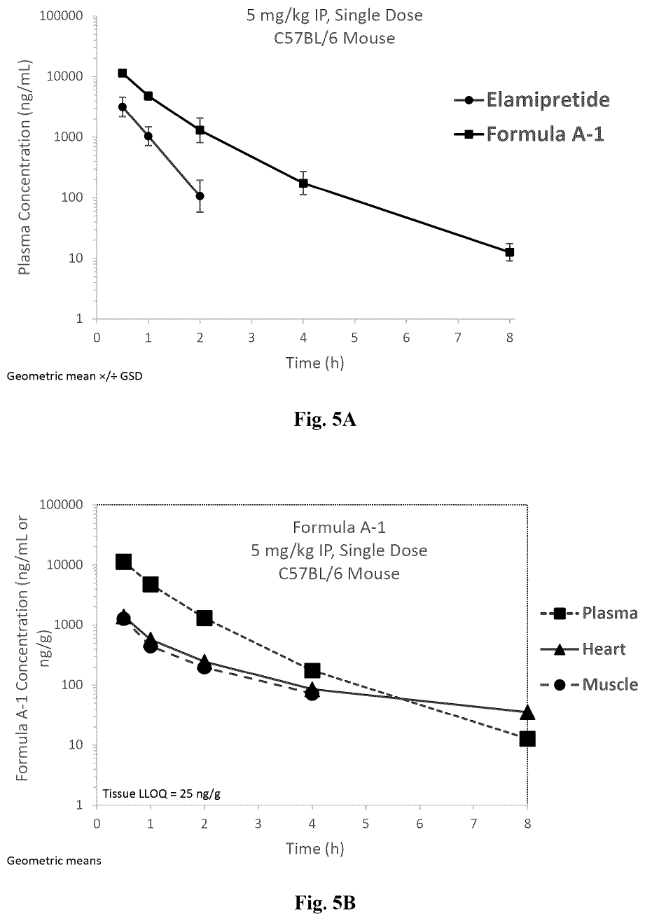

Example 1 provides a comparative assessment of the pharmacokinetics of elamipretide and the peptide of Formula A-1 based on the plasma concentration of the two peptides over time based on a single administered dose to mice at 5 mg/kg (once) of each peptide intraperitoneally (IP). The data presented in FIG. 5A for this Example 1, demonstrates that at approximately 1 hour, the concentration of the peptide of Formula A-1 is 3-4 higher in the plasma and is still detectable in the plasma after 8 hours (whereas the elamipretide is not). This data suggests that the peptide of Formula A-1 is metabolized/cleared more slowly (at least in this species of mouse) as compared with elamipretide and therefore may be available to produce a greater therapeutic effect when administered to a subject at the same concentration as elamipretide.

The data presented in FIG. 5B for this Example 1, the plasma concentration data from FIG. 5A is reproduced but also provided is the concentration data for the peptide of Formula A-1 in heart and muscle tissue over time. These data demonstrate that there is good uptake of the peptide of Formula A-1 in heart and muscle (albeit about 10× lower than in the plasma), without any significant difference observed in uptake between the two tissues (i.e., muscle and heart). Whilst still about 10 times lower than the plasma concentration, the peptide of Formula A-1 provides reasonable penetration into muscle and heart tissue sufficient to potentially produce an efficacious drug effect when dosed at 5 mg/kg. Furthermore, the data collected indicated that peptide of Formula A-1 was well tolerated in these mice when dosed at this level.

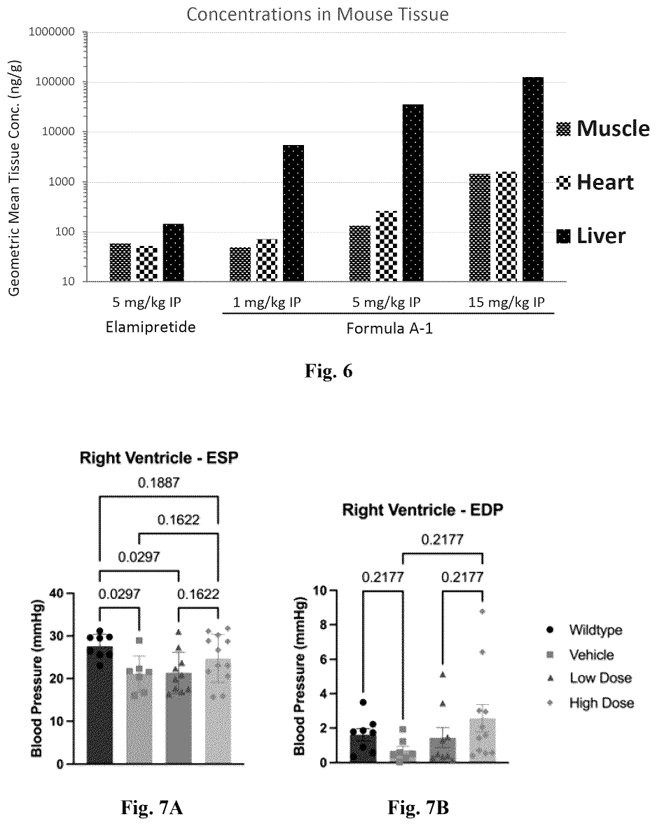

With reference to Example 2 and FIG. 6, data is presented in bar graph form for the concentrations of elamipretide and the peptide of Formula A-1 in muscle, heart and liver tissues where the elamipretide was administered at 5 mg/kg IP, and the peptide of Formula A-1 was administered at 1 mg/kg, 5 mg/kg or 15 mg/kg IP; in all cases repeated doses over a five day period. As can be seen with reference to FIG. 6, the concentration of the peptide of Formula A-1 administered at 1 mg/g is roughly equivalent to the concentration of elamipretide administered at 5 mg/kg in heart and muscle, thereby substantially confirming the data presented in Example 1 regardless of the difference between single and repeated dosing (compare with FIGS. 5A and 5B). In comparison, the concentration of the peptide of Formula A-1 is many fold higher in the liver when administered at 1 mg/kg as compared with the concentration of elamipretide in the liver when administered at 5 mg/kg. With reference to FIG. 6, tissue uptake of the peptide of Formula A-1 increased in response to the amount dosed. Furthermore, the data collected indicated that elamipretide and the peptide of Formula A-1 was well tolerated in these mice at all dosing levels examined. Collectively, the data collected in this Example 2 provides a sound basis to expect that the peptide of Formula A-1 may perform at least equal to or substantially better than elamipretide in treating a condition involving heart and/or muscle dysfunction, such as muscular dystrophy (include DMD or BMD).

With reference to Example 3 and FIGS. 7A and 7B, data is presented in bar graph for invasive hemodynamics of the right ventricle, as measured by end-systolic pressure (ESP; FIG. 7A) and end-diastolic pressure (EDP; FIG. 7B) in wildtype mice or D2.mdx mice following 4 weeks of daily subcutaneous injections of the peptide of Formula A-1 (1 mg/kg (low dose) or 5 mg/kg (high dose)) or vehicle control. Compared to wildtype mice, vehicle-treated D2.mdx mice showed significantly reduced right ventricular ESP and a trend towards reduced right ventricular EDP. Treatment with high dose (5 mg/kg) of the peptide of Formula A-1 showed a trend for improved right ventricular function compared to vehicle-treated control. Collectively, this data further supports a sound basis to expect that the peptide of Formula A-1 may perform at least equal to or substantially better than elamipretide in treating a condition involving heart (cardiac) dysfunction, such as is observed in muscular dystrophy (such as DMD or BMD).

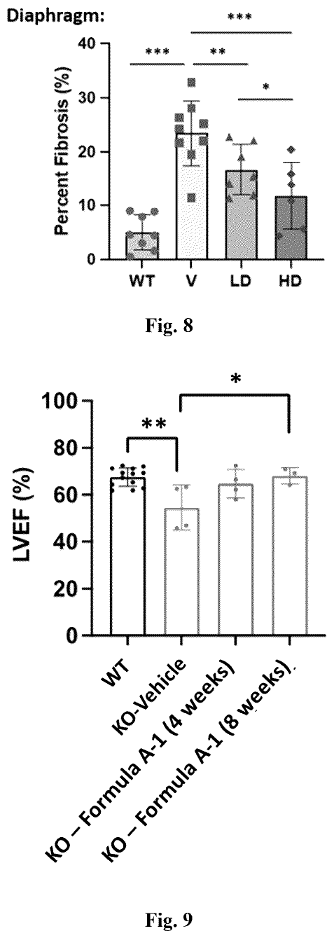

With reference to Example 3 and FIG. 8, data is presented in bar graph format for fibrosis observed in diaphragm tissue from wildtype mice, and D2.mdx mice (a well-known, established DMD mouse model) treated with vehicle, D2.mdx mice treated with low dose (1 mg/kg) peptide of Formula A-1, or D2.mdx mice treated with high dose (5 mg/kg) peptide of Formula A-1. With reference to FIG. 8, the wildtype mice provide the expected pattern/amount of fibrosis of the diaphragm expected in a subject not having DMD. As can be seen in FIG. 8, the vehicle-treated D2.mdx mice had significantly increased fibrosis in the diaphragm muscle as compared with the wildtype mice. By comparison, there was a significant reduction in diaphragm muscle fibrosis in D2.mdx mice administered low dose peptide of Formula A-1 and an even more significant improvement (decrease) in fibrosis of the diaphragm muscle in the D2.mdx mice administered high dose of the peptide of Formula A-1 as compared with the vehicle treated D2.mdx mice. Collectively, this data shows a dose response to administration of the peptide of Formula A-1 to the D2.mdx mice with respect to a muscle involved in the respiratory system, thereby demonstrating the efficacy of the peptide of Formula A-1 for addressing muscular dysfunction in subjects suffering from muscular dystrophy such as DMD.

With reference to Example 4 and FIG. 9, data is presented in bar graph format for the left ventricle ejection fraction measured in wild-type rabbits and mutant knock-out rabbits (DMD-KO) that manifest many genetically introduced phenotypic features associated with Duchenne muscular dystrophy (DMD), including a reduced left ventricle ejection fraction (LVEF). The data presented in FIG. 9 is a bar graph presentation of the ejection fraction measured in wild-type (WT) rabbits, DMD-KO rabbits administered vehicle only (i.e., untreated), DMD-KO rabbits treated with the peptide of Formula A-1 for 4 weeks, and DMD-KO rabbits treated with the peptide of Formula A-1 for 8 weeks. With reference to FIG. 9, the ejection fraction for the wildtype (WT) rabbits sets the expectation for a normal LVEF. As can be seen, the LVEF of the DMD-KO rabbits treated with vehicle was significantly reduced. Albeit not statistically significant, the L VEF of the DMD-KO rabbits treated for 4 weeks with the peptide of Formula A-1 appear to be improved, and very close to that observed for the WT rabbits. However, after treatment of the rabbits for 8 weeks with the peptide of Formula A-1, a statistically significant improvement in LVEF was observed in the DMD-KO rabbits-essentially returning the LVEF to the level observed in the WT cohort. This data suggests that administration of the peptide of Formula A-1 to the DMD-KO rabbits was effective in treating, preventing, ameliorating, inhibiting, delaying the onset of, or possibly even reversing the genetically induced LVEF deficit observed in these mutant rabbits; thereby again demonstrating the efficacy of the peptide of Formula A-1 in addressing the dysfunction associated with a subject having muscular dystrophy, such as DMD.

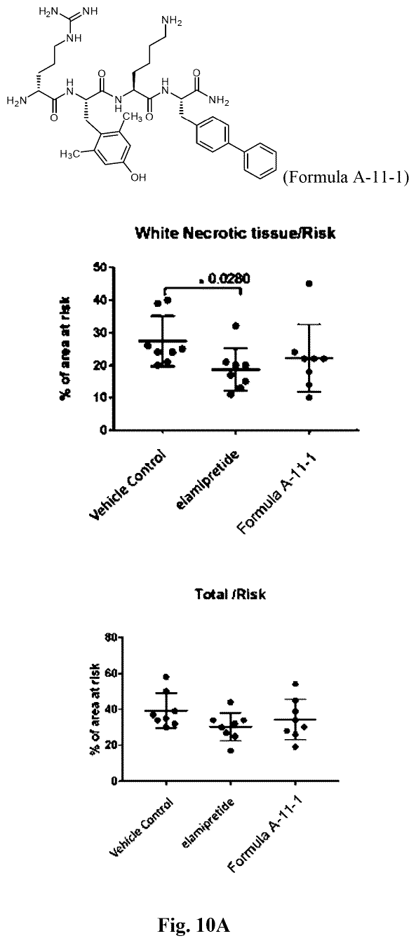

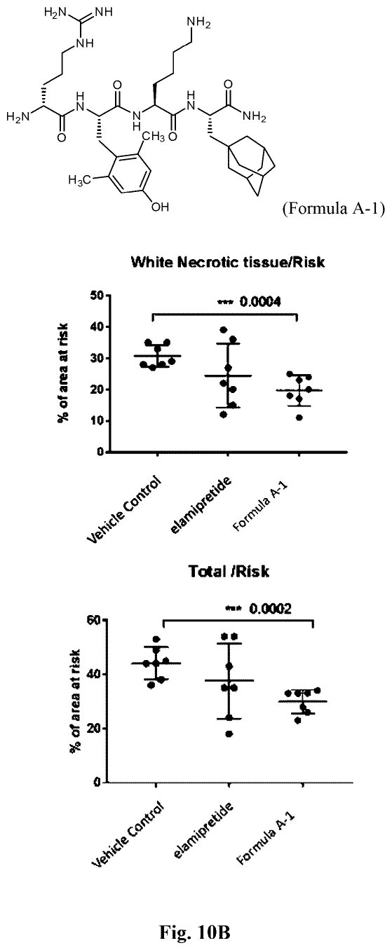

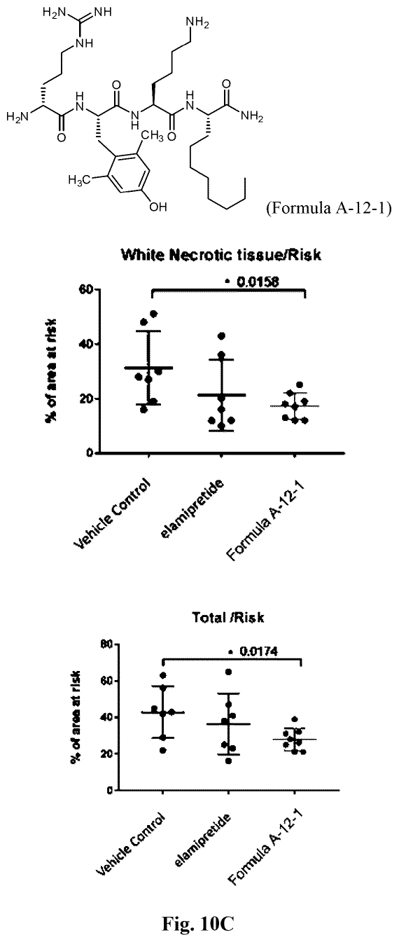

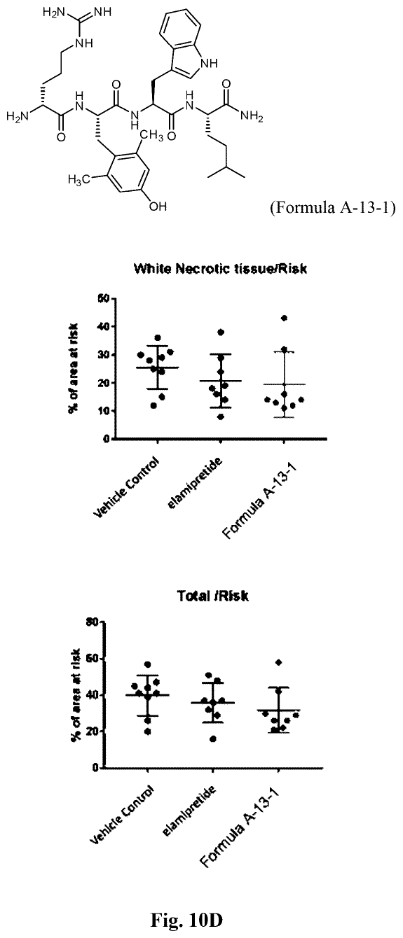

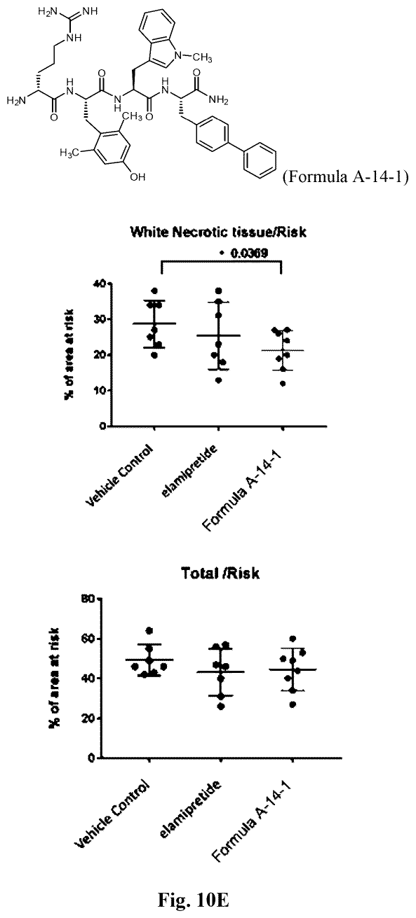

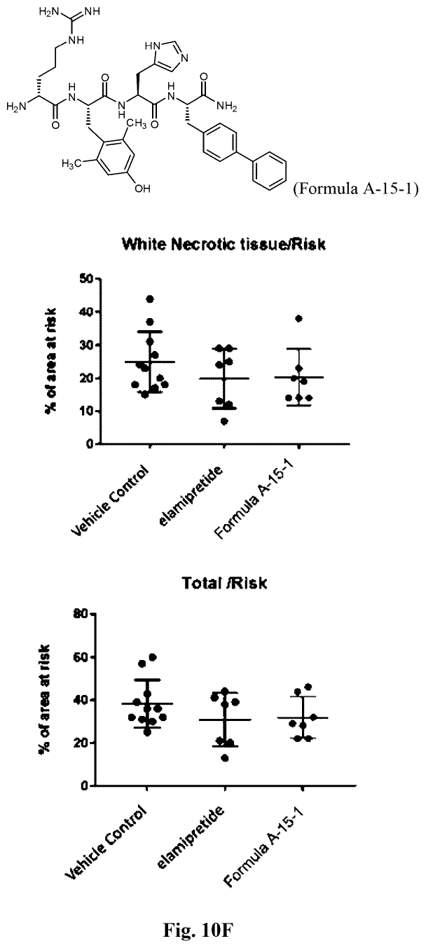

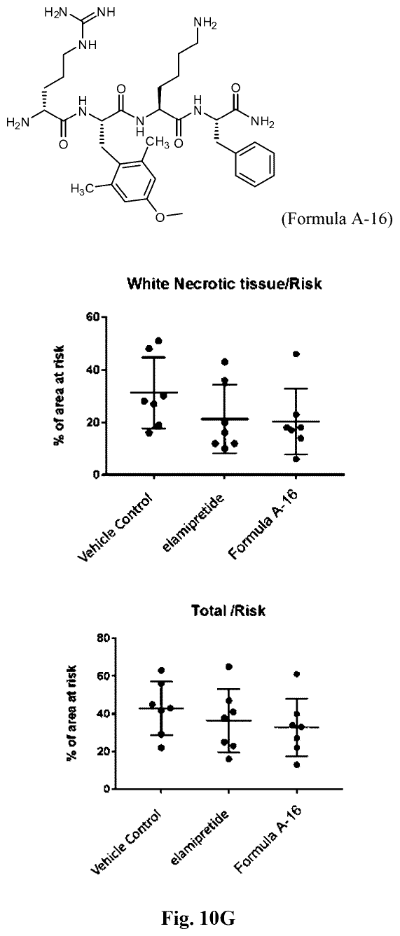

With reference to Example 5 and FIGS. 10A-10G, data is presented in bar graph form demonstrating the efficacy of peptides of Formulas A-1, A-11-1, A-12-1, A-13-1, A-14-1, A-15-1 and A-16, in exhibiting cardioprotective effects in a Langendorff study of induced ischemia-reperfusion injury. The modifications of the peptides used in Example 5 are representative of features/amino acids found in the peptide of Formula A. Generally, the peptides described in Example 5 all performed similar to or slightly better than did elamipretide in the ex-vivo Langendorff assay.

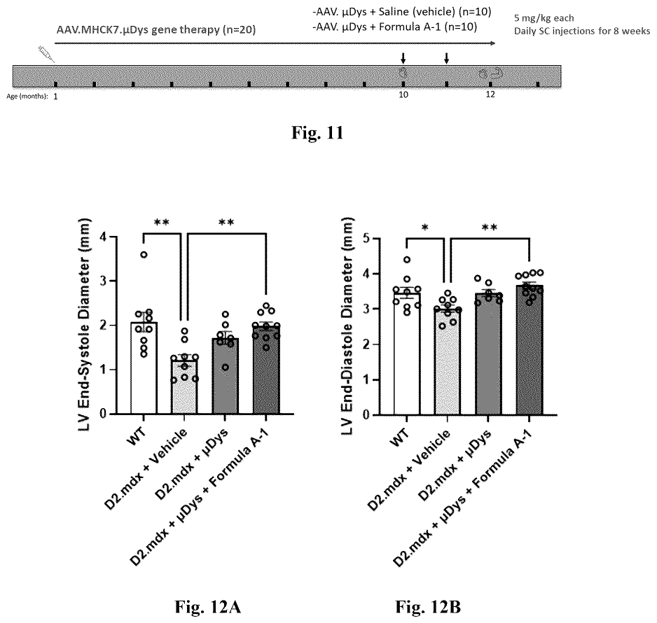

With respect to Example 6 and FIGS. 11-16C, data is presented for the treatment of D2.mdx mice (a well-studied mouse model of Duchenne muscular dystrophy), with an AAV vector modified PMO (identified as “micro-dystrophin gene therapy” or “μDys gene therapy” in the Example), optionally in combination with the peptide of Formula A-1. In this example, the effect of the treatments were examined and compared with respect to both skeletal muscle (EDL) and cardiac function. Collectively, the data suggests that the combination therapy (treatment with both μDys gene therapy and the peptide of Formula A-1) was superior in many ways as compared with treatment with the μDys gene therapy alone. In particular, the combination therapy was more cardioprotective as well as providing additional benefits to the performance of skeletal muscles. These results were not a priori expected.

### Methods

As can be deduced from the Examples and Figs. presented, the peptide of Formula A (such and the peptide of Formula A-1) can be prepared and administered to mammalian subjects such that it is well tolerated and can be delivered in a efficacious dose to the heart and other muscles of the subject (FIGS. 5A, 5B, and 6). Indeed, the data suggests that the peptide of Formula A-1 may be superior in its ability to penetrate heart and muscle tissue as compared with a peptide (i.e., elamipretide) considered for its potential in addressing cardiomyopathies (See: Daubert et al., Novel Mitochondria-Targeting Peptide in Heart Failure Treatment: A Randomized, Placebo-Controlled Trial of Elamipretide, Circ Heart Fail. 2017; 10: e001389, Gandhi et al., Cardiomyopathy in Duchenne Muscular Dystrophy and the Potential for Mitochondrial Therapeutics to Improve Treatment Response, Cells, 2024, 13, 1168 and Thompson et al., Long-term efficiency and safety of elamipretide in patients with Barth syndrome: 168-week open-label extension results of TAZPOWER, Genetics in Medicine (2024) 26, 101138). The data presented in FIG. 7A and FIG. 7B demonstrates an improvement in cardiac dysfunction of the right ventricle associated with administration of the peptide of Formula A-1 in a well-known, often studied mouse model of DMD. The data in FIG. 8 further demonstrates an improvement (decrease) in fibrosis of the diaphragm of this well-known, often studied mouse model of DMD and further demonstrates a dose effect associated with the administration of the peptide of Formula A-1. The data in FIG. 9 further supports treating the cardiomyopathy associated with DMD in a different animal model as well as demonstrates an improvement in efficacy associated with extended treatment over time. The data in FIG. 10A-10G demonstrates that variations of amino acids according to the peptide of Formula A appear to be similar to or somewhat superior to elamipretide, at least with respect to performance in the Langendorff assay-which assay does not involve systemic administration of the peptide to a living mammal.

Therefore, in some embodiments, this disclosure pertains to a method for treating, preventing, inhibiting, ameliorating or delaying the onset of muscular dystrophy in a mammalian subject in need thereof, comprising administering to the subject a therapeutically effective amount of a peptide of Formula A, or a mixture of peptides of Formula A:

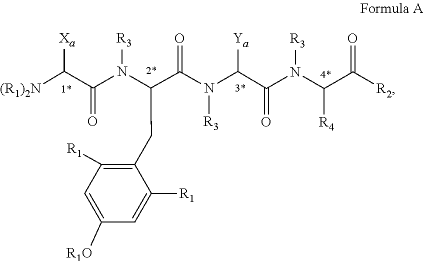

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

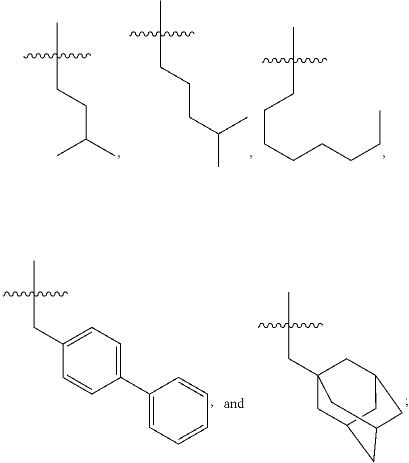

Xₐ is selected from

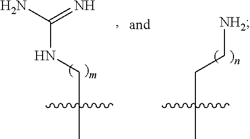

Yₐ is selected from

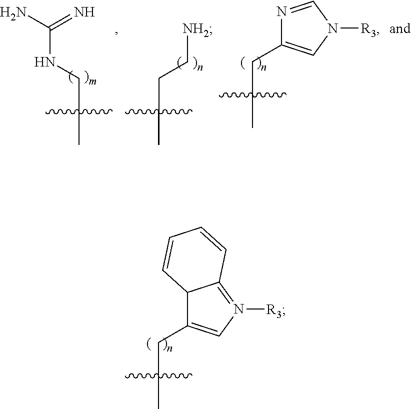

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom. In some embodiments, the peptide or peptides of Formula A is/are a peptide/peptides of Formula A-1 or Formula A-2:

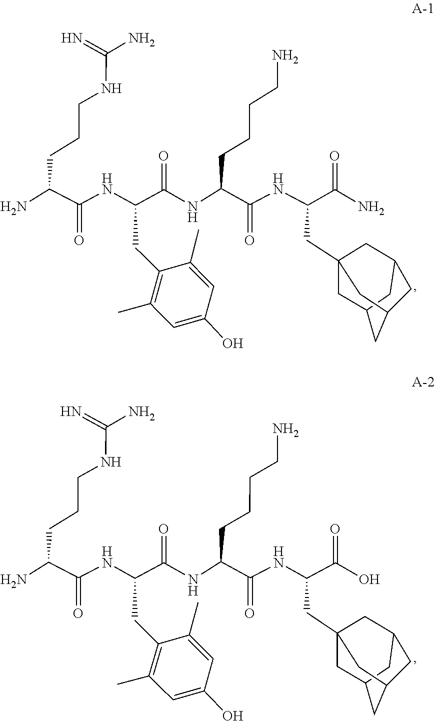 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

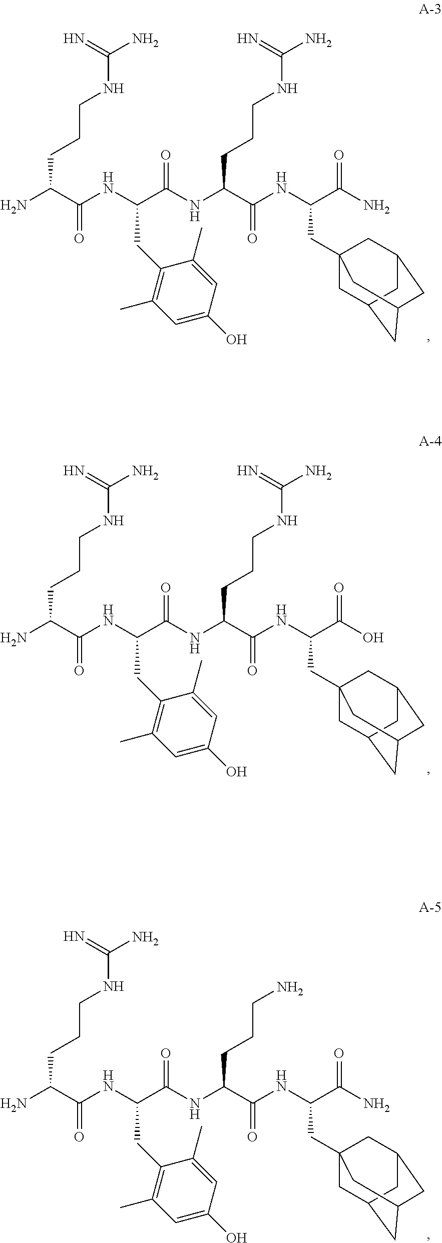 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

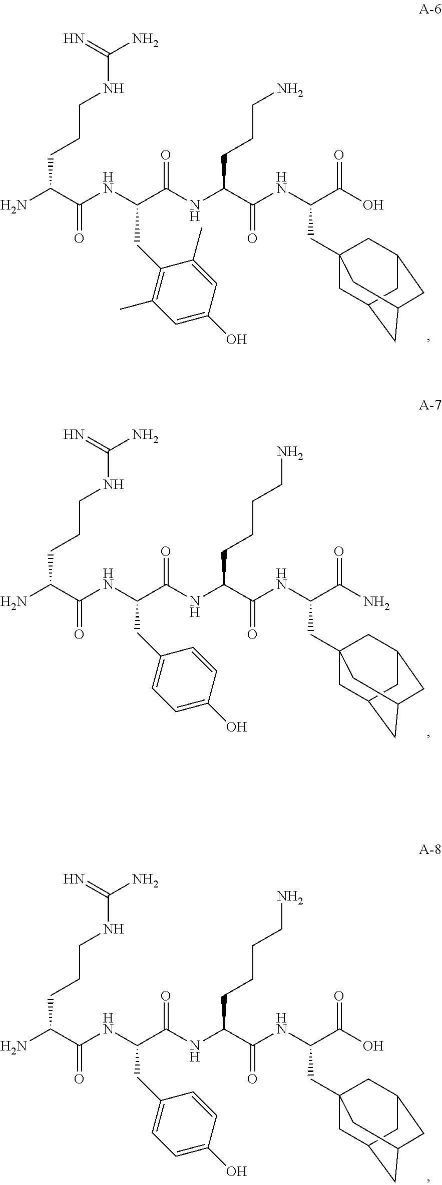 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

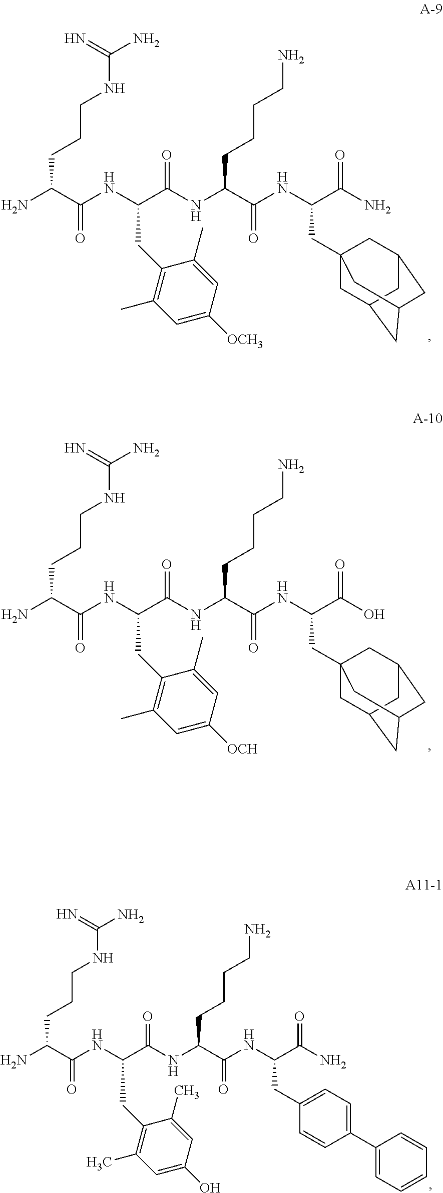

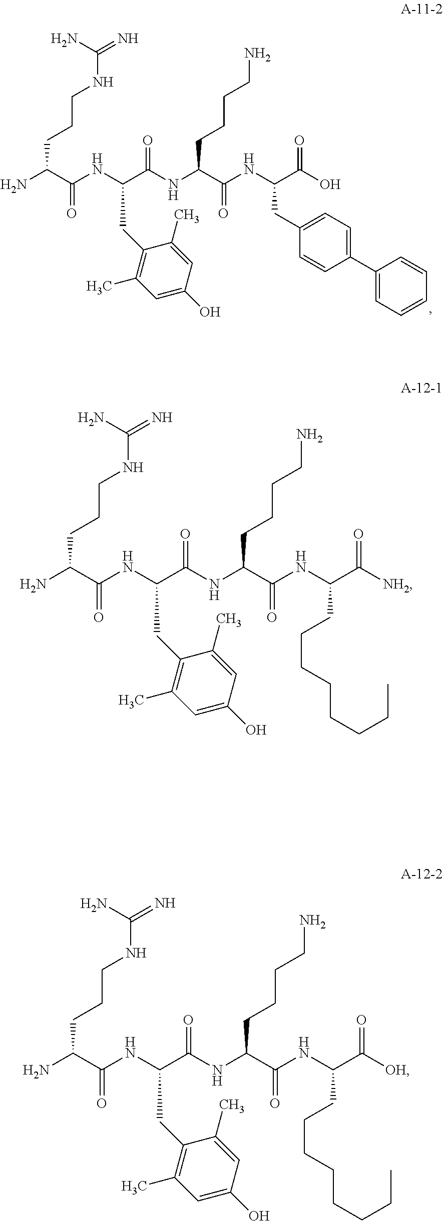 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

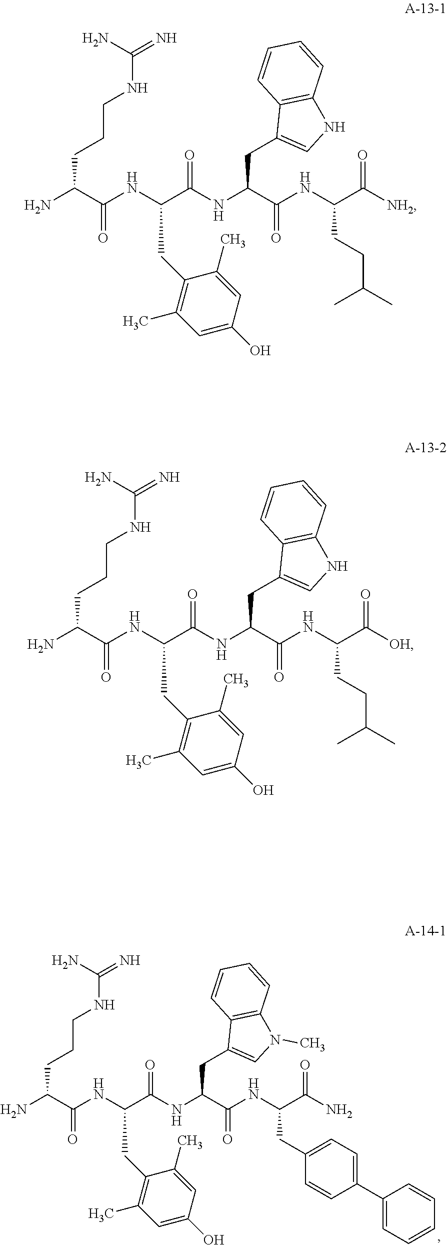 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O]

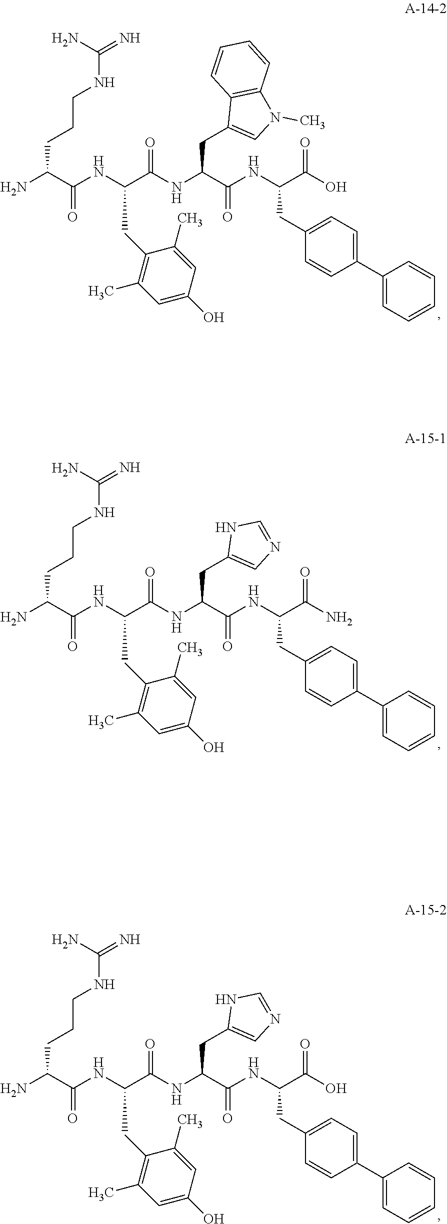 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A (e.g., peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2) are optionally substituted with a deuterium or fluorine atom.

In some embodiments of the method, administration of the peptide ameliorates, inhibits, delays the onset of, and/or delays the progression of one or more signs or symptoms of muscular dystrophy in the subject as compared with an untreated control subject or untreated control group that is not administered the peptide or peptides.

In some embodiments of the method, administration of the peptide ameliorates, inhibits, delays the onset of, and/or delays the progression of a cardiomyopathy associated with muscular dystrophy in the subject. In some embodiments of the method, the cardiomyopathy is systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fraction shortening, heart (cardiac) failure, or a combination of any two or more of the foregoing in the subject.

In some embodiments of the method, administration of the peptide or peptides ameliorates, inhibits, delays the onset of, and/or delays the progression of muscular fibrosis in the subject as compared with an untreated control subject or untreated control group that is not administered the peptide or peptides. In some embodiments of the method, administration of the peptide or peptides ameliorates, delays the onset of, and/or delays the progression of muscular fibrosis of the diaphragm of the subject as compared with an untreated control subject or untreated control group that is not administered the peptide or peptides. In some embodiments of the method, administration of the peptide or peptides ameliorates, delays the onset of, and/or delays the progression of muscular fibrosis of skeletal muscle of the subject as compared with an untreated control subject or untreated control group that is not administered the peptide or peptides.

In some embodiments of the method, administration of the peptide or peptides increases muscle force of a muscle or muscle group the subject can exert as compared with an untreated control subject or untreated control group that is not administered the peptide or peptides. For example, the muscle/muscle group can be the extensor digitorum longus (EDL).

In some embodiments of the method, administration of the peptide ameliorates, inhibits, delays the onset of, and/or delays progression of contraction-induced damage in muscle tissue, locomotor-skeletal muscle weakness, respiratory muscle weakness, cardiac muscle weakness, sarcolemmal weakening, or sarcolemmal tearing in the subject as compared with an untreated control subject or untreated control group that is not administered the peptide or peptides.

In some embodiments of the method, the peptide is administered daily for: (i) 12 weeks or more; (ii) 24 weeks or more; (iii) 48 weeks or more; (iv) 72 weeks or more; or (v) 96 weeks or more. In some embodiments of the method, the peptide is administered subcutaneously or intravenously. In some embodiments of the method, the peptide is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the method, the subject is human.

In some embodiments of the method, the muscular dystrophy is Duchenne muscular dystrophy. In some embodiments of the method, the muscular dystrophy is Becker's muscular dystrophy.

In some embodiments of the method, practice of the method further comprises separately, sequentially, or simultaneously administering an additional therapeutic agent to the subject.

In some embodiments of the method, the additional therapeutic agent is a phosphorodiamidate morpholino oligomer (PMO) or a peptide-conjugated phosphorodiamidate morpholino oligomer (PPMO). In some embodiments of the method, the PMO is selected from the group consisting of eteplirsen (Exondys 51®), golodirsen (Vyondys 53®), viltolarsen (Viltepso®), and casimersen (Amondys 45™) or the PPMO is selected from vesleteplirsen (a.k.a., SRP-5051) and PGN-EDO51 (a.k.a., CONNECT1-ED051).

In some embodiments of the method, the additional therapeutic agent is an adeno-associated virus (AAV) vector-based gene therapy. In some embodiments of the method, the adeno-associated virus (AAV) vector-based gene therapy is delandistrogene moxeparvovec-rokl (Elevidys®). These results are supported by the results seen in Example 6 and FIGS. 11-16C.

In some embodiments of the method, the additional therapeutic agent is a corticosteroid, an angiotensin-converting enzyme (ACE) inhibitor, an angiotensin receptor blocker (ARB), a beta blocker, a diuretic, or a mineralocorticoid receptor antagonist. In some embodiments of the method, the corticosteroid is vamorolone (aGamree®), prednisone, or deflazacort. In some embodiments of the method, the angiotensin-converting enzyme (ACE) inhibitor is captopril, enalapril, lisinopril, benazepril, or ramipril. In some embodiments of the method, the angiotensin receptor blocker (ARB) is azilsartan, candesartan, eprosartan, irbesartan, telmisartan, valsartan, losartan, olmesartan, Entresto® (sacubitril/valsartan) or Byvalson™ (nebivolol/valsartan). In some embodiments of the method, the beta blocker is carvedilol, bisoprolol, metoprolol succinate, atenolol, esmolol, nebivolol, or propranolol. In some embodiments of the method, the diuretic is bumetanide, furosemide, or torsemide. the mineralocorticoid receptor antagonist is spironolactone or eplerenone.

In some embodiments of the method, the additional therapeutic agent is mavacamten (Camzyos®).

In some embodiments of the method, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the method, the peptide or peptides is/are formulated for administration from its/their tris —HCl salt.

In another aspect, the present disclosure pertains to a method for treating, preventing, inhibiting, ameliorating or delaying the onset of a cardiomyopathy in a mammalian subject in need thereof, wherein the subject is suffering from muscular dystrophy, comprising administering to the subject a therapeutically effective amount of a peptide of Formula A, or a mixture of peptides of Formula A:

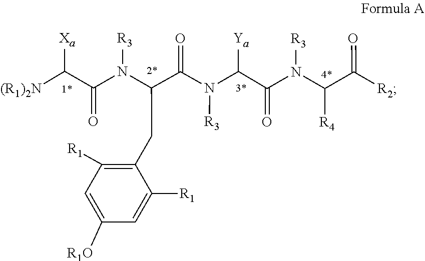

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

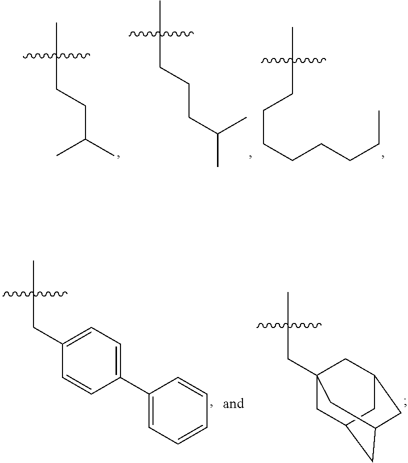

Xₐ is selected from

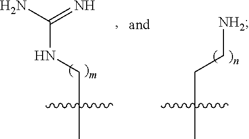

Yₐ is selected from

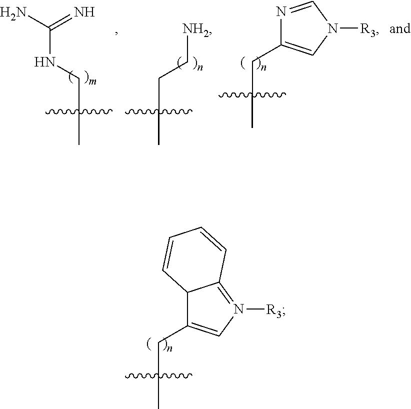

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is substituted with a deuterium or fluorine atom. In some embodiments of the method, the peptide or peptides of Formula A is/are a peptide/peptides of Formula A-1 or Formula A-2:

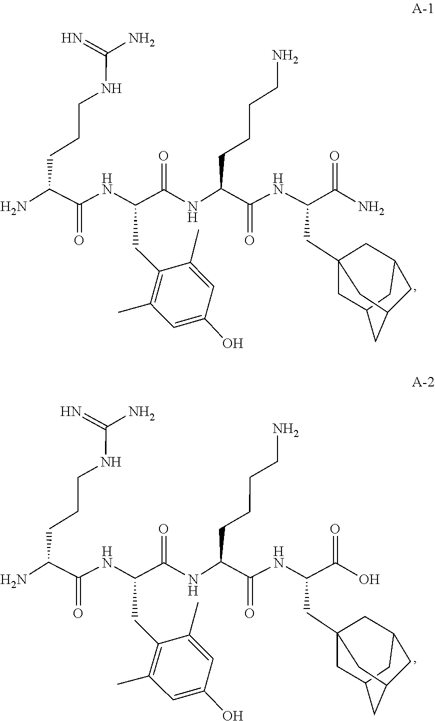 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the method, the peptide or peptides of Formula A is/are a peptide of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

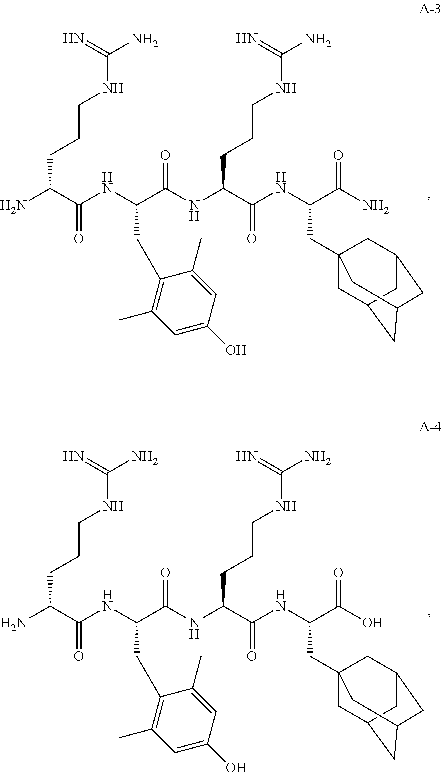 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O]

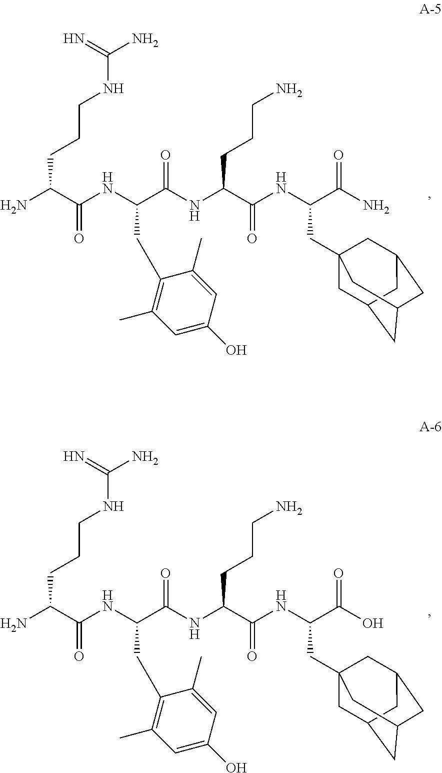 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

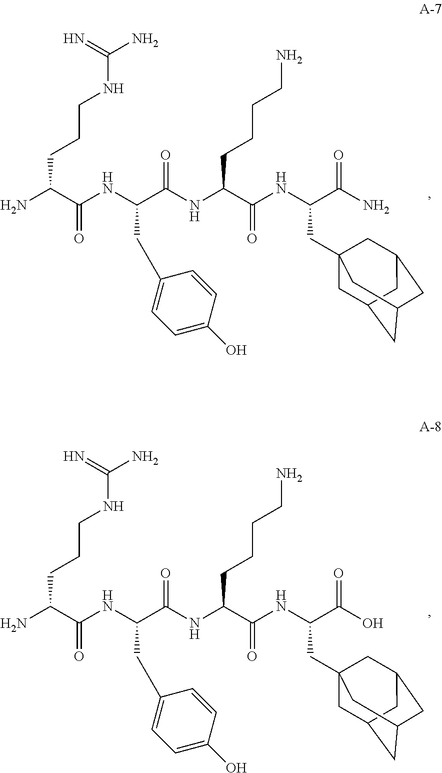 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

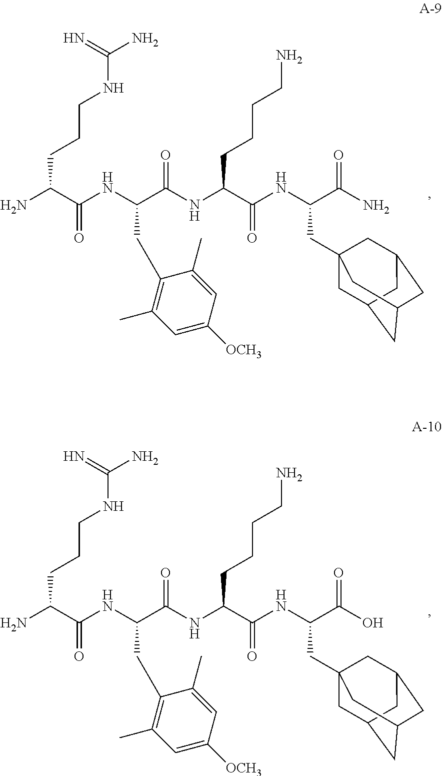 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

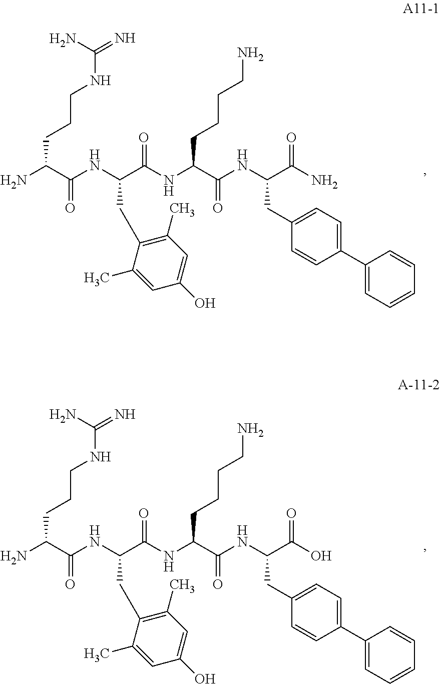 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

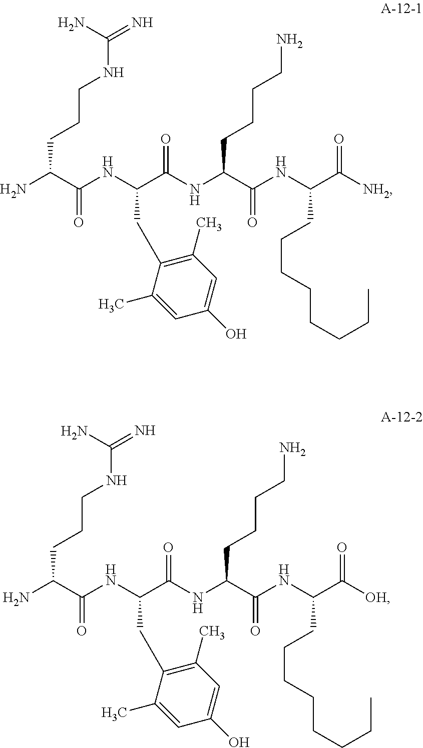 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

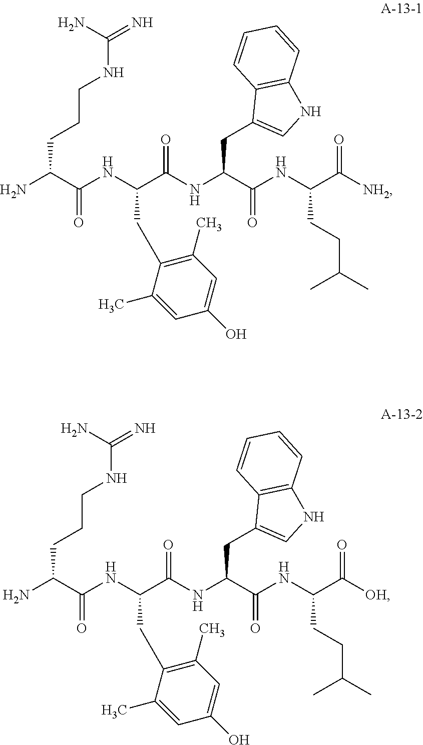 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

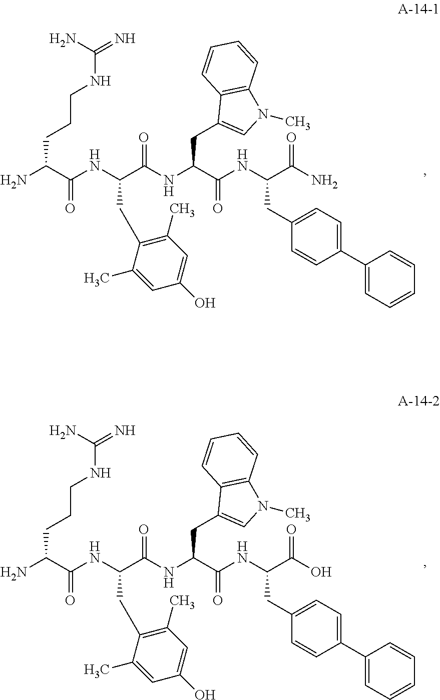 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

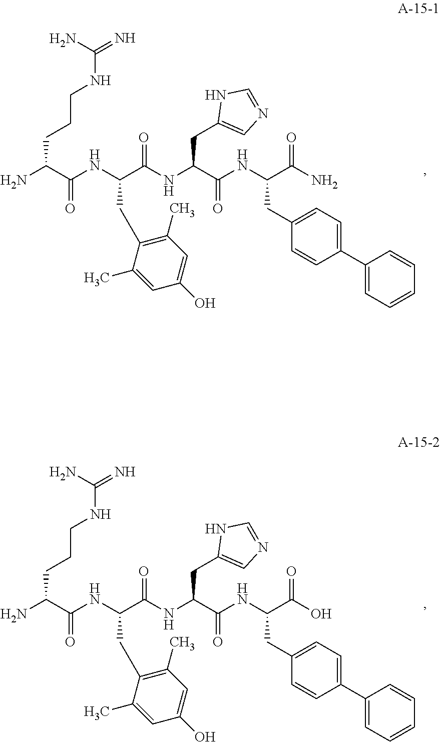 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A (e.g., peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2) are optionally substituted with a deuterium or fluorine atom.

In some embodiments of the method, administration of the peptide ameliorates, inhibits, delays the onset of, and/or delays the progression of calcium overload, systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, or heart (cardiac) failure in the subject as compared with an untreated control subject or untreated control group that is not administered the peptide or peptides.

In some embodiments of the method, administration of the peptide ameliorates, inhibits, delays the onset of, and/or delays the progression of calcium overload, systolic disfunction, or diastolic dysfunction in the subject as compared with an untreated control subject or untreated control group that is not administered the peptide or peptides.

In some embodiments of the method, administration of the peptide ameliorates, inhibits, delays the onset of, and/or delays the progression of ventricular remodeling, including left ventricular remodeling, in the subject as compared with an untreated control subject or untreated control group that is not administered the peptide or peptides.

In some embodiments of the method, administration of the peptide ameliorates, inhibits, delays the onset of and/or delays the progression of cardiac (myocardial) fibrosis in the subject as compared with an untreated control subject or untreated control group that is not administered the peptide or peptides.

In some embodiments of the method, administration of the peptide ameliorates, inhibits, delays the onset of, and/or delays the progression of fatty acid infiltration in the subject as compared with an untreated control subject or untreated control group that is not administered the peptide or peptides.

In some embodiments of the method, administration of the peptide ameliorates, inhibits, delays the onset of, and/or delays the progression of hypertrophic cardiomyopathy in the subject as compared with an untreated control subject or untreated control group that is not administered the peptide or peptides.

In some embodiments of the method, wherein administration of the peptide ameliorates, inhibits, delays the onset of, and/or delays the progression of dilated cardiomyopathy in the subject as compared with an untreated control subject or untreated control group that is not administered the peptide or peptides.

In some embodiments of the method, administration of the peptide ameliorates, inhibits, delays the onset of, and/or delays the progression of an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia) in the subject as compared with an untreated control subject or untreated control group that is not administered the peptide or peptides. In some embodiments of the method, administration of the peptide ameliorates, delays the onset of, and/or delays the progression of reduced left ventricular ejection fraction in the subject as compared with an untreated control subject or untreated control group that is not administered the peptide or peptides.

In some embodiments of the method, administration of the peptide ameliorates, inhibits, delays the onset of, and/or delays the progression of fractional shortening in the subject.

In some embodiments of the method, administration of the peptide ameliorates, inhibits, delays the onset of, and/or delays the progression of heart (cardiac) failure in the subject.

In some embodiments of the method, administration of the peptide results in normalization of the ejection fraction, fractional shortening, stroke volume, and/or cardiac output of the subject as compared with an untreated control subject or control group that is not administered the peptide or peptides.

In some embodiments of the method, the peptide is administered daily for: (i) 12 weeks or more; (ii) 24 weeks or more; (iii) 48 weeks or more; (iv) 72 weeks or more; or (v) 96 weeks or more. In some embodiments of the method, the peptide is administered subcutaneously or intravenously. In some embodiments of the method, the peptide is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the method, the subject is human.

In some embodiments of the method, the muscular dystrophy is Duchenne muscular dystrophy.

In some embodiments of the method, the muscular dystrophy is Becker's muscular dystrophy.

In some embodiments of the method, practice of the method further comprises separately, sequentially, or simultaneously administering an additional therapeutic agent to the subject.

In some embodiments of the method, the additional therapeutic agent is a phosphorodiamidate morpholino oligomer (PMO) or a peptide-conjugated phosphorodiamidate morpholino oligomer (PPMO). In some embodiments of the method, the PMO is selected from the group consisting of eteplirsen (Exondys 51®), golodirsen (Vyondys 53®), viltolarsen (Viltepso®), and casimersen (Amondys 45®) or the PPMO is selected from vesleteplirsen (SRP-5051) and PGN-EDO51 (a.k.a., CONNECT1-ED051).

In some embodiments of the method, the additional therapeutic agent is an adeno-associated virus (AAV) vector-based gene therapy. In some embodiments of the method, the adeno-associated virus (AAV) vector-based gene therapy is delandistrogene moxeparvovec-rokl (Elevidys®).

In some embodiments of the method, the additional therapeutic agent is a corticosteroid, an angiotensin-converting enzyme (ACE) inhibitor, an angiotensin receptor blocker (ARB), a beta blocker, a diuretic, or a mineralocorticoid receptor antagonist. In some embodiments of the method, the corticosteroid is vamorolone (aGamree®), prednisone, or deflazacort. In some embodiments of the method, the angiotensin-converting enzyme (ACE) inhibitor is captopril, enalapril, lisinopril, benazepril, or ramipril. In some embodiments of the method, the angiotensin receptor blocker (ARB) is azilsartan, candesartan, eprosartan, irbesartan, telmisartan, valsartan, losartan, olmesartan, Entresto® (sacubitril/valsartan) or Byvalson™ (nebivolol/valsartan). In some embodiments of the method, the beta blocker is carvedilol, bisoprolol, metoprolol succinate, atenolol, esmolol, nebivolol, or propranolol. In some embodiments of the method, the diuretic is bumetanide, furosemide, or torsemide. In some embodiments of the method, the mineralocorticoid receptor antagonist is spironolactone or eplerenone.

In some embodiments of the method, the additional therapeutic agent is mavacamten (Camzyos®).

In some embodiments of the method, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the method, the peptide or peptides is/are formulated for administration from its/their tris —HCl salt.

In another aspect, the present disclosure provides methods for treating the signs, symptoms, or severity of muscular dystrophy (e.g., DMD or BMD) in a mammalian subject in need thereof, wherein the subject is suffering from muscular dystrophy, comprising administering to a subject a therapeutically effective amount of a therapeutically active peptide or mixture of peptides and optionally one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) optionally in combination with one or more of the following additional therapeutic agents: a corticosteroid, an angiotensin-converting enzyme (ACE) inhibitor, an angiotensin receptor blocker (ARB), a beta blocker, a diuretic, or a mineralocorticoid receptor antagonist. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection.

In another aspect, the present disclosure provides methods for inhibiting the signs, symptoms, or severity of muscular dystrophy (e.g., DMD or BMD) in a mammalian subject in need thereof, wherein the subject is suffering from muscular dystrophy, comprising administering to the subject a therapeutically effective amount of a therapeutically active peptide or mixture of peptides and optionally one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) optionally in combination with one or more of the following additional therapeutic agents: a corticosteroid, an angiotensin-converting enzyme (ACE) inhibitor, an angiotensin receptor blocker (ARB), a beta blocker, a diuretic, or a mineralocorticoid receptor antagonist. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection.

In still another aspect, the present disclosure provides methods for preventing the signs, symptoms, or severity of muscular dystrophy (e.g., DMD or BMD) in a mammalian subject in need thereof, wherein the subject is suffering from muscular dystrophy, comprising administering to the subject a therapeutically effective amount of a therapeutically active peptide or mixture of peptides and optionally one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) optionally in combination with one or more of the following additional therapeutic agents: a corticosteroid, an angiotensin-converting enzyme (ACE) inhibitor, an angiotensin receptor blocker (ARB), a beta blocker, a diuretic, or a mineralocorticoid receptor antagonist. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection.

In yet another aspect, the present disclosure provides methods for ameliorating the signs, symptoms, or severity of muscular dystrophy (e.g., DMD or BMD) in a mammalian subject in need thereof, wherein the subject is suffering from muscular dystrophy, comprising administering to the subject a therapeutically effective amount of a therapeutically active peptide or mixture of peptides and optionally one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) optionally in combination with one or more of the following additional therapeutic agents: a corticosteroid, an angiotensin-converting enzyme (ACE) inhibitor, an angiotensin receptor blocker (ARB), a beta blocker, a diuretic, or a mineralocorticoid receptor antagonist. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

In still a further aspect, the present disclosure provides methods for delaying the onset of the signs, symptoms, or severity of muscular dystrophy (e.g., DMD or BMD) in a mammalian in need thereof, wherein the subject is subject suffering from muscular dystrophy, comprising administering to the subject a therapeutically effective amount of a therapeutically active peptide or mixture of peptides and optionally one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) optionally in combination with one or more of the following additional therapeutic agents: a corticosteroid, an angiotensin-converting enzyme (ACE) inhibitor, an angiotensin receptor blocker (ARB), a beta blocker, a diuretic, or a mineralocorticoid receptor antagonist. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

In yet another aspect, the present disclosure provides methods for delaying the onset of muscular dystrophy (e.g., DMD or BMD) in a mammalian subject in need thereof, wherein the subject is suffering from muscular dystrophy, comprising administering to the subject a therapeutically effective amount of a therapeutically active peptide or mixture of peptides and optionally one or more PMOs and/or PPMOs as described in more detail herein, or a pharmaceutically acceptable salt, hydrate, solvate and/or tautomer thereof. In some embodiments, the subject harbors a genetic permutation that affects the production and/or function of dystrophin protein. In some embodiments, this method further comprises administering the peptide or mixture of peptides and one or more PMOs and/or PPMOs (as defined herein) optionally in combination with one or more of the following additional therapeutic agents: a corticosteroid, an angiotensin-converting enzyme (ACE) inhibitor, an angiotensin receptor blocker (ARB), a beta blocker, a diuretic, or a mineralocorticoid receptor antagonist. In some embodiments, co-administration is simultaneous, such as by simultaneous administration by IV injection. In some embodiments, co-administration is simultaneous, but by different routes of administration, such as by administering the one or more PMOs and/or PPMOs by IV injection (or other route of administration of a long-term systemic release depot formulation) while the peptide or mixture of peptides is/are administered by, for example, subcutaneous injection (or other route of administration of a long-term systemic release depot formulation).

### Compositions, Formulations & Medicaments And Their Use

This disclosure further relates to compositions (i.e., formulations or medicaments) that can be used in the disclosed methods wherein the composition comprises at least one peptide of Formula A (e.g., a peptide of Formula: A-1, A-2, A-3, A-4, A-5, A-6, A-7, A-8, A-9, A-10, A-11-1, A-11-2, A-12-1, A-12-2, A-13-1, A-13-2, A-14-1, A-14-2, A-15-1 and A-15-2), and optionally may also include or more of the following additional therapeutic agents such as for example: (i) a phosphorodiamidate morpholino oligomer (PMO), (ii) a peptide-conjugated phosphorodiamidate morpholino oligomer (PPMO), (iii) an adeno-associated virus (AAV) vector-based gene therapy, (iv) an angiotensin-converting enzyme (ACE) inhibitor, (v) an angiotensin receptor blocker (ARB), (vi) a beta blocker, (vii) a diuretic, or (viii) a mineralocorticoid receptor antagonist. Such a composition can be formed, for example, by dissolving or suspending the selected compound(s)/peptide (or mixture of peptides) in water, buffer, detergent, excipient, organic solvent or a mixture of two or more of the foregoing. In some embodiments, the composition can be prepared by dissolving or suspending the selected compound(s)/peptide(s) in water. In some embodiments, the composition can be prepared by dissolving or suspending the selected compound(s)/peptide(s) in buffer (e.g., saline or phosphate buffered saline). In some embodiments, the composition can be prepared by dissolving or suspending the selected compound(s)/peptide(s) in excipient. In some embodiments, the composition can be prepared by dissolving or suspending the selected compound(s)/peptide(s) in a pharmaceutically acceptable carrier. In some embodiments, the composition is a formulation or is a medicament. Generally, as used herein the terms composition, formulation and medicament are interchangeable. Other more specific types of formulations and methods for preparation are discussed in more detail below.

The peptide or mixture of peptides and optionally other therapeutic agents may be administered per se (neat) or in the form of a pharmaceutically acceptable salt. When used in medicine the salts may be pharmaceutically acceptable, but non-pharmaceutically acceptable salts may conveniently be used to prepare pharmaceutically acceptable salts thereof. When compounds/peptides of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts are known in the art and some are disclosed herein. When compounds/peptides of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts are known in the art and some are disclosed herein.

Certain specific compounds of the present disclosure may contain both basic and acidic functionalities that allow the compounds to be converted into either base or acid addition salts (see, e.g., FIG. 2, FIG. 3). These salts may be prepared by methods known to those skilled in the art. Other pharmaceutically acceptable carriers known to those of skill in the art are suitable for use with the present technology.

In some embodiments, the compositions/formulations can be used as medicaments for use in the practice of the methods disclosed herein.

The compositions (and methods of the present disclosure) may be used to treat an individual/subject in need thereof. In certain embodiments, the individual is a mammal such as a human, or a non-human mammal. When administered to an animal, such as a human, the composition or the compound/peptide(s) is/are preferably administered as a pharmaceutical composition comprising, for example, a peptide or mixture of peptides or optionally other therapeutic agent(s) and an excipient or pharmaceutically acceptable carrier. When the peptide or mixture of peptides is used in combination with other therapeutic agent(s), the other therapeutic agent(s) might, in some embodiments, be formulated in the same vehicle as the peptide or mixture of peptides or, in some embodiments, the peptide or mixture of peptides may be formulated independently based on, for example, the nature of those agents and the preferred administration route or timing of administration.

As stated above, an “effective amount” refers to any amount of the active compound/therapeutic agent (e.g., a peptide, mixture of peptides and/or optionally other therapeutic agent(s); alone or as formulated) that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic (i.e., preventative) or therapeutic treatment regimen can be planned which does not cause substantial unwanted toxicity and yet is effective to treat the particular condition or disease of a particular subject. The effective amount for any particular indication can vary depending on such factors as the disease or condition being treated, the particular compound of the present application being administered, the size of the subject, or the severity of the disease or condition. The effective amount may be determined during pre-clinical trials and/or clinical trials by methods familiar to physicians and clinicians. One of ordinary skill in the art can empirically determine the effective amount of a particular peptide or mixture of peptides of the present technology and/or other therapeutic agent(s) without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein. A dose may be administered by oneself, by another or by way of a device (e.g., a pump).

For any compound/therapeutic agent/composition (e.g., a peptide, mixture of peptides or other therapeutic agents) described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan. In some embodiments, the daily dose of a peptide of Formula A (e.g., the peptide of Formula A-1) for a subject can be from 0.1 mg/kg to 10 mg/kg (inclusive) of body weight, or be from 1 mg/kg to 10 mg/kg (inclusive) of body weight. In some embodiments, a human daily dose of a peptide of Formula A (e.g., the peptide of Formula A-1) can be from 1 mg to 60 mg administered subcutaneously.

Peptides/compounds (alone or as formulated in a pharmaceutical composition) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing of any of the animal model system known in the art can be used prior to administration to human subjects.

Dosage, toxicity and therapeutic efficacy of any therapeutic peptides, compounds, compositions (e.g., formulations or medicaments), other therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography, optionally coupled with mass spectroscopy detection (e.g. LC/MS).

The effective amount may be determined during pre-clinical trials and clinical trials by methods familiar to physicians and clinicians. An effective amount of the compound(s)/peptide(s) useful in the methods disclosed herein may be administered to a mammal in need thereof by any of a number of well-known methods for administering pharmaceutical compounds. The compound(s)/peptide(s) may be administered systemically or locally.

The skilled artisan will appreciate that certain factors may influence the dosage, mode of administration and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The compound(s)/peptide(s) of a composition (e.g., a formulation or medicament) can be administered by any known or future developed mode of administration. For example, administration can be oral. Administration can be systemic. Administration can be subcutaneous. Administration can be intravenous. Administration can be topical. Administration can be intraperitoneal. Administration can be intradermal. Administration can be transdermal. Administration can be ophthalmical. Administration can be retro-orbital. Administration can be intrathecal. Administration can be intracerebroventricular. Administration can be iontophoretical. Administration can be transmucosal. Administration can be intravitreal Administration can be intranasal Administration can be intramuscular. In some embodiments, peptide, mixture of peptides and/or the other therapeutic agent(s) are separately, sequentially or simultaneously administered. In some embodiments, administration of the peptide or mixture of peptides in combination with other therapeutic agents produces a synergistic effect.

In some embodiments, the peptide or mixture of peptides of a composition and optionally other therapeutic agent(s) is/are administered to the subject for 6 weeks or more. In some embodiments, the peptide, mixture of peptides and/or optionally other therapeutic agent(s) is/are administered to the subject for 12 weeks or more. In some embodiments, the peptide, mixture of peptides and/or optionally other therapeutic agent(s) is/are administered to the subject for 24 weeks or more. In some embodiments, the peptide, mixture of peptides and/or optionally other therapeutic agent(s) is/are administered to the subject for 48 weeks or more. In some embodiments, the peptide mixture of peptides and/or optionally other therapeutic agent(s) is/are administered to the subject for 72 weeks or more. In some embodiments, the peptide, mixture of peptides and/or optionally other therapeutic agent(s) is/are administered to the subject for 96 weeks or more. In some embodiments, the peptide, mixture of peptides and/or optionally other therapeutic agent(s) is/are administered to the subject for 1 year or more. In some embodiments, the peptide, mixture of peptides and/or optionally other therapeutic agent(s) is/are administered to the subject for 2 years or more. In some embodiments, the peptide, mixture of peptides and/or optionally other therapeutic agent(s) is/are administered to the subject for 3 years or more. In some embodiments, the peptide, mixture of peptides and/or optionally other therapeutic agent(s) is/are administered until no continued therapeutic benefit is observed. In some embodiments, the peptide, mixture of peptides and/or optionally other therapeutic agent(s) is/are administered until the end of life of the subject.

The pharmaceutical compositions (e.g., a formulation or medicament) can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Solutions or suspensions (e.g., a formulation or medicament) used for parenteral, intradermal or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 2, 3, 4, 5, 6, 7 days, weeks, months or more of treatment).

Systemic formulations include those designed for administration by injection, e.g., subcutaneous, intravenous, intramuscular, intrathecal or intraperitoneal injection, as well as those designed for transdermal, transmucosal oral or pulmonary administration.

For intravenous and other parenteral routes of administration, a compound (e.g., a peptide or mixture of peptides, and optionally other therapeutic agent(s) of the present technology can be formulated as a lyophilized preparation, as a lyophilized preparation of liposome-intercalated or —encapsulated active compound, as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g., a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and must be preserved against the contaminating action of microorganisms such as bacteria and fungi.

Sterile injectable solutions (e.g., a formulation or medicament) can be prepared by incorporating the active compound (e.g., a peptide, mixture of peptides, and/or optionally other therapeutic agent(s)) in the required amount in an appropriate solvent with one or a combination of ingredients enumerated above, as required, followed by filtered sterilization. Generally, dispersions are prepared by incorporating the active compound into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof. Sterilization can also be accomplished by exposure of the formulation or medicament to gamma radiation.

The therapeutic compounds (e.g., a peptide, a mixture of peptides, and/or optionally other therapeutic agent(s)) of the composition(s), when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions for parenteral administration include aqueous solutions of the active compounds (e.g., a peptide, a mixture of peptides, or optionally other therapeutic agent(s)) in water-soluble form. Additionally, suspensions of the therapeutic compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the therapeutic compounds to allow for the preparation of highly concentrated solutions.

For oral administration, the compounds (e.g., a peptide, mixture of peptides, or optionally other therapeutic agent(s)) of the composition(s) can be formulated readily by combining the active compound(s) with pharmaceutically acceptable carriers well known in the art. Such carriers enable the compounds of the present application to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above may be chemically modified so that oral delivery of the derivative is efficacious. Generally, the chemical modification contemplated is the attachment of at least one moiety to the therapeutic agent(s), ingredient(s), and/or excipient(s), where said moiety permits: (a) inhibition of acid hydrolysis; and (b) uptake into the blood stream from the stomach or intestine. Also desired is the increase in overall stability of the therapeutic agent(s), ingredient(s), and/or excipient(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of the compound of the present application (or derivative) or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic compound (e.g., a peptide, mixture of peptides, or optionally other therapeutic agent(s)) or pharmaceutical composition thereof can be included in the formulation as fine multi-particulates in the form of granules or pellets of particle size about 1-2 mm. The formulation of the material for capsule administration could also be as a powder, lightly compressed plugs or even as tablets. The therapeutic compound or pharmaceutical composition could be prepared by compression.

Colorants and flavoring agents may all be included. For example, the compound or pharmaceutical composition of the present application (or derivative) may be formulated and then further contained within an edible product, such as a refrigerated beverage containing colorants and flavoring agents.

One may dilute or increase the volume of the therapeutic compound or pharmaceutical composition with an inert material. These diluents could include carbohydrates, especially mannitol, «-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic compound or composition into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, fumed silica, pyrogenic silica and hydrated silicoaluminate.

To aid dissolution of the therapeutic compound (e.g., a peptide, mixture of peptides, or optionally other therapeutic agent(s)) or composition thereof into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the present application or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

The compounds, peptides, peptide mixtures, or optionally other therapeutic agent(s), and compositions thereof disclosed herein can be included in a formulation as fine multi-particulates in the form of granules or pellets of particle size about 1 mm. The formulation of the material for capsule administration could also be as a powder, lightly compressed plugs or even as tablets. The formulation could be prepared by compression.

For buccal administration, the compositions may take the form of tablets or lozenges formulated in conventional manner.

For topical administration, a peptide, mixture of peptides, or optionally other therapeutic agent(s)) may be formulated as solutions, gels, ointments, creams, suspensions, etc., as are well-known in the art.

For administration by inhalation, a peptide, mixture of peptides, or optionally other therapeutic agent(s) or compositions thereof (e.g. medicament) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament or therapeutic compound can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic compound and a suitable powder base such as lactose or starch.

Nasal delivery of a therapeutic compound (e.g. a peptide, mixture of peptides, or optionally other therapeutic agent(s)) or pharmaceutical composition of the present application is also contemplated. Nasal delivery allows the passage of a therapeutic compound or pharmaceutical composition of the present application to the blood stream directly after administering the therapeutic compound or pharmaceutical composition to the nose, without the necessity for deposition of the product in the lung. Formulations for nasal delivery include those with dextran or cyclodextran.

For nasal administration, a useful device is a small, hard bottle to which a metered dose sprayer is attached. In some embodiments, the metered dose is delivered by drawing the pharmaceutical composition of the present application solution into a chamber of defined volume, which chamber has an aperture dimensioned to aerosolize and aerosol formulation by forming a spray when a liquid in the chamber is compressed. The chamber is compressed to administer the therapeutic compound or pharmaceutical composition. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic compound or pharmaceutical composition.

Alternatively, the therapeutic compound (e.g. a peptide, mixture of peptides, or optionally other therapeutic agent(s)) or pharmaceutical composition may be in powder form for constitution with a suitable vehicle, e.g., sterile pyrogen-free water, before use.

Also contemplated herein is pulmonary delivery of the compounds, peptide, mixture of peptides, or optionally other therapeutic agents disclosed herein (or salts, hydrates, solvates and/or tautomers thereof). The peptide, mixture of peptides, or optionally other therapeutic agent(s) are delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989) (antitrypsin); Smith et al., 1989, J Clin Invest 84:1145-1146 (α-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of a peptide, mixture of peptides, or optionally other therapeutic agent(s) disclosed herein. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4 (3): 201-9 (1994); and Gregoriadis, “Engineering Liposomes for Drug Delivery: Progress and Problems,” Trends Biotechnol., 13 (12): 527-37 (1995). Mizguchi, et al., Cancer Lett., 100:63-69 (1996), describes the use of fusogenic liposomes to deliver a protein to cells both in vivo and in vitro. Chemically modified compound may also be prepared in different formulations depending on the type of chemical modification or the type of device employed.

Formulations suitable for use with a nebulizer, either jet or ultrasonic, will typically comprise a peptide, mixture of peptides, or optionally other therapeutic agent(s) disclosed herein dissolved in water at a concentration of about 0.1 to 25 mg of biologically active compound (e.g., a peptide, mixture of peptides, or optionally other therapeutic agent(s)) per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing a peptide, mixture of peptides, or optionally other therapeutic agent(s) disclosed herein suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing a peptide, mixture of peptides, or optionally other therapeutic agent(s) disclosed herein and may also include a bulking agent, such as lactose, sorbitol, sucrose, trehalose, or mannitol in amounts which facilitate dispersal of the powder from the device, e.g., 50 to 90% by weight of the formulation. The compound (or derivative) should advantageously be prepared in particulate form with an average particle size of less than 10 micrometers (μm), most preferably 0.5 to 5 μm, for most effective delivery to the deep lung.

In addition to the formulations described above, a peptide, mixture of peptides, or optionally other therapeutic agent(s) may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The peptide, mixture of peptides, or optionally other therapeutic agent(s) may be provided in particles. Particles as used herein means nanoparticles or microparticles/microspheres (or in some instances larger particles) which can consist in whole or in part of the compound or the other therapeutic agent(s) as described herein. Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt. The particles may contain the therapeutic agent(s) in a core surrounded by a coating, including, but not limited to, an enteric coating. The peptide, mixture of peptides, or optionally other therapeutic agent(s) also may be dispersed throughout the particles. The peptide, mixture of peptides, or optionally other therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the peptide, mixture of peptides, or optionally other therapeutic agent(s), any of those materials routinely used in the art of pharmacy and medicine, including, but not limited to, erodible, nonerodable, biodegradable, or nonbiodegradable material or combinations thereof. The particles may be microcapsules which contain the compound in a solution or in a semi-solid state. The particles may be of virtually any shape.

Both non-biodegradable and biodegradable polymeric materials can be used in the manufacture of particles for delivering the peptide, mixture of peptides, or optionally other therapeutic agent(s). Such polymers may be natural or synthetic polymers. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly (butylmethacrylate), poly(isobutyl methacrylate), poly (hexylmethacrylate), poly (isodecyl methacrylate), poly (lauryl methacrylate), poly (phenyl methacrylate), poly(methyl acrylate), poly(isopropyl acrylate), poly(isobutyl acrylate), poly (octadecyl acrylate) and polycaprolactone.

A therapeutic compound (e.g., a peptide, mixture of peptides, or optionally other therapeutic agent(s)) or other therapeutic agent or mixtures thereof can be formulated in a carrier system. The carrier can be a colloidal system. The carrier or colloidal system can be a liposome, a phospholipid bilayer vehicle. In one embodiment, therapeutic compound or other therapeutic agent or mixtures thereof can be encapsulated in a liposome while maintaining integrity of the therapeutic compound or other therapeutic agent or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34 (7-8): 915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound (e.g., a peptide, mixture of peptides, or optionally other therapeutic agent(s)) or other therapeutic agent or mixtures thereof can be embedded in the polymer matrix, while maintaining integrity of the composition. The polymer can be a microparticle or nanoparticle that encapsulates the therapeutic agent or agents. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or poly lactic/glycolic acid (PLGA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8): 915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compound (e.g., a peptide, mixture of peptides, or optionally other therapeutic agent(s)) or other therapeutic agent or mixtures thereof are prepared with carriers that will protect the therapeutic compound, other therapeutic agent or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g., a fibrous mesh constructed from several individual pieces of mesh material) or a plurality of individual compositions where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient for at least 2 days. In some embodiments, the implant or depot formulation is constructed and arranged to deliver therapeutic or prophylactic levels of the active ingredient for at least 7 days. In some embodiments, the implant or depot formulation is constructed and arranged to deliver therapeutic or prophylactic levels of the active ingredient for at least 14 days. In some embodiments, the implant or depot formulation is constructed and arranged to deliver therapeutic or prophylactic levels of the active ingredient for at least 30 days. In some embodiments, the implant or depot formulation is constructed and arranged to deliver therapeutic or prophylactic levels of the active ingredient for at least 60 days. In some embodiments, the implant or depot formulation is constructed and arranged to deliver therapeutic or prophylactic levels of the active ingredient for at least 90 days. In some embodiments, the implant or depot formulation is constructed and arranged to deliver therapeutic or prophylactic levels of the active ingredient for at least 180 days. In some embodiments, the implant or depot formulation is constructed and arranged to deliver therapeutic or prophylactic levels of the active ingredient for at least one year. In some embodiments, the implant or depot formulation is constructed and arranged to deliver therapeutic or prophylactic levels of the active ingredient for 15-30 days. In some embodiments, the implant or depot formulation is constructed and arranged to deliver therapeutic or prophylactic levels of the active ingredient for 30-60 days. In some embodiments, the implant or depot formulation is constructed and arranged to deliver therapeutic or prophylactic levels of the active ingredient for 60-90 days. In some embodiments, the implant or depot formulation is constructed and arranged to deliver therapeutic or prophylactic levels of the active ingredient for 90-120 days. In some embodiments, the implant or depot formulation is constructed and arranged to deliver therapeutic or prophylactic levels of the active ingredient for 120-180 days. In some embodiments, the long-term sustained release implants or depot formulation are well-known to those of ordinary skill in the art and include some of the release systems described above. In some embodiments, such implants or depot formulation can be administered surgically. In some embodiments, such implants or depot formulation can be administered topically or by injection.

Therefore, in still another aspect, the present disclosure pertains to composition or medicament for use in treating, preventing, inhibiting, ameliorating or delaying the onset of muscular dystrophy or its signs or symptoms in a mammalian subject in need thereof, wherein the composition or medicament comprises a peptide of Formula A, or a mixture of peptides of Formula A:

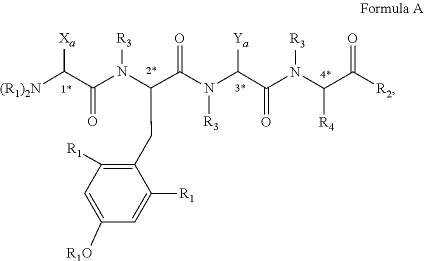

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

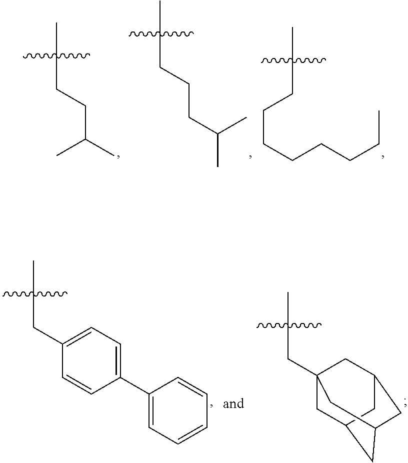

Xₐ is selected from

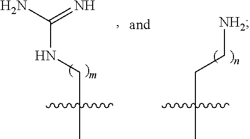

Yₐ each selected from

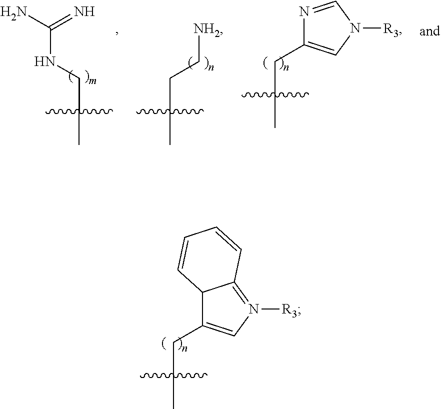

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is substituted with a deuterium or fluorine atom. In some embodiments of the composition or medicament, the peptide or peptides of Formula A is/are a peptide of Formula A-1 or Formula A-2:

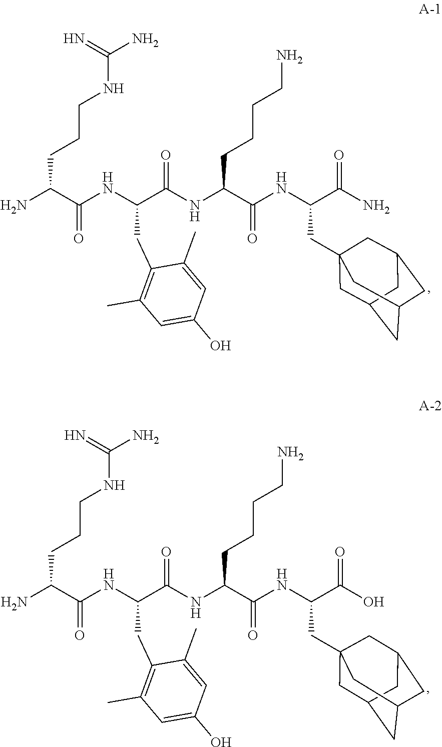 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

is selected from or a pharmaceutically acceptable salt, hydrate, solvate, stereoisomer and/or tautomer thereof. In some embodiments of the composition or medicament, the peptide or peptides of Formula A is a peptide of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

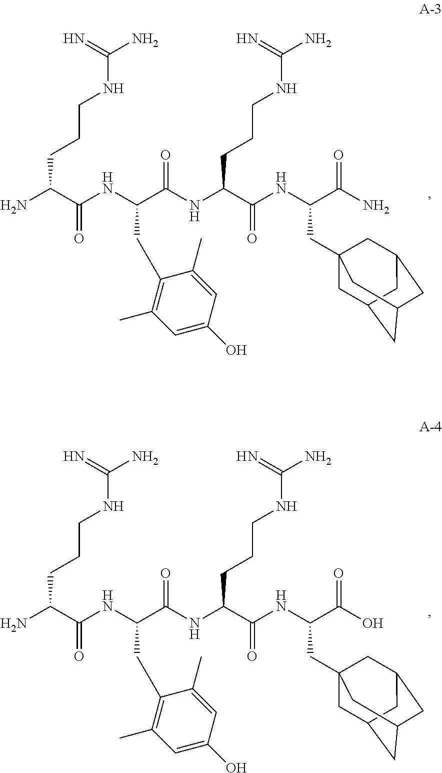 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O]

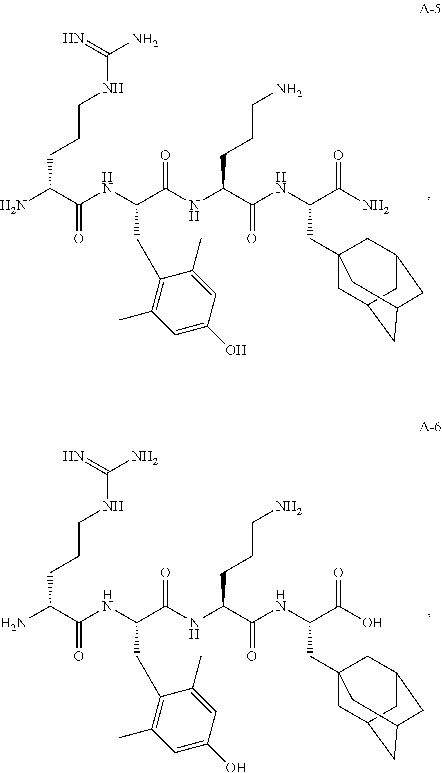 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

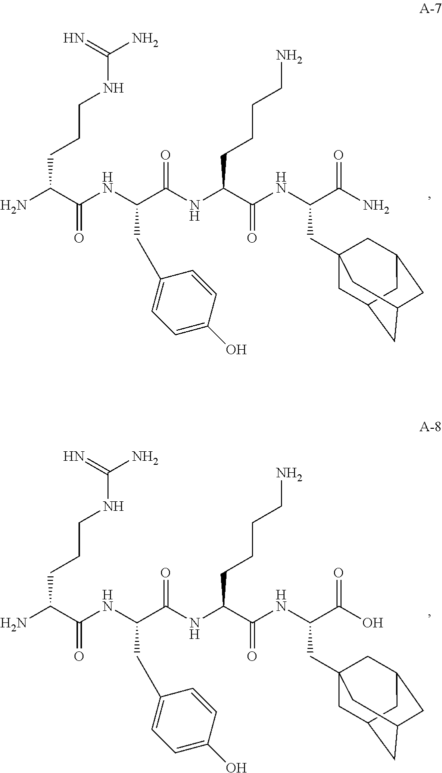 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

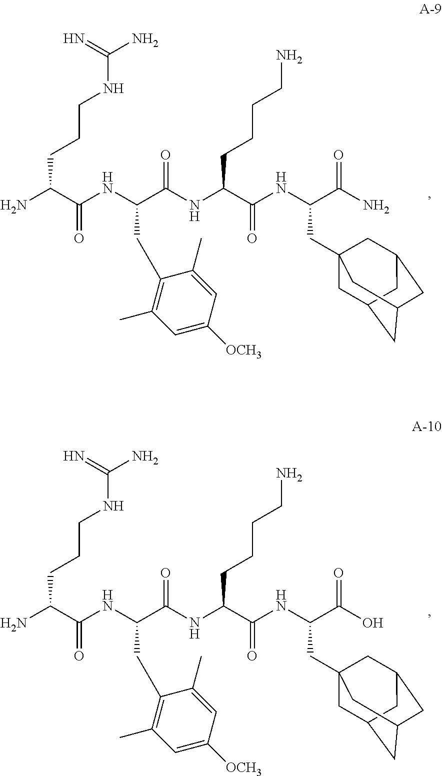 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

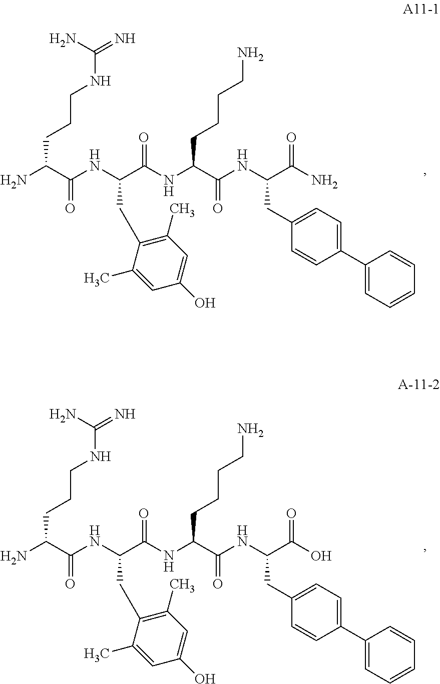 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

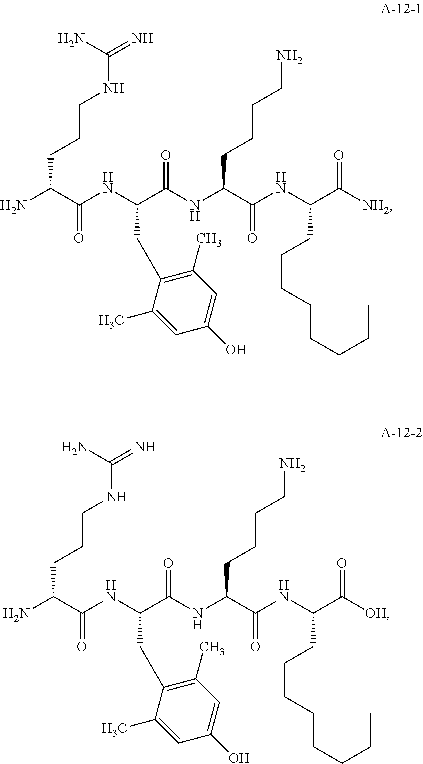 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

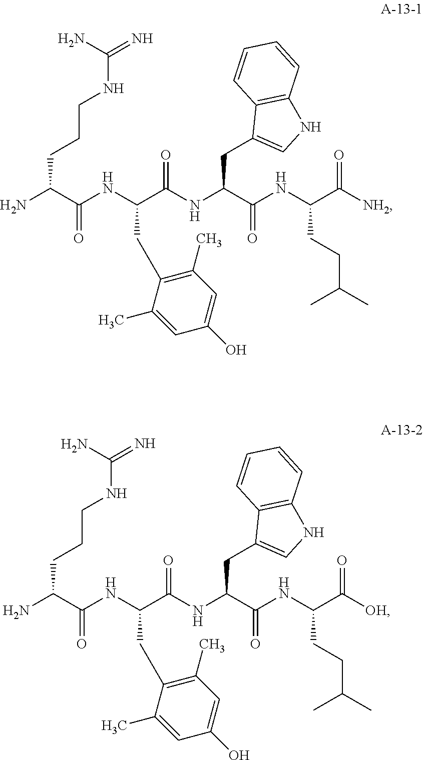 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

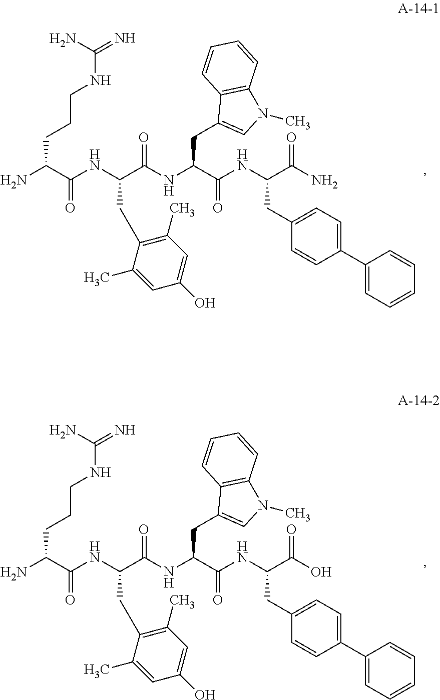 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

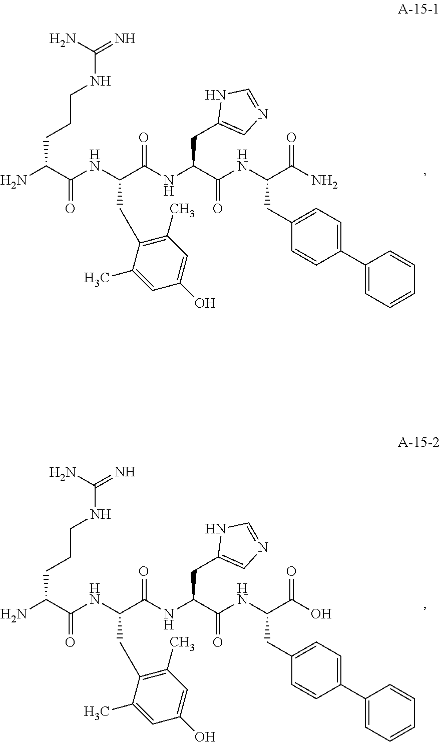 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A (e.g., peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2) are optionally substituted with a deuterium or fluorine atom.

In some embodiments of the composition or medicament, administration of the composition or medicament to the subject ameliorates, delays the onset of, and/or delays the progression of the progression of one or more signs or symptoms of muscular dystrophy in the subject as compared with an untreated control subject or untreated control group that is not administered the composition or medicament.

In some embodiments of the composition or medicament, administration of the composition or medicament to the subject ameliorates, delays the onset of, and/or delays the progression of the progression of a cardiomyopathy associated with muscular dystrophy in the subject as compared with an untreated control subject or untreated control group that is not administered the composition or medicament. In some embodiments of the composition or medicament, administration of the composition or medicament to the subject ameliorates, delays the onset of, and/or delays the progression of calcium overload, systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, heart (cardiac) failure, or any two or more of the foregoing in the subject as compared with an untreated control subject or untreated control group that is not administered the composition or medicament.

In some embodiments of the composition or medicament, administration of the composition or medicament to the subject ameliorates, delays the onset of, and/or delays the progression of contraction-induced damage in muscle tissue, locomotor-skeletal muscle weakness, respiratory muscle weakness, cardiac muscle weakness, sarcolemmal weakening, or sarcolemmal tearing in the subject as compared with an untreated control subject or untreated control group that is not administered the composition or medicament.

In some embodiments of the composition or medicament, the composition or medicament is administered daily for: (i) 12 weeks or more; (ii) 24 weeks or more; (iii) 48 weeks or more; (iv) 72 weeks or more; or (v) 96 weeks or more. In some embodiments of the composition or medicament, the composition or medicament is administered subcutaneously or intravenously. In some embodiments of the composition or medicament, the composition or medicament is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the composition or medicament is administered to a human subject.

In some embodiments of the composition or medicament, the muscular dystrophy is Duchenne muscular dystrophy. In some embodiments of the composition or medicament, muscular dystrophy is Becker's muscular dystrophy.

In some embodiments of the composition or medicament, the pharmaceutically acceptable salt of the peptide or peptides comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, wherein the peptide or peptides of the composition or medicament is/are formulated for administration from its/their tris —HCl salt.

In still another aspect, the present disclosure pertains to composition or medicament for us in treating, preventing, inhibiting, ameliorating or delaying the onset of a cardiomyopathy in a mammalian subject suffering from muscular dystrophy, wherein the composition or medicament comprises a peptide of Formula A, or a mixture of peptides of

### Formula A:

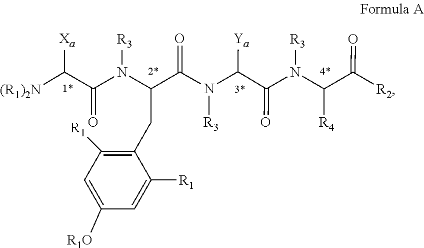

or a pharmaceutically acceptable salt, hydrate, solvate, and/or tautomer thereof, wherein, each R₁ is independently H, —CH₃, or —CH₂CH₃; R₂ is —OH, —NH₂, —NHR₁ or —N(R₁)₂; each R₃ is independently H, or —CH₃; R₄ is selected from:

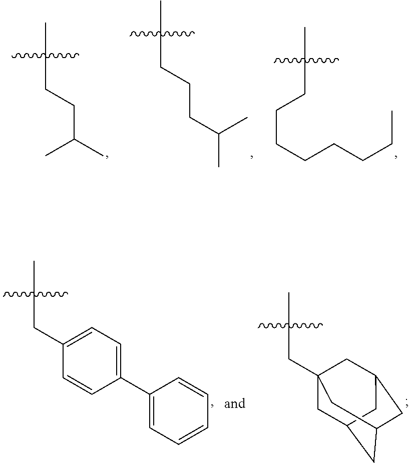

Xₐ is selected from

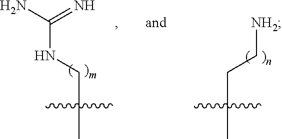

### Yₐ

is selected from

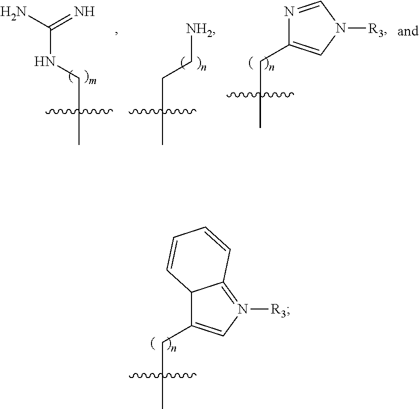

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom. In some embodiments of the composition or medicament, the peptide or peptides of Formula A is/are a peptide of Formula A-1 or Formula A-2:

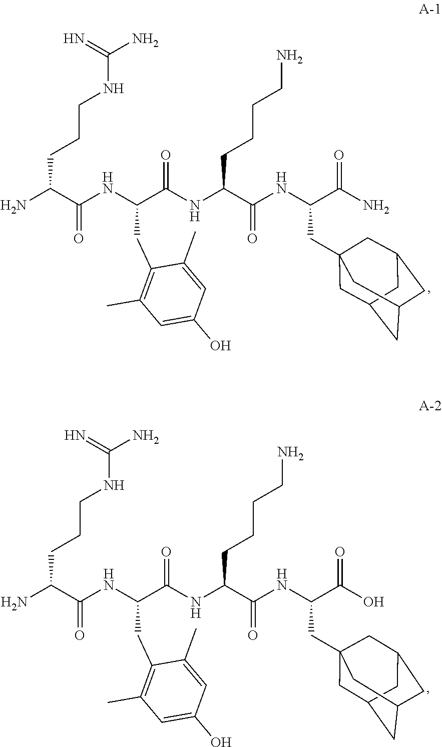 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the composition or medicament, the peptide or peptides of Formula A is a peptide of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

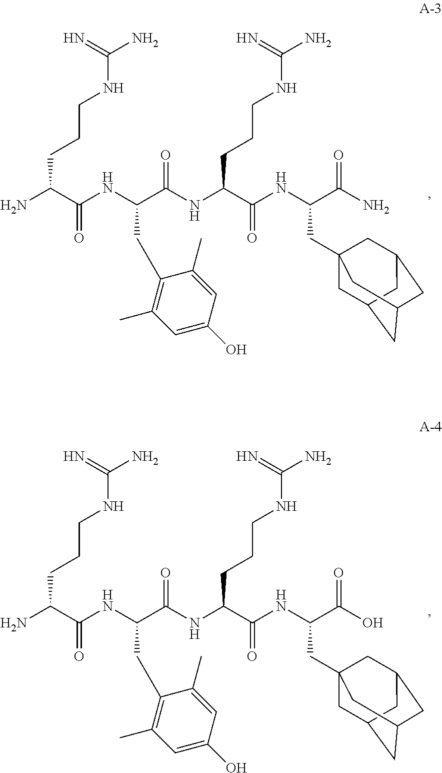 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O]

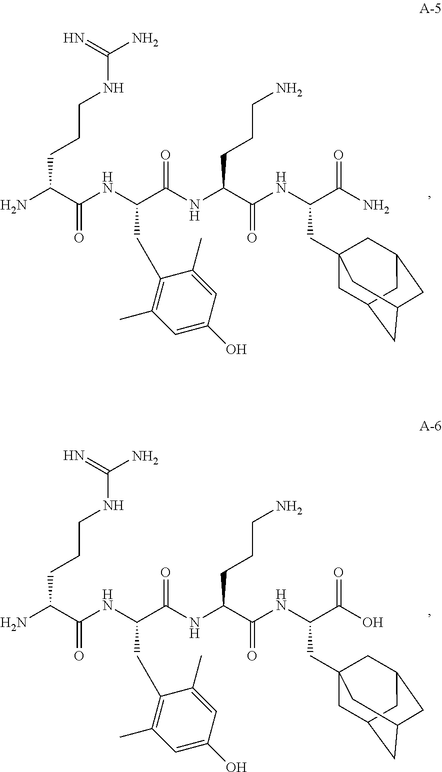 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

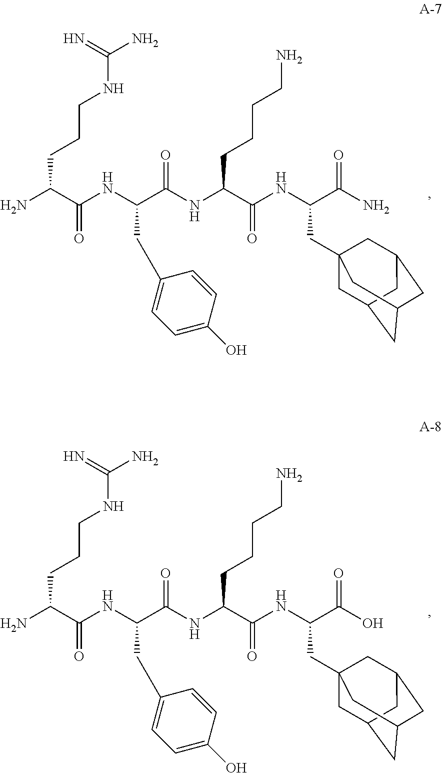 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

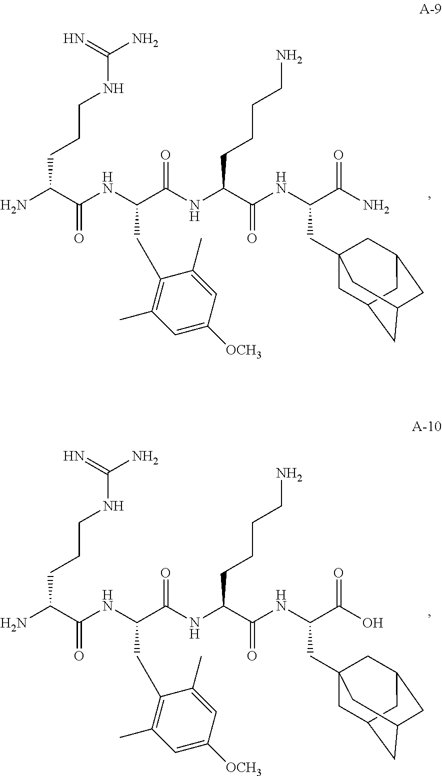 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

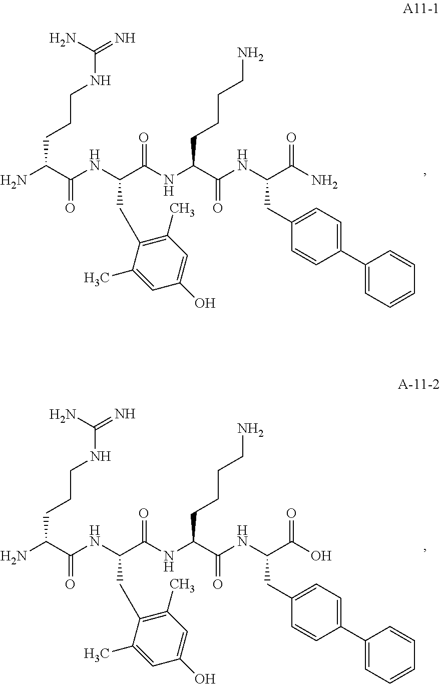 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

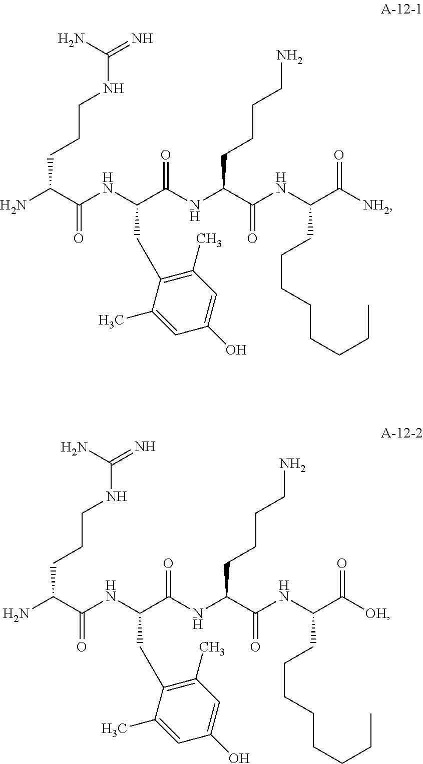 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

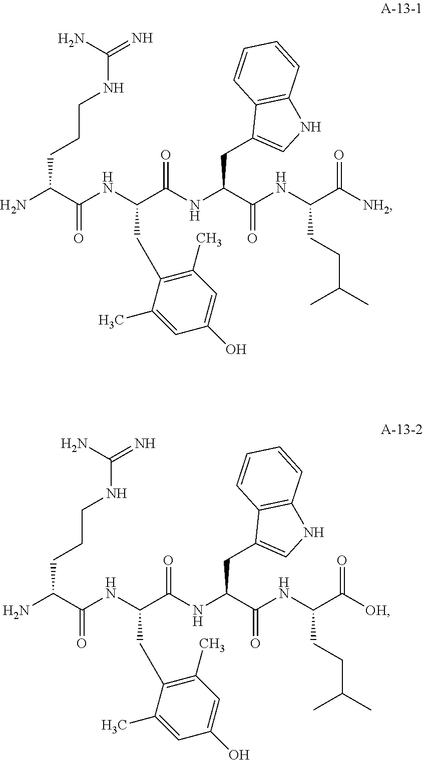 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

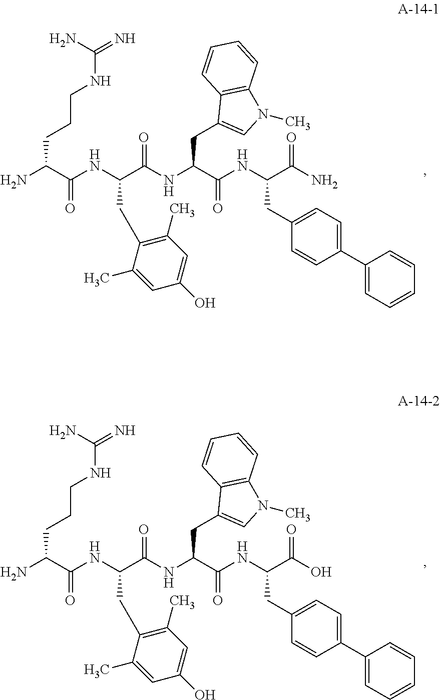 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

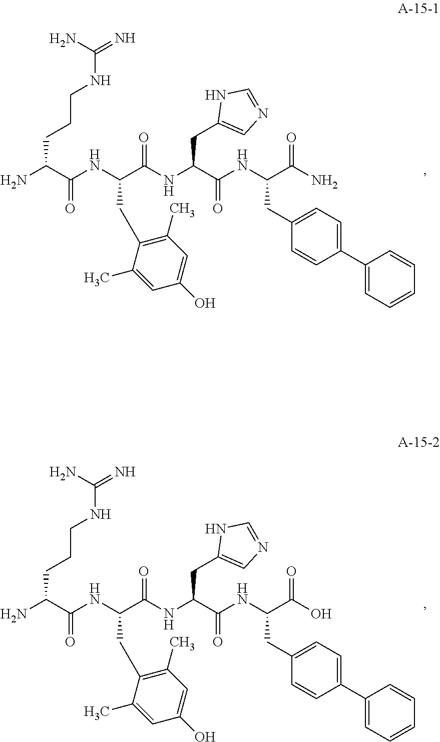 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A (e.g., peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2) are optionally substituted with a deuterium or fluorine atom.

In some embodiments of the composition or medicament, administration of the composition or medicament to the subject ameliorates, inhibits, delays the onset of, and/or delays the progression of calcium overload, systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, heart (cardiac) failure, or any two or more of the foregoing in the subject as compared with an untreated control subject or control group that is not administered the composition or medicament.

In some embodiments of the composition or medicament, administration of the composition or medicament to the subject ameliorates, inhibits, delays the onset of, and/or delays the progression of calcium overload, systolic disfunction, or diastolic dysfunction of the subject as compared with an untreated control subject or control group that is not administered the composition or medicament.

In some embodiments of the composition or medicament, administration of the composition or medicament to the subject ameliorates, inhibits, delays the onset of, and/or delays the progression of ventricular remodeling, including left ventricular remodeling, of the subject as compared with an untreated control subject or control group that is not administered the composition or medicament.

In some embodiments of the composition or medicament, administration of the composition or medicament to the subject ameliorates, inhibits, delays the onset of and/or delays the progression of cardiac (myocardial) fibrosis of the subject as compared with an untreated control subject or control group that is not administered the composition or medicament.

In some embodiments of the composition or medicament, administration of the composition or medicament to the subject ameliorates, inhibits, delays the onset of, and/or delays the progression of fatty acid infiltration of the subject as compared with an untreated control subject or control group that is not administered the composition or medicament.

In some embodiments of the composition or medicament, administration of the composition or medicament to the subject ameliorates, inhibits, delays the onset of, and/or delays the progression of hypertrophic cardiomyopathy of the subject as compared with an untreated control subject or control group that is not administered the composition or medicament.

In some embodiments of the composition or medicament, administration of the composition or medicament to the subject ameliorates, inhibits, delays the onset of, and/or delays the progression of dilated cardiomyopathy of the subject as compared with an untreated control subject or control group that is not administered the composition or medicament.

In some embodiments of the composition or medicament, administration of the composition or medicament to the subject ameliorates, inhibits, delays the onset of, and/or delays the progression of an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia) of the subject as compared with an untreated control subject or control group that is not administered the composition or medicament.

In some embodiments of the composition or medicament, administration of the composition or medicament to the subject ameliorates, inhibits, delays the onset of, and/or delays the progression of reduced left ventricular ejection fraction of the subject as compared with an untreated control subject or control group that is not administered the composition or medicament.

In some embodiments of the composition or medicament, administration of the composition or medicament to the subject ameliorates, inhibits, delays the onset of, and/or delays the progression of fractional shortening of the subject as compared with an untreated control subject or control group that is not administered the composition or medicament.

In some embodiments of the composition or medicament, administration of the composition or medicament to the subject ameliorates, inhibits, delays the onset of, and/or delays the progression of heart (cardiac) failure of the subject as compared with an untreated control subject or control group that is not administered the composition or medicament.

In some embodiments of the composition or medicament, administration of the composition or medicament to the subject results in normalization of the ejection fraction, fractional shortening, stroke volume, and/or cardiac output of the subject as compared with an untreated control subject or control group that is not administered the composition or medicament.

In some embodiments of the composition or medicament, the composition or medicament is administered daily for: (i) 12 weeks or more; (ii) 24 weeks or more; (iii) 48 weeks or more; (iv) 72 weeks or more; or (v) 96 weeks or more. In some embodiments of the composition or medicament, the composition or medicament is administered subcutaneously or intravenously. In some embodiments of the composition or medicament, the composition or medicament is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the composition or medicament is administered to a human subject.

In some embodiments of the composition or medicament, the muscular dystrophy is Duchenne muscular dystrophy. In some embodiments of the composition or medicament, the muscular dystrophy is Becker's muscular dystrophy.

In some embodiments of the composition or medicament, administration of the composition or medicament further comprises separately, sequentially, or simultaneously administering an additional therapeutic agent to the subject.

In some embodiments of the composition or medicament, the additional therapeutic agent is a phosphorodiamidate morpholino oligomer (PMO) or a peptide-conjugated phosphorodiamidate morpholino oligomer (PPMO). In some embodiments of the composition or medicament, the PMO is selected from the group consisting of eteplirsen (Exondys 51®), golodirsen (Vyondys 53®), viltolarsen (Viltepso®), and Casimersen (Amondys 45®) or the PPMO is selected from vesleteplirsen (a.k.a., SRP-5051) and PGN-EDO51 (a.k.a., CONNECT1-ED051).

In some embodiments of the composition or medicament, the additional therapeutic agent is an adeno-associated virus (AAV) vector-based gene therapy. In some embodiments of the composition or medicament, the adeno-associated virus vector-based gene therapy is delandistrogene moxeparvovec-rokl (Elevidys®).

In some embodiments of the composition or medicament, the additional therapeutic agent is a corticosteroid, an angiotensin-converting enzyme (ACE) inhibitor, an angiotensin receptor blocker (ARB), a beta blocker, a diuretic, or a mineralocorticoid receptor antagonist. In some embodiments of the composition or medicament, the corticosteroid is vamorolone (aGamree®), prednisone, or deflazacort. In some embodiments of the composition or medicament, the angiotensin-converting enzyme (ACE) inhibitor is captopril, enalapril, lisinopril, benazepril, or ramipril. In some embodiments of the composition or medicament, the angiotensin receptor blocker (ARB) is azilsartan, candesartan, eprosartan, irbesartan, telmisartan, valsartan, losartan, olmesartan, Entresto® (sacubitril/valsartan) or Byvalson™ (nebivolol/valsartan). In some embodiments of the composition or medicament, the beta blocker is carvedilol, bisoprolol, metoprolol succinate, atenolol, esmolol, nebivolol, or propranolol. In some embodiments of the composition or medicament, the diuretic is bumetanide, furosemide, or torsemide. In some embodiments of the composition or medicament, the mineralocorticoid receptor antagonist is spironolactone or eplerenone.

In some embodiments of the composition or medicament, the additional therapeutic agent is mavacamten (Camzyos®).

In some embodiments of the composition or medicament, the pharmaceutically acceptable salt of the peptide or peptides of the composition or medicament comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, the peptide or peptides of the composition or medicament is/are formulated for administration from its/their tris —HCl salt.

In some embodiments, the compositions or formulations can be used as medicaments or in the preparation of medicaments for: (i) treating the signs, symptoms, or severity of muscular dystrophy (e.g., DMD or BMD) in a mammalian subject in need thereof, wherein the subject is suffering from muscular dystrophy; (ii) inhibiting the signs, symptoms, or severity of muscular dystrophy (e.g., DMD or BMD) in a mammalian subject in need thereof, wherein the subject is suffering from muscular dystrophy; (iii) preventing the signs, symptoms, or severity of muscular dystrophy (e.g., DMD or BMD) in a mammalian subject in need thereof, wherein the subject is suffering from muscular dystrophy; (iv) ameliorating the signs, symptoms, or severity of muscular dystrophy (e.g., DMD or BMD) in a mammalian subject in need thereof, wherein the subject is suffering from muscular dystrophy; (v) delaying the onset of the signs, symptoms, or severity of muscular dystrophy (e.g., DMD or BMD) in a mammalian subject in need thereof, wherein the subject is suffering from muscular dystrophy; (vi) delaying the onset of muscular dystrophy (e.g., DMD or BMD) in a mammalian subject in need thereof, wherein the subject is suspected of having or at risk for developing muscular dystrophy, or (vii) augmenting the production of dystrophin in a mammalian subject in need thereof, wherein the subject is suspected of having or at risk for developing muscular dystrophy (e.g., DMD or BMD). In some embodiments, the compositions are used as medicaments or in the preparation of medicaments for augmenting dystrophin expression in a subject as compared to an untreated subject or as compared to a control subject administered either the peptide or peptide mixture alone or in combination with PMO(s) and/or PPMO(s).

### Combination Therapy

In some embodiments, the peptide or mixtures of peptides disclosed herein may be combined with one or more additional therapeutic agents related to the treatment of (including without limitation the inhibition of, prevention of, amelioration of, or delaying the onset of) muscular dystrophy (e.g., DMD or BMD) in a subject, including a human subject.

Some additional therapeutic agents include, but are not limited to, a phosphorodiamidate morpholino oligomer (PMO) or a peptide-conjugated phosphorodiamidate morpholino oligomer (PPMO). In some embodiments, the PMO is selected from the group consisting of eteplirsen (Exondys 51®), golodirsen (Vyondys 53®), viltolarsen (Viltepso®), and casimersen (Amondys 45™) and the PPMO is selected from vesleteplirsen (a.k.a., SRP-5051) and PGN-EDO51 (a.k.a., CONNECT1-ED051). In some embodiments, the PMO (or the PMO of the PPMO) comprises an appropriately designed exon-skipping oligomer that is relevant to the dystrophin lesion in a subject in need thereof, wherein the subject has been diagnosed with or is suspected of having a muscular dystrophy, such as DMD or BMD. By way of example, but not by limitation, the PMO comprises an antisense oligomer of about 20-50 nucleotides in length, or a pharmaceutically acceptable salt thereof, capable of binding a selected target in human dystrophin pre-mRNA to induce exon skipping in the human dystrophin gene, wherein the antisense oligomer comprises a sequence of bases that specifically hybridizes to a dystrophin exon target region.

Some additional therapeutic agents include, but are not limited to, an adeno-associated virus (AAV) vector-based gene therapy. In some embodiments, the adeno-associated virus (AAV) vector-based gene therapy is delandistrogene moxeparvovec-rokl (Elevidys®).

Some additional therapeutic agents include, but are not limited to, a corticosteroid, an angiotensin-converting enzyme (ACE) inhibitor, an angiotensin receptor blocker (ARB), a beta blocker, a diuretic, or a mineralocorticoid receptor antagonist. For example, the corticosteroid can be vamorolone (aGamree®), prednisone, or deflazacort. For example, the angiotensin-converting enzyme (ACE) inhibitor can be captopril, enalapril, lisinopril, benazepril, or ramipril. For example, the angiotensin receptor blocker (ARB) can be azilsartan, candesartan, eprosartan, irbesartan, telmisartan, valsartan, losartan, olmesartan, Entresto® (sacubitril/valsartan) or Byvalson™ (nebivolol/valsartan). For example, the beta blocker can be carvedilol, bisoprolol, metoprolol succinate, atenolol, esmolol, nebivolol, or propranolol. For example, the diuretic can be bumetanide, furosemide, or torsemide. For example, the mineralocorticoid receptor antagonist can be spironolactone or eplerenone.

In some embodiments, the additional therapeutic agent is mavacamten (Camzyos®)

In some embodiments, when an additional therapeutic agent is administered to a subject a synergistic therapeutic effect is produced. For example, administration of the peptide or mixture of peptides and PMO(s) and/or PPMO(s), optionally with one or more additional therapeutic agents, will have greater than additive effects in the treatment of the disease. For example, lower doses of one or more of any individual therapeutic agent may be used in treating or preventing muscular dystrophy, resulting in increased therapeutic efficacy and decreased side-effects. In some embodiments, the synergistic effect will be improved ambulation (or delay in reduction in ambulation) resulting from the combined effects of increases in muscular dystrophin with increases in muscle function and energy associated with improved mitochondrial health of the subject (and the subject's muscles). In some embodiments, the synergistic effect will be extended life expectancy resulting from the combined effects of increases in muscular dystrophin resulting in improved muscle function (e.g., cardiac function) and energy associated with improved mitochondrial health of the subject (and the subject's muscles).

### Kits

The present technology also provides kits for treating muscular dystrophy (e.g., Duchenne muscular dystrophy or Becker's muscular dystrophy). In some embodiments, the kits comprise at least one peptide of Formula A (e.g., Compound A-1, A-2, A-3, A-4, A-5, A-6, A-7, A-8, A-9, A-10, A-11-1, A-11-2, A-12-1, A-12-2, A-13-1, A-13-2, A-14-1, A-14-2, A-15-1 or A-15-2) or mixtures thereof; alone or as formulated into a composition or medicament) and at least one additional therapeutic agents such as: (i) any suitable PMO or PPMO, and/or (ii) a corticosteroid, an angiotensin-converting enzyme (ACE) inhibitor, an angiotensin receptor blocker (ARB), a beta blocker, a diuretic, or a mineralocorticoid receptor antagonist, packaged in a suitable container and optionally comprising instructions for its/their use.

It will be understood by one of ordinary skill in the relevant arts that other suitable modifications and adaptations to the compositions and methods described herein are readily apparent from the description of the technology contained herein in view of information known to the ordinarily skilled artisan, and may be made without departing from the scope of the present technology or any embodiment thereof. Having now described the present technology in detail, the same will be more clearly understood by reference to the following examples, which are included herewith for purposes of illustration only and are not intended to be limiting of the present technology.

### EXAMPLES

The present technology is further illustrated by the following examples, which should not be construed as limiting in any way. For each of the examples below, any peptide of Formula A (e.g., a compound of Formula A-1, Formula A-2, Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, Formula A-15-2 or mixtures thereof) could be used. Regardless, but not by limitation, the peptide used in the following examples below was the peptide of Formula A-1. The peptide of Formula A-1 was prepared by solution-phase methods by first making the protected Boc-D-Arg-DMT-OH dimer (41; See: FIG. 4) and the protected H2N-Lys (Boc)-ADM-NH₂ dimer (42; See: FIG. 4), followed by condensing the two dimers and deprotecting the tetrapeptide as shown in FIG. 4; Also see: WO2020/131283. All other peptides of Formula A can be prepared using the generic process discussed in the Scheme at FIG. 4 by selecting the appropriate amino acids to form the appropriate peptide dimers.

### Example 1-ASSESSMENT OF THE PHARMACOKINETICS OF ELAMIPRETIDE AND the Peptide of Formula α-1 Following Intraperitoneal Dose Administration to Male C57Bl/6 Mice

### a) Dosing Protocol:

Animals: Male/C57BL/6 mice/-18-25 grams were received from an approved vendor.
An untreated control group of n=3 mice (Group 3) was used to provide control plasma for Group 1 and Group 2, and control tissues for Group 2
Fasting was not required for this study.
Body weights were recorded prior to dose administration. The volume of each dose delivered (mL/kg) was based on each individual animal's body weight.
Doses were administered in accordance with test facility standard operating procedures.
All dose syringes were weighed prior to and following dosing to gravimetrically determine the amount of formulation administered.
All animals were observed at dosing and each scheduled collection. Any abnormalities were recorded.
Elamipretide and the peptide of Formula A-1 were formulated as follows:
   Elamipretide was provided in powder form and formulated for administration by the contract research organization (CRO) vendor. Elamipretide was removed from the refrigerator and the un-opened bottle was allowed to equilibrate at room temperature for a minimum of 2 hours. Then, elamipretide was dissolved in 0.9% (wt.//vol.) saline at a concentration of 1.5 mg/mL, using a correction factor of 83.3% to account for salt content and HPLC purity. The drug formulation was filtered through 0.2 μm polyether sulfone (PES) filter and stored at 2-8° C. until use.
   The peptide of Formula A-1 was provided in powder form and formulated for administration by the CRO vendor. The peptide of Formula A-1 was removed from the refrigerator and the un-opened bottle was allowed to equilibrate at room temperature for a minimum of 2 hours. Then, the peptide of Formula A-1 was dissolved in 0.9% (wt.//vol.) saline at a concentration of 1.5 mg/mL, using a correction factor of 85.3% to account for salt content and HPLC purity. The drug formulation was filtered through 0.2 μm polyether sulfone (PES) filter and stored at 2-8° C. until use.
Study Design is set forth in Table 1, below.

TABLE 1
Study Design

| | Number | | Dose | Dose | Dose | | |
|---|---|---|---|---|---|---|---|
| Group | of | Test | Level | Conc. | Volume | | |
| Number | Animals | Article | (mg/kg) | (mg/mL) | (mL/kg) | Vehicle | Route |
| 1 | 15M | Elam | 5.0 | 1.5 | 3.33 | 0.9% saline | IP |
| 2 | 15M | A-1 | 5.0 | 1.5 | 3.33 | 0.9% saline | IP |
| 3 | 3M | Untreated | N/A | N/A | N/A | N/A | N/A |

15M = 15 male mice;
3M = 3 male mice;
Elam = elamipretide;
A-1 = the peptide of Formula A-1;
N/A = not applicable;
IP = intraperitoneal

b) Sample collection-Post Dosing:
   Terminal blood samples were collected via cardiac puncture following inhalation of anesthesia in accordance with test facility standard operating procedures. Group 1 animals were sacrificed following cardiac puncture.
   Blood samples were collected into tubes with appropriate anticoagulant as specified in Table 2. The tubes were stored on wet ice until processed to plasma by centrifugation (3500 rpm at 5° C. for 10 minutes) within 20 minutes of collection. Sample aliquots of 100 μL were transferred into individual uniquely labeled matrix tubes and stored at nominal −80° C. until transferred to analytical chemistry for analysis by LC/MS/MS.
   Immediately following each terminal blood collection for Groups 2 and 3, animals were sacrificed and perfused with cold PBS. The following tissues were collected: heart and muscle in accordance with Table 2.
   Plasma and tissue samples were processed for analysis by LC/MS/MS to determine the amounts/concentrations reported in FIG. 5A and FIG. 5B.

TABLE 2
Sample Collection Design

| | Terminal Blood | Tissue Collections |
|---|---|---|
| Group | Collections Post-Dose | (n = 3 per |
| Number | (N = 3 per dose timepoint) | dose timepoint) |
| 1 | 0.5, 1, 2, 8, and 24 hours | N/A |
| | (n = 3 per time point) | |
| 2 | 0.5, 1, 2, 8, and 24 hours | Saline perfusion |
| | (n = 3 per time point) | followed by collection |
| | | of heart and muscle |
| 3 | 0 hours | Saline perfusion |
| | | followed by collection |
| | | of heart and muscle |
| Anticoagulant | K2 EDTA | N/A |
| Volume/ | Max obtainable (terminal) | N/A |
| Timepoint | | |

Results: The results are presented in graphic form in FIG. 5A and FIG. 5B. With reference to FIG. 5A, the plasma exposure of the peptide of Formula A-1 is compared with the plasma exposure of elamipretide. The data indicates that when both are administered in the same manner and at the same dose, the peptide of Formula A-1 is roughly 3-4 times more concentrated in the plasma at 30 minutes to one hour as compared with elamipretide and is still present in the plasma at greater than 100 ng/ml after 4 hours whereas elamipretide is present at 100 ng/ml at 2 hours and generally dropped below the Ing/mL lower limit of quantification at 4 hours.

With reference to FIG. 5B, data is presented for the concentration of the peptide of Formula A-1 in plasma, heart and muscle. The data demonstrates that the there is good uptake of the peptide of Formula A-1 in heart and muscle, without any significant difference observed in uptake between the two tissues (i.e., muscle and heart). Whilst tissue concentrations from 30 minutes to 2 hours were about 10 times lower than the plasma concentrations (assuming a tissue density of 1 g/mL), the peptide of Formula A-1 provides reasonable penetration into muscle and heart tissue sufficient to potentially produce an efficacious drug effect. By 8 hours, the concentration of the peptide of Formula A-1 present in the tissues (heart and muscle) is higher than that in the plasma. It is also worth noting that administration of the peptides (elamipretide and Formula A-1) was well tolerated by the animals.

### Example 2-ASSESSMENT OF THE PHARMACOKINETICS OF ELAMIPRETIDE AND Dose Escalation of the Peptide of Formula α-1 Following Intraperitoneal Administration to Male C57Bl/6 Mice

### a) Dosing Protocol:

Animals: Male/C57BL/6 mice/-18-25 grams were received from an approved vendor and allowed to acclimate to the test facility for 2 days prior to the start of the study. Group 4 animals were monitored on days 1 and 2 for 2 hours post dosing.
Fasting was not required for this study.
Body weights were recorded prior to dose administration. The volume of each dose delivered (mL/kg) was based on each individual animal's body weight.
Doses were administered in accordance with test facility standard operating procedures.
This was a multiple dose terminal study dosed once per day over 5 days. Mice were taken down following their 5th and final dose.
All dose syringes were weighed prior to and following dosing to gravimetrically determine the amount of formulation administered.
All animals were observed at dosing and each scheduled collection. Any abnormalities were recorded.
Elamipretide and the peptide of Formula A-1 were formulated as follows:
   Elamipretide was provided in powder form. Elamipretide was removed from the refrigerator and the un-opened bottle was allowed to equilibrate at room temperature for a minimum of 2 hours. Then, elamipretide was dissolved in 0.9% saline (wt.//vol.) at a concentration of 1.5 mg/mL, using a correction factor of 83.3% to account for slat content and HPLC purity. The drug formulation was filtered through 0.2 μm polyether sulfone (PES) filter and stored at 2-8° C. until use.
   The peptide of Formula A-1 was provided in powder form. The peptide of Formula A-1 was removed from the refrigerator and the un-opened bottle was allowed to equilibrate at room temperature for a minimum of 2 hours. Then, the peptide of Formula A-1 was dissolved in 0.9% (wt.//vol.) saline at a concentration of 0.3 mg/mL (Group 2) 1.5 mg/mL (Group 3) and 4.5 mg/mL (Group 4), in each case using a correction factor of 85.3% to account for salt content and HPLC purity. The drug formulation was filtered through 0.2 μm PES filter and stored at 2-8° C. until use.
Study Design is set forth in Table 3, below.

TABLE 3
Study Design

| | Number | | Dose | Dose | Dose | | | |
|---|---|---|---|---|---|---|---|---|
| Group | of | Test | Level | Conc. | Volume | | | |
| Number | Animals | Article | (mg/kg) | (mg/mL) | (mL/kg) | Dose Schedule | Vehicle | Route |
| 1 | 3M | Elam | 5.0 | 1.5 | 3.33 | 1x daily for 5 days | 0.9% saline | IP |
| 2 | 3M | A-1 | 1.0 | 0.3 | 3.33 | 1x daily for 5 days | 0.9% saline | IP |
| 3 | 3M | A-1 | 5.0 | 1.5 | 3.33 | 1x daily for 5 days | 0.9% saline | IP |
| 4 | 3M | A-1 | 15.0 | 4.5 | 3.33 | 1x daily for 5 days | 0.9% saline | IP |

3M—3 male mice;
Elam = elamipretide;
A-1—the peptide of Formula A-1;
IP = intraperitoneal

### b) Sample Collection-Post Dosing:

Terminal blood samples were collected via cardiac puncture following inhalation of anesthesia in accordance with test facility standard operating procedures.
Immediately following each terminal blood collection for all groups, animals were sacrificed and perfused with cold PBS. The following tissues were collected: heart, gastrocnemius muscle, and liver in accordance with Table 4.
Plasma and tissue samples were processed for analysis by LC/MS/MS to determine the amounts/concentrations reported in the FIG. 6.

TABLE 4
Sample Collection Design

| | Terminal Blood | |
|---|---|---|
| | Collections Post-Dose | |
| Group | (N = 3 per | |
| Number | dose timepoint) | Tissue Collections |
| 1-4 | 2 hours following 5^{th} | Cold PBS perfusion followed |
| | and final dose | by collection of heart, |
| | | gastrocnemius muscle, |
| | | and liver |
| Anticoagulant | K2 EDTA | N/A |
| Volume/ | Max obtainable | N/A |
| Timepoint | (terminal) | |

### Results:

With reference to FIG. 6, the concentration (measured in ng test article/gram of tissue) of test articles (elamipretide and the peptide of Formula A-1) is presented in a bar graph. The graph compares the concentration in muscle (gastrocnemius), heart, and liver of elamipretide dosed at 5 mg/kg IP as compared with the peptide of Formula A-1, dosed at 1 mg/kg, 5 mg/kg and 15 mg/kg, each by IP. With reference to FIG. 6, the tissue concentrations of elamipretide administered at 5 mg/kg was roughly equivalent to the tissue concentrations of the peptide of Formula A-1 administered at a fivefold lower amount (i.e., 1 mg/kg). By 8 hours, the concentration of the peptide of Formula A-1 present in the tissues (heart and muscle) is higher than that in the plasma. The data further demonstrates that the peptide of Formula A-1 appears to penetrate all tissues, therefore suggesting that the compound of Formula A-1 can be delivered to tissues in an amount to produce an efficacious drug effect, including possibly delivering an efficacious dose at a 5× lower amount or concentration. This result is surprising in that nothing would suggest such a significant improvement in uptake between elamipretide and the compound of Formula A-1. It is also worth noting that administration of the peptides (elamipretide and the peptide of Formula A-1) was well tolerated by the animals.

### Example 3-ASSESSMENT OF HEART FUNCTION IN a MOUSE MODEL OF Duchenne Muscular Dystrophy (DMD) Treated with a Low and A High Dose of the Peptide of Formula α-1

This example was performed to examine the effect of the treatment of a Duchenne muscular dystrophy (DMD) mouse model with low and high doses of the peptide of Formula

A-1 in the heart and diaphragm of the test subjects.

### Methods:

Male D2.mdx mice originated from breeding colonies maintained at York University (Toronto, Canada) and sourced from The Jackson Laboratory (Bar Harbor, United States). DBA/2J wild type mice were purchased from The Jackson Laboratory at 4-5 weeks of age. D2.mdx mice received vehicle control or low dose (1 mg/kg) or high dose (5 mg/kg) of the peptide of Formula A-1 via daily subcutaneous injections for 4 weeks. After 4 weeks of treatment, invasive hemodynamics were measured. An open-chest approach was chosen based on the ease of RV catheterization, minimization of breathing artifacts on RV pressure-volume (P-V) loops, and increased control of respiration. After being anesthetized with isoflurane (3%-4% induction, 1%-2% maintenance), mice were tracheotomized, intubated, and placed on a ventilator (Harvard Apparatus Inc., Holliston, MA) with respiration rates (RR, min⁻¹) and tidal volume (Vt, mL) optimized for mice and to limit the effects of mechanical ventilation using the following formulas (M=mouse weight):

$$RR = 53 : 5 \times {M}^{- 0.26} {min}^{- 1} and Vt = 6 : 2 \times {M}^{101} mL$$

Core temperature was maintained between 36.9 and 37.3° C. and was continually monitored by a rectal probe. After insertion of a polyethylene tube into the right external jugular vein to allow fluid and drug administration, a subxiphoid incision extending toward the sternum was made, exposing the diaphragm, which was then resected. Bleeding was controlled with electrocautery. The chest wall and lungs were then retracted to expose the heart. Without removing the pericardium, the LV or RV apex was punctured with a 26- or 27-gauge hypodermic needle, which allowed for insertion of a 1.2-Fr P-V admittance catheter (Scisense, London, ON) while preventing bleeding from the heart. Positioning of the P-V catheter along the longitudinal axis of each chamber was determined by analyzing the pressure versus magnitude loops (i.e., blood conductance) and phase signals (e.g., muscle conductance), with admittance volume mathematically calculated once an optimal range of magnitude (900-2,000 μS) and phase) (2-8° signals were achieved. Further, inferior vena cava occlusion (IVCO) was performed by pulling a small 5-0 suture placed around the vessel before recording to ensure the catheter did not become entrapped or that electrodes were not in contact with intracardiac structures during load-insensitive measurements.

The order of LV and RV measurements were randomized and measured in each animal, with steady-state measurements completed in each chamber. Prior to P-V data acquisition, echocardiographically derived SV for each animal was used for calibration of the catheter-derived admittance signal. Following positioning of the catheter in the ventricle, mice were allowed to stabilize for 10-15 minutes before recording pressure and volume in real time as well as the magnitude and phase of the electrical admittance impedance for 10 min (ADVantage; Scisense) at 1,000 Hz and stored for offline analysis (Labscribe3; iWorx, Dover, NH) to generate the P-V curves. Next, an IVCO was performed (five times per animal, 2 min between IVCO) for determining end-systolic and end-diastolic pressure relationships. The IVCO was limited to 1-2 s in duration to avoid excessive reflex responses (e.g., baroreflex) that would alter heart rate (HR) and/or pressure.

Diaphragm muscle was carefully excised while the animal was anesthetized with medical-grade isoflurane. The diaphragm was preserved in 10% neutral-buffered formalin (Millipore Sigma, Burlington, MA, USA) and stored for 24 hours at room temperature. Diaphragms were then transferred to a separate 15 mL conical centrifuge tube containing 70% ethanol and stored at 4° C. Once ready for tissue processing, diaphragms were individually placed in plastic paraffin-embedding cassettes (Simport Scientific, Saint-Mathieu-de-Beloeil, QC, Canada) where they were dehydrated using a gradient of ethanol concentrations (70% to 99%), followed by two 100% xylene incubations. Cassettes were placed overnight in an oven heated to 54-57° C., which contained melting Type H paraffin wax (Thermo Fisher Scientific, USA), in a process intended to allow paraffin wax to penetrate and embed the samples. The following morning, after cassettes were removed from the oven and paraffin wax had solidified at room temperature, paraffin within the cassettes were again heated to melting point, and samples were transferred to disposable base molds. Paraffin was once again re-introduced to the cassettes and left to solidify at room temperature before the samples were ready to be sectioned at 5 μm thickness on a frontal (4-chamber) plane using a microtome and hot water bath. Finally, sections were carefully placed on Fisherbrand SuperFrost Plus positively charged microscope slides (Thermo Fisher Scientific) for microscopy.

Fibrosis was assessed on formalin-fixed paraffin-embedded sections with picrosirius red (PSR), which stains collagen. Sections were deparaffinized with xylene incubations and subsequently rehydrated with ethanol incubations (100% to 70%, followed by distilled water). Sections were then incubated with PSR for 1 hour at room temperature, followed by washes in acidified water. Prior to mounting, sections were submerged in ethanol concentrations (95% to 100%), followed by xylene incubations, and then subsequently mounted with Permount mounting medium (Thermo Fisher Scientific). Sections were imaged with Brightfield microscopy on the EVOS M7000 imaging system (Thermo Fisher Scientific). Fibrotic (collagenous) regions were expressed as a percentage against standardized total regions of interest (anatomically consistent between samples). Artefacts (such as irregular tissue folds) were avoided during the blinded analysis. Fibrosis was quantified using ImageJ (National Institute of Health).

Data obtained by performing this Example is presented graphically in FIG. 7A, FIG. 7B and FIG. 8.

### Results:

Right ventricle end-systolic pressure (ESP-FIG. 7A) and end-diastolic pressure (EDP-FIG. 7B) from wildtype mice, D2.mdx mice treated with vehicle, D2.mdx mice treated with low dose (1 mg/kg) peptide of Formula A-1, or D2.mdx mice treated with high dose (5 mg/kg) peptide of Formula A-1 are presented in bar graph format in FIG. 7A and FIG. 7B. With reference to FIG. 7A, compared to wildtype mice, vehicle-treated D2.mdx mice and low dose (1 mg/kg) peptide of Formula A-1-treated mice had significantly decreased right ventricle ESP. There was an apparent improvement in the ESP for the D2.mdx mice treated with high dose (5 mg/kg) peptide of Formula A-1, albeit not significant in a statistical sense. Similarly with reference to FIG. 7B, compared to wildtype mice, vehicle-treated D2.mdx mice had significantly decreased right ventricle EDP. By comparison, there was an apparent improvement in EDP as the dose of the peptide of Formula A-1 was increased albeit not significant in a statistical sense. In all cases, treatment with high dose (5 mg/kg) of the peptide of Formula A-1 showed a trend for improved right ventricular ESP and EDP compared to vehicle-treated control, again, not significant in a statistical sense. This example therefore demonstrates that administration of the peptide of Formula A-1 can improve cardiac function in an animal model known to experience cardiac dysfunction.

Diaphragm fibrosis from wildtype mice, D2.mdx mice treated with vehicle, D2.mdx mice treated with low dose (1 mg/kg) peptide of Formula A-1, or D2.mdx mice treated with high dose (5 mg/kg) peptide of Formula A-1 are presented in bar graph format in FIG. 8. With reference to FIG. 8, the wildtype mice provide the expected pattern/amount of fibrosis of the diaphragm expected in a subject not having DMD. As can be seen in FIG. 8, the vehicle-treated D2.mdx mice had significantly increased fibrosis in the diaphragm muscle as compared with the wildtype mice. By comparison, there was a significant reduction in diaphragm muscle fibrosis in D2.mdx mice administered low dose peptide of Formula A-1 and an even more significant improvement (decrease) in fibrosis of the diaphragm muscle in the D2.mdx mice administered high dose of the peptide of Formula A-1 as compared with the vehicle treated D2.mdx mice or the D2.mdx mice treated with low dose (1 mg/kg) peptide of Formula A-1. Accordingly, this data shows a dose response to administration of the peptide of Formula A-1 to the D2.mdx mice with respect to a muscle involved in the respiratory system, thereby demonstrating the efficacy of the peptide of Formula A-1 for addressing muscular dysfunction in subjects suffering from muscular dystrophy such as DMD.

Collectively, this data demonstrates that the peptide of Formula A-1, or a pharmaceutically acceptable salt, hydrate, solvate, and/or tautomer thereof, is useful in methods for treating, preventing, inhibiting, ameliorating or delaying the onset of muscular dystrophy in a mammalian subject in need thereof.

### Example 4-Assessment of Heart (Cardiac) Function in a Rabbit Model Of Duchenne Muscular Dystrophy (DMD) with the Peptide of Formula α-1 Over Time

This Example demonstrates the use of the peptide of Formula A-1 in the treatment/amelioration/inhibition of cardiomyopathy progression in a DMD-KO rabbit model.

### Rabbits:

The DMD-KO rabbits used in this study were obtained as described in Sui et al., Disease Models & Mechanisms 11 (2018). These DMD-KO rabbits possess an engineered mutation in exon 51. These DMD-KO rabbits exhibit several of the human characteristics of DMD pathology, including disruption of dystrophin expression, impaired physical activity (loss of ambulation), lower body mass, shorter lifespan, elevated serum creatine kinase (CK) levels, and most significantly, a progressive cardiomyopathy leading to heart failure similar to that observed in humans. New Zealand rabbits were used as wild type (WT) controls.

### Methods:

All experiments involving rabbits in this study were performed in accordance with all applicable laws and regulations.

### Body Weight:

The body weight of age- and sex-matched WT and DMD-KO rabbits were measured weekly and a minimum of three individual animals of each genotype was used in all experiments.

### Serum Biochemical Analysis:

The blood samples were collected into serum separator tubes (MiniCollect) from the ear vein, and sera were prepared by centrifugation. Serum CK levels will be measured using a CK test kit (N-acetyl-L-cysteine method).

### Activity Measurement:

Physical activity of rabbits was monitored using a video recording system. Throughout the observation period, the rabbits were housed in a floor pen designed to provide ample space for unrestricted movement. The video camera was positioned overhead to capture their activity. Movement data, including walking steps, was recorded and logged over a 1-hour duration.

### Echocardiology:

Echocardiography recording was performed as described previously (Han et al., 2007; Xu et al., 2015a). Briefly, two-dimensional and M-mode transthoracic echocardiography was performed as previously described on WT and DMD-KO rabbits (n≥3 per group) by the Vevo 2100 ultrasound system (FUJIFILM VisualSonics, Inc., Toronto, Canada). Rabbits were studied in right lateral recumbency from parasternal long and short axis views. The rabbits were held in the right position by restraining their limbs with people. A linear array probe and center frequency of 10.0 MHz was used. Key cardiac measurements were obtained, including left ventricular dimensions during diastole and systole (LVIDd, LVIDs). All measurements were averaged over three consecutive cardiac cycles to minimize variability. The percentage of left ventricular ejection fraction (LVEF) was calculated by VevoLab analysis software (v3.0).

### Histology:

Cardiac tissue was collected from DMD-KO and WT rabbits (euthanized at 8 months of age). The tissues were embedded in optimal cutting temperature (OCT) compound and rapidly frozen in pre-cooled 2-methylbutane with liquid nitrogen. Tissue blocks were sectioned into 10 μm thin slices using a cryostat set at approximately −20° C. Fibrosis of cardiac muscle was assessed with Masson's Trichrome staining using standard practices. The stained sections were imaged with an Axio observer 7 Zeiss microscope.

### Study Design:

At approximately 24 weeks of age, rabbits were administered the peptide of Formula A-1 dissolved in sterile saline at a dose of 5 mg/kg, once daily via subcutaneous (SC) injection. The control group was given placebo (sterile saline). Rabbits were treated daily with peptide or saline vehicle control, with treatment lasting 8 weeks. Echocardiograms were collected after 4 and 8 weeks of treatment. At the time of death, tissues were harvested for analysis.

### Statistical Analysis:

Data analysis was performed using GraphPad Prism software. Statistical analysis was performed using one-way analysis of variance (ANOVA) followed by Tukey multiple comparisons test. P<0.05 was considered statistically significant.

### Results:

There were no other remarkable or statistically significant results observed in the DMD-KO rabbits treated with the peptide of Formula A-1 as compared with untreated control rabbits other than for the Echocardiogram results. With reference to FIG. 9, the cardiac (electrocardiogram) left ventricle ejection fraction (LVEF %) data collected in the study is presented in bar graph format. The LVEF for the wild type (WT) rabbits sets the expected/desired normal LVEF result. As can be seen in FIG. 9, the untreated (vehicle treated) DMD-KO rabbits exhibited a statistically significant decrease in LVEF as compared with the wildtype control rabbits. While administration of the peptide of Formula A-1 to the DMD-KO rabbits for 4 weeks produced an apparent improvement in LVEF as compared to the vehicle treated DMD-KO rabbits, this increase was not statistically significant. However, continued treatment of the DMD-KO rabbits for a total of 8 weeks produced a statistically significant improvement in LVEF as compared with the untreated DMD-KO rabbits. Indeed, data appears to indicate a complete amelioration/restoration of the LVEF deficit attributable to the genetic mutation in the rabbits thereby returning the LVEF values to roughly the equivalent of the WT rabbits. This is indeed a surprising result.

Accordingly, these results demonstrated that administration of the peptide of Formula A-1 for a period of at least 8 weeks produced a statistically significant improvement in cardiac left ventricle function in the DMD-KO rabbit model—an improvement of which substantially (or completely) alleviated the deficit overserved with the untreated cohort as compared with the wild type rabbits. These data suggest that the peptide of Formula A-1, or a pharmaceutically acceptable salt, hydrate, solvate, and/or tautomer thereof, can significantly impact/improve cardiac dysfunction associated with a disease state, such as Duchenne Muscular Dystrophy (DMD), and is useful in methods for treating, preventing, inhibiting, ameliorating or delaying the onset of muscular dystrophy in a mammalian subject in need thereof.

### Example 5-LANGENDORFF STUDY

### Ischemia-Reperfusion Injury-Langendorff Heart Preparation Protocol:

An infarction study was performed according to the Langendorff technique as described previously (Kuka J, Vilskersts R, Cirule H, Makrecka M, Pugovics O, Kalvinsh I, et al. The cardioprotective effect of mildronate is diminished after co-treatment with L-carnitine. J Cardiovasc Pharmacol Ther.2012; 17:215-222), with some modifications. Rats were anaesthetized with sodium pentobarbital (60 mg/kg) and heparin was administered intraperitoneally. For the infarction studies, the hearts were perfused with oxygenated (95% 02-5% CO2) Krebs-Henseleit (KH) buffer solution (118 mmol/L NaCl, 4.7 mmol/L KCl, 1.24 mmol/L CaCl2, 1.64 mmol/L MgCl2, 24.88 mmol/L NaHCO₃, 1.18 mmol/L KH₂PO₄, and 0.05 mmol/L EDTA; pH 7.3-7.5; 36.8-37.0° C.) supplemented with 10 mM glucose at a constant perfusion pressure of 60 mmHg. A water-ethanol mixture (1:1)-filled balloon connected to a physiological pressure transducer (ADInstruments) was inserted into the left ventricle, and the baseline end-diastolic pressure set at 5-10 mmHg. The heart rate (HR), flow, left-ventricle developed pressure (LVDP), contractility (+dp/dt) were continuously recorded using a PowerLab 8/35 system from ADInstruments. The isolated rat hearts were adapted for 20 min and the left anterior descending coronary artery (LAD) was subsequently occluded for 30 min followed by 120 min of reperfusion. KH perfusion solution with or without added compound of interest (vehicle or 1 mM concentration of elamipretide or of peptides of Formula A-1, A-11-1, A-12-1, A-13-1, A-14-1, A-15-1 or A-16) was used for the whole time of isolated heart perfusion.

### Compounds of Interest:

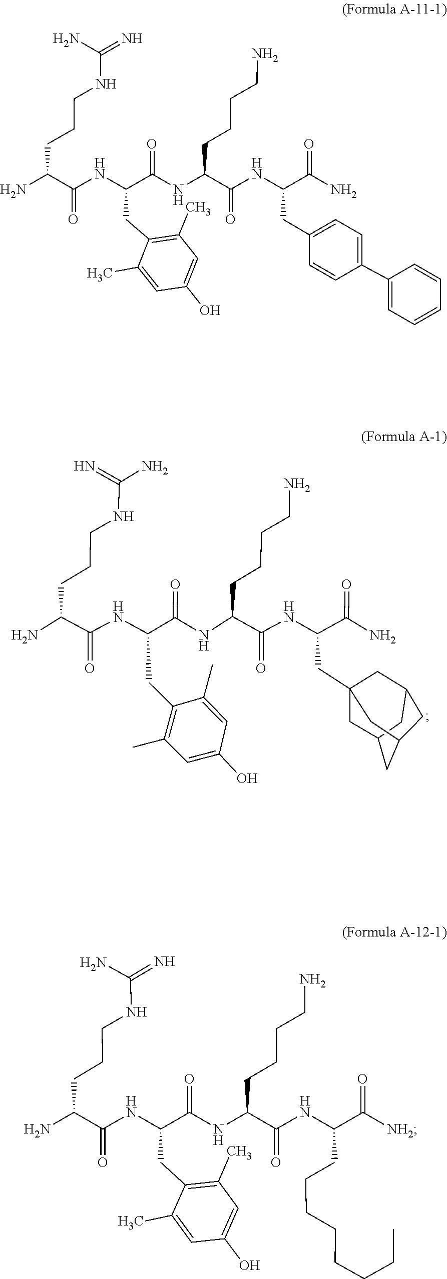 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O]

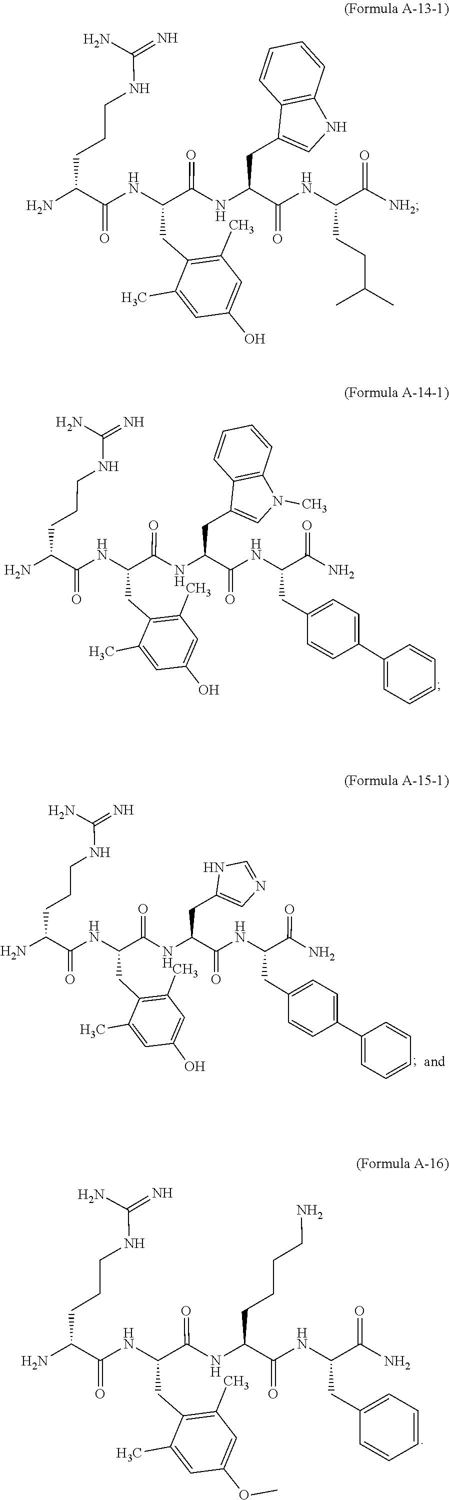 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; COc1cc(C)c(C[C@H](NC(=O)[C@H](N)CCCNC(=N)N)C(=O)N[C@@H](CCCCN)C(=O)N[C@@H](Cc2ccccc2)C(N)=O)c(C)c1]

Occlusion was confirmed by ~ 40% drop in coronary flow. The infarct size was determined as described previously (Kuka J, Vilskersts R, Cirule H, Makrecka M, Pugovics O, Kalvinsh I, Dambrova M, Liepinsh E. The cardioprotective effect of mildronate is diminished after co-treatment with L-carnitine. J Cardiovasc Pharmacol Ther. 2012 June; 17 (2): 215-22.doi: 10.1177/1074248411419502.; Liepinsh E, Kuka J, Dambrova M. Troubleshooting digital macro photography for image acquisition and the analysis of biological samples. J Pharmacol Toxicol Methods. 2013 March-Apr;67 (2): 98-106. doi: 10.1016/j.vascn.2012.11.00L). Briefly, at the end of the reperfusion, the LAD was re-occluded, and the heart was perfused with 0.1% methylene blue dissolved in KH buffer solution. Afterwards, hearts were sectioned transversely from the apex to the base in 6 slices (5 if smaller heart) of 2 mm thickness and incubated in 1% triphenyl-tetrazolium chloride in phosphate buffer (pH 7.4, 37° C.) for 10 min to stain viable tissue red and necrotic tissue white. The planimetric analysis of cross-sectional images was performed using Image-Pro Plus v6.3 software to determine the area at risk (AR) and area of necrosis (AN), each expressed as a percentage of cross-sectional slice area. The obtained values were then used to calculate the infarct size (IS) as a percentage of the risk area according to the formula:

$$IS ( \% ) = AN / AR \times 100 \% .$$

Area of necrosis was determined by combining areas of the white necrotic and pink tissue.
Study outline:
20 min. adaptation+30 min. ischemia (LAD ligation)+120 min. reperfusion (vehicle or compound 1 mM)
Endpoints: HR, flow, LVDP, +dP/dt, infarct size-area of necrosis
CTRL (vehicle)+up to 4 test compounds (n=8 per treatment) tested per set

Results are presented graphically in FIGS. 10A-10G. These results demonstrate that the peptides examined were all cardioprotective, all being roughly equivalent or superior to elamipretide in this ex-vivo assay.

### Example 6-Assessment of Skeletal Muscle and Heart (Cardiac) Function in a Mouse Model of Duchenne Muscular Dystrophy (DMD) Treated with an Adeno-Associated Virus (AAV) Vector-Based Gene Therapy, Optionally in Combination with the Peptide of Formula A-1

This Example demonstrates that use of the peptide of Formula A-1 in combination with AAV-aided micro-dystrophin gene therapy provides improvements over just AAV-aided micro-dystrophin therapy: (i) to skeletal muscle and (ii) cardio protection in male D2.mdx mice.

### Methods:

Study Protocol: All animal procedures were approved by the IACUC of the University of Florida. FIG. 11 provides an illustrative summary of the study protocol. Mice were treated with the AAV-aided micro-dystrophin gene therapy (2×10¹⁴ vg/kg via tail vein injection) at wean (one month of age as signified by the needle in FIG. 11). At 10 months of age, the AAV-aided micro-dystrophin (μDys) gene therapy-treated mice were separated into groups; one group receiving vehicle (Saline-n=10 mice) and the other group treated with the peptide of Formula A-1 (n=10 mice). The arrow in FIG. 11 at 10 months indicates the starting date of treatment with vehicle alone or the peptide of Formula Al. FIG. 11 also indicates that the first echocardiogram (baseline echocardiogram) was obtained for the mice at 10 month of age (illustrated as the heart in FIG. 11). The mice were treated with daily subcutaneous injections of vehicle (saline) or the peptide of Formula Al (at 5 mg/kg (peptide/body weight) using a solution containing 1 mg/mL peptide/saline) for two months (8 weeks beginning at 40 weeks of age). At 12 months of age, echocardiogram, ex vivo EDL, and diaphragm functional testing were performed (as signified by the heart and muscle in FIG. 11). The mice were sacrificed and serum, skeletal muscles, hearts and livers were collected.

Study Animals: This study used male D2.WT (The Jackson Laboratory, stock no. 000671) and D2.mdx (The Jackson Laboratory, stock no. 013141) mice from colonies originally obtained from The Jackson Laboratory. Mice were housed 1-5 mice per cage; randomly assigned into groups; provided ad libitum access to food (NIH-31 Open formulation diet; Envigo, 7917), water, and enrichment; and maintained on a 12-hour light/dark system. In this study, the data for WT and D2.mdx+vehicle data for comparison with the AAV micro-dystrophin (abbreviated in the Figs. as “μDys”) gene therapy treated mice were obtained from a companion study where the peptide of Formula A-1 was delivered to animals via a Alzet pump instead of by subcutaneous injection. That study did not produce definitive results.

Microdystrophin constructs and vector production: AAV-packaged, codon-optimized human microdystrophin was synthesized by Genscript. Human micro-dystrophin open-reading frame (ORF) was placed behind a modified skeletal and cardiac muscle-specific MHCK7 promoter that lacks an enhancer element for the alpha-myosin heavy chain. This gene and promoter were packaged into an AAV serotype rh74 capsid using the triple-transfection method, as previously described (Ai et al. & Sena-Esteves et al.).

Refs:
Ai J, et al. Adeno-associated virus serotype rh. 10 displays strong muscle tropism following intraperitoneal delivery. Sci Rep. 2017; 7:40336
Sena-Esteves M, Gao G. Introducing genes into mammalian cells: viral vectors. Cold Spring Harb Protoc. 2020; 2020 (8): 095513

Muscle mechanics: Maximal tetanic tension and eccentric damage assessments of the EDL and diaphragm were evaluated as previously described in Morine et al. and Barton-Davis et al. (full references provided below) by the Muscle Physiology Core at the University of Pennsylvania. Briefly, the muscles of anesthetized mice were dissected and placed in physiological Ringer's solution gas equilibrated with 95% O₂/5% CO₂. After determining optimal length, muscles were subjected to 3 isometric contractions (stimulated at 120 Hz for 500 ms for EDL or 100 Hz for 800 ms for diaphragm) to determine maximal tetanic tension (Po). Subsequently, a series of 5 eccentric contractions with (stimulated at 80 Hz for 700 ms) a stretch of 10% optimal length was imposed on the muscle in the last 200 ms of each contraction. Each contraction was separated by a 5-minute rest period. The specific force was determined by normalizing the force from the tetanic contraction against the cross sectional area. Following experimental procedures, muscles were weighed, frozen embedded in optimal cutting temperature (O.C.T.) compound or snap-frozen, and stored at −80° C. until further use. Force measurements and dissections were performed by investigators blinded to the treatment groups.

### Refs

Morine K J, Bish L T, Pendrak K, Sleeper M M, Barton E R, Sweeney H L. Systemic myostatin inhibition via liver-targeted gene transfer in normal and dystrophic mice. PLOS One. 2010; 5 (2): e9176.
Barton-Davis E R, Shoturma D I, Musaro A, Rosenthal N, Sweeney H L. Viral mediated expression of insulin-like growth factor I blocks the aging-related loss of skeletal muscle function. Proc Natl Acad Sci USA. 1998;95 (26): 15603-7.

Echocardiography and electrocardiograms: Electrocardiograms and transthoracic echocardiograms were performed using the Vevo 3100 preclinical imaging system (Fujifilm Visualsonics). Mice were anesthetized using 3% isoflurane and maintained at 1.5%-2% to keep heart and respiration rates consistent among treatment groups. Body temperature was maintained at 37° C. throughout imaging. Electrocardiograms were imported into LabCharts (ADInstruments) for analysis. Four images were acquired for each animal: B-mode parasternal long axis (LAX), B-mode short axis (SAX), M-mode SAX, and apical 4-chamber view with color Doppler and pulsed-wave Doppler. M-mode SAX images were acquired at the level of the papillary muscle. Flow through the mitral valve was sampled at the point of highest velocity, as indicated by aliasing, with the pulsed-wave angle matching the direction of flow. Images were imported into Vevo LAB for analysis. Measurements of M-mode SAX and pulsed-wave Doppler images were made from 3 consecutive cardiac cycles between respirations.

Statistics: Statistical analysis was performed using unpaired, 2-tailed Welch's t test (a α=0.05) or a one-way ANOVA followed by Tukey's multiple comparisons post hoc tests (a α=0.05). A P value less than 0.05 was considered significant. Data are displayed as mean±SEM. Results of the various skeletal muscle and cardiac testing are reported illustratively in FIGS. 12A-16C.

### Results:

With reference to FIG. 12A, echocardiogram data is presented for Left Ventricle (LV) End-Systole Diameter (mm) for the various groups of mice. As can be seen from the graph, there was a statistically significant decrease in Left Ventricle (LV) End-Systole Diameter between the WT mice and the D2.mdx mice treated with only vehicle. There was a noticeable trend towards an increase in Left Ventricle (LV) End-Systole Diameter in the D2.mdx mice treated with the μDys gene therapy and vehicle (albeit not statistically significant) as compared with the D2.mdx mice treated with only vehicle. However, the combined treatment of the peptide of Formula A-1 with the μDys gene therapy provided a statistically significant improvement in Left Ventricle (LV) End-Systole Diameter over the D2.mdx mice treated with only vehicle; an improvement that resembles the result for this cardiac parameter as seen in the WT mice. This result is not, a priori, an expected result.

With reference to FIG. 12B, echocardiogram data is presented for Left Ventricle (LV) End-Diastole Diameter (mm) for the various groups of mice. As can be seen from the graph, there was a statistically significant decrease in Left Ventricle (LV) End-Diastole Diameter between the WT mice and the D2.mdx mice treated with only vehicle. There was a noticeable trend towards an increase in Left Ventricle (LV) End-Diastole Diameter in the D2.mdx mice treated with the μDys gene therapy and vehicle (albeit not statistically significant) as compared with the D2.mdx mice treated with only vehicle. However, the combined treatment of the peptide of Formula A-1 with the μDys gene therapy provided a statistically significant improvement in Left Ventricle (LV) End-Diastole Diameter as compared with the D2.mdx mice treated with only vehicle; an improvement that resembles the result for this cardiac parameter as seen in the WT mice. This result is not, a priori, an expected result.

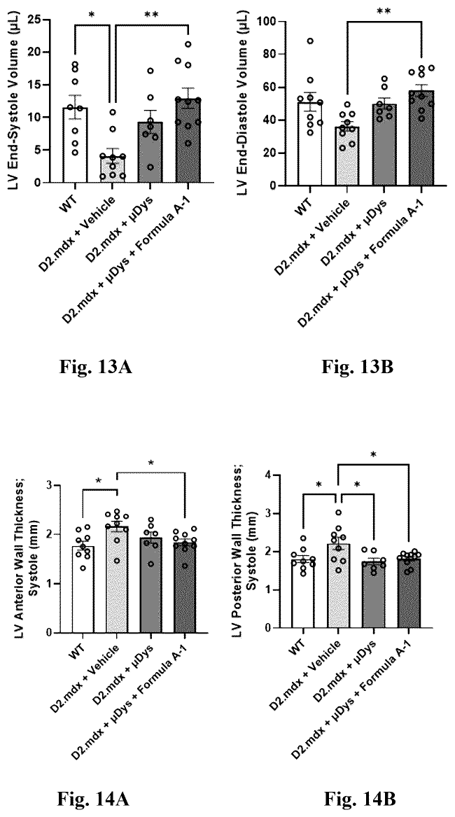

With reference to FIG. 13A, echocardiogram data is presented for Left Ventricle (LV) End-Systole Volume (μL) for the various groups of mice. As can be seen from the graph, there was a statistically significant decrease in Left Ventricle (LV) End-Systole Volume between the WT mice and the D2.mdx mice treated with only vehicle. There was a noticeable trend towards an increase in Left Ventricle (LV) End-Systole Volume in the D2.mdx mice treated with the μDys gene therapy and vehicle (albeit not statistically significant) as compared with the D2.mdx mice treated with only vehicle. However, the combined treatment of the peptide of Formula A-1 with the μDys gene therapy provided a statistically significant improvement in Left Ventricle (LV) End-Systole Volume as compared with the D2.mdx mice treated with only vehicle; an improvement that resembles the result for this cardiac parameter as seen in the WT mice. This result is not, a priori, an expected result.

With reference to FIG. 13B, echocardiogram data is presented for Left Ventricle (LV) End-Diastole Volume (μL) for the various groups of mice. As can be seen from the graph, there was a decrease (albeit not statistically significant) in Left Ventricle (LV) End-Diastole Volume between the WT mice and the D2.mdx mice treated with only vehicle. There was a noticeable trend towards an increase in Left Ventricle (LV) End-Diastole Volume in the D2.mdx mice treated with the μDys gene therapy and vehicle (albeit not statistically significant) as compared with the D2.mdx mice treated with only vehicle. However, the combined treatment of the peptide of Formula A-1 with the μDys gene therapy provided a statistically significant improvement in Left Ventricle (LV) End-Diastole Volume as compared with the D2.mdx mice treated with only vehicle; an improvement that resembles the result for this cardiac parameter as seen in the WT mice. This result is not, a priori, an expected result.

With reference to FIG. 14A, echocardiogram data is presented for Left Ventricle (LV) Anterior Wall Thickness; Systole (mm) for the various groups of mice. As can be seen from the graph, there is a statistically significant increase in Left Ventricle (LV) Anterior Wall Thickness; Systole between the WT mice and the D2.mdx mice treated with only vehicle. There is a noticeable trend towards a decrease in Left Ventricle (LV) Anterior Wall Thickness; Systole in the D2.mdx mice treated with the μDys gene therapy and vehicle (albeit not statistically significant) as compared with the D2.mdx mice treated with only vehicle. However, the combined treatment of the peptide of Formula A-1 with the μDys gene therapy provided a statistically significant improvement (in this case a decrease) in Left Ventricle (LV) Anterior Wall Thickness; Systole as compared with the D2.mdx mice treated with only vehicle; an improvement that resembles the result for this cardiac parameter as seen in the WT mice. This result is not, a priori, an expected result.

With reference to FIG. 14B, echocardiogram data is presented for Left Ventricle (LV) Posterior Wall Thickness; Systole (mm) for the various groups of mice. As can be seen from the graph, there is a statistically significant increase in Left Ventricle (LV) Posterior Wall Thickness; Systole between the WT mice and the D2.mdx mice treated with only vehicle. There is a statistically significant decrease in Left Ventricle (LV) Posterior Wall Thickness; Systole in the D2.mdx mice treated with the μDys gene therapy and vehicle as compared with the D2.mdx mice treated with only vehicle. In this case the combined treatment of the peptide of Formula A-1 with the μDys gene therapy also provided a statistically significant improvement (in this case a decrease) in Left Ventricle (LV) Posterior Wall Thickness; Systole as compared with the D2.mdx mice treated with only vehicle; an improvement that resembles the result for this cardiac parameter as seen in the WT mice. This result is not, a priori, an expected result but this result does not appear to be a significant improvement in this cardiac parameter as compared with treatment with the μDys gene therapy only.

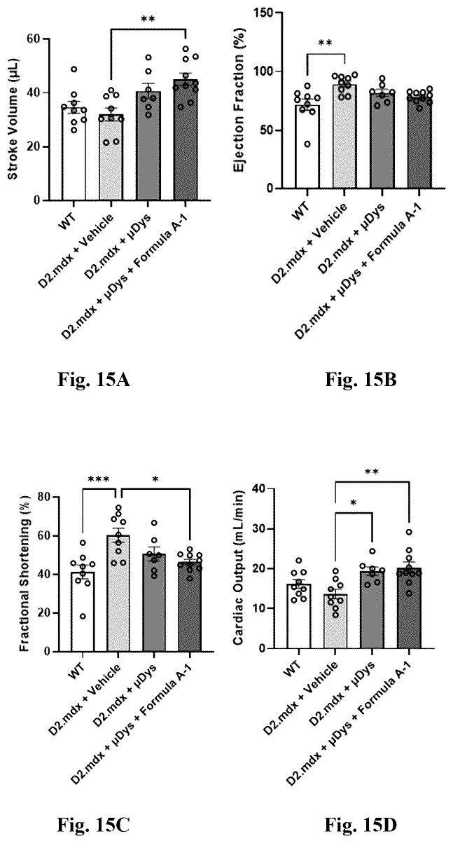

With reference to FIG. 15A, echocardiogram data is presented for Stroke Volume (μL) for the various groups of mice. As can be seen from the graph, there is a decrease in Stroke Volume (albeit not statistically significant) between the WT mice and the D2.mdx mice treated with only vehicle. There is a noticeable trend towards an increase in Stroke Volume in the D2.mdx mice treated with the μDys gene therapy and vehicle (albeit not statistically significant) as compared with the D2.mdx mice treated with only vehicle. However, the combined treatment of the peptide of Formula A-1 with the μDys gene therapy provided a statistically significant improvement (in this case an increase) in Stroke Volume as compared with the D2.mdx mice treated with only vehicle; an improvement that appears to surpass this cardiac parameter as seen in the WT mice. This result is not, a priori, an expected result.

With reference to FIG. 15B, echocardiogram data is presented for Ejection Fraction (%) for the various groups of mice. As can be seen from the graph, there is a statistically significant increase in Ejection Fraction (%) between the WT mice and the D2.mdx mice treated with only vehicle. There is a trend towards normalization (albeit not statistically significant) of Ejection Fraction (%) in the D2.mdx mice treated with the μDys gene therapy and vehicle as compared with the D2.mdx mice treated with only vehicle. The combined treatment of the peptide of Formula A-1 with the μDys gene therapy also exhibited a trend towards a decrease (albeit not statistically significant) in Ejection Fraction (%) as compared with the D2.mdx mice treated with only vehicle; which result may have been slightly better than what was seen in the mice treated with μDys gene therapy and vehicle, albeit not a statistically different result. Regardless, the data suggests that treatment with the peptide of Formula A-1 in combination with μDys gene therapy results in a trend towards normalization of the ejection fraction to that observed in WT mice. This result is not, a priori, an expected result.

With reference to FIG. 15C, echocardiogram data is presented for Fractional Shortening (%) for the various groups of mice. As can be seen from the graph, there is a statistically significant increase in Fractional Shortening (%) between the WT mice and the D2.mdx mice treated with only vehicle. There is a noticeable trend towards a decrease in Fractional Shortening (%) in the D2.mdx mice treated with the μDys gene therapy and vehicle (albeit not statistically significant) as compared with the D2.mdx mice treated with only vehicle. However, the combined treatment of the peptide of Formula A-1 with the μDys gene therapy provided a statistically significant improvement (in this case a decrease) in Fractional Shortening (%) as compared with the D2.mdx mice treated with only vehicle; an improvement that resembles the result for this cardiac parameter as seen in the WT mice. Therefore, the data suggests that treatment with the peptide of Formula A-1 in combination with μDys gene therapy results in a statistically significant normalization of the Fractional Shortening (%) to that observed in WT mice. This result is not, a priori, an expected result.

With reference to FIG. 15D, echocardiogram data is presented for Cardiac Output (mL/min) for the various groups of mice. As can be seen from the graph, there is a trend towards a decrease (albeit not statistically significant) in Cardiac Output between the WT mice and the D2.mdx mice treated with only vehicle. In contrast, there is a statistically significant increase in Cardiac Output in the D2.mdx mice treated with the μDys gene therapy and vehicle as compared with the D2.mdx mice treated with only vehicle. Similarly, the combined treatment of the peptide of Formula A-1 with the μDys gene therapy also provided a statistically significant improvement in Cardiac Output as compared with the D2.mdx mice treated with only vehicle. This result is not, a priori, an expected result.

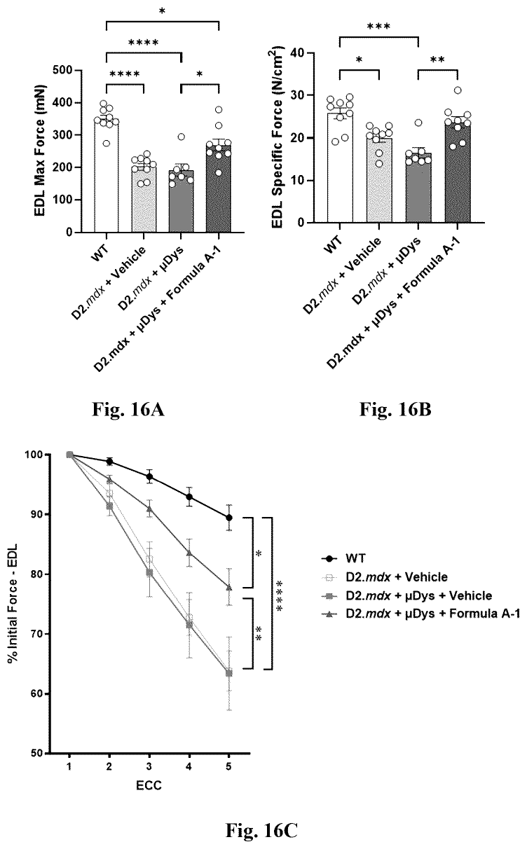

With reference to FIG. 16A, data is presented for extensor digitorum longus (EDL-a skeletal muscle) Max Force (mN) for the various groups of mice. As can be seen from the graph, there is a statistically significant decrease in muscle force between the WT mice and the D2.mdx mice treated with only vehicle. However, there seems to be little difference in the force observed between the D2.mdx mice treated with the μDys gene therapy and vehicle and the D2.mdx mice treated with only vehicle. This result does demonstrate a statistically significant decrease in force as compared with WT mice. However, the combined treatment of the peptide of Formula A-1 with the μDys gene therapy exhibited a statistically significant improvement in muscle force as compared with the D2.mdx mice treated with μDys and vehicle. This result appears to be a substantial improvement over treatment with μDys and vehicle, but still significantly (statistically significantly) lower than observed in the WT mice. Nevertheless, it speaks to the significant improvement associated with the combination therapy over treatment with the μDys gene therapy only. This result is not, a priori, an expected result.

With reference to FIG. 16B, data is presented for EDL Specific Force (N/cm²) for the various groups of mice. As can be seen from the graph, there is a statistically significant decrease in this “specific” muscle force between the WT mice and the D2.mdx mice treated with only vehicle. Surprisingly, there seems to be a trend towards further decrease (albeit not statistically significant) in this “specific” muscle force between the D2.mdx mice treated with the μDys gene therapy and vehicle and the D2.mdx mice treated with only vehicle However, the combined treatment of the peptide of Formula A-1 with the μDys gene therapy exhibited a statistically significant improvement in this “specific” muscle force as compared with the D2.mdx mice treated with μDys gene therapy and vehicle. This result appears to be a substantial improvement over treatment with μDys gene therapy only. Albeit an improvement, the result appears to trend lower than the “specific” muscle force observed in the WT mice, albeit not a statistically significant difference.

With reference to FIG. 16C, data is presented for % of Initial Force-EDL for the various groups of mice. As seen in FIG. 16C, repeated contraction of the EDL of WT mice produced about a 10% decrease in force over 5 contractions. By comparison, both the D2.mdx mice treated with vehicle alone and those mice treated with μDys gene therapy and vehicle exhibited approximately the same 35% reduction in force over 5 contractions; which result was statistically significant. However, the D2.mdx mice treated with μDys gene therapy and the peptide of Formula A-lexhibited only about a 20% reduction in force over the 5 contractions. This result was lower than WT in a statistically significant manner but also higher (in a statistically significant manner) than was seen for the D2.mdx mice treated with vehicle alone and those mice treated with μDys gene therapy and vehicle. These results again demonstrate a clear superiority provided by the combination therapy over treatment with the μDys gene therapy alone. This result is not, a priori, an expected result.

### Discussion & Summary:

Male D2. WT and D2.mdx mice were initially allowed to age without treatment with the compound of Formula A-1 because at 10 months of age the D2.mdx mice will be nearing a phase of cardiac/diastolic dysfunction. That is why treatment with the peptide of Formula A-1 was not initiated until the mice reached 10 months of age. In this study, the diaphragm and EDL muscles were examined in addition to performing an extensive panel of cardiac parameters. There were no significant findings with respect to the diaphragm muscle except that treatment with the peptide of Formula A-1 may have negated the beneficial effect of the μDys gene therapy treatment. Conversely, the combination of μDys gene therapy treatment with peptide of Formula A-1 treatment on the EDL demonstrated a statistically significant improvement over μDys gene therapy treatment alone for the majority of cardiac parameters examined. Similarly, although μDys gene therapy treatment alone in many cases appears to have trend towards improvement in cardiac function, in almost all cases, the combination of μDys gene therapy treatment with peptide of Formula A-1 treatment exceeded the results seen for treatment with μDys gene therapy alone, and in many cases, the improvement from the untreated D2.mdx mouse model (treatment with saline alone) was statistically significant, which was often not true when the mice were treated with μDys gene therapy alone. Collectively, these data demonstrate a clear improvement in many parameters tested that was associated with the combination therapy treatment (i.e., treatment with μDys gene therapy and the peptide of Formula A-1) over treatment with the μDys gene therapy alone. The results of this Example could not have been, a priori, expected.

### EQUIVALENTS

The present technology is not to be limited in terms of the particular embodiments described herein, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Various embodiments are set forth within the following claims.

What is claimed is:

1. A method for treating, preventing, inhibiting, ameliorating, or delaying the onset of muscular dystrophy in a mammalian subject in need thereof, comprising administering to the subject a therapeutically effective amount of a peptide of Formula A, or a mixture of peptides of Formula A: 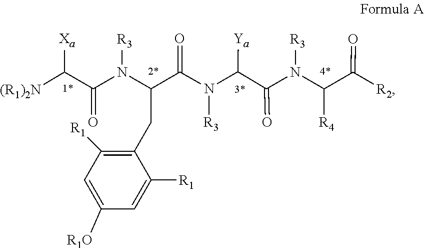
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 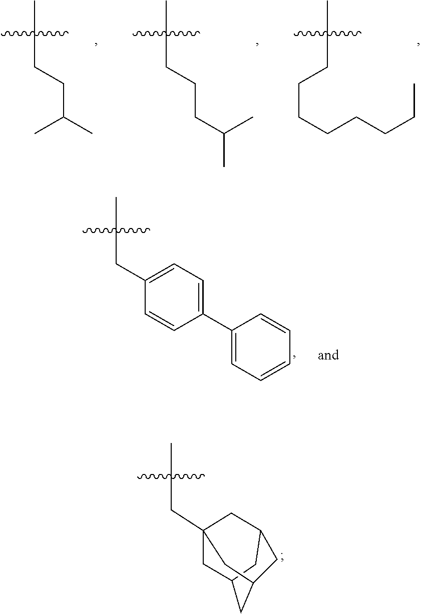
Xₐ is selected from 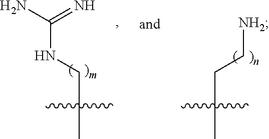
Yₐ is selected from 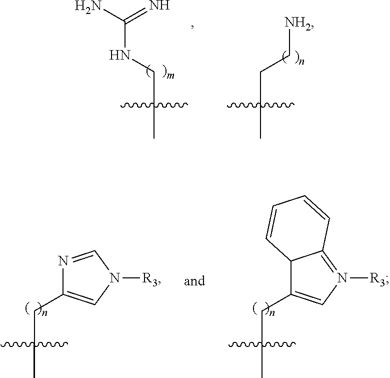
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

2. The method of claim 1, wherein the peptide or peptides of Formula A is/are a peptide/peptides of Formula A-1 or Formula A-2: 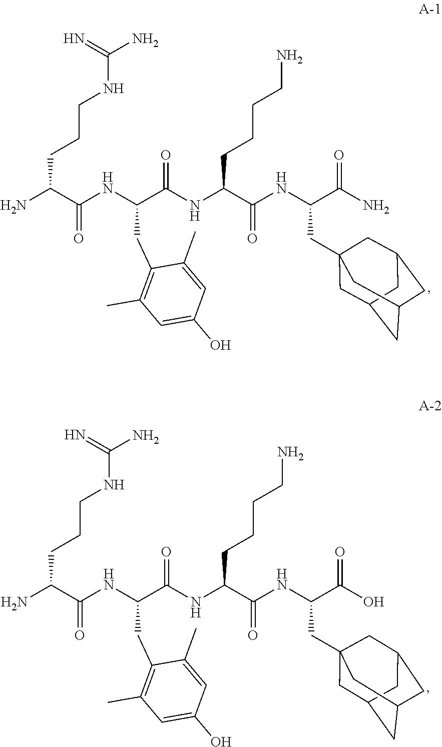 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

3. The method of claim 1, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 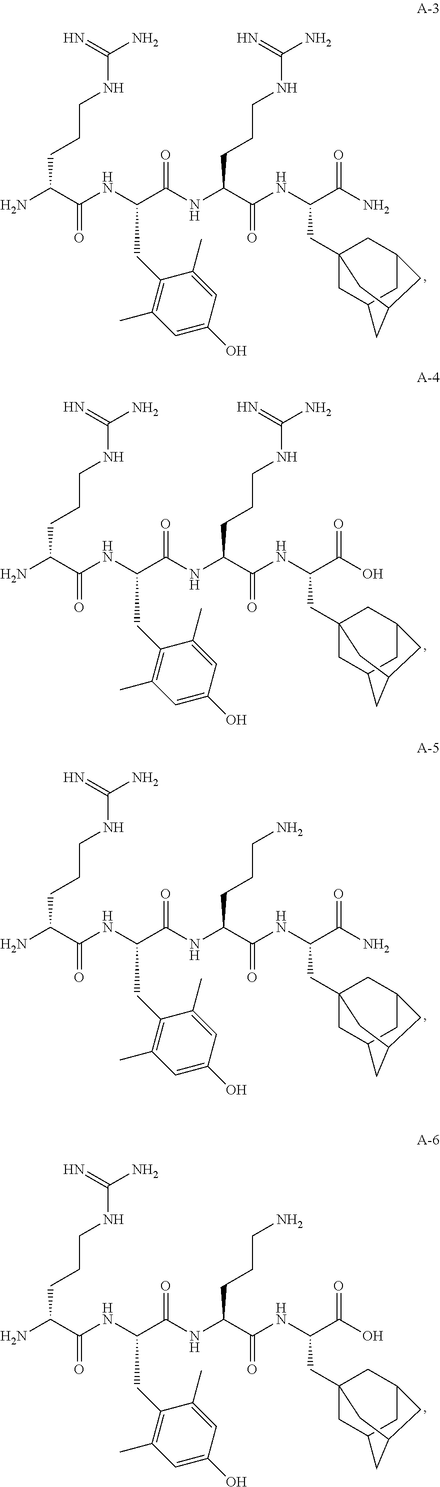 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 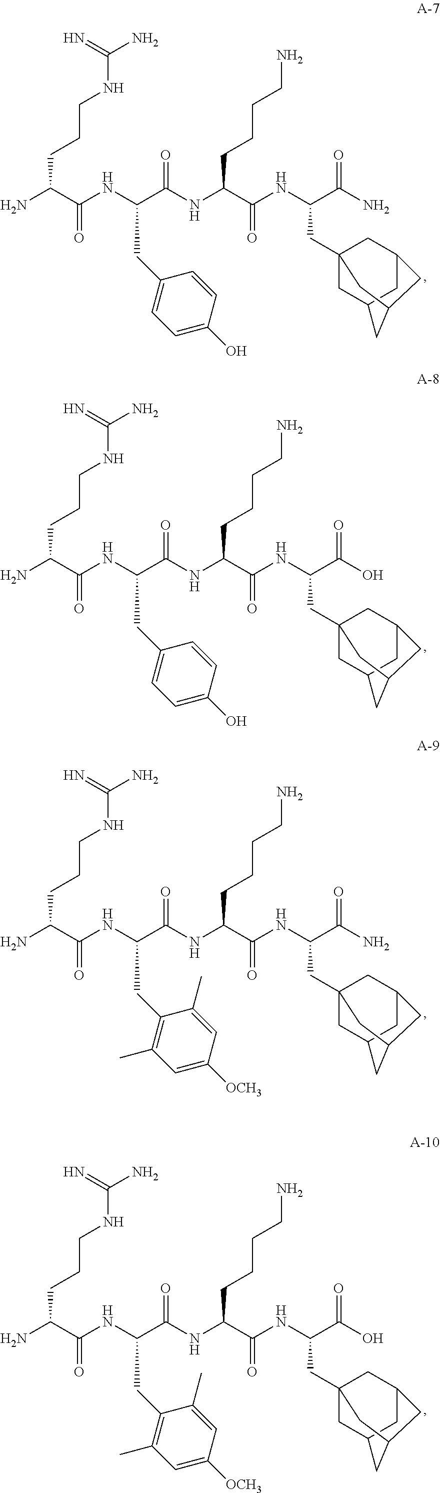 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 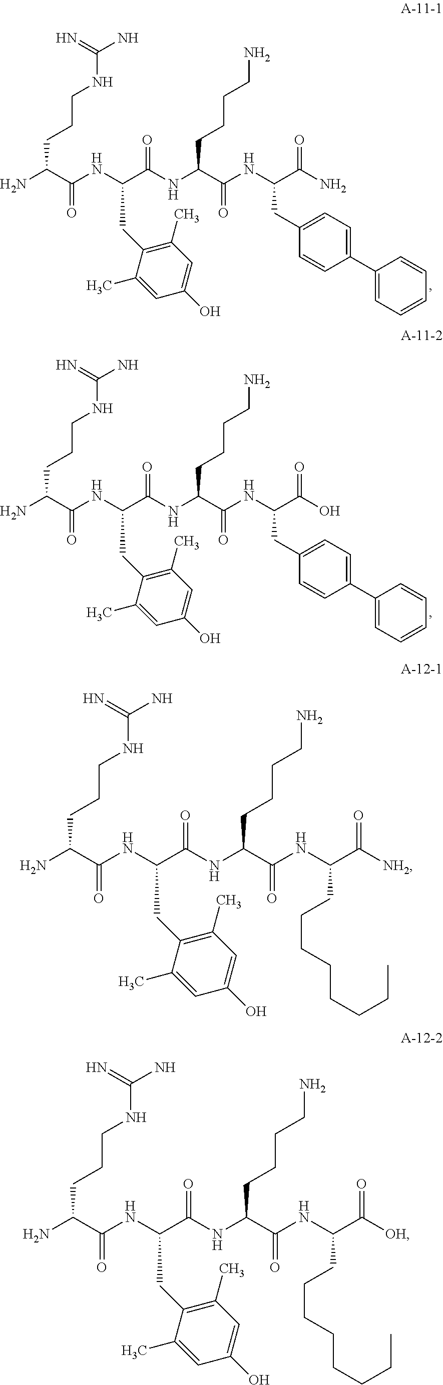 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 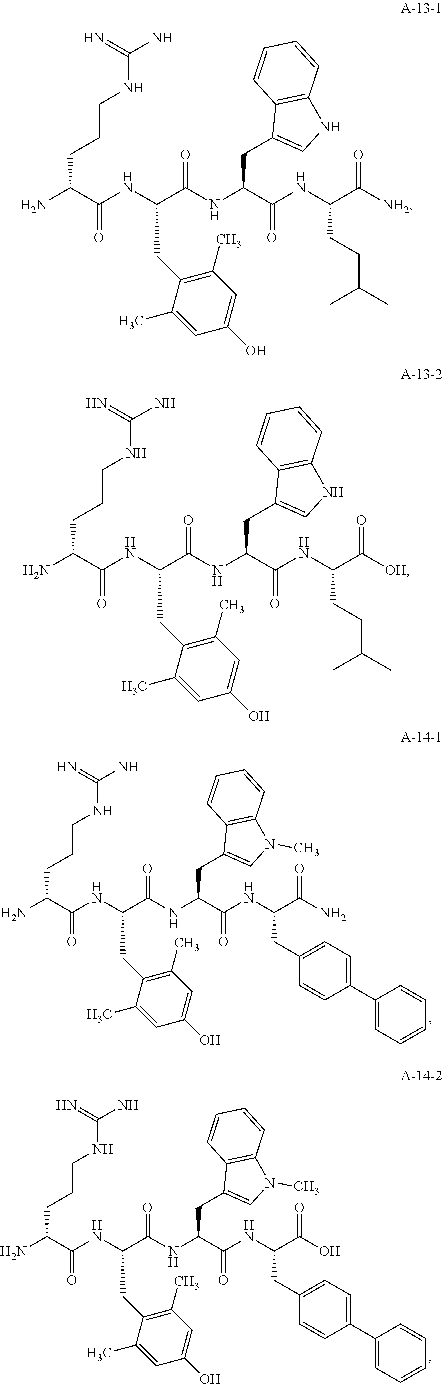 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O] 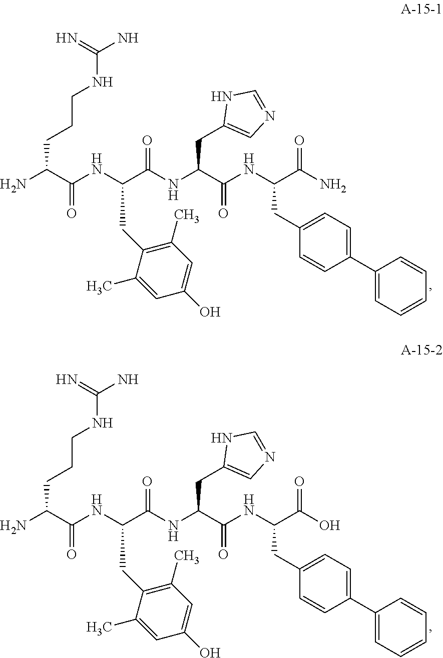 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

4. The method of any one of claims 1 to 3, wherein administration of the peptide or peptides ameliorates, inhibits, delays the onset of, and/or delays the progression of one or more signs or symptoms of muscular dystrophy in the subject as compared with an untreated control subject or untreated control group that is not administered the peptide or peptides.

5. The method of any one of claims 1 to 3, wherein administration of the peptide or peptides ameliorates, inhibits, delays the onset of, and/or delays the progression of a cardiomyopathy associated with muscular dystrophy in the subject as compared with an untreated control subject or untreated control group that is not administered the peptide or peptides.

6. The method of claim 5, wherein the cardiomyopathy is systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, heart (cardiac) failure, or a combination of any two or more of the foregoing in the subject.

7. The method of any one of claims 1 to 3, wherein administration of the peptide or peptides ameliorates, inhibits, delays the onset of, and/or delays the progression of muscular fibrosis in the subject as compared with an untreated control subject or untreated control group that is not administered the peptide or peptides.

8. The method of claim 7, wherein the administration of the peptide or peptides ameliorates, inhibits, delays the onset of, and/or delays the progression of muscular fibrosis of the diaphragm of the subject as compared with an untreated control subject or untreated control group that is not administered the peptide or peptides.

9. The method of claim 7, wherein the administration of the peptide or peptides ameliorates, inhibits, delays the onset of, and/or delays the progression of muscular fibrosis of skeletal muscle of the subject as compared with an untreated control subject or untreated control group that is not administered the peptide or peptides.

10. The method of any one of claims 1 to 3, wherein the administration of the peptide or peptides increases muscle force the subject can exert as compared with an untreated control subject or untreated control group that is not administered the peptide or peptides.

11. The method of any one of claims 1 to 3, wherein administration of the peptide or peptides ameliorates, inhibits, delays the onset of, and/or delays progression of contraction-induced damage in muscle tissue, locomotor-skeletal muscle weakness, respiratory muscle weakness, cardiac muscle weakness, sarcolemmal weakening, or sarcolemmal tearing in the subject as compared with an untreated control subject or untreated control group that is not administered the peptide or peptides.

12. The method of any one of claims 1 to 11, wherein the peptide(s) is/are administered daily for: (i) 12 weeks or more; (ii) 24 weeks or more; (iii) 48 weeks or more; (iv) 72 weeks or more; or (v) 96 weeks or more.

13. The method of any one of claims 1 to 12, wherein the peptide(s) is/are administered subcutaneously or intravenously.

14. The method of any one of claims 1 to 12, wherein the peptide(s) is/are administered orally, topically, systemically, intraperitoneally, intradermally, transdermally, ophthalmically, intrathecally, intracerebroventricularly, iontophoretically, transmucosally, intravitreally, intranasally, or intramuscularly.

15. The method of any one of claims 1 to 14, wherein the subject is human.

16. The method of any one of claims 1 to 15, wherein the muscular dystrophy is Duchenne muscular dystrophy.

17. The method of any one of claims 1 to 15, wherein the muscular dystrophy is Becker's muscular dystrophy.

18. The method of any one of claims 1 to 17, further comprising separately, sequentially, or simultaneously administering an additional therapeutic agent to the subject.

19. The method of claim 18, wherein the additional therapeutic agent is a phosphorodiamidate morpholino oligomer (PMO) or a peptide-conjugated phosphorodiamidate morpholino oligomer (PPMO).

20. The method of claim 19, wherein the PMO is selected from the group consisting of eteplirsen (Exondys 51®), golodirsen (Vyondys 53®), viltolarsen (Viltepso®), and casimersen (Amondys 45™) and the PPMO is selected from vesleteplirsen (a.k.a., SRP-5051) and PGN-EDO51 (a.k.a., CONNECT1-ED051).

21. The method of claim 18, wherein the additional therapeutic agent is an adeno-associated virus (AAV) vector-based gene therapy.

22. The method of claim 21, wherein the adeno-associated virus (AAV) vector-based gene therapy is delandistrogene moxeparvovec-rokl (Elevidys®).

23. The method of claim 18, wherein the additional therapeutic agent is a corticosteroid, an angiotensin-converting enzyme (ACE) inhibitor, an angiotensin receptor blocker (ARB), a beta blocker, a diuretic, or a mineralocorticoid receptor antagonist.

24. The method of claim 23, wherein the corticosteroid is vamorolone (aGamree®), prednisone, or deflazacort.

25. The method of claim 23, wherein the angiotensin-converting enzyme (ACE) inhibitor is captopril, enalapril, lisinopril, benazepril, or ramipril.

26. The method of claim 23, wherein the angiotensin receptor blocker (ARB) is azilsartan, candesartan, eprosartan, irbesartan, telmisartan, valsartan, losartan, olmesartan, Entresto® (sacubitril/valsartan), or Byvalson™ (nebivolol/valsartan).

27. The method of claim 23, wherein the beta blocker is carvedilol, bisoprolol, metoprolol succinate, atenolol, esmolol, nebivolol, or propranolol.

28. The method of claim 23, wherein the diuretic is bumetanide, furosemide, or torsemide.

29. The method of claim 23, wherein the mineralocorticoid receptor antagonist is spironolactone or eplerenone.

30. The method of claim 18, wherein the additional therapeutic agent is mavacamten (Camzyos®).

31. The method of any one of claims 1 to 30, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

32. The method of any one of claims 1 to 30, wherein the peptide or peptides is/are formulated for administration from its/their tris —HCl salt.

33. A method for treating, preventing, inhibiting, ameliorating, or delaying the onset of a cardiomyopathy in a mammalian subject in need thereof, wherein the subject is suffering from muscular dystrophy, comprising administering to the subject a therapeutically effective amount of a peptide of Formula A, or a mixture of peptides of Formula A: 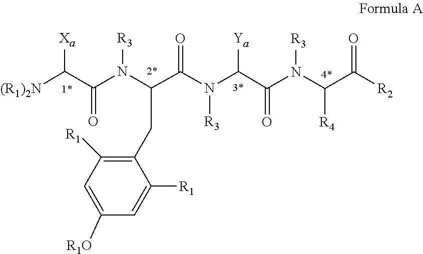
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 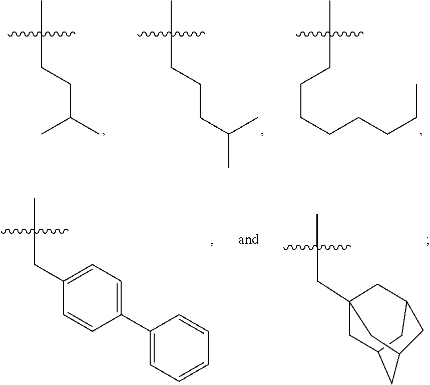
Xₐ is selected from 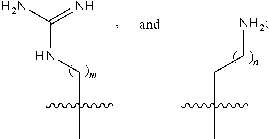
Yₐ is selected from 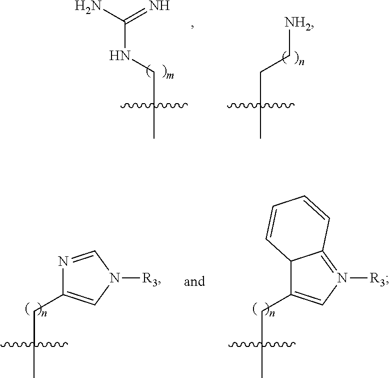
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

34. The method of claim 33, wherein the peptide or peptides of Formula A is is/are a peptide/peptides of Formula A-1 or Formula A-2: 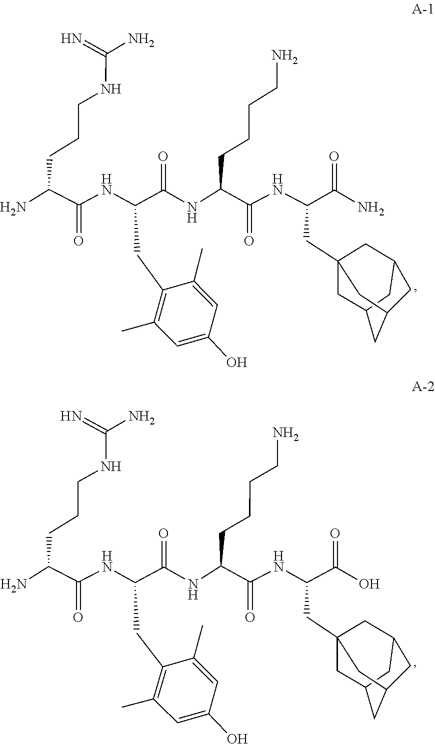 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

35. The method of claim 33, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 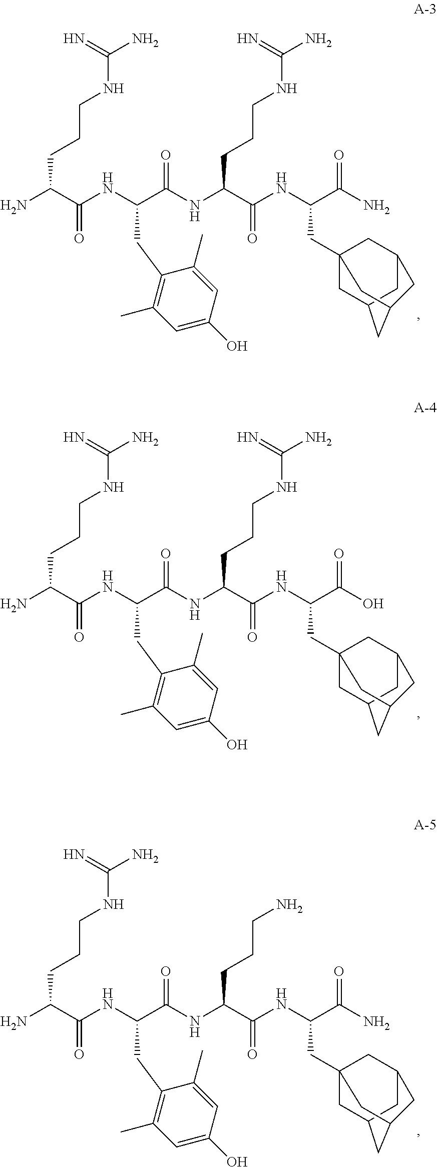 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O] 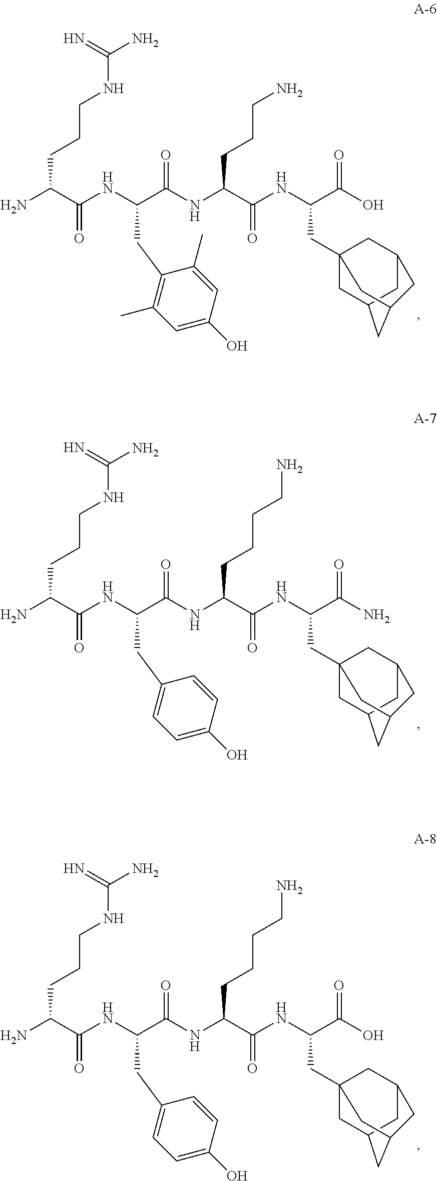 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O] 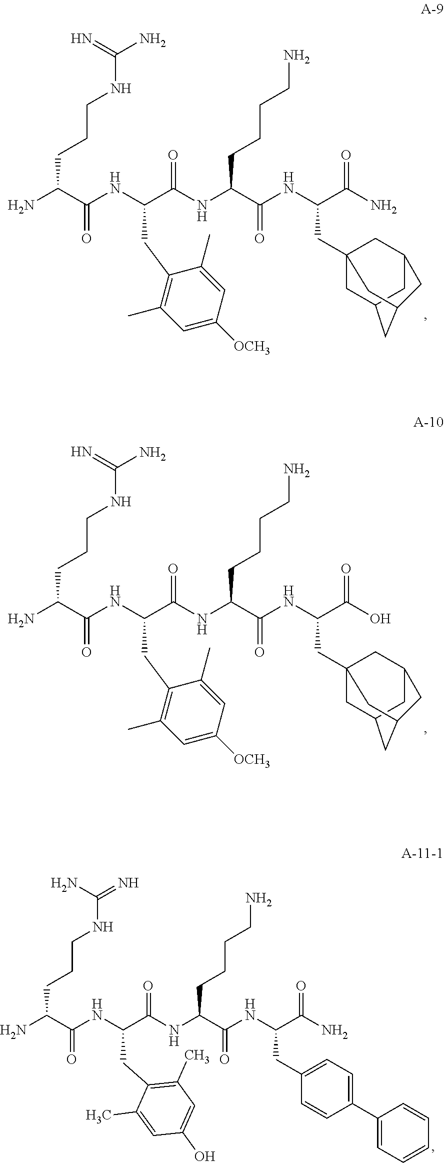 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O] 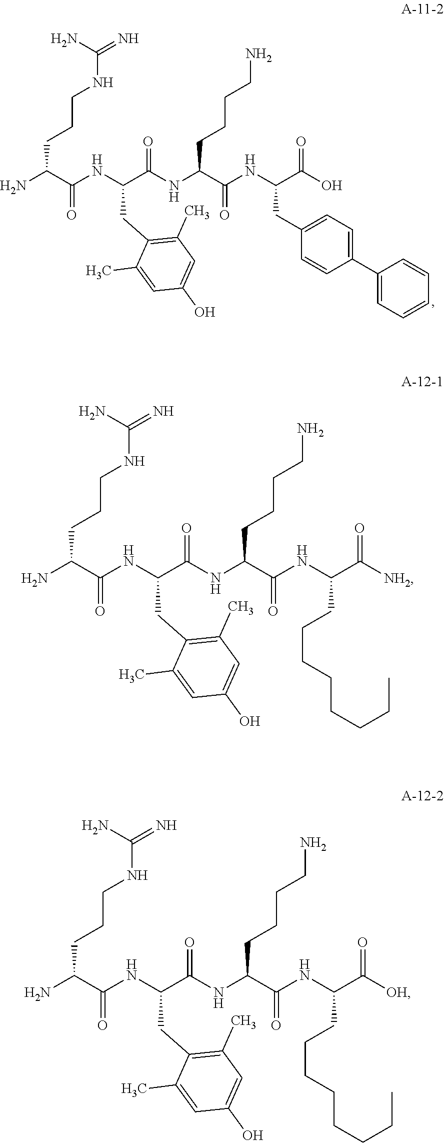 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 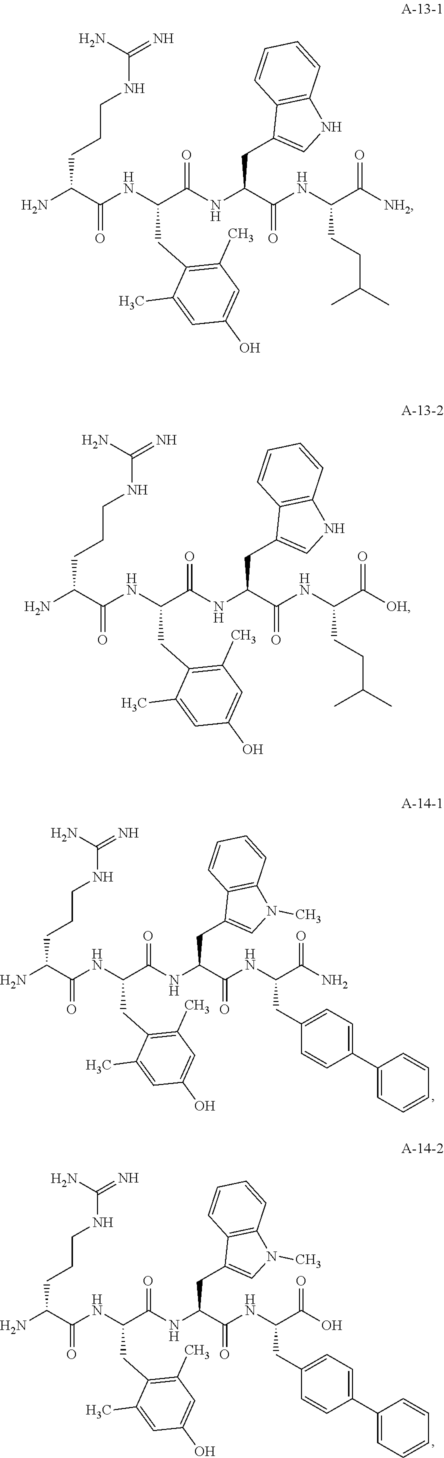 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O] 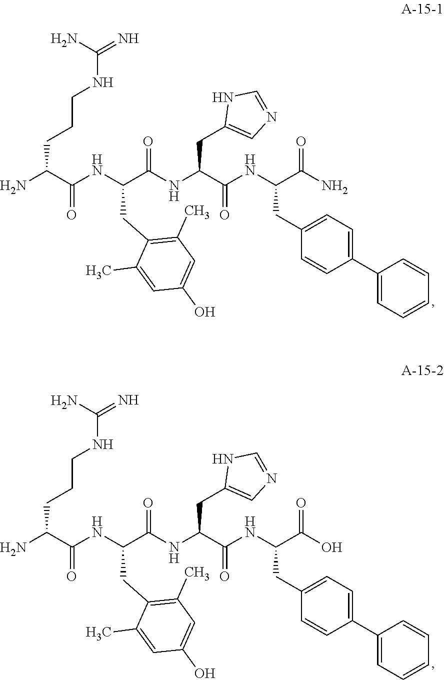 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

36. The method of any one of claims 32 to 35, wherein administration of the peptide or peptides ameliorates, inhibits, delays the onset of, and/or delays the progression of calcium overload, systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, or heart (cardiac) failure in the subject as compared with an untreated control subject or untreated control group that is not administered the peptide or peptides.

37. The method of claim 36, wherein administration of the peptide or peptides ameliorates, inhibits, delays the onset of, and/or delays the progression of calcium overload, systolic disfunction, or diastolic dysfunction in the subject as compared with an untreated control subject or untreated control group that is not administered the peptide or peptides.

38. The method of claim 36, wherein administration of the peptide or peptides ameliorates, inhibits, delays the onset of, and/or delays the progression of ventricular remodeling, including left ventricular remodeling, in the subject as compared with an untreated control subject or untreated control group that is not administered the peptide or peptides.

39. The method of claim 36, wherein administration of the peptide or peptides ameliorates, inhibits, delays the onset of and/or delays the progression of cardiac (myocardial) fibrosis in the subject as compared with an untreated control subject or untreated control group that is not administered the peptide or peptides.

40. The method of claim 36, wherein administration of the peptide or peptides ameliorates, inhibits, delays the onset of, and/or delays the progression of fatty acid infiltration in the subject as compared with an untreated control subject or untreated control group that is not administered the peptide or peptides.

41. The method of claim 36, wherein administration of the peptide or peptides ameliorates, inhibits, delays the onset of, and/or delays the progression of hypertrophic cardiomyopathy in the subject as compared with an untreated control subject or untreated control group that is not administered the peptide or peptides.

42. The method of claim 36, wherein administration of the peptide or peptides ameliorates, inhibits, delays the onset of, and/or delays the progression of dilated cardiomyopathy in the subject as compared with an untreated control subject or untreated control group that is not administered the peptide or peptides.

43. The method of claim 36, wherein administration of the peptide or peptides ameliorates, inhibits, delays the onset of, and/or delays the progression of an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia) in the subject as compared with an untreated control subject or untreated control group that is not administered the peptide or peptides.

44. The method of claim 36, wherein administration of the peptide or peptides ameliorates, inhibits, delays the onset of, and/or delays the progression of reduced left ventricular ejection fraction in the subject as compared with an untreated control subject or untreated control group that is not administered the peptide or peptides.

45. The method of claim 36, wherein administration of the peptide or peptides ameliorates, inhibits, delays the onset of, and/or delays the progression of fractional shortening in the subject as compared with an untreated control subject or untreated control group that is not administered the peptide or peptides.

46. The method of claim 36, wherein administration of the peptide or peptides ameliorates, inhibits, delays the onset of, and/or delays the progression of heart (cardiac) failure in the subject as compared with an untreated control subject or untreated control group that is not administered the peptide or peptides.

47. The method of any one of claims 33 to 35, wherein administration of the peptide or peptides results in normalization of the ejection fraction, fractional shortening, stroke volume, and/or cardiac output of the subject as compared with an untreated control subject or control group that is not administered the peptide or peptides.

48. The method of any one of claims 33 to 47, wherein the peptide or peptides is/are administered daily for: (i) 12 weeks or more; (ii) 24 weeks or more; (iii) 48 weeks or more; (iv) 72 weeks or more; or (v) 96 weeks or more.

49. The method of any one of claims 33 to 48, wherein the peptide or peptides is/are administered subcutaneously or intravenously.

50. The method of any one of claims 33 to 48, wherein the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

51. The method of any one of claims 33 to 50, wherein the subject is human.

52. The method of any one of claims 33 to 51, wherein the muscular dystrophy is Duchenne muscular dystrophy.

53. The method of any one of claims 33 to 51, wherein the muscular dystrophy is Becker's muscular dystrophy.

54. The method of any one of claims 33 to 53, further comprising separately, sequentially, or simultaneously administering an additional therapeutic agent to the subject.

55. The method of claim 54, wherein the additional therapeutic agent is a phosphorodiamidate morpholino oligomer (PMO) or a peptide-conjugated phosphorodiamidate morpholino oligomer (PPMO).

56. The method of claim 55, wherein the PMO is selected from the group consisting of eteplirsen (Exondys 51®), golodirsen (Vyondys 53®), viltolarsen (Viltepso®), and casimersen (Amondys 45®) or a PPMO selected from vesleteplirsen (SRP-5051) and PGN-EDO51 (a.k.a., CONNECT1-ED051).

57. The method of claim 54, wherein the additional therapeutic agent is an adeno-associated virus (AAV) vector-based gene therapy.

58. The method of claim 57, wherein the adeno-associated virus (AAV) vector-based gene therapy is delandistrogene moxeparvovec-rokl (Elevidys®).

59. The method of claim 54, wherein the additional therapeutic agent is a corticosteroid, an angiotensin-converting enzyme (ACE) inhibitor, an angiotensin receptor blocker (ARB), a beta blocker, a diuretic, or a mineralocorticoid receptor antagonist.

60. The method of claim 59, wherein the corticosteroid is vamorolone (aGamree®), prednisone, or deflazacort.

61. The method of claim 59, wherein the angiotensin-converting enzyme (ACE) inhibitor is captopril, enalapril, lisinopril, benazepril, or ramipril.

62. The method of claim 59, wherein the angiotensin receptor blocker (ARB) is azilsartan, candesartan, eprosartan, irbesartan, telmisartan, valsartan, losartan, olmesartan, Entresto® (sacubitril/valsartan), or Byvalson™ (nebivolol/valsartan).

63. The method of claim 59, wherein the beta blocker is carvedilol, bisoprolol, metoprolol succinate, atenolol, esmolol, nebivolol, or propranolol.

64. The method of claim 59, wherein the diuretic is bumetanide, furosemide, or torsemide.

65. The method of claim 59, wherein the mineralocorticoid receptor antagonist is spironolactone or eplerenone.

66. The method of claim 54, wherein the additional therapeutic agent is mavacamten (Camzyos®).

67. The method of any one of claims 33 to 66, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

68. The method of any one of claims 33 to 66, wherein the peptide or peptides is/are formulated for administration from its/their tris —HCl salt.

69. A composition or medicament for use in treating, preventing, inhibiting, ameliorating or delaying the onset of muscular dystrophy or its signs or symptoms in a mammalian subject in need thereof, wherein the composition or medicament comprises a peptide of Formula A, or a mixture of peptides of Formula A: 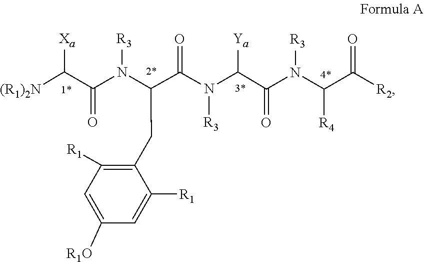
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 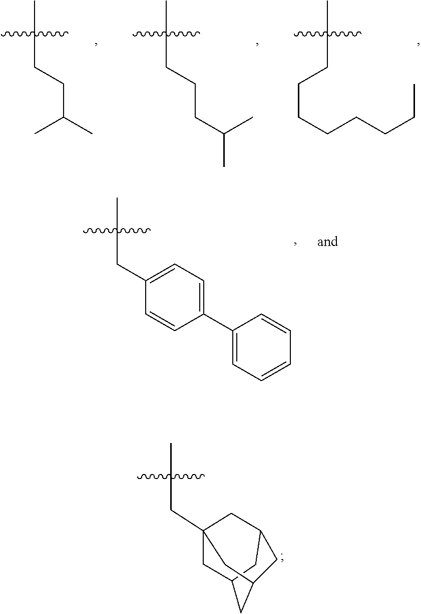
Xₐ is selected from 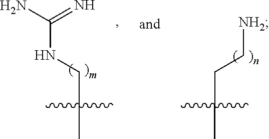
Yₐ is selected from 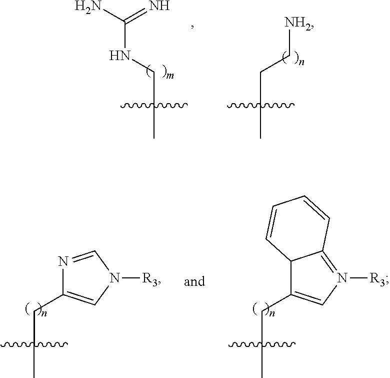
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

70. The composition or medicament of claim 69, wherein the peptide or peptides of Formula A is/are a peptide/peptides of Formula A-1 or Formula A-2: 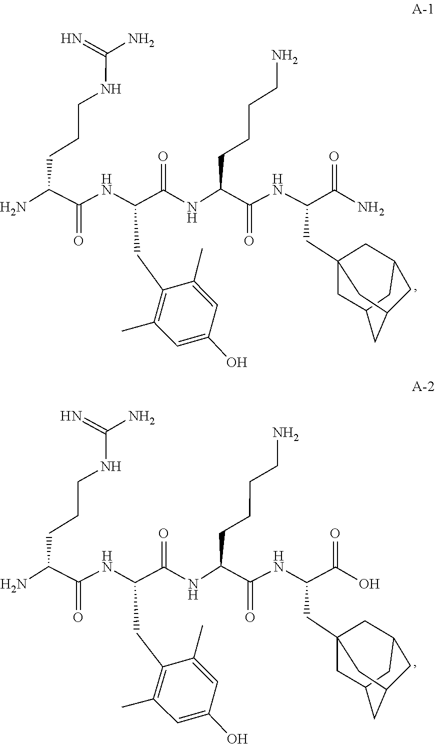 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

71. The composition or medicament of claim 69, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 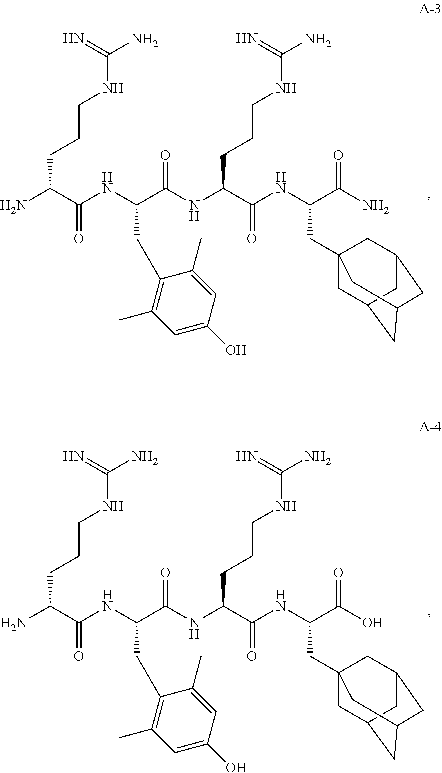 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O] 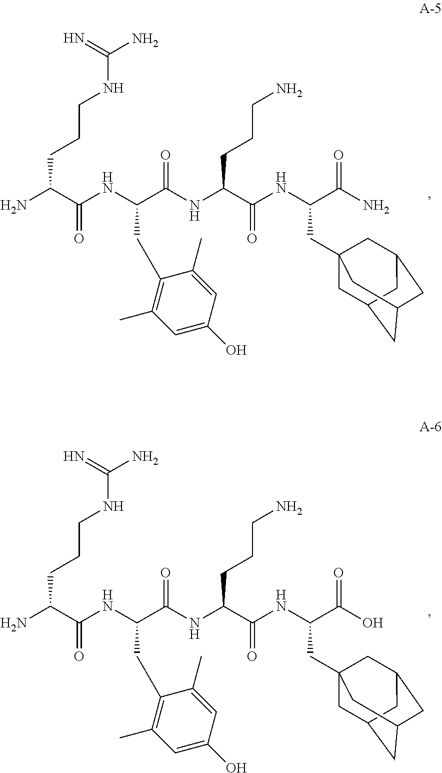 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 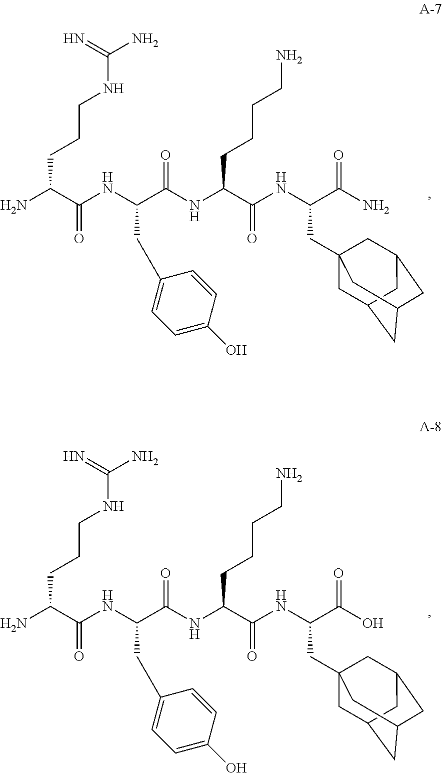 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O] 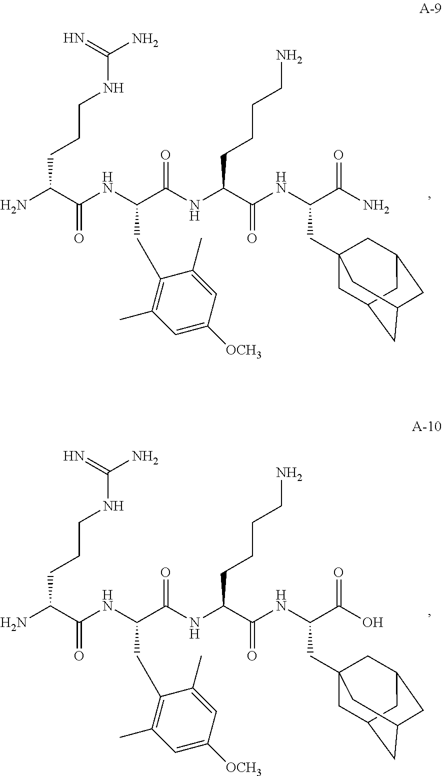 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 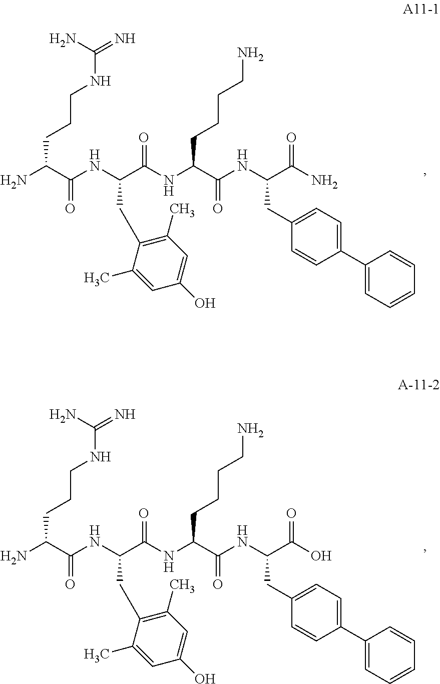 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O] 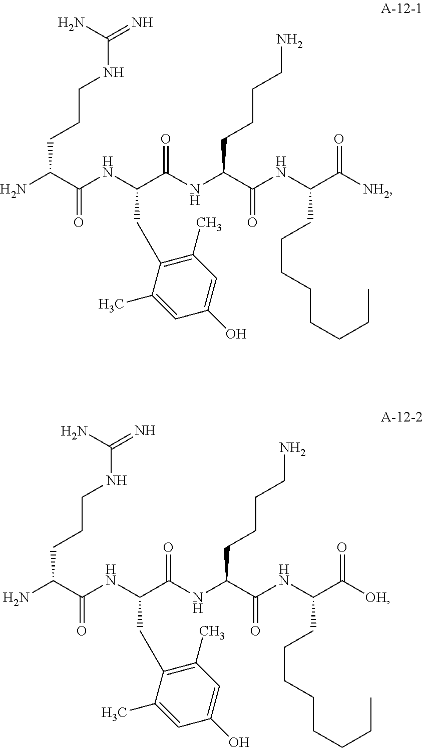 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 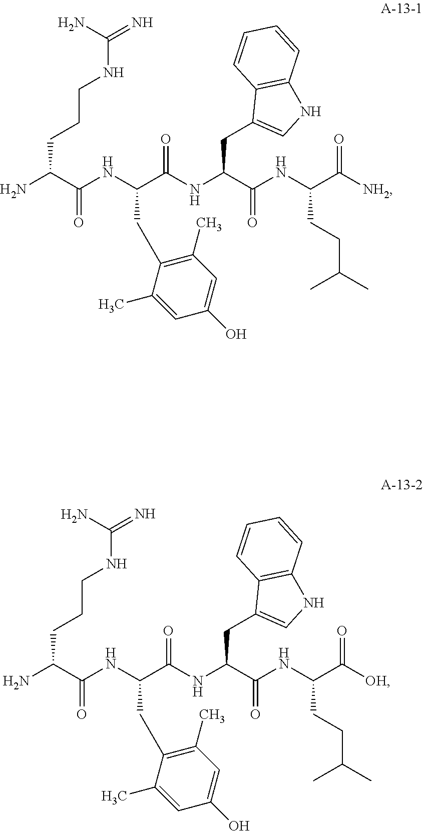 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O] 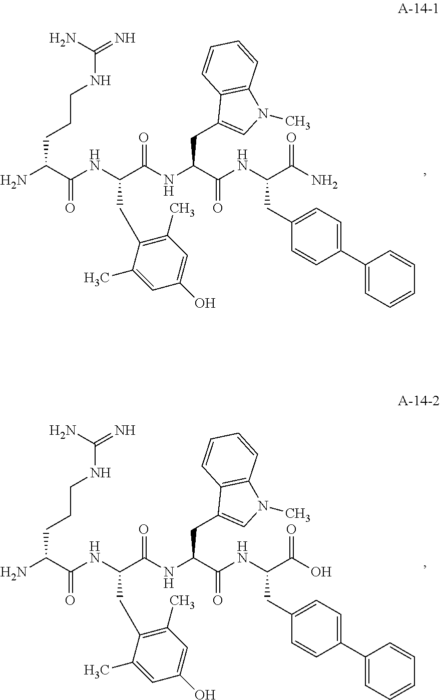 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O] 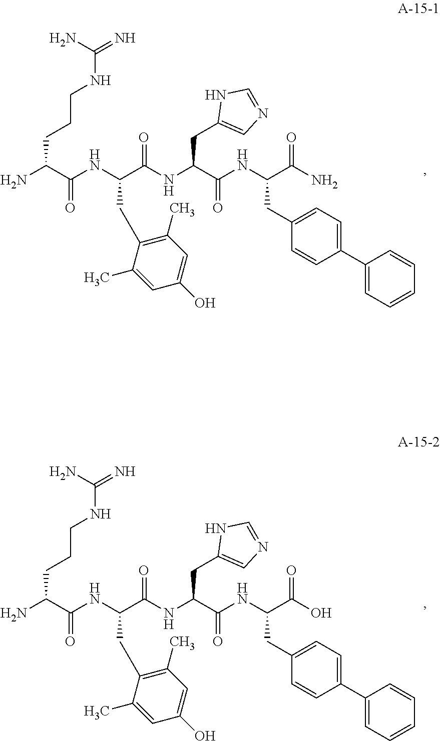 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

72. The composition or medicament of any one of claims 69 to 71, wherein administration of the composition or medicament to the subject ameliorates, inhibits, delays the onset of, and/or delays the progression of one or more signs or symptoms of muscular dystrophy in the subject as compared with an untreated control subject or untreated control group that is not administered the composition or medicament.

73. The composition or medicament of any one of claims 69 to 71, wherein administration of the composition or medicament to the subject ameliorates, inhibits, delays the onset of, and/or delays the progression of a cardiomyopathy associated with muscular dystrophy in the subject as compared with an untreated control subject or untreated control group that is not administered the composition or medicament.

74. The composition or medicament of claim 73, wherein administration of the composition or medicament to the subject ameliorates, inhibits, delays the onset of, and/or delays the progression of calcium overload, systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, heart (cardiac) failure, or any two or more of the foregoing in the subject as compared with an untreated control subject or untreated control group that is not administered the composition or medicament.

75. The composition or medicament of any one of claims 69 to 71, wherein administration of the composition or medicament to the subject ameliorates, inhibits, delays the onset of, and/or delays the progression of contraction-induced damage in muscle tissue, locomotor-skeletal muscle weakness, respiratory muscle weakness, cardiac muscle weakness, sarcolemmal weakening, or sarcolemmal tearing in the subject as compared with an untreated control subject or untreated control group that is not administered the composition or medicament.

76. The composition or medicament of any one of claims 69 to 75, wherein the composition or medicament is administered daily for: (i) 12 weeks or more; (ii) 24 weeks or more; (iii) 48 weeks or more; (iv) 72 weeks or more; or (v) 96 weeks or more.

77. The composition or medicament of any one of claims 69 to 76, wherein the composition or medicament is administered subcutaneously or intravenously.

78. The composition or medicament of any one of claims 69 to 76, wherein the composition or medicament is administered orally, topically, systemically, intraperitoneally, intradermally, transdermally, ophthalmically, intrathecally, intracerebroventricularly, iontophoretically, transmucosally, intravitreally, intranasally, or intramuscularly.

79. The composition or medicament of any one of claims 69 to 78, wherein the subject is human.

80. The composition or medicament of any one of claims 69 to 79, wherein the muscular dystrophy is Duchenne muscular dystrophy.

81. The composition or medicament of any one of claims 69 to 79, wherein the muscular dystrophy is Becker's muscular dystrophy.

82. The composition or medicament of any one of claims 69 to 81, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

83. The composition or medicament of any one of claims 69 to 81, wherein the peptide or peptides of the composition or medicament is/are formulated for administration from its/their tris —HCl salt.

84. A composition or medicament for use in treating, preventing, inhibiting, ameliorating or delaying the onset of a cardiomyopathy in a mammalian subject in need thereof, wherein the subject is suffering from muscular dystrophy, wherein the composition or medicament comprises a peptide of Formula A, or a mixture of peptides of Formula A: 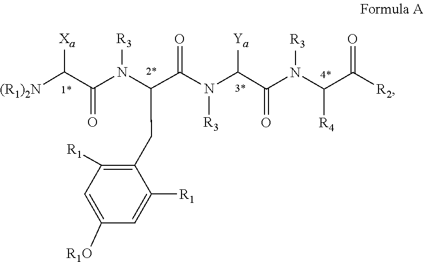
or a pharmaceutically acceptable salt, hydrate, solvate, and/or tautomer thereof, wherein, each R₁ is independently H, —CH₃, or —CH₂CH₃; R₂ is —OH, —NH₂, —NHR₁ or —N(R₁)₂; each R₃ is independently H, or —CH₃; R₄ is selected from: 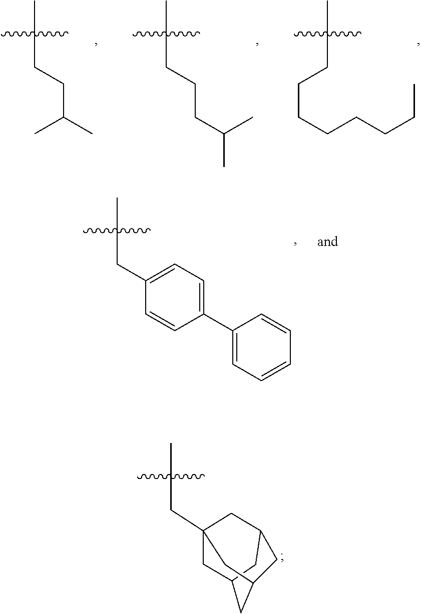
Xₐ is selected from 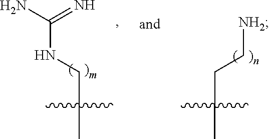
Yₐ is selected from 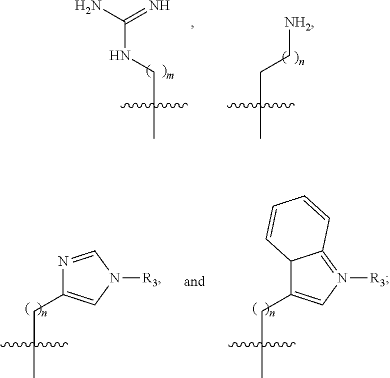
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

85. The composition or medicament of claim 84, wherein the peptide or peptides of Formula A is/are a peptide/peptides of Formula A-1 or a peptide of Formula A-2: 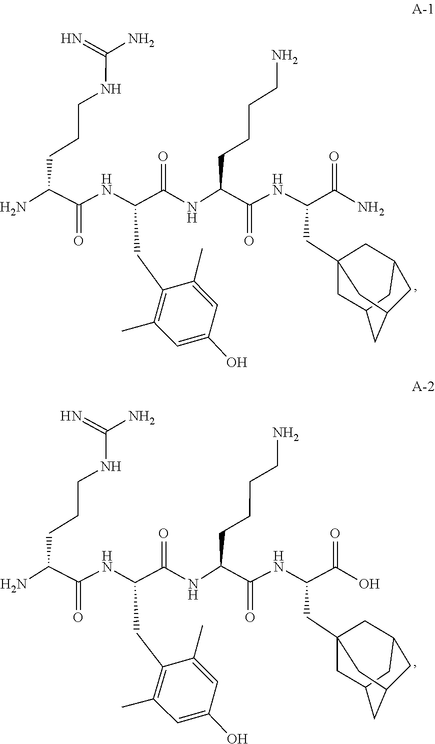 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

86. The composition or medicament of claim 84, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 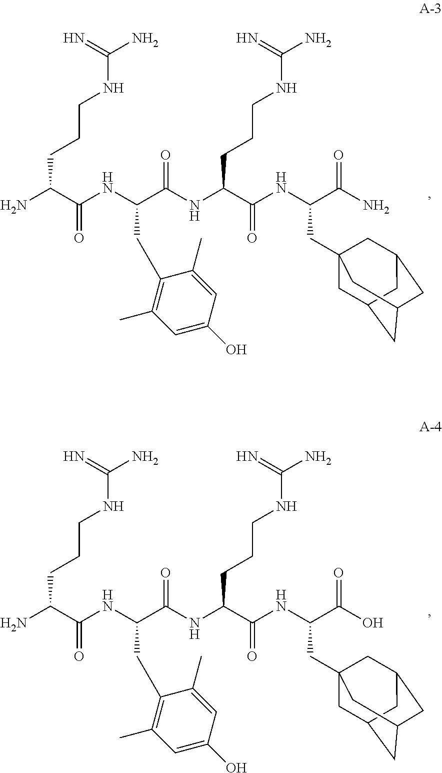 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O] 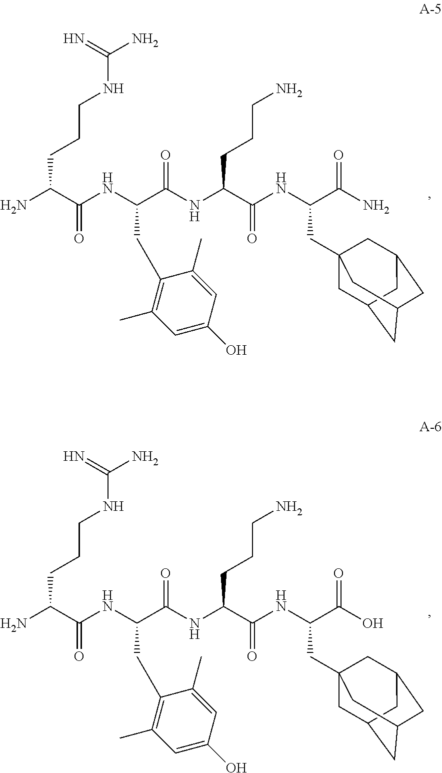 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 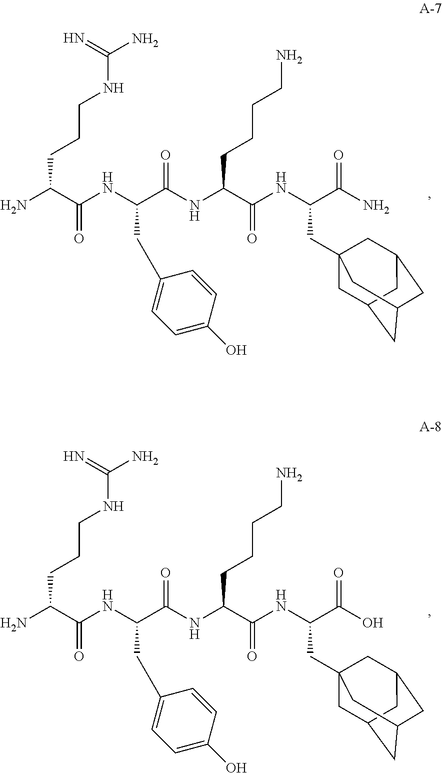 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O] 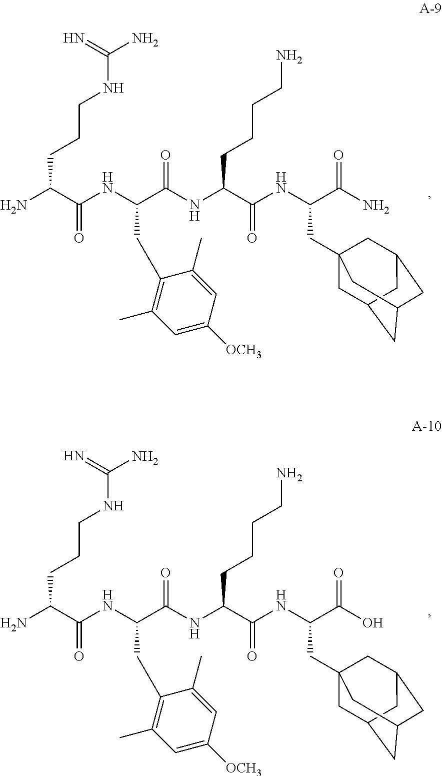 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 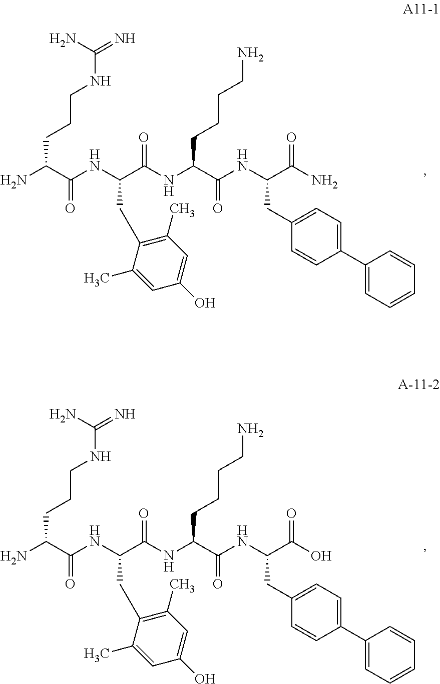 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O] 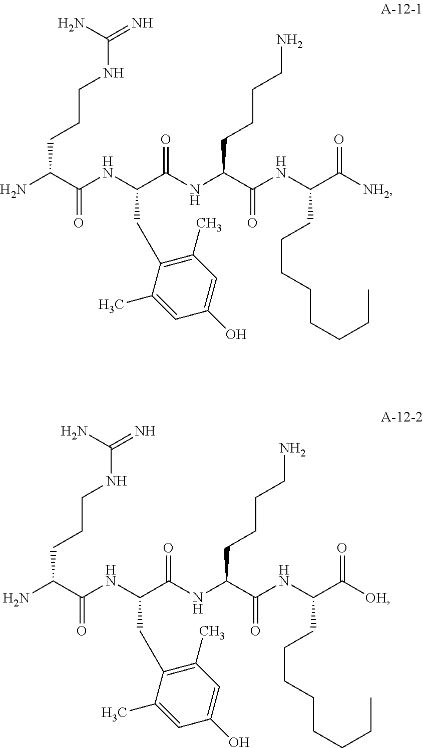 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 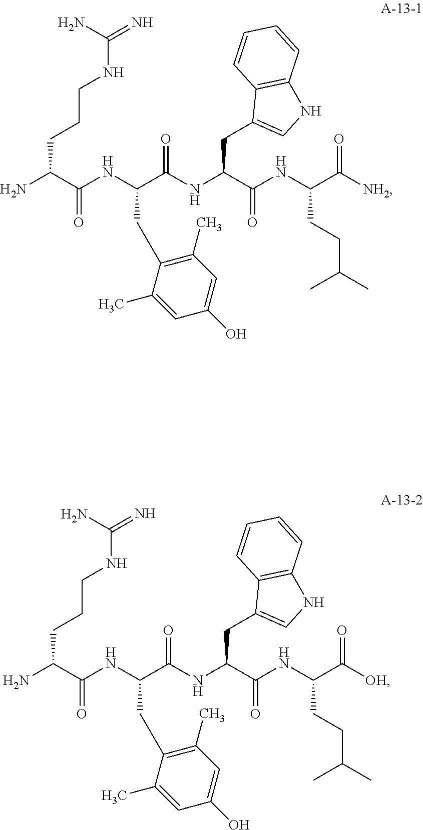 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O] 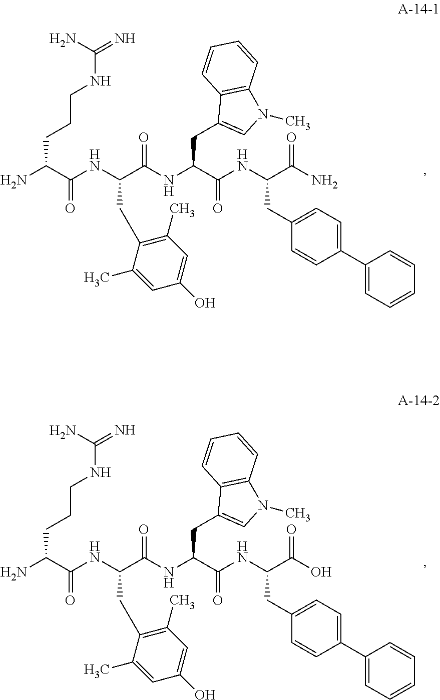 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O] 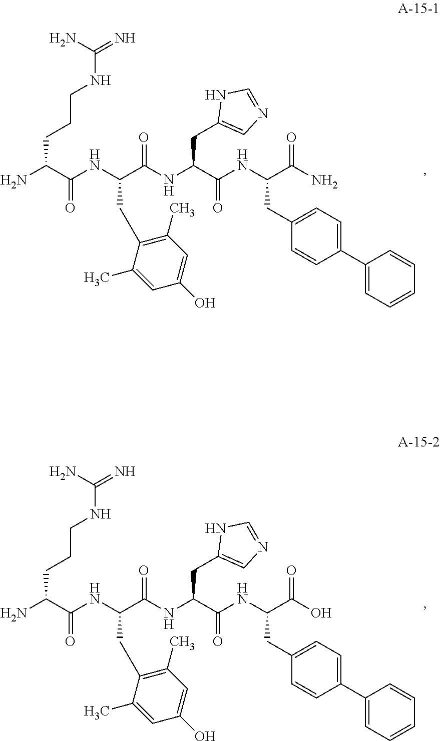 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

87. The composition or medicament of any one of claims 84 to 86, wherein administration of the composition or medicament to the subject ameliorates, inhibits, delays the onset of, and/or delays the progression of calcium overload, systolic disfunction, diastolic dysfunction, ventricular remodeling, cardiac (myocardial) fibrosis, fatty acid infiltration, hypertrophic cardiomyopathy, dilated cardiomyopathy, an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia), reduced left ventricular ejection fraction, fractional shortening, heart (cardiac) failure, or any two or more of the foregoing, in the subject as compared with an untreated control subject or control group that is not administered the composition or medicament.

88. The composition or medicament of claim 87, wherein administration of the composition or medicament to the subject ameliorates, inhibits, delays the onset of, and/or delays the progression of calcium overload, systolic disfunction, or diastolic dysfunction of the subject as compared with an untreated control subject or control group that is not administered the composition or medicament.

89. The composition or medicament of claim 87, wherein administration of the composition or medicament to the subject ameliorates, inhibits, delays the onset of, and/or delays the progression of ventricular remodeling, including left ventricular remodeling, of the subject as compared with an untreated control subject or control group that is not administered the composition or medicament.

90. The composition or medicament of claim 87, wherein administration of the composition or medicament to the subject ameliorates, inhibits, delays the onset of and/or delays the progression of cardiac (myocardial) fibrosis of the subject as compared with an untreated control subject or control group that is not administered the composition or medicament.

91. The composition or medicament of claim 87, wherein administration of the composition or medicament to the subject ameliorates, inhibits, delays the onset of, and/or delays the progression of fatty acid infiltration of the subject as compared with an untreated control subject or control group that is not administered the composition or medicament.

92. The composition or medicament of claim 87, wherein administration of the composition or medicament to the subject ameliorates, inhibits, delays the onset of, and/or delays the progression of hypertrophic cardiomyopathy of the subject as compared with an untreated control subject or control group that is not administered the composition or medicament.

93. The composition or medicament of claim 87, wherein administration of the composition or medicament to the subject ameliorates, inhibits, delays the onset of, and/or delays the progression of dilated cardiomyopathy of the subject as compared with an untreated control subject or control group that is not administered the composition or medicament.

94. The composition or medicament of claim 87, wherein administration of the composition or medicament to the subject ameliorates, inhibits, delays the onset of, and/or delays the progression of an arrhythmia (e.g., atrial fibrillation, atrial premature contractions, atrial couplets, atrioventricular (AV) block, ventricular tachycardia, ventricular fibrillation, ventricular premature contractions, ventricular couplets, or supraventricular tachycardia) of the subject as compared with an untreated control subject or control group that is not administered the composition or medicament.

95. The composition or medicament of claim 87, wherein administration of the composition or medicament to the subject ameliorates, inhibits, delays the onset of, and/or delays the progression of reduced left ventricular ejection fraction of the subject as compared with an untreated control subject or control group that is not administered the composition or medicament.

96. The composition or medicament of claim 87, wherein administration of the composition or medicament to the subject ameliorates, inhibits, delays the onset of, and/or delays the progression of fractional shortening of the subject as compared with an untreated control subject or control group that is not administered the composition or medicament.

97. The composition or medicament of claim 87, wherein administration of the composition or medicament to the subject ameliorates, inhibits, delays the onset of, and/or delays the progression of heart (cardiac) failure of the subject as compared with an untreated control subject or control group that is not administered the composition or medicament.

98. The composition or medicament of any one of claims 84 to 86, wherein administration of the composition or medicament results in normalization of the ejection fraction, fractional shortening, stroke volume, and/or cardiac output of the subject as compared with an untreated control subject or control group that is not administered the composition or medicament.

99. The composition or medicament of any one of claims 84 to 98, wherein the composition or medicament is administered daily for: (i) 12 weeks or more; (ii) 24 weeks or more; (iii) 48 weeks or more; (iv) 72 weeks or more; or (v) 96 weeks or more.

100. The composition or medicament of any one of claims 84 to 99, wherein the composition or medicament is administered subcutaneously or intravenously.

101. The composition or medicament of any one of claims 84 to 99, wherein the composition or medicament is administered orally, topically, systemically, intraperitoneally, intradermally, transdermally, ophthalmically, intrathecally, intracerebroventricularly, iontophoretically, transmucosally, intravitreally, intranasally, or intramuscularly.

102. The composition or medicament of any one of claims 84 to 101, wherein the subject is human.

103. The composition or medicament of any one of claims 84 to 102, wherein the muscular dystrophy is Duchenne muscular dystrophy.

104. The composition or medicament of any one of claims 84 to 102, wherein the muscular dystrophy is Becker's muscular dystrophy.

105. The composition or medicament of any one of claims 84 to 104, further comprising separately, sequentially, or simultaneously administering an additional therapeutic agent to the subject.

106. The composition or medicament of claim 105, wherein the additional therapeutic agent is a phosphorodiamidate morpholino oligomer (PMO) or a peptide-conjugated phosphorodiamidate morpholino oligomer (PPMO).

107. The composition or medicament of claim 106, wherein the PMO is selected from the group consisting of eteplirsen (Exondys 51®), golodirsen (Vyondys 53®), viltolarsen (Viltepso®), and Casimersen (Amondys 45®) or the PPMO is selected from vesleteplirsen (a.k.a., SRP-5051) and PGN-EDO51 (a.k.a., CONNECT1-ED051).

108. The composition or medicament of claim 105, wherein the additional therapeutic agent is an adeno-associated virus (AAV) vector-based gene therapy.

109. The composition or medicament of claim 108, wherein the adeno-associated virus vector-based gene therapy is delandistrogene moxeparvovec-rokl (Elevidys®).

110. The composition or medicament of claim 105, wherein the additional therapeutic agent is a corticosteroid, an angiotensin-converting enzyme (ACE) inhibitor, an angiotensin receptor blocker (ARB), a beta blocker, a diuretic, or a mineralocorticoid receptor antagonist.

111. The composition or medicament of claim 110, wherein the corticosteroid is vamorolone (aGamree®), prednisone, or deflazacort.

112. The composition or medicament of claim 110, wherein the angiotensin-converting enzyme (ACE) inhibitor is captopril, enalapril, lisinopril, benazepril, or ramipril.

113. The composition or medicament of claim 110, wherein the angiotensin receptor blocker (ARB) is azilsartan, candesartan, eprosartan, irbesartan, telmisartan, valsartan, losartan, olmesartan, Entresto® (sacubitril/valsartan) or Byvalson™ (nebivolol/valsartan).

114. The composition or medicament of claim 110, wherein the beta blocker is carvedilol, bisoprolol, metoprolol succinate, atenolol, esmolol, nebivolol, or propranolol.

115. The composition or medicament of claim 110, wherein the diuretic is bumetanide, furosemide, or torsemide.

116. The composition or medicament of claim 110, wherein the mineralocorticoid receptor antagonist is spironolactone or eplerenone.

117. The composition or medicament of claim 105, wherein the additional therapeutic agent is mavacamten (Camzyos®).

118. The composition or medicament of any one of claims 84 to 7 wherein the pharmaceutically acceptable salt of the peptide or peptides of the composition or medicament comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

119. The composition or medicament of any one of claims 84 to 117, wherein the peptide or peptides of the composition or medicament is/are formulated for administration from its/their tris —HCl salt.

120. Use of a peptide, mixture of peptides, or a composition or medicament comprising the peptide, or the mixture of peptides, for treating, preventing, inhibiting, ameliorating or delaying the onset of muscular dystrophy in a mammalian subject in need thereof, wherein the peptide or peptides of the mixture is/are a peptide of Formula A: 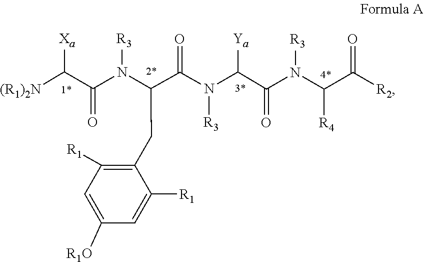
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 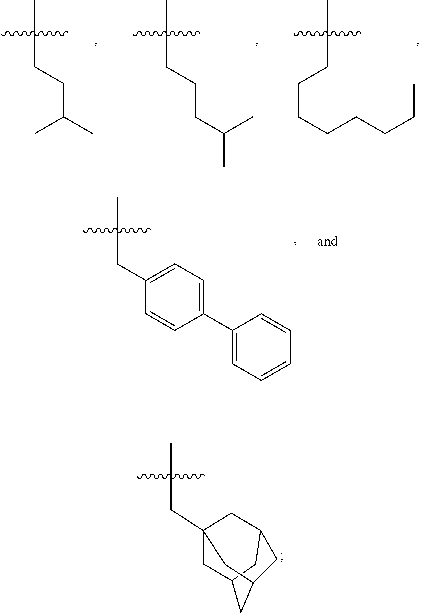
Xₐ is selected from 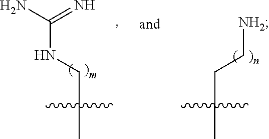
Yₐ is selected from 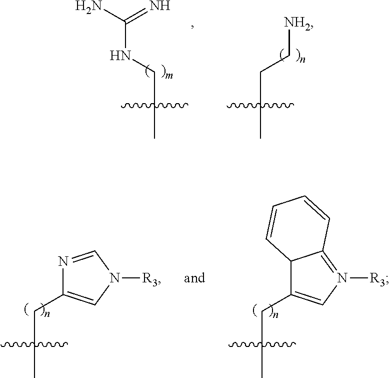
each m is independently 1, 2, 3 or 4; each n is independently 1, 2, or 3; and the absolute stereochemistry at each of stereocenters 1*, 2*, 3*, and 4* is independently R or S, and optionally one or more hydrogen atoms of the peptide or peptides of Formula A is/are substituted with a deuterium or fluorine atom.

121. Use of a peptide, mixture of peptides, or a composition or medicament comprising the peptide, or the mixture of peptides, for treating, preventing, inhibiting, ameliorating or delaying the onset of a cardiomyopathy in a mammalian subject in need thereof, wherein the subject is suffering from muscular dystrophy, wherein the peptide or peptides of the mixture is/are a peptide of Formula A: 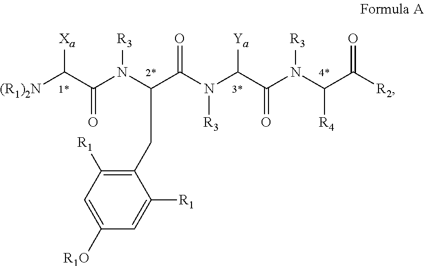
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 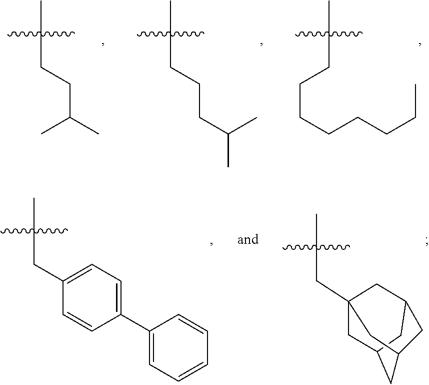
Xₐ is selected from 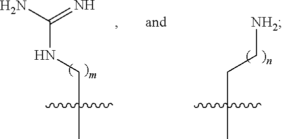
Yₐ is selected from 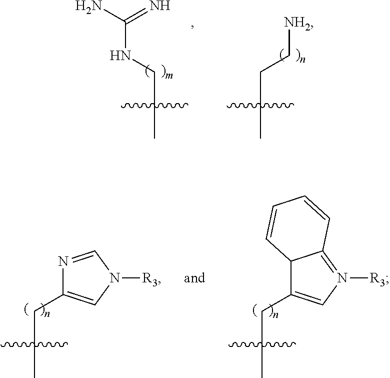
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide or peptides of Formula A is/are substituted with a deuterium or fluorine atom.

122. A method for treating, preventing, inhibiting, ameliorating or delaying the onset of loss of ambulation in a mammalian subject in need thereof, wherein the subject is having, or suspected of having, muscular dystrophy, comprising administering to the subject a therapeutically effective amount of a peptide of Formula A, or a mixture of peptides of Formula A: 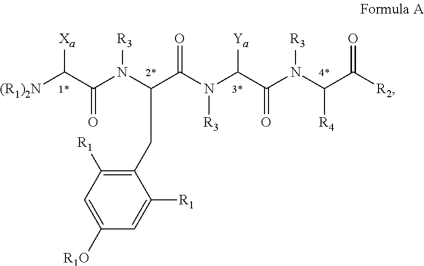
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 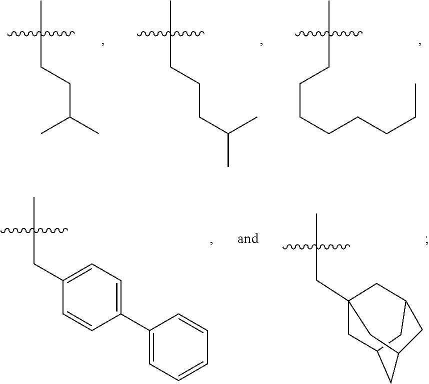
Xₐ is selected from 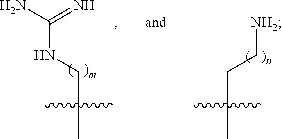
Yₐ is selected from 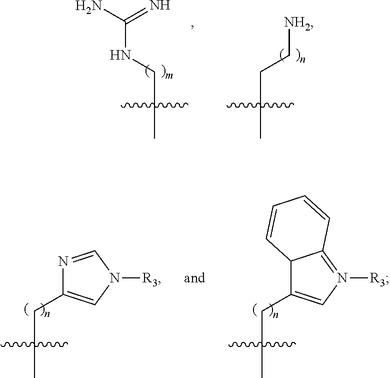
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

123. The method of claim 122, wherein the peptide or peptides of Formula A is/are a peptide/peptides of Formula A-1 or Formula A-2: 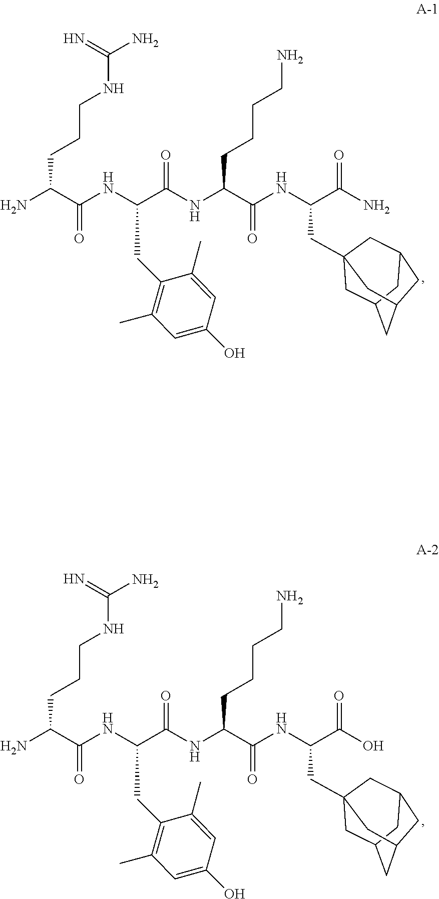 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

124. The method of claim 122, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 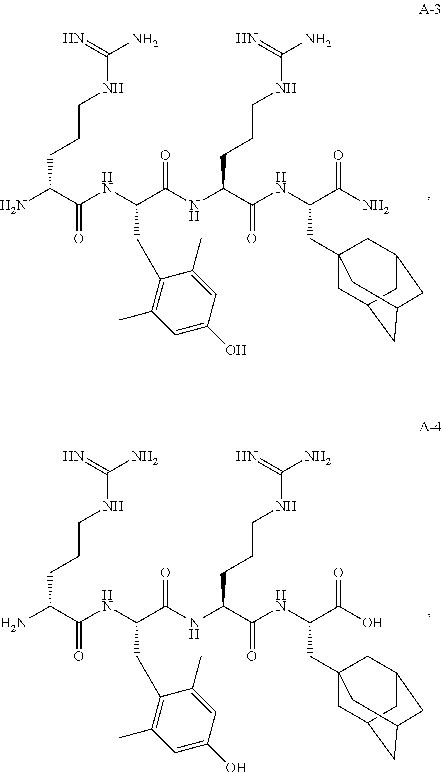 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O] 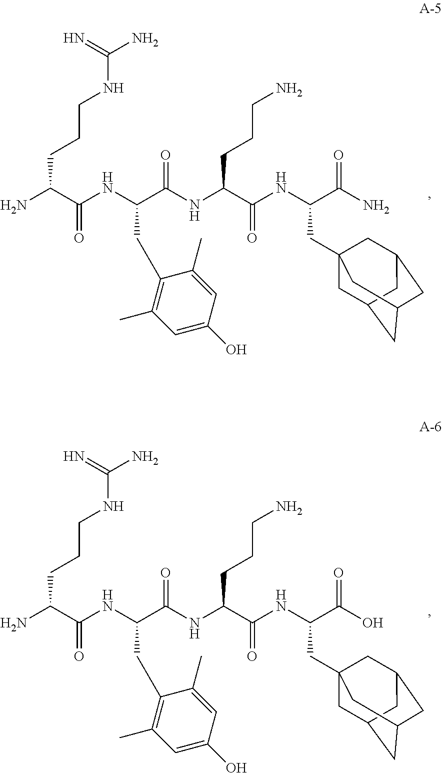 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 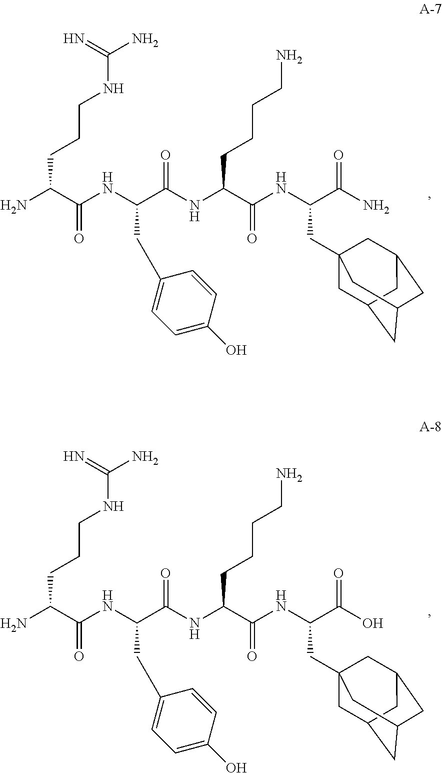 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O] 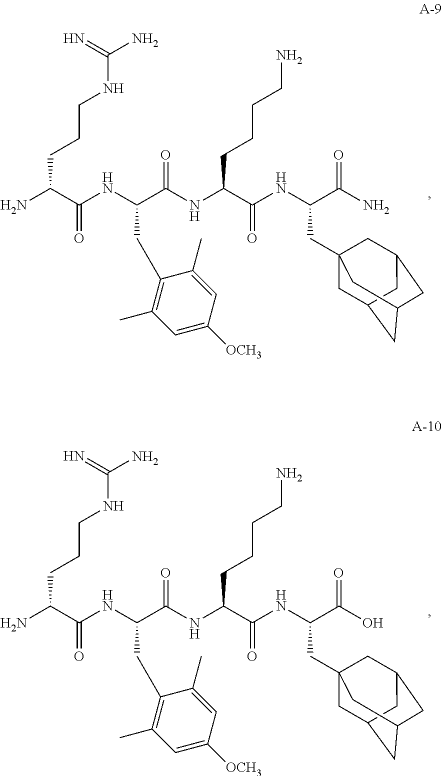 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 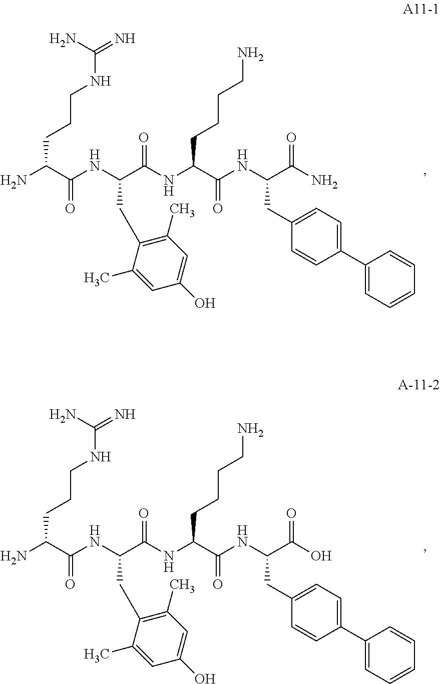 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O] 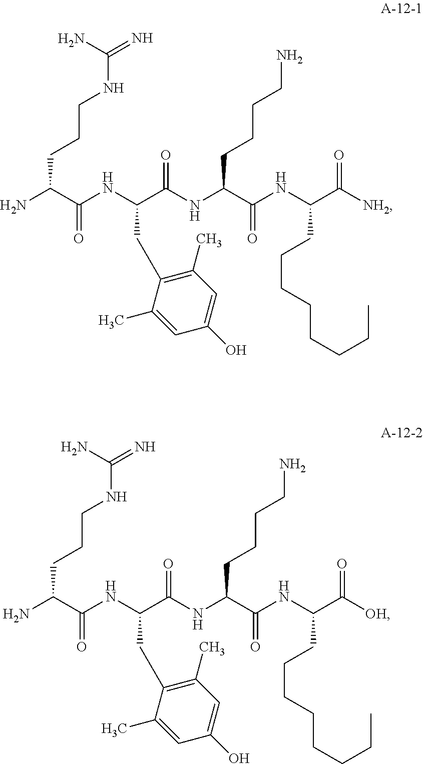 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 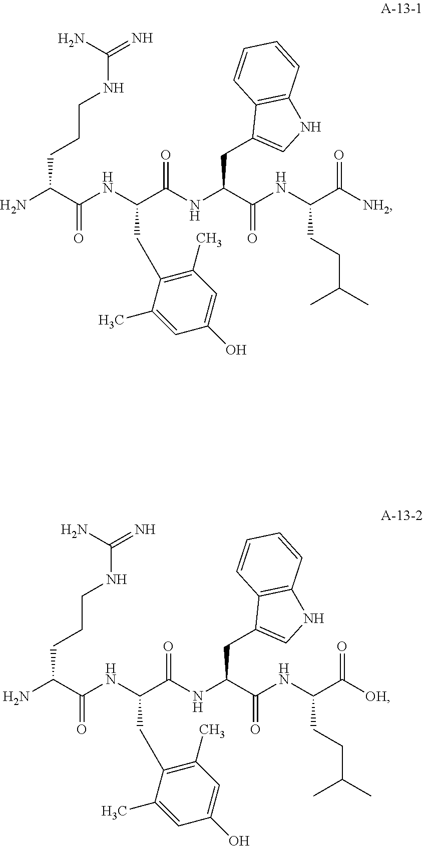 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O] 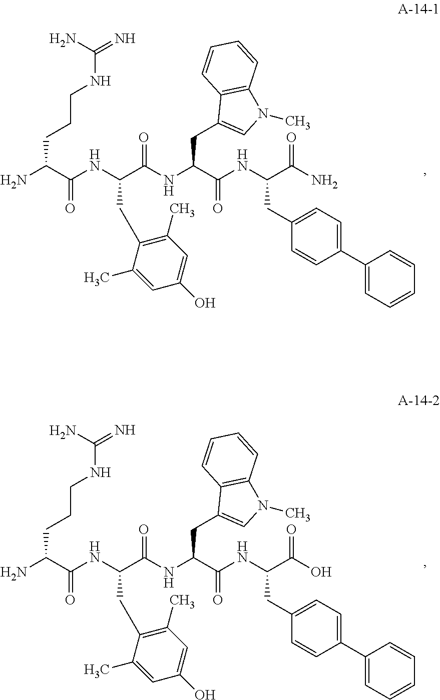 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O] 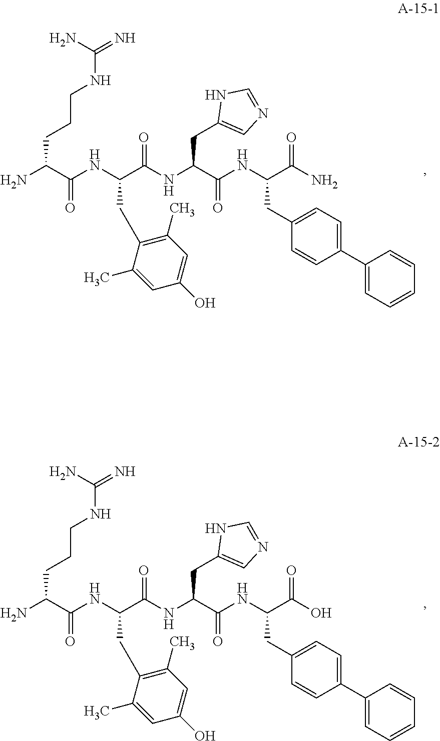 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

125. A method for treating, preventing, inhibiting, ameliorating or delaying the onset of respiratory distress in a mammalian subject in need thereof, wherein the subject is having, or suspected of having, muscular dystrophy, comprising administering to the subject a therapeutically effective amount of a peptide of Formula A, or mixture of peptides of Formula A: 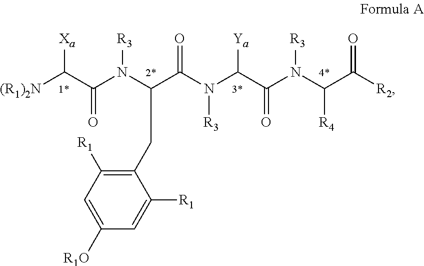
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 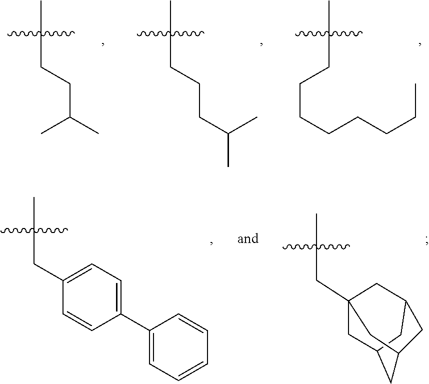
Xₐ is selected from 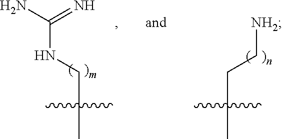
Yₐ is selected from 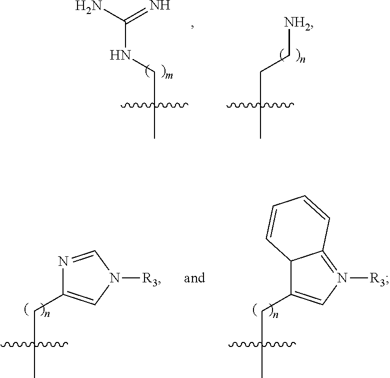
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

126. The method of claim 125, wherein the peptide or peptides of Formula A is a peptide of Formula A-1 or A-2: 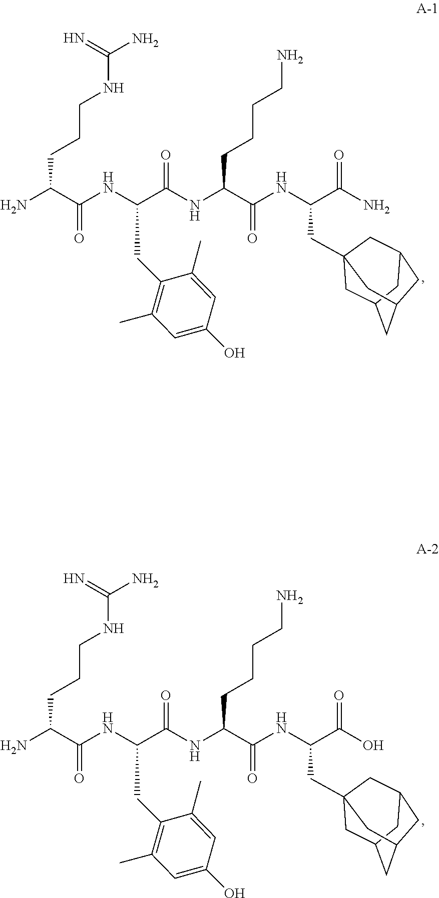 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

127. The method of claim 125, wherein the peptide or peptides of Formula A is a peptide of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 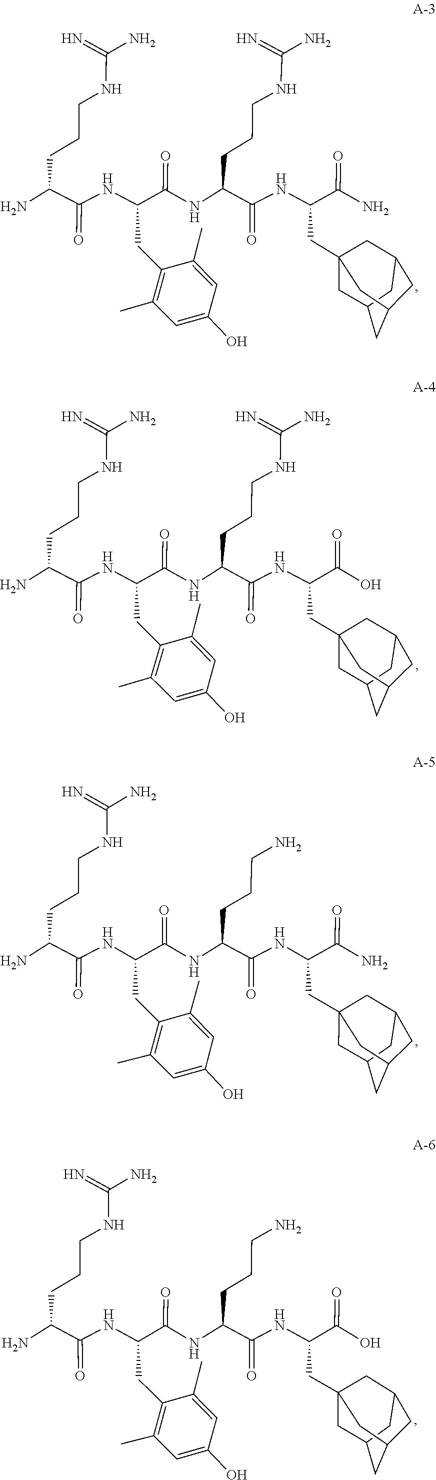 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 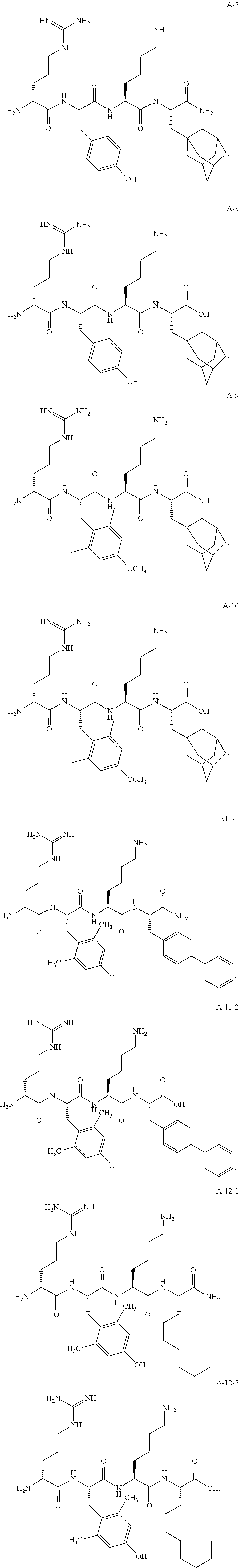 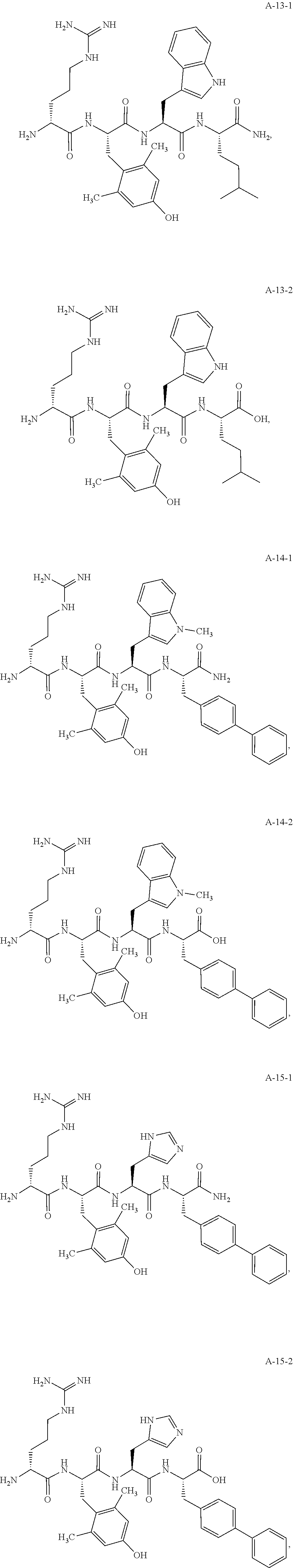
or a pharmaceutically acceptable salt, hydrate, solvate, stereoisomer and/or tautomer thereof.
